(19) United States
(12) Patent Application Publication
(10) Pub. No.: US 2010/0086647 A1
(43) Pub. Date: Apr. 8, 2010

(54) FEED OR FOOD PRODUCTS COMPRISING FUNGAL MATERIAL

(75) Inventor: Bjorn Kristiansen, Frederikstad (NO)

Correspondence Address: BROWDY AND NEIMARK, P.L.L.C.;624 NINTH STREET, NW, SUITE 300, WASHINGTON, DC, 20001-5303, US

(73) Assignee: MEDIMUSH AS, HORSHOLM (DK)

(21) Appl. No.: 11/914,318

(22) PCT Filed: May 11, 2006
(86) PCT No.: PCT/DK06/00253

§ 371 Date: Dec. 1, 2009

Related U.S. Application Data
(63) Continuation-in-part of application No. PCT/DK2005/000498, filed on Jul. 15, 2005.
(60) Provisional application No. 60/690,496, filed on Jun. 15, 2005.
(60) Provisional application No. 60/761,744, filed on Jan. 25, 2006.
(60) Provisional application No. 60/690,496, filed on Jun. 15, 2005.

(30) Foreign Application Priority Data
May 13, 2005 (DK) PA 2005 00710
Jun. 15, 2005 (DK) PA 2005 00880
Jul. 15, 2005 (DK) PCT/DK05/00498
Jan. 25, 2006 (DK) PA 2006 00117

(51) Int. Cl. A23L 1/28; A23K 1/18; A23K 1/16; C12P 19/04; A01K 61/00

(52) U.S. Cl. 426/62; 426/2; 119/230

(57) ABSTRACT

The present invention relates to feed and food compositions comprising material obtained by fermenting fungi of the Basidiomycetes family in a liquid medium. Interestingly, Basidiomycetes grown in a liquid culture produce compounds which, when used in feed or food compositions enhance survival and/or support growth of normal, healthy animals. Furthermore, the compounds may modulate the microbial population in the digestive tract after oral intake.

All patent and non-patent references cited in the present application, are hereby incorporated by reference in their entirety.

### FIELD OF INVENTION

The present invention relates to feed and food compositions comprising material obtained by fermenting fungi of the Basidiomycetes family in a liquid medium. Interestingly, Basidiomycetes grown in a liquid culture produce compounds which, when used in feed or food compositions enhance survival and/or support growth of normal, healthy animals. Furthermore, the compounds may modulate the microbial population in the digestive tract after oral intake.

### BACKGROUND OF INVENTION

It is known that health-promoting effects are attributed to glucans from fungi and yeasts. “Shiitake” fungus (Lentinus edodes) has been attributed effects which can be exploited for many medicinal purposes such as immunostimulation, anti-virus, anti-tumour, etc. Studies of lentinan have shown that it stimulates the immune system of the host in a variety of ways, such as activation of T helper cells, increased production of Interleukin 1 and Interleukin 2, increased antibody production in various forms of cancer, and decreasing the cholesterol level in the blood. (Herbs for Health, January/February, 1997; K. Jones, “Shiitake: Medicine in a mushroom”, p. 40-50, 54; Anticancer Res, Vol. 17(4A), 1997; H. Matsouka, “Lentinan potentiates immunity and prolongs the survival time of some patients”, p. 2751-2755; Adv Appl Microbiol, Vol. 39, 1993; S. C. Jong, “Medicinal and therapeutic value of the shiitake mushroom”, p. 153-184, Int J Immunopharmacol, Vol. 14, 1992; K. Irinoda, “Stimulation of microbiocidal host defense mechanism against aerosol influenza virus infection by lentinan, p. 971-977., Jpn J Cancer Res, Vol. 76(1), 1985; D. Herlyn, “Monoclonal antibody-dependent murine macrophage-mediated cytotoxicity against human tumors is stimulated by lentinan, p. 37-42).

One active ingredient of Lentinus edodes is termed lentinan, a polysaccharide based compound described as a beta-(1,3) glucan backbone with beta-(1,6) side chains.

“Solid-state” reactors are routinely used for culturing fungi such as Lentinus edodes.

This is a technology which is used for many purposes such as composting, production of biological products such as enzymes, soy sauce, acetic acid, and the like. For the production of lentinan, Lentinus edodes can be cultivated on a suitable solid matrix provided by stems of tree or chips of wood to which is often added chemical compounds supporting the growth of mycelium and development of the fruiting bodies, where most of the lentinan is localised. The fruiting bodies are harvested, either by hand or mechanically, and are subsequently dried and ground to a powder which can be used as it is, or used in tablets, or sent for further processing such as extraction of lentinan. It has been suggested that extracts of mushrooms may be a growth promoter when fed to chickens (Guo et al., British Poultry Science, 2004, 45:684-694). However, Lobanok et al., 2003, Applied Biochemistry and Microbiology, 39:60-64, have described that mycelium from submerged fermentation of Lentinus edodes provides no growth advantage to laboratory animals.

The patent application WO03/020944 describes a number of methods of purifying extracellular immunostimulating compounds from fungi grown in liquid culture.

Hatvani et al., describes a method of purifying a compound, which may be Lenthionine from the culture medium of Lentinus edodes grown in liquid culture. The document describes that the purified compound in vitro has antibacterial and anticandida effect against B. megaterium, S. staphylococcus, S. pyogenes and C. albicans. It is postulated that it has no effect on a number of other microorganisms including E. coli and C. jejuni.

Low survival rates of fish bred in fish farms is a major problem. In particular, it has proven to be excessively difficult to breed white fish, such as cod, in fish farms. However, other fish, such as salmon and trout, which are routinely bred in fish farms also have a high lethal rate. The same applies to shrimps and prawns bred in farms.

For producers of farm animals, it is frequently desirable to obtain the largest possible weight gain in animals. Also lethality may be a problem.

Health or well-being may be influenced by the microbial population in the digestive tract.

Accordingly, there is a need for provision of feed and food products which may improve health, modulate the microbial population in the digestive tract, improve survival rates of animals and/or improve growth.

### SUMMARY OF INVENTION

One object of the present invention is to provide feed and food products, which may improve survival.

Another object of the invention is to provide feed and food products, which may improve growth.

Yet another object of the invention is to provide feed and food products, which may modulate the microbial population in the digestive tract.

In another aspect of the invention, a food product is provided which is a functional food, preferably either for improving or maintaining health. Said functional food is preferably suitable for consumption by human beings.

The above-mentioned feed and food products comprise a bioactive agent such as a survival enhancing agent.

### FIGURE LEGENDS

FIG. 1: positive effect of different amounts of the bioactive agent in feed on the weight increase of NOROC piglets. Feeds with 0.02, 0.2 and 2.0 mg bioactive agent/kg feed (see Table 4) were given to weaned piglets and feed without the bioactive agent was used as control. The weight increase of each animal was measured weekly and the average weight increase was calculated (see Table 5). The experiment is described in detail in Example 5b.

FIG. 2: positive effect of different amounts of the bioactive agent in feed on the feed factor of NOROC piglets. Feeds with 0.02, 0.2 and 2.0 mg bioactive agent/kg feed (see Table 4) were given to weaned piglets and feed without the bioactive agent was used as control. The feed factor was determined for weeks 1, 1-2. 1-3 and 1-4 (see Table 6). The experiment is described in detail in Example 5b.

FIG. 3: bacteriostatic effect of different dilutions (1:10, 1:20 and 1:40) of the bioactive agent obtained by the method as described in example 1 on E. coli K12. A culture without the bioactive agent was used as control (Ref). The experiment is described in detail in Example 6.

FIG. 4: cancer-cell specific cytotoxicity of different concentrations of Lentinex—comprising an embodiment of the bioactive agent of the present invention—on 4 different human and mouse cancer cell lines. The MRC-5 cell line from normal human fetal lung fibroblasts was used as control. The experiment is described in detail in Example 7.

### DEFINITIONS

Mycelium: Mass of hyphae constituting the body (thallus) of the fungus.
Agaricus sp.: A basidiomycetous fungal species of the genus agaricus of the family agaricaceae and the order agaricales and the subclass agaricomycetidae.
Schizophyllum sp.: A basidiomycetous fungal species of the genus schizophyllum of the family schizophyllaceae and the order agaricales and the subclass agaricomycetidae.
Lentinus sp.: A basidiomycetous fungal species of the genus lentinus of the family polyporaceae and the order polyporales and the subclass agaricomycetidae. L. edodes is also termed Lentinula edodes, which is placed in the family Marasmiaceae, in the order Agaricales and the subclass agaricomycetidae.
Trametes sp.: A basidiomycetous fungal species of the genus trametes of the family polyporaceae and the order polyporales and the subclass agaricomycetidae.
Ganoderma sp.: A basidiomycetous fungal species of the genus ganoderma of the family ganodermataceae and the order polyporales and the subclass agaricomycetidae.
Grifola sp.: A basidiomycetous fungal species of the genus grifola of the family meripilaceae and the order polyporales and the subclass agaricomycetidae.
Fruiting bodies or fruit bodies: Any one of a variety of complex, spore-bearing fungal structures.
Basidiomycete cell: A cell from a fungus of the class Basidiomycete of the Phylum Basidiomycota, wherein the cell can be derived from any part of the fungus, such as fruiting body, hyphae, spores and mycelium. The Basidiomycete cell can be a single hyphae, spores, aggregates of mycelium, or partly differentiated mycelium, or comprised in fungal mycelium.
Bioactive agent: Any agent, drug, compound, composition of matter or mixture which provides a beneficial pharmacological effect that can be demonstrated in-vivo or in vitro. This includes beneficial pharmacological effects which can be demonstrated in an individual on a diet comprising an edible food, a food supplement, such as a composition of vitamins, a nutrient, or a nutraceutical comprising the bioactive agent. Also, the beneficial pharmacological effect can be observed in an individual being administered a medicament (drug), a combination of medicaments, a vaccine, or other beneficial agents comprising the bioactive agent. The bioactive agent can be provided in isolated and/or purified form, or in a solid or liquid composition, such as e.g. a solid composition comprising Basidiomycete biomass resulting from a submerged cultivation (i.e. when the bioactive agent is produced intracellularly), or a liquid composition, such as e.g. extracellular growth medium comprising said bioactive agent (i.e. when the bioactive agent is secreted to the extracellular medium). The extracellular growth medium can be separated from the biomass, or from a part of said biomass, by e.g. filtration or centrifugation. There is also provided an Basidiomycete whole cell fermentation culture comprising both biomass and extracellular growth medium, said whole cell culture comprising said bioactive agent.
Bioactive agents comprising a survival enhancing activity: Survival enhancement is an important parameter when e.g. treating an individual for a disease, or when rearing domestic animals or raising fish in industrial fish farms. Bioactive agents comprising a survival enhancing effect are able to improve the survival. Thus such a bioactive agent is also called a survival enhancing agent.
Functional food: A functional food is a food that goes beyond simple nutrition and has at least one specific targeted action to improve the health and/or well being of the host and/or prevent pathological states in the host.
Probiotics: specific live microorganisms that have a beneficial effect on the host.
Prebiotics: ingredients or compounds that have a beneficial effect on the microflora in the host itself.
Synbiotics: mixtures of probiotics and prebiotics.
Probiotic shots: Probiotic shots contain concentrated doses of ‘good’ bacteria that help to boost the immune system and aid in digestion. They are typically sold in multipacks of single-serve bottles of just over 3-ounces, each one intended to be consumed in a single sitting.
Polysaccharides: the term “polysaccharide” as used herein covers polysaccharides as well as polysaccharides containing and/or covalently linked to peptides, polypeptides or the like, such as proteopolysaccharides.
Polysaccharides comprising monosaccharides: A polysaccharide is said to comprise monosaccharides, wherein said monosaccharides are covalently linked to form said polysaccharide. Hydrolysing a polysaccharide will yield the monosaccharides that formed said polysaccharide in free form. The monosaccharide content of a polysaccharide can thus be determined by hydrolysing the polysaccharide and measuring the presence of individual monosaccharides. The monosaccharide content of a mixture of polysaccharides is determined by determining the monosaccharide content of the entire mixture.
Ratio: A polysaccharide or a mixture of polysaccharides are said to comprise galactose, mannose, and glucose in a given ratio, when hydrolysation of said polysaccharide or said mixture of polysaccharide yields galactose, mannose and glucose in said given ratio. Galactose, mannose, and glucose in the ratio 1:a to b:c to d, means that for every part galactose, mannose is present in the range of a to b parts and glucose is present in the range of c to d parts, wherein a, b, c and d indicates numerical values. Thus, by way of example a polysaccharide mixture comprising galactose, mannose, and glucose in the ratio 1:5 to 25:1 to 50, means that for every part galactose, the polysaccharide mixture comprises in the range of 5 to 25 parts mannose and in the range if 1 to 50 part glucose.
Molecular weight: Every polysaccharide of a composition is said to have a molecular weight of at least a given value, when said composition has been purified using a filtration step resulting in a molecular weight cut-off of said given value. Similarly, every polysaccharide of a composition is said to have a molecular weight within a given range, when said composition has been subjected to one or more filtration steps resulting in a lower molecular weight cut-off which is the lower value of the range and an upper molecular weight cut-off which is the upper value of the range. Said filtration step may for example be ultrafiltration, microfiltration, ultracentrifugation or gel filtration. However, a composition wherein every polysaccharide has a molecular weight of at least a given value or every polysaccharide is said to have a molecular weight within a given range may also be prepared by other methods.
Polypeptide: the term “polypeptide” as used herein covers proteins, peptides and polypeptides, wherein said proteins, peptides or polypeptides may or may not have been post-translationally modified. Post-translational modification may for example be phosphorylation, methylation, glucosylation,

When used herein, the terms “feed” or “food”, include feed or food additives, feed or food supplements or feed or food premixes.

When used herein, the term “liquid culture” is used to indicate all forms of non-solid culture, including submerged culture and suspension culture.

“Fermentation”, cultivation” and “culturing” are used interchangeably herein.

When used herein the terms “biomass” and “extracellular” are intended to described the cell-associated and non-cell-associated fractions of the liquid culture, respectively.

“removal of the biomass” indicates that a substantial part of the biomass is removed, preferably more than half, such as more than 90%, i.e. more than 96%, such as more than 99% of the biomass is removed

When used herein, the term “animal” in intended to include zooplankton, as well as artemia and rotifers, but not microorganisms.

### DETAILED DESCRIPTION OF THE INVENTION

### Food or Feed Product

In one embodiment of the invention the food or feed product comprises extracellular material comprising a survival enhancing agent derived from a liquid culture of a fungus of the class of Basidiomycetes. Preferred Basidiomycetes to be used with the present invention are described herein below in the section “Fungi”. Suitable methods for cultivating Basidiomycetes in liquid culture are described herein below in the section “Methods of liquid cultivation”. The extracellular material may for example be the extracellular liquid obtained after removal of biomass or an extracellular composition isolated from the extracellular liquid comprising a survival enhancing agent. Methods of isolating compositions comprising a survival enhancing agent are described herein below in the section “Isolating a composition comprising a survival enhancing agent”. In one embodiment of the invention, the feed or food product does not comprise living animals, however, the product may comprise living microorganisms.

In another embodiment of the invention the feed product may comprise biomass derived from a liquid culture of a fungus of the class of Basidiomycetes. Useful Basidiomycetes and methods of cultivating them are described herein below. This embodiment is particularly relevant for fish feed products, farm animal feed products or shell fish products.

The food or feed product may be any product suitable for oral consumption, preferably food, feed, drink or a supplement for food or feed. Thus the food or feed product should preferably have a taste acceptable to the animal species for which it is intended. Food products for human consumption preferably have a pleasant taste. Pleasant taste may for example be determined by a test panel.

Depending on the animal for which the feed or food product is intended it will have a different form.

In one embodiment of the invention the feed product is an aquatic animal feed product, such as a fish feed product or a shellfish product. A fish feed product may be any conventional fish feed further comprising the above-mentioned biomass, extracellular liquid or extracellular composition. Thus for example fish feed may consist of compressed pellets or a dry powder. For fish larvae the feed may be a fine powder, such as a powder with a particle size in the range of 80 to 500 μm, depending on the size of the larvae. Fish feed may preferably comprise in the range of 40 to 80%, such as 70 to 80% proteins, in the range of 5 to 40%, such as 5 to 15% lipids and in the range of 5 to 40%, such as 5 to 15% carbohydrates. Fish feed may be prepared from a number of different sources, for example from fish meal, meal of other marine species and/or soya. Many freshwater fish, such as salmon or trout, are fed this kind of fish feed, when bred in a fish farm. Certain aquatic animals prefer life food for at least part of their life cycle, in particular young fish larvae may prefer life food. This is in particular true for marine fish, such as cods, turbot, haddock, sea bass or sea bream. Young cod larvae, preferably eat live feed roughly until day 35-40 after hatching and thus the feed product may in one embodiment be comprised within a living microorganism. Said living microorganism may for example be plankton, such as zoo plankton, for example it may be selected from the group consisting of artemia, rotifers (rotatoria) and Calanus. Later in life marine fish feed may be the feed described above.

The aquatic animal feed product, may also for example be feed for Crustacea, such as for Malacostraca, for example Eumalacostraca, such as Eucarida, such as Decapoda, for example Natantia, such as Penaeoidea, for example penaeidae, for example Penaeus. Certain Crustacea, such as crustaceans of the family Penaeida may also prefer live feed at least during part of their life cycle. Thus, for example larvae of Penaeida preferably eat live feed, such as microorganisms, for example plankton, such as zoo plankton, for example artemia, rotifer or Calanus. Later in life Penaeida may be fed with dry feed, for example feed similar to the fish feed described above.

Thus in on embodiment of the invention, the feed product is a zooplankton feed product, such as an artemia or rotifer or Calanus feed product. Zooplankton are very small organisms and hence zoo plankton feed products in general consist of very small particles. Zooplankton feed products may be an emulsion of an organic phase in an aqueous phase. Preferably, the organic phase comprises the survival enhancing agent. The organic phase may be any organic solvent, preferably an organic solvent which is not toxic to zooplankton. The organic phase may thus for example be marine oil, such as fish oil or train oil, such as cod liver oil or whale oil or vegetable oil, such as soy oil or calamus oil. The aqueous phase may for example be water, such as sea water or lake water. Illustrative examples of methods for preparing such emulsion are described herein below in the section “Preparing feed or food product”

In another embodiment of the invention the feed product is a farm animal feed product. By the term “farm animal” is meant animals bred on farms mainly for production purposes, for example for the production of meat, milk, eggs or wool. Examples of farm animals include cattle, pigs, sheep, goat, poultry, such as turkey, chickens or ducks.

Pig feed may be in the form of conventional concentrates prepared from various plant products, including beets, grains, such as barley, wheat or oat, soy, such as soy proteins or vegetable oil/fat. Other sources may also be available. The survival enhancing agent may be admixed with the feed as a dry powder or dry pellets or it may be admixed with the feed in liquid form. The biomass may also be directly mixed with the feed

Poultry feed products, such as chicken feed products, frequently comprise various vegetarian products such as corn, maize, grains, such as wheat, barley or oat and/or soybean meal, as well as animal products such as fish meal and/or animal fat. It is preferred that the growth medium for growing the fungus, such as Lentinin, for producing a feed product, such as a poultry feed product, is not obtained from washing grain.

In one embodiment of the invention the product is a food product. The food product may for example be a nutritional supplement. The nutritional supplement could be in the form of a liquid or a solid, such as a pill, lozenge or tablet. The liquid could be intended for direct intake or it could be intended for adding to drinks or food. The liquid may in one preferred embodiment be the crude extracellular liquid obtained after fermentation and removal of biomass. The solid could be a dry powder for example prepared as described herein below in the section “Preparing food or feed product”.

In yet another embodiment of the invention the product is a pet feed product, such as a nutritional supplement for pets. The nutritional supplement for pets could be similar to nutritional supplements for human beings. Herein the term “pet” is used to designate animals, which are kept in captivity by human beings for other purposes than production. The pet feed product will be dependent on the pet. In general the extracellular liquid, the extracellular composition and/or the biomass may be added to conventional food for said pet. The pet may preferably be a mammal, for example dogs, cats, horses, hamsters, rabbits or guinea pigs. However, the pet may also be fish, birds, reptiles or other animals.

In yet another embodiment of the invention the product is a feed product for an animal used in competitions. In this embodiment, it is preferred that the feed product may enhance the performance of animals in competitions and optionally reduce stress. Examples of animals used in competitions include camels, horses, such as racing horses or polo horses or dogs, such as greyhounds. Feed for these animals will depend on the nature of the animal, but may generally comprise the extracellular liquid, the extracellular composition, the biomass and/or the compositions isolated from biomass as described by the present invention added to a conventional feed for said pet.

### Survival Enhancing Agent

The invention relates to food or feed products comprising a bioactive agent, such as a survival enhancing agent. In some embodiments the survival enhancing agent may also be one or more of the following:

Growth enhancing agent
Health enhancing agent (for example in a functional food, as described herein)
Modulator of a microbial population (for example in a functional food, as described herein)
Longevity enhancing agent

The survival enhancing agent preferably comprises polysaccharides and/or polypeptides, more preferably both polysaccharides and polypeptides.

In one embodiment of the invention it is preferred that the survival enhancing agent comprises one or more polypeptides and a mixture of polysaccharides. Said polypeptides may optionally be covalently linked to polysaccharides. It is however also comprised within the present invention that said polypeptides are either not associated with said polysaccharides or that said polypeptides are associated with said polysaccharides in a non-covalent manner.

Thus the survival enhancing agent preferably comprises polysaccharides which may or may not be proteopolysaccharides, or the survival enhancing agent may comprise a mixture of both.

The survival enhancing agent may be comprised within a crude extracellular liquid, obtained directly by removal of biomass after cultivation of a Basidiomycete in liquid culture. However, the survival enhancing agent may also be comprised within isolated or purified extracellular compositions, i.e. they have been subjected to one or more purification steps. In a preferred embodiment they have been purified from liquid growth medium of a fungal mycelium using at least one purification step comprising a size fractionation. Methods of purification are described in more detail below.

In a preferred embodiment, the survival enhancing agent essentially consists of polysaccharides and optionally polypeptides, more preferably the survival enhancing agent essentially consists of polysaccharides and polypeptides. Frequently, the extracellular composition is a liquid composition, and it is then preferred that the composition essentially consists of polysaccharides and optionally polypeptides dissolved in an aqueous solution optionally comprising salts and buffer.

The polysaccharides of the survival enhancing agent preferably comprise the monosaccharides glucose, mannose and galactose.

In a preferred embodiment the survival enhancing agent comprises polysaccharides, wherein the majority of the polysaccharides, preferably at least 60%, more preferably at least 70%, even more preferably at least 80%, yet more preferably at least 90%, even more preferably at least 95%, yet more preferably essentially all polysaccharides, most preferably every polysaccharide has a molecular weight above 10,000 Da, preferably above 30,000 Da, more preferably above 40,000 Da, even more preferably above 50,000 Da, for example at least 100,000 Da, such as at least 300,000 Da, for example at least 1,000,000 Da. In one embodiment of the invention the majority of polysaccharides, preferably every polysaccharide of the survival enhancing agent has a molecular weight within the range of 10,000 to 3,000,000 Da, for example within the range of 30,000 to 3,000,000, such as within the range of 40,000 to 3,000,000, for example within the range of 50,000 to 3,000,000, such as in the range of 50,000 to 100,000, for example in the range of 100,000 to 300,000, such as in the range of 300,000, to 1,000,000, for example in the range of 1,000,000 to 3,000,000.

Preferably, the survival enhancing agent has been purified by a method involving at least one size fractionation step. Thus it is preferred, that the survival enhancing agent has been purified using at least one size fractionation step wherein molecules, such as polysaccharides with a nominal molecular weight above a given cut-off are separated from molecules, such as polysaccharides with a nominal molecular weight below said cut-off. By way of example, if the size fractionation is ultrafiltration or microfiltration a membrane with said cut-off may be used. If the size fractionation is gel filtration a gel with said molecular weight cut off may be chosen or a particular elution fraction may be used. In one embodiment of the invention the larger molecular weight fraction is used, wherein the cut-off preferably is 10,000 Da, more preferably 30,000 Da, even more preferably 40,000 Da, yet more preferably 50,000 Da.

In another embodiment of the invention, the survival enhancing agent has been purified by a method involving one or more size fractionation steps, wherein a resulting fraction comprises polysaccharides with a nominal molecular weight below a given cut-off and above a given cut-off. By way of example, if the size fractionation is ultrafiltration or microfiltration, then first one fractionation step using a membrane with the lower molecular weight cut off may be performed. The larger molecular weight fraction may be collected and subjected to a second ultrafiltration or microfiltration using a membrane with the upper molecular weight cut off. After the second filtration step the lower molecular weight fraction may be collected. If gel filtration is used a particular elution fraction may be used. If ultracentrifugation is used, a membrane with the lower molecular cut-off and a membrane with the upper molecular weight cut-off may be used.

The survival enhancing agent according to the present invention may comprise a mixture of polysaccharides, wherein said mixture comprises the monosaccharides galactose, mannose, and glucose in the ratio 1:5 to 25:1 to 50. Thus, the ratio reflects the ratio within the entire mixture of polysaccharides. It is thus feasible that each individual polysaccharide within the mixture comprises a different ratio of the monosaccharides.

The ratio may in general be determined by degrading the entire mixture of polysaccharides into monosaccharides and subsequently determining the concentration of each of said monosaccharides. Polysaccharides may be degraded to their constituent monosaccharides by hydrolysis, for example by hydrolysis in a strong acid, such as HCl. The hydrolysate may be analysed by any conventional method available to the skilled person, for example by HPLC, mass spectrometry or NMR.

In one embodiment of the invention the polysaccharides comprise the monosaccharides glucose and mannose. In another embodiment of the invention the polysaccharides comprise the monosaccharides glucose and galactose. In a preferred embodiment of the invention, the polysaccharides of the survival enhancing agent comprise the monosaccharides galactose, mannose and glucose.

It is preferred that the mixture of polysaccharides comprises in the range of 5 to 25, preferably in the range of 5 to 20, more preferably in the range of 5 to 17, even more preferably in the range of 6 to 15, yet more preferably in the range of 7 to 14, such as in the range of 10 to 17, for example in the range of 11 to 16, such as in the range of 12 to 15, for example in the range of 13 to 14, such as approximately 13.4+/−0.4, for example 13.4+/−0.4 parts mannose for every part galactose.

It is preferred that the mixture of polysaccharides comprises in the range of 1 to 50, preferably in the range of 1 to 40, more preferably in the range of 1 to 30, even more preferably in the range of 1 to 25, yet more preferably in the range of 1 to 20, even more preferably in the range of 2 to 15, yet more preferably in the range of 2 to 14, such as in the range of 8 to 17, for example in the range of 9 to 16, such as in the range of 10 to 15, for example in the range of 11 to 14, such as approximately 12.6+/−1.3, for example 12.6+/−1.3 parts glucose for every part galactose.

It is even more preferred that the mixture comprises a ratio of mannose to galactose as indicated herein above and a ratio of glucose to galactose in a ratio as indicated herein above.

In embodiments of the invention, wherein the polysaccharides comprise mannose and glucose it is preferred that they comprise in the range of 0.1 to 30, such as in the range of 0.1 to 0.25, for example in the range of 0.25 to 0.5, such as in the range of 0.5 to 0.75, for example in the range of 0.75 to 1, such as in the range of 1 to 5, for example in the range of 5 to 10, such in the range of 10 to 20, for example in the range of 20 to 30 parts glucose for every part mannose. Preferably the polysaccharides comprise in the range of 0.5 to 2 parts glucose for every part mannose, more preferably 13.4+/−0.4 parts mannose 12.6+/−1.3 part glucose.

In a very preferred embodiment of the invention the survival enhancing agent comprises a mixture of polysaccharides comprising the monosaccharides galactose, mannose, and glucose in the ratio 1:5 to 25:1 to 50, more preferably 1:13.4+/−0.4:12.6+/−1.3.

In another embodiment of the invention the survival enhancing agent comprises a mixture of polysaccharides, wherein the polysaccharides within said mixture having a molecular weight in the range of 50,000 to 100,000 comprises in the range of 3 to 15, preferably in the range of 4 to 14, more preferably in the range of 5 to 13, even more preferably in the range of 6 to 12, yet more preferably in the range of 7 to 11, even more preferably in the range of 7.9 to 9.9, for example approximately 8.9 parts mannose for every part galactose. In this embodiment it is preferred that the polysaccharides having a molecular weight in the range of 50,000 to 100,000 comprises in the range of 1 to 5, preferably in the range of 2 to 4, even more preferably in the range of 2.5 to 3.5, such as approximately 2.9 parts glucose for every part galactose. It is preferred that the polysaccharides of said survival enhancing agent having a molecular weight in the range of 50,000 to 100,000 Da comprise the monosaccharides galactose, mannose, and glucose in the ratio 1:4 to 14:1 to 5, preferably the ratio is approximately 1:8.9:2.9.

In another embodiment of the invention the survival enhancing agent comprises a mixture of polysaccharides, wherein the polysaccharides within said mixture having a molecular weight in the range of 100,000 to 300,000 comprises in the range of 3 to 15, preferably in the range of 3 to 14, more preferably in the range of 3 to 13, even more preferably in the range of 3 to 12, yet more preferably in the range of 4 to 11, even more preferably in the range of 5 to 10, yet more preferably in the range of 6.3 to 8.3, for example approximately 7.3 parts mannose for every part galactose. In this embodiment it is preferred that the polysaccharides having a molecular weight in the range of 100,000 to 300,000 comprising in the range of 1 to 5, preferably in the range of 2 to 4, even more preferably in the range of 2.5 to 3.5, such as approximately 2.9 parts glucose for every part galactose. It is preferred that the polysaccharides of said survival enhancing agent having a molecular weight in the range of 50,000 to 100,000 Da comprise the monosaccharides galactose, mannose, and glucose in the ratio 1:3 to 13:1 to 5, preferably the ratio is approximately 1:7.3:2.9.

In another embodiment of the invention the survival enhancing agent comprises a mixture of polysaccharides, wherein the polysaccharides within said mixture having a molecular weight in the range of 300,000 to 1,000,000 comprising in the range of 3 to 16, preferably in the range of 5 to 15, more preferably in the range of 5 to 14, even more preferably in the range of 6 to 13, yet more preferably in the range of 7 to 12, even more preferably in the range of 8.9 to 10.9, for example approximately 9.9 parts mannose for every part galactose. In this embodiment it is preferred that the polysaccharides having a molecular weight in the range of 300,000 to 1,000,000 comprising in the range of 1 to 5, preferably in the range of 2 to 4, even more preferably in the range of 2.5 to 3.5, such as approximately 3.1 parts glucose for every part galactose. It is preferred that the polysaccharides of said survival enhancing agent having a molecular weight in the range of 300,000 to 1,000,000 Da comprise the monosaccharides galactose, mannose, and glucose in the ratio 1:5 to 15:1 to 5, preferably the ratio is approximately 1:9.9:3.1.

In another embodiment of the invention the survival enhancing agent comprises a mixture of polysaccharides, wherein the polysaccharides within said mixture having a molecular weight of at least 1,000,000 comprising in the range of 3 to 17, preferably in the range of 4 to 16, more preferably in the range of 5 to 15, even more preferably in the range of 6 to 14, yet more preferably in the range of 7 to 13, even more preferably in the range of 8 to 12, even more preferably in the range of 9.3 to 11.3, for example approximately 10.3 parts mannose for every part galactose. In this embodiment it is preferred that the polysaccharides having a molecular weight of at least 1,000,000 comprising in the range of 1 to 5, preferably in the range of 2 to 4, even more preferably in the range of 2.5 to 3.5, such as approximately 2.9 parts glucose for every part galactose. It is preferred that the polysaccharides of said survival enhancing agent having a molecular weight in the range of at least 1,000,000 Da comprise the monosaccharides galactose, mannose, and glucose in the ratio 1:4 to 1:5 to 15:1 to 5, preferably the ratio is approximately 1:10.3:2.9.

Determination of the monosaccharide content of polysaccharides with a molecular weight within a given range may be determined by fractionating the liquid comprising the survival enhancing agent according to size for example by ultrafiltration, microfiltration, ultracentrifugation or gelfiltration.

The survival enhancing agent according to the invention preferably comprises polypeptides. The term polypeptide as used herein covers both proteins, peptides and polypeptides. Said polypeptides may be in free form, they may be covalently linked to a polysaccharide or they may be non-covalently associated with a polysaccharide or a mixture of the aforementioned.

It is preferred that the survival enhancing agent comprises sufficient polypeptide in order to allow for oral administration of the survival enhancing agent. If the survival enhancing agent comprises too little polypeptide, then no or little immune modulation is obtained in an individual after oral administration of the survival enhancing agent to said individual. It is therefore preferred that the survival enhancing agent of the invention comprises at least 10 μg/L, more preferably at least 20μ/L, even more preferably at least 25 μg/L, for example in the range of 10 to 1000 μg/L, such as in the range of 20 to 1000 μg/L, for example in the range of 25 to 1000 μg/L, such as in the range of 25 to 100 μg/L, for example in the range of 25 to 35 μg/L polypeptide, preferably soluble polypeptide. It is preferred that the survival enhancing agent comprising the aforementioned concentration of polypeptide, comprises in the range of 0.1 to 2, more preferably in the range of 0.5 to 1.5, even more preferably around 1 mg/ml polysaccharide. If the survival enhancing agent comprises more or less polysaccharide it is preferred that the amount of polypeptide is proportionally reduced or enhanced.

In one embodiment of the invention the extracellular liquid and/or the survival enhancing agent comprises in the range of 90 to 99% glucose, such as approximately 98.9% glucose, in the range of 1 to 10% mannose, such as approximately 1% mannose, very small amounts of galactose, such as approximately 0.1% galactose. It is comprised within this embodiment that at least some of the sugars are present as a monomer, thus up to 80%, such as in the range of 60 to 80% may be free glucose. In this embodiment the extracellular liquid and/or the survival enhancing agent preferably also comprises in the range of 20 to 400 mg/l protein, such as in the range of 50 to 150 mg/l, for example in the range of 80 to 100 mg/l, such as approximately 90 mg/l protein.

In one preferred embodiment of the present invention, such as in a functional food, the survival enhancing agent is not eritadenine.

In one embodiment, the survival enhancing agent is a polysaccharide, such as a polysaccharide having a molar ratio of galactose:mannose:glucose of 1:10 to 20:30 to 50, such as 1:12 to 18:35 to 45; for example 1:14 to 16:38 to 42, such as 1: about 15: about 40, for example 1:15:40.

Accordingly, in one embodiment of the present invention, the survival enhancing agent comprises one or more polypeptides and/or a mixture of polysaccharides, wherein the majority of the polysaccharides of the agent has a molecular weight of at least 10,000 Da and wherein said one or more polypeptides and/or said mixture of polysaccharides comprises the monosaccharides galactose, mannose and glucose in the ratio (galactose:mannose:glucose) of 1:0 to 25:1 to 50, such as 1:10 to 20:30 to 50, such as 1:12 to 18:35 to 45; for example 1:14 to 16:38 to 42, such as 1: about 15: about 40, for example 1:15:40.

In another embodiment, the survival enhancing agent according to the present invention has a molar ratio of galactose:mannose:glucose of 1:0.5 to 5:6 to 12, such as 1:1 to 4:7 to 11; for example 1:1.5 to 3.5:7.5 to 10, such as 1:2.0 to 3.0:7.5 to 9.5, for example 1:2.2 to 2.8:8.0 to 9.0, such as 1: about 2.5:8.0 to 9.0, for example 1:2.5:8.0 to 9.0, such as 1:2.5:8.6.

Accordingly, in another embodiment of the present invention, the survival enhancing agent according to the invention comprises one or more polypeptides and/or a mixture of polysaccharides, wherein the majority of the polysaccharides of the composition has a molecular weight of at least 10,000 Da and wherein said one or more polysaccharides and/or said mixture of polysaccharides comprises the monosaccharides galactose, mannose and glucose in the ratio (galactose:mannose:glucose) of 1:0 to 25:1 to 50, for example 1:0.5 to 5:6 to 12, such as 1:1 to 4:7 to 11; for example 1:1.5 to 3.5:7.5 to 10, such as 1:2.0 to 3.0:7.5 to 9.5, for example 1:2.2 to 2.8:8.0 to 9.0, such as 1: about 2.5:8.0 to 9.0, for example 1:2.5:8.0 to 9.0, such as 1:2.5:8.6.

Helicobacter pylori

Helicobacter is a gram-negative bacterium with polar flagella, using oxygen as an electron acceptor, which cannot utilize carbohydrates as an energy source. Helicobacter is used herein interchangeably with “Helicobacter sp.”. In a preferred embodiment the Helicobacter sp. is Helicobacter pylori.

In one embodiment, the present invention provides methods for preventing or inhibiting or reducing the growth of Helicobacter by administering the bioactive agent according to the present invention. The bioactive agent can be administered to an individual in need thereof alone or in combination with other therapeutic agents like antibiotics and inhibitors of acid secretion. By the phrase “in combination” with therapeutic agents is meant herein that one or more bioactive agent(s) according to the present invention is administered to the individual thus treated before and/or during (including concurrently with) and/or after treatment of an individual with one or more therapeutic agents. In all cases of combination treatment described herein, the bioactive agent can be administered in the form of food. In all cases of combination treatment described herein, the combination may be in the form of kit-in-part systems, wherein the combined active substances may be used for simultaneous, sequential or separate administration. In all cases, it is preferred that any of the herein-mentioned medicaments are administered in pharmaceutically effective amounts, i.e. an administration involving a total amount of each active component of the medicament or pharmaceutical composition or method that is sufficient to show a meaningful patient benefit. The combination of a bioactive agent according to the present invention and therapeutic agents provide improvements over therapy with the therapeutic agent alone, in particular for patients that do not respond to therapy with the therapeutic agent alone or in combination with other treatment regimes.

Thus, the present invention provides a method of treating an infection with Helicobacter in a subject, particularly human subjects, comprising administering a therapeutically effective amount of a bioactive agent according to the present invention alone or in combination with other therapeutic agents.

In one embodiment, the other therapeutic agent is an antibiotic. In another embodiment the antibiotic is amoxicillin. In a further embodiment the antibiotic is clarithromycin. In yet another embodiment the antibiotic is metronidazole. In another embodiment the therapeutic agent is an inhibitor of acid secretion like an H₂ inhibitor or a proton pump inhibitor. In a further embodiment the H₂ inhibitor is omeprazol. Further embodiments of the invention provide methods where one or more antibiotic is co-administered with an inhibitor of acid secretion.

In one embodiment of the invention the subject having a Helicobacter infection is suffering from a peptic ulcer. Peptic ulcers, as contemplated in the current invention include, but are not limited to, circumscribed breaks in the continuity of the mucosal layer of the gastrointestinal tract. These breaks in the continuity of the mucosal layer can include breaks that do not extend below the epithelium, also referred to as “erosions” or breaks that do extend below the epithelium. The peptic ulcers may be acute, or chronic. Further, peptic ulcers can be located in any part of the gastrointestinal tract that is exposed to acid-pepsin gastric juice, including esophagus, stomach, duodenum and after gastroenterostomy, the jejunum.

In another embodiment the subject having the Helicobacter infection is suffering from, or at risk of developing, cancer of the gastrointestinal tract. As stated above, the portions of the gastrointestinal tract where cancer may be present or may develop are any areas where the gastrointestinal tract is exposed to acid-pepsin gastric juice, including esophagus, stomach, duodenum and after gastroenterostomy, the jejunum. As used herein the term “cancer of the gastrointestinal tract” is used as one of ordinary skill in the art would recognize the term. Examples of “cancer of the gastrointestinal tract” include, but are not limited to, neoplasias (or neoplasms), hyperplasias, dysplasias, metaplasias or hypertrophies. The neoplasms may be benign or malignant, and they may originate from any cell type, including but not limited to epithelial cells of various origin, muscle cells and endothelial cells.

The treatment can be used for patients with a pre-existing Helicobacter infection, or for patients pre-disposed to a Helicobacter infection. Additionally, the bioactive agent of the present invention can be used to alleviate symptoms of a Helicobacter infection in patients, or as a preventative measure in patients.

As used herein, the phrase Helicobacter infection is used to mean an interaction between Helicobacter and the host organism (subject). The infections may be localized, meaning that the Helicobacter grows and remains near the point of initial interaction. The infection may also be generalized, where the Helicobacter may become more widespread beyond the initial point of interaction, including spreading to the surrounding tissue or organ and even being distributed and growing throughout the entire host organism. As used herein the term interaction (of a host and Helicobacter) is used to mean a process where the Helicobacter grows in or around a particular tissue. Helicobacter is considered to have infected the subject if the bacteria is able to penetrate the surface of cells of a particular tissue and grow within the cells of the tissue. An example of this type of infection includes, but is not limited to Helicobacter penetrating and growing within the epithelial cells lining the lumen of the stomach. Additionally, the Helicobacter can also be said to have infected the host organism by growing extracellularly to the tissue cells.

The method of the current invention comprises administering an antibacterially effective amount of the bioactive agent to treat a Helicobacter infection. As used herein, “an antibacterially effective amount to treat a Helicobacter infection” is intended to mean an amount affective to prevent, inhibit, retard or reverse the growth of Helicobacter, and/or reduce the number of viable Helicobacter cells within the stomach or at a site of infection. “Antibacterially effective amount to treat a Helicobacter infection” is also used to mean an amount effective to kill, reduce or ameliorate any existing infections of Helicobacter. Thus, according to the present invention, an “antibacterially effective amount to treat a Helicobacter infection” of the bioactive agent of the present invention can be used as a treatment of a pre-existing Helicobacter infection. Effective amounts for use in these treatments can completely or partially prevent a pre-existing Helicobacter infection from spreading to surrounding tissue and beyond, and they can also be used to slow the growth and/or spread rate of the Helicobacter in the subject. Furthermore, the “antibacterially effective amounts to treat a Helicobacter infection” of the bioactive agent of the current invention can prevent a Helicobacter infection in subjects. Another aspect of an “antibacterially effective amount to treat a Helicobacter infection”, as used in the current invention, means that the bioactive agent administered to the subject is capable of preventing or reducing the cellular or physiological damage to the infected or surrounding tissue, caused by the toxins produced by the Helicobacter.

In still another aspect, the phrase “antibacterially effective amount to treat a Helicobacter infection” can be used to mean an amount of the administered bioactive agent that can reduce or prevent the formation or efficacy of the virulence of the Helicobacter. By virulence is meant the ability of the Helicobacter to combat the host organism's or cells natural defenses to the Helicobacter infection.

### Antibody Therapy

In one embodiment, the present invention provides methods for enhancing the antitumor activity of antibody therapy by administering a bioactive agent according to the present invention in combination with the antibody therapy. By the phrase “in combination” with antibody therapy is meant herein that one or more bioactive agent(s) according to the present invention is administered to the individual thus treated before and/or during (including concurrently with) and/or after treatment of an individual with a therapeutic antibody. In all cases of combination treatment described herein, the bioactive agent can be administered in the form of food. In all cases of combination treatment described herein, the combination may be in the form of kit-in-part systems, wherein the combined active substances may be used for simultaneous, sequential or separate administration. In all cases, it is preferred that any of the herein-mentioned medicaments are administered in pharmaceutically effective amounts, i.e. an administration involving a total amount of each active component of the medicament or pharmaceutical composition or method that is sufficient to show a meaningful patient benefit. The combination of a bioactive agent according to the present invention and therapeutic monoclonal antibodies provide improvements over monoclonal antibody therapy alone, in particular for patients that do not respond to monoclonal antibody therapy alone or in combination with other treatment regimes.

Thus, the present invention provides a method of treating cancer in a subject, particularly human subjects, comprising co-administering a therapeutically effective amount of a monoclonal antibody and a therapeutically effective amount of a bioactive agent according to the present invention.

In one embodiment, the monoclonal antibody is an anti-CD20 monoclonal antibody. In another embodiment, the monoclonal antibody is rituximab. In another embodiment, methods of the present invention treat non-Hodgkin's lymphoma. Further embodiments of the present invention provide methods where monoclonal antibody rituximab and a bioactive agent according to the present invention are administered once weekly for e.g. up to eight consecutive weeks. In another embodiment, the rituximab is administered once weekly and the a bioactive agent according to the present invention is administered up to five times weekly for up to eight consecutive weeks. Another embodiment of present invention provides that the bioactive agent dose is from 10 to 500 [ mu]g/kg/dose. In certain embodiments of the present invention, the patient has previously been treated with rituximab and showed no appreciable tumor remission or regression. In other embodiments, the patient has relapsed after receiving rituximab therapy.

In another aspect, the present invention provides a method of treating cancer in a subject comprising co-administering a therapeutically effective amount of an anti-CD20 monoclonal antibody and a therapeutically effective amount of a bioactive agent according to the present invention, wherein administering the bioactive agent results in an optimal immunological response.

In another aspect, the present invention provides a method for treating cancer in a subject comprising co-administering a monoclonal antibody that binds to a Her-2/neu receptor and a bioactive agent according to the present invention. In one embodiment, the subject is a human patient. The monoclonal antibody can e.g. be trastuzumab.

One aspect of the present invention provides a method of treating cancer in a subject comprising co-administering a monoclonal antibody that binds to a cytotoxic T lymphocyte-associated antigen 4 (CTLA-4) and a bioactive agent according to the present invention. In certain embodiments, the subject is a human patient. In one embodiment of the present invention, the anti-CTLA-4 monoclonal antibody is administered at a dose of 3 mg/kg every three weeks for four cycles and the bioactive agent is administered one to five times weekly for up to eight weeks. The present invention also provides embodiments where the dose of the bioactive agent is from 10 to 500 [ mu]g/kg/dose.

One of the mechanisms associated with the antitumor activity of monoclonal antibody therapy is antibody dependent cellular cytotoxicity (ADCC). In ADCC, monoclonal antibodies bind to a target cell (e.g. cancer cell) and specific effector cells expressing receptors for the monoclonal antibody (e.g. NK cells, monocytes and granulocytes) bind the monoclonal antibody/target cell complex resulting in target cell death. A bioactive agent according to the present invention is believed to enhance effector cell function, thereby increasing monoclonal antibody therapy efficacy. Thus, the dose and schedule of bioactive agent administration in combination with MAbs can be based on the ability of the bioactive agent to elevate parameters associated with differentiation and functional activity of cell populations mediating ADCC, including but not limited to, NK cells, macrophages and neutrophils. These parameters can be evaluated using assays of NK, macrophage and neutrophil cell cytotoxicity, ADCC (NK cell fraction or total mononuclear cells, or effector molecules essential to the ability of cells to implement ADCC (e.g., FasL, granzymes and perforin).

Combination therapy with a bioactive agent according to the present invention and a monoclonal antibody may in one embodiment be indicated when a first line treatment has failed and may be considered as a second line treatment. However, based on the enhanced antitumor activity of the bioactive agent in combination with a monoclonal antibody, the present invention also provides using the combination as a first line treatment in patient populations that are newly diagnosed and have not been previously treated with anticancer agents “de novo patients” and patients that have not previously received any monoclonal antibody therapy “naive patients.”

A bioactive agent according to the present invention is also useful in combination therapy with monoclonal antibodies in the absence of any direct antibody mediated ADCC of tumor cells. Antibodies that block an inhibitory signal in the immune system can lead to augmented immune responses. Examples include (1) antibodies against molecules of the B7R family that have inhibitory function such as, cytotoxic T lymphocyte-associated antigen 4 (CTLA-4), programmed death-1 (PD-1), B and T lymphocyte attenuator (BTLA); (2) antibodies against inhibitory cytokines like IL-10, TGFP; and (3) antibodies that deplete or inhibit functions of suppressive cells like anti-CD25 or CTLA-4. For example, anti-CTLA4 mAbs in both mice and humans are thought to either suppress function of immune-suppressive regulatory T cells (Tregs) or inhibit the inhibitory signal transmitted through binding of CTLA-4 on T cells to B7-1 or B7-2 molecules on APCs or tumor cells. CTLA-4 is expressed transiently on the surface of activated T cells and constitutively expressed on Treg cells. Cross-linking CTLA-4 leads to an inhibitory signal on activated T cells, and antibodies against CTLA-4 block the inhibitory signal on T cells leading to sustained T cell activation (Phan et al., PNAS, 100:8372-8377, 2003.)

Preferred antibodies for use in the combination therapy: Although monoclonal antibodies are preferred, any of the embodiments described herein may also use polyclonal antibodies instead of, or in combination with, monoclonal antibodies. In one embodiment of the combination invention, naked antibodies (i.e. antibodies without any drug or radioactive material attached to them) are used. In another embodiment of the present invention, conjugated antibodies are used (joined e.g. to one or more of: a chemotherapy drug, a radioactive particle, or a toxin). For example, the antibody used may be a conjugated monoclonal antibody. Another preferred embodiment uses one or more of: a chemolabeled monoclonal antibody, a monoclonal antibody with radioactive particles attached, an immunotoxin.

Preferred immunotoxins include, but are not restricted to, an antibody attached to one or more of: a bacterial toxins such as diphtherial toxin (DT) or pseudomonal exotoxin (PE40), a plant toxin such as ricin A or saporin. Preferred is e.g. gemtuzumab ozogamicin (Mylotarg) or other antibodies attached to calicheamicin, or BL22.

It is preferred that the antibody is targeted to a molecule known to be associated with cancerous processes. For example, the antibody may bind specifically one or more of the following targets: vascular endothelial growth factor-A (VEGF-A), epidermal growth factor receptor (EGFR), CD20 antigen, the HER2 protein, the CD52 antigen, the VEGF protein, erbB-2, EGFR, erbB-2, cathepsin L, cyclin E, Ras, p53, BCR-ABL, Bcl-2, caspase-3.

Table 1 is a non-exclusive list of monoclonal antibodies approved or being tested for which combination therapy with a bioactive agent according to the present invention is possible. Other preferred antibodies may be selected from, but are not restricted to, the group consisting of:

Alemtuzumab (Campath), bevacizumab (Avastin, Genentech Inc.), OncoScint (such as for colorectal and ovarian cancer), ProstaScint (such as for prostate cancer), Tositumomab (Bexxar),
Cetuximab (Erbitux, ImClone Systems Inc.), Gemtuzumab ozogamicin (Mylotarg), Rituximab (Rituxan, Roche/Genentech), anti-erbB-2 scFv, Ibritumomab tiuxetan (Zevalin), Panitumumab (formerly known as “ABX-EGF”, Abgenix, Fremont Calif.), Ibritumomab tiuxetan (Zevalin), EMD 72000 (Vanhoefer et al., J Clin Oncol 2004; 22:175-184), Ibritumomab Tioxetan, and Trastuzumab (Herceptin).

Further suitable antibodies and protocols for use of any of the antibodies described herein can be found in e.g. US patent applications no. US2005/0244413 (Adolf et al.) and us20050265966 (Wane et al.), U.S. Pat. No. 5,338,661 (Jensenius), and “Recombinant Polyclonal Antibodies for Cancer Therapy” (Sharon et al., Journal of Cellular Biochemistry 96:305-313 (2005)), both of which are incorporated herein by reference.

TABLE 1

| Target | Drug Name | Clinical Indication | Company |
|---|---|---|---|
| IL-2Rα(CD25) | Zenapax | transplant | Roche |
| IL-1R | AMG108 | osteoarthritis | Amgen |
| RANK-L | AMG162 | osteoporosis | Amgen |
| Blys | LympoSTAT-B | SLE, RA | HGS |
| CD40L (CD39) | InitiatedAID | Celltech/IDEC | |
| TRAIL-R1 | HGS-ETR1 | cancers | HGS |
| TRAIL-R2 | HGS-ETR2 | solid tumors | HGS |
| CD30 | SGN30 | NHL | Seattle Genetics |
| CD40 | SGN40 | MM | Seattle Genetics |
| HER2 | Herceptin | Breast cancer | Genentech |
| EGF-R | ABX-EGF | CRC, NSCLC, RCC | Abgenix |
| | EMD72000 | solid tumors | Merck |
| | MDX-214 | EGF-R-positive | Medarex |
| | | tumors | |
| | Erbitux | CRC | Imclone |
| VEGF-R | CDP791 | solid tumors | Celltech |
| PDGF-R | CDP860 | solid tumors | Celltech/ZymoGenetics |
| CD11a (αL) | Raptiva | psoriasis | Genentech |
| α4-Integrin | Antegrin | CD, MS | PDL, Biogen-IDEC |
| α4β7 Integrin | MLM02 | CD, UC | Millenium |
| α5β3 Integrin | Vitaxin | psoriasis, prostate cancer | AME/Lilly |
| CD2 (LFA3/Fc) | Amevive | psoriasis | Biogen/IDEC |
| CD152 | CTLA-4/Ig | RA | Bristol Meyers |
| CD152 | CTLA-4 | cancers | Medarex |
| CD49a | Integrin α1 | RA/Luous | Biogen/IDEC |
| CD49e | Integrin α5 | cancers | Protein Design Labs |
| MUC1 | | | Theragyn |
| MUC18 (TIM-like) | ABX-MA1 | melanoma | |
| TAG-72 Mucin | Anatumomab | cancers | |
| CD3 | Ecromeximab | melanoma | Kyowa Hakko |
| | TRX4 | type1 IDDM | TolerRx |
| | Nuvion | UC | PDL |
| | OrthoCloneOKT3 | organ transplant | Ortho biotech |
| CD4 | HuMax-CD4 | T-cell lymphoma | GenMab |
| CD19 | MT103 | NHL | Medimmune |
| CD64 (Fc GR1) | AntiCD64 | cancers | Medarex |
| CD33 | MyloTarg | AML | Celltch/Whyeth |
| | ZAmyl | AML | Protein Design Labs |
| CD22 | lymphocide | NHL, AID | Immunomedics |
| CEA | CEA-Cide | cancers | Immunomedics |
| CD20 | Rituxan | NHL | Genentech |
| CD52 | Campath | MS, NHL, T-cell lymph | Genzyme, IDEX |
| CD44 | Bivatuzumab | cancers | Boehringer Ingetheim |
| CD23 (Fc Ep R) | IDEC152 | allerhic asthma, rhinitis | Biogen/IDEC |
| LRR: | | | |
| CD14 | ICOSIC14 | sepsis | ICOS |
| EpCAM | Panorex | colorectal cancer | Centocor |
| Lewis-Y-Ag | SGN15 | cancers | Seattle Genetics |
| CD80 | B7.1 | psoriasis/NHL | Biogen/IDEC |

Dosage of the bioactive agent may be varied as known to one skilled in the art and as disclosed in detail elsewhere herein. Preferably, administration is intravenous administration or oral administration. Antibodies may also be given intravenously in one embodiment, for example co-formulated with the bioactive agent. For example, the antibody and/or bioactive agent may be given at a dosage of 5 mg/kg, every other week, or may be administered with a 400 mg/m² loading dose and weekly doses of 250 mg/m² over 1 hour.

### Cytochrome P450

It has been shown, that the polysaccharide Lentinan from Lentinus edodes and polysaccharides from Agaricus blazei can suppress the expression of cytochrome P450s (CYPs) and thus can prevent cancer (Hashimoto et al. Biosci. Biotechnol. Biochem. 2004, 66 (7) 1610-1614 and Okamoto et al. Biofactors 2004 21 (1-4) 407-09 both of which are incorporated herein by reference). P450s are a class of drug- and xenobiotic-metabolizing enzymes mainly expressed in the liver. Carcinogens such as polyaromatic hydrocarbons and heterocyclic amines are metabolized to their carcinogenic forms by CYPs. Moreover the suppression of P450 caused by polysaccharides, such as Lentinan, is advantageous for chemotherapy patients, as it prolongs the duration and intensifies the action of drugs.

Thus in one embodiment the present invention is directed to a bioactive agent capable of suppressing the expression of P450s. In a further embodiment the bioactive agent of the present invention is used in a combination therapy with a chemotherapeutic drug. In all cases of combination therapy described herein, the bioactive agent can be administered in the form of food.

### Dendritic Cells

It has been demonstrated that chemoimmunotherapy using S-1, an oral fluoropyrimidine anticancer drug, combined with lentinan is effective in modifying dendritic cells (DCs) in vivo and in vitro (Mushiake et al. Cancer Immunol. Immunother. 2005 February; 54 (2) 120-128).

The survival period of Colon-26-bearing mice treated with S-1 and Lentinan was significantly more prolonged than that of mice treated with S-1 alone (P<0.05).

The frequency of CD86⁺DCs infiltrated into Colon-26 was increased in mice treated with S-1 and lentinan, and splenic DCs harvested from mice treated with S-1+LNT showed more potent T-cell proliferation activity than that of DCs from mice treated with S-1 alone (P<0.05).

Furthermore, the activity of cytotoxic T lymphocytes (CTLs) in splenocytes of mice treated with S-1 and Lentinan was specific and more potent than that of CTLs from mice treated with S-1 alone (P<0.05).

The results suggest that modulation of specific immunity with Lentinan has a significant role in enhanced anti-tumour effects through the modification of DC function. The combination therapy of S-1 and bioactive agents according to the invention presents a promising chemoimmunotherapy, which may lead to better survival for cancer patients. Thus in one embodiment the present invention is directed to a combination therapy of S-1 and the bioactive agent according to this invention in cancer patients. In all cases of combination therapy described herein, the bioactive agent can be administered in the form of food.

### Functional Food

In one embodiment of the present invention, the food is a functional food, preferably suitable for human beings. Said functional food comprises any of the agents described herein, preferably a survival enhancing agent, a longevity enhancing agent, a health enhancing agent and/or a modulator of a microbial population. More preferably, said agent is a health enhancing agent and/or a modulator of a microbial population. It is preferred that the functional food is suitable for at least weekly oral intake, such as for daily oral intake. Alternatively, said functional food product may be suitable for use in parenteral or enteral nutrition, preferably in combination with formulations comprising other nutrients known to one skilled in the art.

Products according to the invention may be used for promoting health of human beings, for example for maintaining, strengthening or promoting bone or cardiovascular health. In one preferred embodiment of the present invention, the functional food can be used for the prevention or reduction of osteoporosis. In another preferred embodiment of the present invention, regular consumption of said functional food, such as for example once a day, twice a day, or three times a day, leads to a reduction of the risk of diseases such as colds, coughs and reduces tiredness and fatigue.

While the method of administration or consumption may vary, the functional food is preferably ingested by a human as an ingredient of his or her daily diet. Any of the agents described herein can be combined with a liquid vehicle, such as water, milk, vegetable oil, juice and the like, or with an ingestible solid or semi-solid foodstuff. For example, they may be mixed into foods such as milk shakes, milk shake mixes, breakfast drinks, juices, flavored drinks, flavored drink mixes, yogurts, puddings, ice creams, ice milks, frostings, frozen yogurts, cheesecake fillings, candy bars, including “health bars” such as granola and fruit bars, gums, hard candy, mayonnaise, pastry fillings such as fruit fillings or cream fillings, cereals, breads, stuffings, dressings and instant potato mixes. The present invention thus relates to a method of producing a functional food composition, comprising mixing any of the agents described herein (for example lentinan) with a foodstuff.

For example, said functional food product may be selected from the group of meal replacers, dietary supplements, ice-cream, sauces, dressing, spreads, bars, sweets, snacks, cereals and beverages.

In another preferred embodiment, said functional food is dietary supplement, preferably suitable for ingestion in pill, capsule, tablet or liquid form.

In one embodiment, products according to the invention are prepared whereby any of the agents described herein (such as an agent from Lentinus) is added to the food product such that the level of the agent is between 5 to 5000 mg per 100 g product.

### Dairy Product

In one preferred embodiment of the present invention, the functional food is a dairy product. Thus, said functional food may for example be selected from any of the following:

cultured dairy products, yogurts, cottage cheese, cream cheese, dairy dips, sour cream, milkshakes, Butter, Margarine, Low-fat spreads, Cheese, Cottage cheese, Cheese spread, Cheese “strings” for children. Cheese slices, yogurt, Yogurt-based carbonated drinks, drinkable yogurts, low-fat yogurts, refrigerated dips, sour cream, Ice cream, Cream, Low-fat cream-replacement, Fermented milk such as kefir.

In one preferred embodiment of the present invention, said dairy product is a cheese-based product, such as selected from low-fat cheese, hard cheese, soft cheese, cottage cheese, cheese spread, cheese “strings” for children or cheese slices suitable for sandwiches.

In another preferred embodiment of the present invention, said dairy product is a yogurt-based product, such as selected from a set yogurt, a runny or pourable yogurt, a yogurt-based carbonated drink, a drinking or drinkable yogurt, a low-fat yogurt. Said yogurt-based product may for example be fermented with Lactobacillus bulgaricus and/or Streptococcus thermophilus.

In another preferred embodiment of the present invention, said dairy product is a cultured dairy product, such as a cultured fluid (for example drinkable yogurt/yogurt smoothies, kefir, probiotic shots); a non-drinkable yogurt (for example in a cup or tubes); and/or another non-pourable cultured dairy product (for example cottage cheese, cream cheese, dairy dips or sour cream).

In another preferred embodiment of the present invention, said dairy product is another type of dairy product, such as selected from the group consisting of: refrigerated dips and sour cream, ice cream, cream, low-fat cream-replacement, fermented milk such as kefir, fermented beverages, such as drinkable yogurt and kefir.

### Health Drink

In another preferred embodiment of the present invention, the functional food according to the present invention is a health drink. Said health drink is in one embodiment fruit juice-based, which may be concentrated as a “squash”, to be diluted to taste. Said fruit juice or squash preferably comprises concentrated fruit juice. Preferred fruit juices include, but are not restricted to, citrus fruit juices such as orange, grapefruit, lemon or lime, or combinations thereof. In another preferred embodiment, said fruit juice or squash comprises (preferably concentrated) berry juice(s), such as from raspberries, strawberries, blackberries, loganberries, cranberries, redcurrants, blackcurrants, blueberries, or combinations thereof, and/or combinations with citrus fruit juices. In another preferred embodiment, said fruit juice or squash comprises juice(s) from one or more of Pineapple, Passion Fruit, Mango, apple, pear, apricot, Pomegranate, guava, tomato and/or combinations with any other types of fruit juices. Preferred juice bases are selected from the following group:
Apple
Apricots
Banana
Blackberries
Blueberries
Carambola (Starfruit)
cherries
Dates
Figs
fruit cocktail
grape
grapefruit
Kiwi Fruit
Lemons
Mandarin Orange
Mangos
melon
Nectarines
Orange
Papaya
Peaches
Pear
Pineapple
Plantain
Plum
Raspberries
strawberries
Tangerines
watermelon
   or combinations thereof.

Further preferred juice bases are selected from the following group:
Apple
Carrot
Cranberry
Grape
Grapefruit (pink or white)
Lemon
Lime
Orange
Pineapple
Pineapple
Prune
Tangerine
Tomato
   or combinations thereof.

Said health drink may also be water-based, such as a mineral water-based product, such as flavoured mineral water-based products. Said flavouring is preferably from fruit juices and/or other natural products.

In one preferred embodiment of the present invention, said health drink is an energy shot comprising sugars and other energy-providing products, such as comprised in an 25 or 30 cl bottle.

In another preferred embodiment of the present invention, said health drink is an alcoholic beverage, such as a dairy-based alcoholic beverage.

In another preferred embodiment of the present invention, said health drink is a meal replacement drinks.

It is envisaged that the health drink of the present invention may also be manufactured as a concentrate or premix, ready for making up to the drink at a later stage, preferably by the consumer.

### Solid Functional Food

In one preferred embodiment of the present invention, the functional food is a solid functional food, such as selected from the group consisting of: Biscuits/crackers, breakfast cereal, soup, muesli, Chewing gum, Sweets (such as boiled sweets), fresh bakery products (fresh bread, cakes, muffins, waffles etc.), dry bakery products (crispbread, biscuits, crackers etc.), cereal products (breakfast cereals, fibre and sterol enriched flours, mueslis, cereal based and muesli bars, such bars possibly containing chocolate, pasta products, snacks etc.), bran products (granulated and/or toasted bran products, flavoured and/or sterol coated bran products and bran-bran mixes etc.).

In another preferred embodiment of the present invention, said solid functional food is a ready mix (preferably in powder form), either for baking (e.g. breads, cakes, muffins, waffles, pizzas, pancakes) or for cooking (e.g. soups, sauces, desserts, puddings) to be used in preparing or manufacturing of foods

In another preferred embodiment of the present invention, said solid functional food is a meat product (sausages, meat-balls, cold cuts etc.)

In another preferred embodiment of the present invention, said solid functional food is a bread or morning product/bakery snack. Thus, said bread may be white, brown or wholemeal bread. In another preferred embodiment of the present invention, said bread may be selected from the following bread types: malted wheats, milk breads, bran-enriched and mixed grain breads. The bread may be any shape, such as e.g. cob, coburg, cottage, cholla, bloomer, barrel, batch, sandwich, tin, vienna or farmhouse. In one preferred embodiment of the present invention, said bread is selected from any of the following bread types:

Wholemeal bread
Brown bread
Wheatgerm bread (bread containing added processed wheatgerm of no less than 10%)
Softgrain bread (made from white flour with additional grains of softened rye and wheat to increase the fibre content (preferably by 30%) compared with conventional white bread)
Granary breads
Malt breads

In another preferred embodiment of the present invention, said bread is selected from any of the following bread types:

### Ciabatta

pitta
naan
cholla

### Focaccia

Soda Bread or brown soda bread (made using wholemeal flour)
rye breads
baguette or French stick
croissants
bagel

In another preferred embodiment of the present invention, said bread is a flat bread, such as selected from any of the following bread types: Chapattis, Paratas and Roti, Mexican tortilla, flat “wrap” or flour tortilla, pancakes.

In another preferred embodiment of the present invention, the functional food is a morning snack or bakery product. Said bakery product may be either sweet or savoury, for example savoury.

Preferred bakery products include, but are not restricted to: rolls and baps, toasting products such as muffins, crumpets and pikelets, scones, teacakes, buns and other fruited products, hot plate products such as pancakes and griddle scones, waffles and potato cakes, hot cross buns. croissants, brioches, pain-au-chocolat, bagels, American sweet muffins and other semi-sweet bread products.

### Vegetable Oil-Based Product

In another preferred embodiment of the present invention, the functional food is a vegetable oil-based product (spreads, salad oils, mayonnaise etc.)

### Frozen Confectionery Products

In another preferred embodiment of the present invention, the functional food is a frozen confectionary product. For the purpose of the invention the term frozen confectionery product includes milk containing frozen confections such as ice-cream, frozen yogurt, sherbet, sorbet, ice milk and frozen custard, water-ices, granitas and frozen fruit purees.

Preferably the level of solids in the frozen confection (e.g. sugar, fat, flavouring etc) is more than 3 wt %, more preferred from 10 to 70 wt %, for example 40 to 70 wt %.

Ice-cream will typically comprise 2 to 20 wt % of fat, 0 to 20 wt % of sweeteners, 2 to 20 wt % of non-fat milk components and optional components such as emulsifiers, stabilisers, preservatives, flavouring ingredients, vitamins, minerals, etc, the balance being water. Typically ice-cream will be aerated e.g. to an overrun of 20 to 400%, more general 40 to 200% and frozen to a temperature of from −2 to −200 degrees C., more general −10 to −30 degrees C. Ice-cream normally comprises calcium at a level of about 0.1 wt %.

A typical size of an average serving of frozen confectionery material is 66 g. The agent according to the present invention may be encapsulated or combined with emulsifiers, detergents or other agents to ensure solubilisation and stabilisation of the substance in the product.

### Meal Replacers

In another preferred embodiment of the present invention, the functional food is a meal replacer. Meal replacer drinks are typically based on a liquid base which may for example be thickened by means of gums or fibers and whereto a cocktail of minerals and vitamins are added. The drink can be flavoured to the desired taste e.g. fruit or choco flavour. A typical serving size may be 330 ml or 330 g. The agent according to the present invention may be encapsulated or combined with emulsifiers, detergents or other agents to ensure solubilisation and stabilisation of the substance in the beverage.

Meal replacer snacks or bars often comprise a matrix of edible material wherein the agent according to the present invention can be incorporated. For example the matrix may be fat based (e.g. couverture or chocolate) or may be based on bakery products (bread, dough, cookies etc) or may be based on agglomerated particles (rice, grain, nuts, raisins, fruit particles). A typical size for a snack or meal replacement bar could be 20-200 g, generally from 40 to 100 g. Further ingredients may be added to the product e.g. flavouring materials, vitamins, minerals etc.

### Combinations

In one aspect of the present invention, the functional food comprises an agent according to the present invention in combination with another survival enhancing agent, longevity enhancing agent, health enhancing agent and/or a modulator of a microbial population.

For example, one preferred embodiment of said functional food is a food comprising one or more of the agents according to the present invention and a probiotic, such as in a probiotic “shot”. Another preferred embodiment of the functional food is a food comprising the compounds according to the present invention and a prebiotic, such as in a prebiotic “shot”. Another preferred embodiment of the functional food is a food comprising the compounds according to the present invention and a symbiotic, such as in a symbiotic “shot”. In one preferred embodiment of the present invention, preferred bacteria for use in the above-mentioned shots are any of the following: Lactobacillus sp., such as L. acidophilus, L. casei, L. fermentum, L. johnsonii, L. lactis, L. plantarum, L. reuteri, L. rhamnosus and/or L. salivarius. In another preferred embodiment of the present invention, preferred bacteria for use in the above-mentioned shots are any of the following: Bifidobacterium sp., such as B. bifidium, B. breve, B. lactis, and/or B. longum. In another preferred embodiment of the present invention, preferred bacteria for use in the above-mentioned shots are any of the following: Enterococcus faecalis. Escherichia coli, Saccharomyces boulardii, Saccharomyces cerevisiae and/or Streptococcus thermophilus.

The agent(s) according to the present invention may be also combined with other ingredients in a dietary supplement, such as e.g. botanical supplements and/or in a vitamin E capsules, or in a selenium pill. Further preferred combination in said dietary supplements may be with e.g. one or more of the following: antioxidant(s), vitamin C, vitamin E, beta-carotene

The functional food of the invention can further encompass other healthy components such as for example vitamins A, B, C, D, E, minerals such as calcium, potassium, magnesium, iron, copper, zinc, selenium and anti-oxidants such as tocopherols, polyphenols. For example, the functional food may comprise an agent according to the invention (such as lentinan) together with vitamin C, the combination capable of causing a reduction in colds and flu in the individual ingesting said functional food.

In a preferred embodiment, compositions of the invention may comprise further ingredients which are believed to reduce or prevent osteoporosis. Examples of such ingredients are calcium, vitamin D, magnesium etc.

Preferred embodiments of suitable functional foods of the invention are described herein below:

Beverage comprising any of the agents described herein in an amount of 0.1-5%, preferably 0.5-1%.

Fresh bakery product comprising any of the agents described herein in an amount of 0.9-16%, preferably 2.4-10%, and more preferably 3-5%.

Dry bakery product comprising any of the agents described herein in an amount of 1.0-20%, preferably 3.2-15% and more preferably 4.4-10%

Cereal product comprising any of the agents described herein in an amount of 0.8-20%, preferably 1.6-16%, more preferably 2-10%

Bran product comprising any of the agents described herein in an amount of 4%-25%, preferably 6-20%

Dairy or non-dairy product (e.g. fermented cereal product) comprising any of the agents described herein in an amount of 0.1-20%, preferably 0.8-8%

Vegetable oil based product comprising any of the agents described herein in an amount of 0.6-16%, preferably 2.6-10%, more preferably 2.6-5%

Meat product comprising any of the agents described herein in an amount of 0.1-16%, preferably 0.2-5%.

Dairy product comprising: any of the agents described herein in an amount of 0.1-16%, preferably 0.2-5%.

Thus, in one embodiment, the present invention is concerned with use of any of the agents described herein in the manufacture of a functional food, such as any of the functional foods described herein.

### Survival

In one embodiment of the invention it is preferred that the food or feed product is capable of improving survival, preferably after oral intake. Thus when the feed product according to the invention is fed to one group of animals, the lethal rate should be lower than in a similar control group fed on a similar diet. The lethal rate is determined as the percentage of dead animals after a predetermined time.

Preferably, the lethal rate is at least 1.2, such as 1.5, for example 2, such as 3, for example 5, such as 10 times lower in animals fed with a feed product according to the present invention.

In particular, the above-mentioned reduction in lethal rate is observed in aquatic animals, preferably in cods, salmonids, gadids or penaeids, more preferably in cods after 1 week, such as after 2 weeks, for example after 3 weeks, such as after approximately 30-45 days, wherein said aquatic animal has been fed with a feed product according to the invention during the entire period.

In another embodiment of the invention the above-mentioned reduction in lethal rate is observed in a farm animal, preferably chicken or pigs after 2 weeks, such as 1 months, such as 2 months, for example 3 months, wherein said farm animal has been fed with the product for at least 2 weeks, such as for the entire period.

In particular, the above-mentioned reduction in lethal rate is preferably observed in chicken 35 days after hatching, wherein the chickens have been fed the feed product according to the invention continuously since hatching.

In another embodiment the above-mentioned reduction in lethal rate is preferably observed in piglets 28 days after weaning, wherein the piglets have been fed the feed product according to the invention continuously since weaning.

Methods for investigating survival are described in the Examples below.

### Enhanced Growth

In one embodiment of the invention it is preferred that the food or feed product is capable of improving growth, preferably after oral intake. In particular it is preferred that the food or feed product is capable of improving growth in young animals.

Preferably, when the feed product according to the present invention is fed to one group of animals, the average weight gain is higher than in a similar control group fed on a similar diet. The average weight gain may be determined as the difference in weight gain between the groups divided by the average weight in the control group. The weight may be the weight of the living animals or the market weight.

More preferably, the average weight gain is at least 13%, more preferably at least 15%, such as at least 20%, for example at least 25%

Preferably, above mentioned weight gain is obtained in farm animals, such as chicken or pig, at the age of 2 weeks, such as 3 weeks, for example 4 weeks, such as 2 months, wherein the animals are fed the feed product continuously from day 0, such as from day 1, for example from day 4, such as from day 7 after birth/hatching

In particular, the above-mentioned average weight gain is preferably observed in chicken 35 days after hatching, wherein the chickens have been fed the feed product according to the invention continuously since hatching.

In another embodiment the above-mentioned weight gain is preferably observed in piglets 28 days after weaning, wherein the piglets have been fed the feed product according to the invention continuously since weaning.

In one embodiment animals fed with the feed product according to the present invention obtain a larger weight gain than animals fed on another nutritionally similar diet comprising traditional growth stimulators, such as antibiotics, for example virginiamycin. Preferably the weight gain is at least 5%, such as at least 10%, for example at least 15% larger.

### Modulation of Microbial Population

In one embodiment of the invention it is preferred that the food or feed product is capable of modulating the microbial population in at least part of the digestive tract, in particular after oral intake. The digestive tract may depending on the animal species comprise the crop, oesophagus, proventriculus, gizzard, duodenum, jejunum, ileum and caecae. Preferably the food or feed product is capable of at least modulating the microbial population in the intestine.

Modulation of the microbial population in the intestine may include one or more of the following (A-G), wherein the percent modulation may be in comparison with either the animal/human being before being fed the feed or food product according to the invention or another similar animal or human being, which is not fed the feed or food product, preferably an animal or human being which is on a similar diet lacking the survival enhancing agent according to the invention:

A. Reduction of the overall number of bacteria in the intestine, preferably the reduction is to 90% or less, such as to 80% or less, for example to 70% or less, such as to 60% or less, for example to 50%. This may for example be determined by preparing an intestinal sample and determining the size of the bacterial population.
B. Reduction of the number of Salmonella, such as Salmonella enterica, preferably reduction in the number of Salmonella in the intestine. Preferably the reduction is to 90% or less, such as to 80% or less, for example to 70% or less, such as to 60% or less, for example to 50% or less, such as 40% or less, for example to 30% or less, such as 20% or less for example to 10% or less, such as 5% or less, for example to 1%. This may for example be determined by preparing an intestinal sample and determining presence of Salmonella.
C. Reduction of the number of Clostridium perfringens, preferably reduction in the number of Clostridium perfringens in the intestine. Preferably the reduction is to 90% or less, such as to 80% or less, for example to 70% or less, such as to 60% or less, for example to 50% or less, such as 40% or less, for example to 30% or less, such as 20% or less for example to 10% or less, such as 5% or less, for example to 1%. This may for example be determined by preparing an intestinal sample and determining presence of Clostridium perfringens.
D. Reduction of the number of Camphylobacter jejuni, preferably reduction in the number of Camphylobacter jejuni in the intestine. Preferably the reduction is to 90% or less, such as to 80% or less, for example to 70% or less, such as to 60% or less, for example to 50% or less, such as 40% or less, for example to 30% or less, such as 20% or less for example to 10% or less, such as 5% or less, for example to 1%. This may for example be determined by preparing an intestinal sample and determining presence of Camphylobacter jejuni.
E. Reduction of the number of coccids, preferably reduction in the number of coccids in the intestine. Preferably the reduction is to 90% or less, such as to 80% or less, for example to 70% or less, such as to 60% or less, for example to 50% or less, such as 40% or less, for example to 30% or less, such as 20% or less for example to 10% or less, such as 5% or less, for example to 1%. This may for example be determined by preparing an intestinal sample and determining presence of coccids.
F. No or minor reduction in the number of Lactobacillus sp., preferably reduction to no less than 80%, such as no less than 90%, for example to no less than 95%, such as to no less than 98%. This may for example be determined by preparing an intestinal sample and determining presence of Lactobacillus.
G. No or minor reduction in the number of Bifidobacterium sp., preferably reduction to no less than 80%, such as no less than 90%, for example to no less than 95%, such as to no less than 98%. This may for example be determined by preparing an intestinal sample and determining presence of Bifidobacterium.

### Fungi

In a preferred embodiment of the present invention the survival enhancing agent disclosed herein have been produced by a fungus. Preferably, the survival enhancing agent has been purified from the extracellular environment of a fungus. Even more preferably the fungus, preferably a fungal mycelium, has been cultivated in a liquid growth medium and said survival enhancing agent has been purified from said liquid growth medium.

It is thus preferred that the survival enhancing agent of the invention has been produced by a method comprising the steps of
i) cultivating a fungus, such as a fungal mycelium, in a liquid growth medium, and
ii) isolating the composition from said liquid growth medium

By fungal mycelium is intended any fungal biomass, which can be grown in a submerged culture. The fungal biomass may be in the form of single hyphae, spores, aggregates of mycelium, and partly differentiated mycelium.

The liquid growth medium may be any of the liquid growth media described herein below.

The fungus may be any fungus, preferably a fungus forming a fungal mycelium, more preferably the fungus is a filamentous fungus. Even more preferably, the fungus may be selected from the group consisting of Agaricus sp., such as Agaricus bisporus, Cordiceps sinensis, Flammulina velutipes, Ganoderma lucidum, Grifola frondosa, Lentinus edodes, Pleurotus ostreatus, Schizophyllum commune, Sclerotina sclerotium, Trametes (Coriolus) versicolor, Tremella fuciformis, Agaricus blazei, Agrocybe aegerita, Agrocybe cylindracea, Albatrellus confluens, Armillariella mellea, Auricularia auricula-judae, Auricularia polytricha, Collybia maracula, Cordiceps militari, Dendropolyporus umbellatus, Fomes fomentarius, Fomes pinicola, Ganoderma applanatum, Ganoderma tsugae, Hericium erinaceus, Hypsizygus marmoreus, Inonotus obliquus, Laetiporus sulphurous, Lenzites betulinus, Leucopaxilllus giganteus, Lyophyllum cinerascens, Omphalina epichysium, Oudemansiella mucida, Panellus serotinus, Piptoporus betulinus, Phellinus linteus, Phellinus pini, Pholiota nameko, Pleurotus citrinopileatus, Pleurotus pulmonarius, Sarcedon asparatus, Trametes suavolens, Volvariella volvacea and Wolfiporia cocos.

Yet more preferably, the fungus is selected from the group consisting of Agaricus sp., such as Agaricus bisporus, Agaricus blazei, Cordiceps sinensis, Flammulina velutipes, Ganoderma lucidum, Grifola frondosa, Lentinus edodes, Pleurotus ostreatus, Schizophyllum commune, Sclerotina sclerotium, Trametes (Coriolus) versicolor and Tremella fuciformis.

In a very preferred embodiment of the present invention the fungus belongs to the class of basidiomycetes, such as a fungus of the genus Lentinus, such as Lentinus edodes.

Lentinus edodes deposited under IHEM 18992 with the Belgian Coordinated Collections of Microorganism (BCCM), 14 Rue J. Wytsman, B-1050 Bruxelles, Belgium, represents one preferred strain of Lentinus edodes. Further strains of Lentinus edodes are available from culture collections such as ATCC (American Type Culture Collection, P.O. Box 1549, Manassas, Va. 20108, USA), CBC (Centraalbureau voor Schimmelcultures, PO Box 85167, 3508 AD Utrecht, THE NETHERLANDS) and DSMZ (Deutsche Sammlung von Mikroorganismen and Zellkulturen GmbH, Mascheroder Weg 1b, 38124 Braunschweig GERMANY).

Additionally relevant Lentinus species, besides Lentinus edodes, includes Lentinus species such as: Lentinus albovelutinus G. Stev. (1964)=Rhodocybe albovelutina (G. Stev.) E. Horak (1971); Lentinus anthocephalus (Lév.) Pegler; Lentinus badius Bres.; Lentinus castoreus Fr. (1838)=Lentinellus ursinus (Fr.) Kühner (1926); Lentinus chrysopeplus Berk. & M. A. Curtis (1869)=Cyptotrama asprata (Berk.) Redhead & Ginns (1980); Lentinus cochleatus Fr.; Lentinus concinnus Pat.; Lentinus delicatus G. Stev. (1964)=Marasmius delicatus (G. Stev.) E. Horak (1971); Lentinus fasciatus Berk.; Lentinus hepatotrichus Berk. (1859)=Lentinellus ursinus (Fr.) Kühner (1926); Lentinus hyracinus Kalchbr. (1880)=Lentinellus ursinus (Fr.) Kühner (1926); Lentinus lepideus sensu Colenso (1891); Lentinus lepideus (Fr.) Fr. (1825)=Lentinus suffrutescens (Brot.) Fr. (1825); Lentinus novaezelandiae Berk. (1855)=Lentinellus ursinus (Fr.) Kuhner (1926); Lentinus pulvinulus Berk. (1859)=Lentinellus pulvinulus (Berk.) Pegler (1965); Lentinus punctaticeps Berk. & Broome (1883); Lentinus punctaticeps cf. sensu Petersen, Nicholl & Hughes (1997); Lentinus pygmaeus Colenso (1887)=Lentinus zelandicus Sacc. & Cub. (1887); Lentinus sajor-caju (Fr.) Fr.; Lentinus squarrulosus Mont.; Lentinus strigosus (Schwein.) Fr. (1825); Lentinus suffrutescens (Brot.) Fr. (1825); and Lentinus tuber-regium Fr.; Lentinus zelandicus Sacc. & Cub. (1887) (Ref: http://nzfungi.landcareresearch.co.nz).

### Methods of Liquid Cultivation

Preferably, the Basiomyctes, such as Lentinus sp. or Agaricus sp. are cultivated in a liquid growth medium.

Cultivating the fungus in a liquid growth medium in general involves dissolving nutrient compounds required for growth of said fungus in water, transferring the solution to a bioreactor and inoculating the bioreactor with cells or spores of the fungus, such as a fungal mycelium, or fractions thereof, to be cultivated. This is done under sterile conditions and with control of the environment in order to give the fungus a suitable chemical and physical environment. Cultivating fungi in liquid growth medium is also termed “liquid state” cultivation.

During “liquid-state” cultivation the medium with the fungal biomass is preferably agitated to reduce the occurrence of gradients and to ensure oxygen availability to the submerged cells. When fungi are grown in a bioreactor, oxygen may be supplied to the liquid medium and the level of dissolved oxygen may be controlled by known methods.

The liquid growth medium is an aqueous solution, preferably sterile water, comprising nutrient compounds. The liquid medium supports fungal growth and preferably stimulates the production of extracellular compounds, such as immune modulating agents. The liquid growth medium may comprise one or more typical ingredients required for growth of microbial organisms such as malt extract, yeast extract, peptone, glucose, sucrose, sucrose, salts providing phosphate, magnesium and potassium, corn-steep liquor and vitamins such as thiamine. More preferably, the medium comprises sucrose, corns steep liquor, phosphate and magnesium for mycelium growth and production of polysaccharides.

In a preferred embodiment for liquid cultivation the medium comprises malt extract. This embodiment is in particular relevant for production of food or feed products comprising biomass or a composition isolated from biomass. More preferably the medium may comprise malt extract, a sugar source and an amino acid source, even more preferably malt extract, glucose, yeast extract and peptone. The malt extract may preferably be at a concentration in the range of 1 to 20, such as 1 to 10, for example 2 to 4 g/l. Glucose may preferably be at a concentration of less than 18 g/l, such as in the range of 10 to 18, for example in the range of 13 to 17 g/l. Peptone may preferably be at a concentration of less than 9, such as in the range of 1 to 9, for example in the range of 3 to 7 g/l. Yeast extract may preferably be in a concentration of in the range of 1 to 10, preferably around 3 g/l.

For inoculation of the growth medium, fungal mycelium, such as Lentinus edodes mycelium from agar plates containing for example malt extract, yeast extract, peptone and glucose can be used. Fungi can initially be cultivated on agar plates comprising the above nutrient compounds supporting the growth of the fungus. The plates are inoculated with mycelium and incubated at least until a visible growth is evident on the plates. Dependent on the fungus, this usually can take from about 7 days to about 24 days or from about 10 to 30 days, typically 14 days or up to 20 days, at a temperature in the range of from 18 to 32° C., preferably in the area of from 22 to 30° C., such as a temperature of about 23° C. to 27° C., such as around 25° C.

As an alternative to inoculation with mycelium from agar plates, inoculation of the growth medium can be carried out by using mycelium from a fermentation broth in e.g. a shake flask medium comprising nutrient compounds supporting cell growth.

Shake flasks for cultivating fungal mycelium can initially be inoculated with the mycelium which is cultivated on agar plates. The mycelium is taken from the plates and transferred aseptically to shake flasks containing sterile water comprising dissolved nutrient compounds and nutrient salts supporting the growth of the fungal mycelium. A typical growth medium contains sucrose, corn steep liquor, phosphate and a magnesium. The amount of inoculation material which gives the highest production of extracellular lentinan can be selected following initial experiments.

The time for incubation of the shake flasks depends on the specific fungus. Typically, the shake flasks can be incubated by shaking for 6 to 21 days, preferably from 7 to 18 days, more preferably from 8 to 14 days at a temperature in the range of from 18 to 32° C., preferably in the area of from 22 to 30° C., such as a temperature of about 23° C., for example 24° C., such as 25° C., for example 26° C., such as 27° C., for example 28° C., such as 29° C., for example 30° C. The shake flasks may also be incubated from 8-25 days, more preferably from 10-20 days, more preferably from 12-18 days. The temperature may also be from 18 to 37° C., preferably from 23 to 32° C. such as about 25° C.

The content of the shake flasks can be used for inoculating a bioreactor. In that case, the reactor comprises a sterile solution of nutrient compounds and nutrient salts in water for mono-culture cultivation of basidiomycete fungal mycelium, or fractions thereof, such as Lentinus fungal mycelium, such as Lentinus edodes.

The bioreactor fermentation period is typically in the range of from 50 hours to 300 hours, preferably in the range of from 80 hours to 270 hours, and the temperature is kept constant in the range of 18 to 32° C., preferably in the area of from 22 to 31° C., such as a temperature of about 23° C., for example 24° C., such as 25° C., for example 26° C., such as 27° C., for example 28° C., such as 29° C., for example 30° C. The temperature may also be from 18 to 37° C., preferably from 23 to 32° C. such as about 25° C.

The reactor is fitted with an inlet for supplying air to the fermentation broth, and the fermentation broth is preferably kept under continuous agitation either as a result of the addition of air, or by means of a mixer device suitable for providing a good mixing of the content of the reactor.

It is preferred to adjust the pH of the growth medium to from about 3 to about 7, such as a pH of from about 4.5 to about 6.5, for example a pH of about 6, before the growth medium is inoculated with fungal mycelium, or fractions thereof, such as L. edodes mycelium. After the initial adjustment, pH may be dropped naturally during the course of the fermentation, or controlled at a particular value in the range pH 3 to 7, using addition of suitable pH-control agents, such as acid and base. The temperature of the growth medium is preferably in the range of from 18 to 32° C., preferably in the area of from 22 to 31° C., such as a temperature of about 23° C., for example 24° C., such as 25° C., for example 26° C., such as 27° C., for example 28° C., such as 29° C., for example 30° C. The temperature may also be from 18 to 37° C., preferably from 23 to 32° C. such as about 25° C.

Samples can be obtained from the bioreactor and analysed for biomass, metabolic products and nutrient compounds, the determinations of which can assist the operator of the bioreactor in the running of the fermentation process. Typical analyses routinely carried out are determination of biomass, residual sugar concentration and extracellular polysaccharide concentration. A person skilled in the art knows the methods for analysis which can be employed in this respect.

### Isolating a Composition Comprising a Survival Enhancing Agent

Preferably, the method for preparing the products according to the invention involves a step of purifying the extracellular fraction of the liquid growth medium from the fungal mycelium. The extracellular fraction of the liquid fermentation medium is also termed the supernatant and this fraction can be separated from the fungal mycelium by e.g. centrifugation or filtration, or indeed by any other means available for obtaining a liquid fraction essentially without any fungal mycelium present therein. The term “essentially without any fungal mycelium present therein” shall denote that the concentration of fungal mycelium, including fractions thereof, has been reduced at least by a factor of 10³, such as reduced by a factor of at least 10⁴, for example a factor of at least 10⁵, such as reduced by a factor of at least 10⁶.

The methods for preparing the products according to the invention may further comprise isolating an extracellular composition comprising a survival enhancing agent. In preferred embodiments of the invention the isolation comprises at least one size fractionation step. Preferably, this size fractionation step is performed on the extracellular fraction. This size fractionation step may ensure that every polysaccharide of the composition has a molecular weight of at least a given value (see also herein above). The size fractionation step may be any size fraction known to the skilled person, for example ultracentrifugation, ultrafiltration, microfiltration or gelfiltration. Thus in a preferred embodiment of the invention, the composition is purified from a liquid growth medium by a method involving one or more purification steps selected from the group consisting of ultracentrifugation, ultrafiltration, microfiltration and gelfiltration. Preferably, the purification step(s) are selected from the group consisting of ultrafiltration, microfiltration and ultracentrifugation, even more preferably from the group consisting of ultrafiltration and microfiltration.

Ultrafiltration is a membrane process where the membrane fractionates components of a liquid according to size. The membrane configuration is normally cross-flow wherein the liquid containing the relevant components are flowing across the membrane. Some of the liquid, containing components smaller than the nominal pore size of the membrane will permeate through the membrane. Molecules larger than the nominal pore size will be retained. The desired product may be in the retentate or the filtrate. If the ultrafiltration is performed in order to prepare a composition, wherein every polysaccharide within said composition has a molecular weight above a given value, the desired product is in the retentate. If a serial fractionation is made, the product may be in the retentate or filtrate.

Microfiltration is a membrane separation process similar to UF but with even larger membrane pore size allowing larger particles to pass through.

Gel filtration is a chromatographic technique in which particles are separated according to size. The filtration medium will typically be small gel beads which will take up the molecules that can pass through the bead pores. Larger molecules will pass through the column without being taken up by the beads.

Gel-filtration, ultrafiltration or microfiltration may for example be performed as described in R Hatti-Kaul and B Mattiasson (2001), Downstream Processing in Biotechnology, in Basic Biotechnology, eds C Ratledge and B Kristiansen, Cambridge University Press) pp 189.

A non-limiting method of preparing the products according to the invention is described in example 1.

In another embodiment the extracellular composition may be isolated by precipitation, such as precipitation with alcohol, such as ethanol and/or chromatographic methods. This may for example be performed essentially as described in WO2003/020944. It is also comprised within the invention that the extracellular composition is isolated by sequentially performing two or more of above-mentioned methods. By way of example the composition may be isolated by first performing a size fractionation step followed by precipitation.

The feed or food product according to the invention may also be prepared using the biomass, which comprises the fungal mycelium. Biomass may be prepared as described above, except that the fungal mycelium rather than the extracellular material is used.

Once the biomass is obtained it may be employed as such, it may be dried or a composition comprising a survival enhancing agent may be further isolated from the biomass. Said composition may for example be isolated by means of extraction.

In one aspect there is provided a bioactive agent as disclosed in the items herein below:
1. The bioactive agent according to a first item comprises or consists of an agent selected from an oligosaccharide, a polysaccharide and an optionally glycosylated polypeptide.
2. The bioactive agent according to item 1, wherein the bioactive agent comprises or consists of a polysaccharide.
3. The bioactive agent according to item 1, wherein the bioactive agent comprises or consists of an oligosaccharide.
4. The bioactive agent according to item 1, wherein the bioactive agent comprises or consists of an optionally glycosylated polypeptide.
5. The bioactive agent according to item 2, wherein the polysaccharide is a homopolymer.
6. The bioactive agent according to item 2, wherein the polysaccharide is a heteropolymer.
7. The bioactive agent according to items 2, wherein the polysaccharide comprises glucose monosaccharide units, optionally in combination with further monosaccharide units selected from the group of units consisting of glucuronic acid, galactose, mannose, arabinose and xylose, including any combination thereof.
8. The bioactive agent according to item 7, wherein the further monosaccharide units are all glucuronic acid.
9. The bioactive agent according to item 7, wherein the further monosaccharide units are all galactose.
10. The bioactive agent according to item 7, wherein the further monosaccharide units are all mannose.
11. The bioactive agent according to item 7, wherein the further monosaccharide units are all arabinose.
12. The bioactive agent according to item 7, wherein the further monosaccharide units are all xylose.
13. The bioactive agent according to item 7, wherein the further monosaccharide units are glucuronic acid and galactose.
14. The bioactive agent according to item 7, wherein the further monosaccharide units are glucuronic acid and mannose.
15. The bioactive agent according to item 7, wherein the further monosaccharide units are glucuronic acid and arabinose.
16. The bioactive agent according to item 7, wherein the further monosaccharide units are glucuronic acid and xylose.
17. The bioactive agent according to item 7, wherein the further monosaccharide units are galactose and mannose.
18. The bioactive agent according to item 7, wherein the further monosaccharide units are galactose and arabinose.
19. The bioactive agent according to item 7, wherein the further monosaccharide units are galactose and xylose.
20. The bioactive agent according to item 7, wherein the further monosaccharide units are mannose and arabinose.
21. The bioactive agent according to item 7, wherein the further monosaccharide units are mannose and xylose.
22. The bioactive agent according to item 7, wherein the further monosaccharide units are arabinose and xylose.
23. The bioactive agent according to item 7, wherein the further monosaccharide units are glucuronic acid, galactose and mannose.
24. The bioactive agent according to item 7, wherein the further monosaccharide units are glucuronic acid, galactose and arabinose.
25. The bioactive agent according to item 7, wherein the further monosaccharide units are glucuronic acid, galactose and xylose.
26. The bioactive agent according to item 7, wherein the further monosaccharide units are glucuronic acid, mannose and arabinose.
27. The bioactive agent according to item 7, wherein the further monosaccharide units are glucuronic acid mannose and xylose.
28. The bioactive agent according to item 7, wherein the further monosaccharide units are glucuronic acid, arabinose and xylose.
29. The bioactive agent according to item 7, wherein the further monosaccharide units are galactose, mannose and arabinose.
30. The bioactive agent according to item 7, wherein the further monosaccharide units are galactose, mannose and xylose.
31. The bioactive agent according to item 7, wherein the further monosaccharide units are galactose, arabinose and xylose.
32. The bioactive agent according to item 7, wherein the further monosaccharide units are mannose, arabinose and xylose.
33. The bioactive agent according to item 7, wherein the further monosaccharide units are glucuronic acid, galactose, mannose and arabinose.
34. The bioactive agent according to item 7, wherein the further monosaccharide units are glucuronic acid, galactose, mannose and xylose.
35. The bioactive agent according to item 7, wherein the further monosaccharide units are glucuronic acid, galactose, arabinose and xylose.
36. The bioactive agent according to item 7, wherein the further monosaccharide units are glucuronic acid, mannose, arabinose and xylose.
37. The bioactive agent according to item 7, wherein the further monosaccharide units are galactose, mannose, arabinose and xylose.
38. The bioactive agent according to item 2, wherein the backbone of the polysaccharide comprises glucose monosaccharide units in combination with further monosaccharide units selected from the group of units consisting of glucuronic acid, galactose, mannose, arabinose and xylose, including any combination thereof.
39. The bioactive agent according to item 38, wherein the further monosaccharide units are all glucuronic acid.
40. The bioactive agent according to item 38, wherein the further monosaccharide units are all galactose.
41. The bioactive agent according to item 38, wherein the further monosaccharide units are all mannose.
42. The bioactive agent according to item 38, wherein the further monosaccharide units are all arabinose.
43. The bioactive agent according to item 38, wherein the further monosaccharide units are all xylose.
44. The bioactive agent according to item 38, wherein the further monosaccharide units are glucuronic acid and galactose.
45. The bioactive agent according to item 38, wherein the further monosaccharide units are glucuronic acid and mannose.
46. The bioactive agent according to item 38, wherein the further monosaccharide units are glucuronic acid and arabinose.
47. The bioactive agent according to item 38, wherein the further monosaccharide units are glucuronic acid and xylose.
48. The bioactive agent according to item 38, wherein the further monosaccharide units are galactose and mannose.
49. The bioactive agent according to item 38, wherein the further monosaccharide units are galactose and arabinose.
50. The bioactive agent according to item 38, wherein the further monosaccharide units are galactose and xylose.
51. The bioactive agent according to item 38, wherein the further monosaccharide units are mannose and arabinose.
52. The bioactive agent according to item 38, wherein the further monosaccharide units are mannose and xylose.
53. The bioactive agent according to item 38, wherein the further monosaccharide units are arabinose and xylose.
54. The bioactive agent according to item 38, wherein the further monosaccharide units are glucuronic acid, galactose and mannose.
55. The bioactive agent according to item 38, wherein the further monosaccharide units are glucuronic acid, galactose and arabinose.
56. The bioactive agent according to item 38, wherein the further monosaccharide units are glucuronic acid, galactose and xylose.
57. The bioactive agent according to item 38, wherein the further monosaccharide units are glucuronic acid, mannose and arabinose.
58. The bioactive agent according to item 38, wherein the further monosaccharide units are glucuronic acid mannose and xylose.
59. The bioactive agent according to item 38, wherein the further monosaccharide units are glucuronic acid, arabinose and xylose.
60. The bioactive agent according to item 38, wherein the further monosaccharide units are galactose, mannose and arabinose.
61. The bioactive agent according to item 38, wherein the further monosaccharide units are galactose, mannose and xylose.
62. The bioactive agent according to item 38, wherein the further monosaccharide units are galactose, arabinose and xylose.
63. The bioactive agent according to item 38, wherein the further monosaccharide units are mannose, arabinose and xylose.
64. The bioactive agent according to item 38, wherein the further monosaccharide units are glucuronic acid, galactose, mannose and arabinose.
65. The bioactive agent according to item 38, wherein the further monosaccharide units are glucuronic acid, galactose, mannose and xylose.
66. The bioactive agent according to item 38, wherein the further monosaccharide units are glucuronic acid, galactose, arabinose and xylose.
67. The bioactive agent according to item 38, wherein the further monosaccharide units are glucuronic acid, mannose, arabinose and xylose.
68. The bioactive agent according to item 38, wherein the further monosaccharide units are galactose, mannose, arabinose and xylose.
69. The bioactive agent according to item 2, wherein the backbone of the polysaccharide comprises a plurality of monosaccharide units, and wherein the side chains of the polysaccharide comprises further monosaccharide units selected from the group of units consisting of glucuronic acid, galactose, mannose, arabinose xylose and glucose, including any combination thereof.
70. The bioactive agent according to item 69, wherein the further monosaccharide units are all glucuronic acid.
71. The bioactive agent according to item 69, wherein the further monosaccharide units are all galactose.
72. The bioactive agent according to item 69, wherein the further monosaccharide units are all mannose.
73. The bioactive agent according to item 69, wherein the further monosaccharide units are all arabinose.
74. The bioactive agent according to item 69, wherein the further monosaccharide units are all xylose.
75. The bioactive agent according to item 69, wherein the further monosaccharide units are all glucose.
76. The bioactive agent according to item 69, wherein the further monosaccharide units are glucuronic acid and galactose.
77. The bioactive agent according to item 69, wherein the further monosaccharide units are glucuronic acid and mannose.
78. The bioactive agent according to item 69, wherein the further monosaccharide units are glucuronic acid and arabinose.
79. The bioactive agent according to item 69, wherein the further monosaccharide units are glucuronic acid and xylose.
80. The bioactive agent according to item 69, wherein the further monosaccharide units are glucuronic acid and glucose.
81. The bioactive agent according to item 69, wherein the further monosaccharide units are galactose and mannose.
82. The bioactive agent according to item 69, wherein the further monosaccharide units are galactose and arabinose.
83. The bioactive agent according to item 69, wherein the further monosaccharide units are galactose and xylose.
84. The bioactive agent according to item 69, wherein the further monosaccharide units are galactose and glucose.
85. The bioactive agent according to item 69, wherein the further monosaccharide units are mannose and arabinose.
86. The bioactive agent according to item 69, wherein the further monosaccharide units are mannose and xylose.
87. The bioactive agent according to item 69, wherein the further monosaccharide units are mannose and glucose.
88. The bioactive agent according to item 69, wherein the further monosaccharide units are arabinose and xylose.
89. The bioactive agent according to item 69, wherein the further monosaccharide units are arabinose and glucose.
90. The bioactive agent according to item 69, wherein the further monosaccharide units are xylose and glucose.
91. The bioactive agent according to item 69, wherein the further monosaccharide units are glucuronic acid, galactose and mannose.
92. The bioactive agent according to item 69, wherein the further monosaccharide units are glucuronic acid, galactose and arabinose.
93. The bioactive agent according to item 69, wherein the further monosaccharide units are glucuronic acid, galactose and xylose.
94. The bioactive agent according to item 69, wherein the further monosaccharide units are glucuronic acid, galactose and glucose.
95. The bioactive agent according to item 69, wherein the further monosaccharide units are glucuronic acid, mannose and arabinose.
96. The bioactive agent according to item 69, wherein the further monosaccharide units are glucuronic acid mannose and xylose.
97. The bioactive agent according to item 69, wherein the further monosaccharide units are glucuronic acid mannose and glucose.
98. The bioactive agent according to item 69, wherein the further monosaccharide units are glucuronic acid, arabinose and xylose.
99. The bioactive agent according to item 69, wherein the further monosaccharide units are glucuronic acid, arabinose and glucose.
100. The bioactive agent according to item 69, wherein the further monosaccharide units are glucuronic acid, xylose and glucose.
101. The bioactive agent according to item 69, wherein the further monosaccharide units are galactose, mannose and arabinose.
102. The bioactive agent according to item 69, wherein the further monosaccharide units are galactose, mannose and xylose.
103. The bioactive agent according to item 69, wherein the further monosaccharide units are galactose, mannose and glucose.
104. The bioactive agent according to item 69, wherein the further monosaccharide units are galactose, arabinose and xylose.
105. The bioactive agent according to item 69, wherein the further monosaccharide units are galactose, arabinose and glucose.
106. The bioactive agent according to item 69, wherein the further monosaccharide units are galactose, xylose and glucose.
107. The bioactive agent according to item 69, wherein the further monosaccharide units are mannose, arabinose and xylose.
108. The bioactive agent according to item 69, wherein the further monosaccharide units are mannose, arabinose and glucose.
109. The bioactive agent according to item 69, wherein the further monosaccharide units are mannose, xylose and glucose.
110. The bioactive agent according to item 69, wherein the further monosaccharide units are arabinose, xylose and glucose.
111. The bioactive agent according to item 69, wherein the further monosaccharide units are glucuronic acid, galactose, mannose and arabinose.
112. The bioactive agent according to item 69, wherein the further monosaccharide units are glucuronic acid, galactose, mannose and xylose.
113. The bioactive agent according to item 69, wherein the further monosaccharide units are glucuronic acid, galactose, mannose and glucose.
114. The bioactive agent according to item 69, wherein the further monosaccharide units are glucuronic acid, galactose, arabinose and xylose.
115. The bioactive agent according to item 69, wherein the further monosaccharide units are glucuronic acid, galactose, arabinose and glucose.
116. The bioactive agent according to item 69, wherein the further monosaccharide units are glucuronic acid, galactose, xylose and glucose.
117. The bioactive agent according to item 69, wherein the further monosaccharide units are glucuronic acid, mannose, arabinose and xylose.
118. The bioactive agent according to item 69, wherein the further monosaccharide units are glucuronic acid, mannose, arabinose and glucose.
119. The bioactive agent according to item 69, wherein the further monosaccharide units are glucuronic acid, mannose, xylose and glucose.
120. The bioactive agent according to item 69, wherein the further monosaccharide units are glucuronic acid, arabinose, xylose and glucose.
121. The bioactive agent according to item 69, wherein the further monosaccharide units are galactose, mannose, arabinose and xylose.
122. The bioactive agent according to item 69, wherein the further monosaccharide units are galactose, mannose, arabinose and glucose.
123. The bioactive agent according to item 69, wherein the further monosaccharide units are galactose, mannose, xylose and glucose.
124. The bioactive agent according to item 69, wherein the further monosaccharide units are galactose, arabinose, xylose and glucose.
125. The bioactive agent according to item 69, wherein the further monosaccharide units are mannose, arabinose, xylose and glucose.
126. The bioactive agent according to item 69, wherein the further monosaccharide units are glucuronic acid, galactose, mannose, arabinose and xylose.
127. The bioactive agent according to item 69, wherein the further monosaccharide units are glucuronic acid, galactose, mannose, arabinose and glucose.
128. The bioactive agent according to item 69, wherein the further monosaccharide units are glucuronic acid, galactose, mannose, xylose and glucose.
129. The bioactive agent according to item 69, wherein the further monosaccharide units are glucuronic acid, galactose, arabinose xylose and glucose.
130. The bioactive agent according to item 69, wherein the further monosaccharide units are glucuronic acid, mannose, arabinose xylose and glucose.
131. The bioactive agent according to item 69, wherein the further monosaccharide units are galactose, mannose, arabinose xylose and glucose.
132. The bioactive agent according to item 2, wherein the polysaccharide comprises a repetitive backbone macromonomer comprising from 2 to 6, such as 2, 3, 4, 5 or 6 different monosaccharide units and having from 1 to 3 monosaccharide units selected from glucose, mannose and galactose.
133. The bioactive agent according to item 2, wherein the polysaccharide comprises an average of from 1 to 1000 monosaccharide units in the backbone between each branching point, such as from 2 to 1000 monosaccharide units, for example from 3 to 1000 monosaccharide units, such as from 4 to 1000 monosaccharide units, for example from 5 to 1000 monosaccharide units, such as from 6 to 1000 monosaccharide units, for example from 7 to 1000 monosaccharide units, such as from 8 to 1000 monosaccharide units, for example from 9 to 1000 monosaccharide units, such as from 10 to 1000 monosaccharide units, for example from 11 to 1000 monosaccharide units, such as from 12 to 1000 monosaccharide units, for example from 13 to 1000 monosaccharide units, such as from 14 to 1000 monosaccharide units, for example from 15 to 1000 monosaccharide units, such as from 20 to 1000 monosaccharide units, for example from 25 to 1000 monosaccharide units, such as from 30 to 1000 monosaccharide units, for example from 40 to 1000 monosaccharide units, such as from 50 to 1000 monosaccharide units, for example from 60 to 1000 monosaccharide units, such as from 70 to 1000 monosaccharide units, for example from 80 to 1000 monosaccharide units, such as from 90 to 1000 monosaccharide units, for example from 100 to 1000 monosaccharide units, such as from 2 to 500 monosaccharide units, for example from 3 to 500 monosaccharide units, such as from 4 to 500 monosaccharide units, for example from 5 to 500 monosaccharide units, such as from 6 to 500 monosaccharide units, for example from 7 to 500 monosaccharide units, such as from 8 to 500 monosaccharide units, for example from 9 to 500 monosaccharide units, such as from 10 to 500 monosaccharide units, for example from 11 to 500 monosaccharide units, such as from 12 to 500 monosaccharide units, for example from 13 to 500 monosaccharide units, such as from 14 to 500 monosaccharide units, for example from 15 to 500 monosaccharide units, such as from 20 to 500 monosaccharide units, for example from 25 to 500 monosaccharide units, such as from 30 to 500 monosaccharide units, for example from 40 to 500 monosaccharide units, such as from 50 to 500 monosaccharide units, for example from 60 to 500 monosaccharide units, such as from 70 to 500 monosaccharide units, for example from 80 to 500 monosaccharide units, such as from 90 to 500 monosaccharide units, for example from 100 to 500 monosaccharide units, such as from 2 to 250 monosaccharide units, for example from 3 to 250 monosaccharide units, such as from 4 to 250 monosaccharide units, for example from 5 to 250 monosaccharide units, such as from 6 to 250 monosaccharide units, for example from 7 to 250 monosaccharide units, such as from 8 to 250 monosaccharide units, for example from 9 to 250 monosaccharide units, such as from 10 to 250 monosaccharide units, for example from 11 to 250 monosaccharide units, such as from 12 to 250 monosaccharide units, for example from 13 to 250 monosaccharide units, such as from 14 to 250 monosaccharide units, for example from 15 to 250 monosaccharide units, such as from 20 to 250 monosaccharide units, for example from 25 to 250 monosaccharide units, such as from 30 to 250 monosaccharide units, for example from 40 to 250 monosaccharide units, such as from 50 to 250 monosaccharide units, for example from 60 to 250 monosaccharide units, such as from 70 to 250 monosaccharide units, for example from 80 to 250 monosaccharide units, such as from 90 to 250 monosaccharide units, for example from 100 to 250 monosaccharide units, such as from 2 to 100 monosaccharide units, for example from 3 to 100 monosaccharide units, such as from 4 to 100 monosaccharide units, for example from 5 to 100 monosaccharide units, such as from 6 to 100 monosaccharide units, for example from 7 to 100 monosaccharide units, such as from 8 to 100 monosaccharide units, for example from 9 to 100 monosaccharide units, such as from 10 to 100 monosaccharide units, for example from 11 to 100 monosaccharide units, such as from 12 to 100 monosaccharide units, for example from 13 to 100 monosaccharide units, such as from 14 to 100 monosaccharide units, for example from 15 to 100 monosaccharide units, such as from 20 to 100 monosaccharide units, for example from 25 to 100 monosaccharide units, such as from 30 to 100 monosaccharide units, for example from 40 to 100 monosaccharide units, such as from 50 to 100 monosaccharide units, for example from 60 to 100 monosaccharide units, such as from 70 to 100 monosaccharide units, for example from 80 to 100 monosaccharide units, such as from 90 to 100 monosaccharide units, such as from 2 to 50 monosaccharide units, for example from 3 to 50 monosaccharide units, such as from 4 to 50 monosaccharide units, for example from 5 to 50 monosaccharide units, such as from 6 to 50 monosaccharide units, for example from 7 to 50 monosaccharide units, such as from 8 to 50 monosaccharide units, for example from 9 to 50 monosaccharide units, such as from 10 to 50 monosaccharide units, for example from 11 to 50 monosaccharide units, such as from 12 to 50 monosaccharide units, for example from 13 to 50 monosaccharide units, such as from 14 to 50 monosaccharide units, for example from 15 to 50 monosaccharide units, such as from 20 to 50 monosaccharide units, for example from 25 to 50 monosaccharide units, such as from 30 to 50 monosaccharide units, for example from 40 to 50 monosaccharide units, such as from 2 to 25 monosaccharide units, for example from 3 to 25 monosaccharide units, such as from 4 to 25 monosaccharide units, for example from 5 to 25 monosaccharide units, such as from 6 to 25 monosaccharide units, for example from 7 to 25 monosaccharide units, such as from 8 to 25 monosaccharide units, for example from 9 to 25 monosaccharide units, such as from 10 to 25 monosaccharide units, for example from 11 to 25 monosaccharide units, such as from 12 to 25 monosaccharide units, for example from 13 to 25 monosaccharide units, such as from 14 to 25 monosaccharide units, for example from 15 to 25 monosaccharide units, such as from 20 to 25 monosaccharide units, such as from 2 to 20 monosaccharide units, for example from 3 to 20 monosaccharide units, such as from 4 to 20 monosaccharide units, for example from 5 to 20 monosaccharide units, such as from 6 to 20 monosaccharide units, for example from 7 to 20 monosaccharide units, such as from 8 to 20 monosaccharide units, for example from 9 to 20 monosaccharide units, such as from 10 to 20 monosaccharide units, for example from 11 to 20 monosaccharide units, such as from 12 to 20 monosaccharide units, for example from 13 to 20 monosaccharide units, such as from 14 to 20 monosaccharide units, for example from 15 to 20 monosaccharide units, such as from 2 to 18 monosaccharide units, for example from 3 to 18 monosaccharide units, such as from 4 to 18 monosaccharide units, for example from 5 to 18 monosaccharide units, such as from 6 to 18 monosaccharide units, for example from 7 to 18 monosaccharide units, such as from 8 to 18 monosaccharide units, for example from 9 to 18 monosaccharide units, such as from 10 to 18 monosaccharide units, for example from 11 to 18 monosaccharide units, such as from 12 to 18 monosaccharide units, for example from 13 to 18 monosaccharide units, such as from 14 to 18 monosaccharide units, for example from 15 to 18 monosaccharide units, such as from 2 to 16 monosaccharide units, for example from 3 to 16 monosaccharide units, such as from 4 to 16 monosaccharide units, for example from 5 to 16 monosaccharide units, such as from 6 to 16 monosaccharide units, for example from 7 to 16 monosaccharide units, such as from 8 to 16 monosaccharide units, for example from 9 to 16 monosaccharide units, such as from 10 to 16 monosaccharide units, for example from 11 to 16 monosaccharide units, such as from 12 to 16 monosaccharide units, for example from 13 to 16 monosaccharide units, such as from 14 to 16 monosaccharide units, for example from 15 to 16 monosaccharide units, such as from 2 to 14 monosaccharide units, for example from 3 to 14 monosaccharide units, such as from 4 to 14 monosaccharide units, for example from 5 to 14 monosaccharide units, such as from 6 to 14 monosaccharide units, for example from 7 to 14 monosaccharide units, such as from 8 to 14 monosaccharide units, for example from 9 to 14 monosaccharide units, such as from 10 to 14 monosaccharide units, for example from 11 to 14 monosaccharide units, such as from 12 to 14 monosaccharide units, for example from 13 to 14 monosaccharide units, such as from 2 to 12 monosaccharide units, for example from 3 to 12 monosaccharide units, such as from 4 to 12 monosaccharide units, for example from 5 to 12 monosaccharide units, such as from 6 to 12 monosaccharide units, for example from 7 to 12 monosaccharide units, such as from 8 to 12 monosaccharide units, for example from 9 to 12 monosaccharide units, such as from 10 to 12 monosaccharide units, for example from 11 to 12 monosaccharide units, such as from 2 to 10 monosaccharide units, for example from 3 to 10 monosaccharide units, such as from 4 to 10 monosaccharide units, for example from 5 to 10 monosaccharide units, such as from 6 to 10 monosaccharide units, for example from 7 to 10 monosaccharide units, such as from 8 to 10 monosaccharide units, for example from 9 to 10 monosaccharide units, such as from 2 to 8 monosaccharide units, for example from 3 to 8 monosaccharide units, such as from 4 to 8 monosaccharide units, for example from 5 to 8 monosaccharide units, such as from 6 to 8 monosaccharide units, for example from 7 to 8 monosaccharide units in the backbone between each branching point.

134. The bioactive agent according to item 2, wherein the polysaccharide has a molecular weight in the range of from 5,000 g/mol to about 1,000,000 g/mol, such as a molecular weight in the range of from 5,000 g/mol to about 900,000 g/mol, for example a molecular weight in the range of from 5,000 g/mol to about 800,000 g/mol, such as a molecular weight in the range of from 5,000 g/mol to about 900,000 g/mol, for example a molecular weight in the range of from 5,000 g/mol to about 800,000 g/mol, such as a molecular weight in the range of from 5,000 g/mol to about 750,000 g/mol, for example a molecular weight in the range of from 5,000 g/mol to about 700,000 g/mol, such as a molecular weight in the range of from 5,000 g/mol to about 1270,000 g/mol, for example a molecular weight in the range of from 5,000 g/mol to about 600,000 g/mol, such as a molecular weight in the range of from 5,000 g/mol to about 550,000 g/mol, for example a molecular weight in the range of from 5,000 g/mol to about 500,000 g/mol, such as a molecular weight in the range of from 5,000 g/mol to about 450,000 g/mol, for example a molecular weight in the range of from 5,000 g/mol to about 400,000 g/mol, such as a molecular weight in the range of from 5,000 g/mol to about 350,000 g/mol, for example a molecular weight in the range of from 5,000 g/mol to about 300,000 g/mol, such as a molecular weight in the range of from 5,000 g/mol to about 250,000 g/mol, for example a molecular weight in the range of from 5,000 g/mol to about 200,000 g/mol, such as a molecular weight in the range of from 5,000 g/mol to about 100,000 g/mol, for example a molecular weight in the range of from 5,000 g/mol to about 80,000 g/mol, such as a molecular weight in the range of from 5,000 g/mol to about 60,000 g/mol, for example a molecular weight in the range of from 5,000 g/mol to about 50,000 g/mol, such as a molecular weight in the range of from 5,000 g/mol to about 40,000 g/mol, for example a molecular weight in the range of from 5,000 g/mol to about 35.000 g/mol, such as a molecular weight in the range of from 5,000 g/mol to about 30,000 g/mol, for example a molecular weight in the range of from 5,000 g/mol to about 25.000 g/mol, such as a molecular weight in the range of from 5,000 g/mol to about 20,000 g/mol, for example a molecular weight in the range of from 5,000 g/mol to about 15.000 g/mol, such as a molecular weight in the range of from 5,000 g/mol to about 10,000 g/mol, for example a molecular weight in the range of from 10,000 g/mol to about 1,000,000 g/mol, such as a molecular weight in the range of from 10,000 g/mol to about 900,000 g/mol, for example a molecular weight in the range of from 10,000 g/mol to about 800,000 g/mol, such as a molecular weight in the range of from 10,000 g/mol to about 900,000 g/mol, for example a molecular weight in the range of from 10,000 g/mol to about 800,000 g/mol, such as a molecular weight in the range of from 10,000 g/mol to about 750,000 g/mol, for example a molecular weight in the range of from 10,000 g/mol to about 700,000 g/mol, such as a molecular weight in the range of from 10,000 g/mol to about 1270,000 g/mol, for example a molecular weight in the range of from 10,000 g/mol to about 600,000 g/mol, such as a molecular weight in the range of from 10,000 g/mol to about 550,000 g/mol, for example a molecular weight in the range of from 10,000 g/mol to about 500,000 g/mol, such as a molecular weight in the range of from 10,000 g/mol to about 450,000 g/mol, for example a molecular weight in the range of from 10,000 g/mol to about 400,000 g/mol, such as a molecular weight in the range of from 10,000 g/mol to about 350,000 g/mol, for example a molecular weight in the range of from 10,000 g/mol to about 300,000 g/mol, such as a molecular weight in the range of from 10,000 g/mol to about 250,000 g/mol, for example a molecular weight in the range of from 10,000 g/mol to about 200,000 g/mol, such as a molecular weight in the range of from 10,000 g/mol to about 100,000 g/mol, for example a molecular weight in the range of from 10,000 g/mol to about 80,000 g/mol, such as a molecular weight in the range of from 10,000 g/mol to about 60,000 g/mol, for example a molecular weight in the range of from 10,000 g/mol to about 50,000 g/mol, such as a molecular weight in the range of from 10,000 g/mol to about 40,000 g/mol, for example a molecular weight in the range of from 10,000 g/mol to about 35.000 g/mol, such as a molecular weight in the range of from 10,000 g/mol to about 30,000 g/mol, for example a molecular weight in the range of from 10,000 g/mol to about 25.000 g/mol, such as a molecular weight in the range of from 10,000 g/mol to about 20,000 g/mol, for example a molecular weight in the range of from 10,000 g/mol to about 15.000 g/mol, such as a molecular weight in the range of from 15,000 g/mol to about 1,000,000 g/mol, such as a molecular weight in the range of from 15,000 g/mol to about 900,000 g/mol, for example a molecular weight in the range of from 15,000 g/mol to about 800,000 g/mol, such as a molecular weight in the range of from 15,000 g/mol to about 900,000 g/mol, for example a molecular weight in the range of from 15,000 g/mol to about 800,000 g/mol, such as a molecular weight in the range of from 15,000 g/mol to about 750,000 g/mol, for example a molecular weight in the range of from 15,000 g/mol to about 700,000 g/mol, such as a molecular weight in the range of from 15,000 g/mol to about 1270,000 g/mol, for example a molecular weight in the range of from 15,000 g/mol to about 600,000 g/mol, such as a molecular weight in the range of from 15,000 g/mol to about 550,000 g/mol, for example a molecular weight in the range of from 15,000 g/mol to about 500,000 g/mol, such as a molecular weight in the range of from 15,000 g/mol to about 450,000 g/mol, for example a molecular weight in the range of from 15,000 g/mol to about 400,000 g/mol, such as a molecular weight in the range of from 15,000 g/mol to about 350,000 g/mol, for example a molecular weight in the range of from 15,000 g/mol to about 300,000 g/mol, such as a molecular weight in the range of from 15,000 g/mol to about 250,000 g/mol, for example a molecular weight in the range of from 15,000 g/mol to about 200,000 g/mol, such as a molecular weight in the range of from 15,000 g/mol to about 100,000 g/mol, for example a molecular weight in the range of from 15,000 g/mol to about 80,000 g/mol, such as a molecular weight in the range of from 15,000 g/mol to about 60,000 g/mol, for example a molecular weight in the range of from 15,000 g/mol to about 50,000 g/mol, such as a molecular weight in the range of from 15,000 g/mol to about 40,000 g/mol, for example a molecular weight in the range of from 15,000 g/mol to about 35.000 g/mol, such as a molecular weight in the range of from 15,000 g/mol to about 30,000 g/mol, for example a molecular weight in the range of from 15,000 g/mol to about 25.000 g/mol, such as a molecular weight in the range of from 15,000 g/mol to about 20,000 g/mol, for example a molecular weight in the range of from 20,000 g/mol to about 1,000,000 g/mol, such as a molecular weight in the range of from 20,000 g/mol to about 900,000 g/mol, for example a molecular weight in the range of from 20,000 g/mol to about 800,000 g/mol, such as a molecular weight in the range of from 20,000 g/mol to about 900,000 g/mol, for example a molecular weight in the range of from 20,000 g/mol to about 800,000 g/mol, such as a molecular weight in the range of from 20,000 g/mol to about 750,000 g/mol, for example a molecular weight in the range of from 20,000 g/mol to about 700,000 g/mol, such as a molecular weight in the range of from 20,000 g/mol to about 1270,000 g/mol, for example a molecular weight in the range of from 20,000 g/mol to about 600,000 g/mol, such as a molecular weight in the range of from 20,000 g/mol to about 550,000 g/mol, for example a molecular weight in the range of from 20,000 g/mol to about 500,000 g/mol, such as a molecular weight in the range of from 20,000 g/mol to about 450,000 g/mol, for example a molecular weight in the range of from 20,000 g/mol to about 400,000 g/mol, such as a molecular weight in the range of from 20,000 g/mol to about 350,000 g/mol, for example a molecular weight in the range of from 20,000 g/mol to about 300,000 g/mol, such as a molecular weight in the range of from 20,000 g/mol to about 250,000 g/mol, for example a molecular weight in the range of from 20,000 g/mol to about 200,000 g/mol, such as a molecular weight in the range of from 20,000 g/mol to about 100,000 g/mol, for example a molecular weight in the range of from 20,000 g/mol to about 80,000 g/mol, such as a molecular weight in the range of from 20,000 g/mol to about 60,000 g/mol, for example a molecular weight in the range of from 20,000 g/mol to about 50,000 g/mol, such as a molecular weight in the range of from 20,000 g/mol to about 40,000 g/mol, for example a molecular weight in the range of from 20,000 g/mol to about 35.000 g/mol, such as a molecular weight in the range of from 20,000 g/mol to about 30,000 g/mol, for example a molecular weight in the range of from 20,000 g/mol to about 25.000 g/mol, such as a molecular weight in the range of from 25,000 g/mol to about 1,000,000 g/mol, such as a molecular weight in the range of from 25,000 g/mol to about 900,000 g/mol, for example a molecular weight in the range of from 25,000 g/mol to about 800,000 g/mol, such as a molecular weight in the range of from 25,000 g/mol to about 900,000 g/mol, for example a molecular weight in the range of from 25,000 g/mol to about 800,000 g/mol, such as a molecular weight in the range of from 25,000 g/mol to about 750,000 g/mol, for example a molecular weight in the range of from 25,000 g/mol to about 700,000 g/mol, such as a molecular weight in the range of from 25,000 g/mol to about 1270,000 g/mol, for example a molecular weight in the range of from 25,000 g/mol to about 600,000 g/mol, such as a molecular weight in the range of from 25,000 g/mol to about 550,000 g/mol, for example a molecular weight in the range of from 25,000 g/mol to about 500,000 g/mol, such as a molecular weight in the range of from 25,000 g/mol to about 450,000 g/mol, for example a molecular weight in the range of from 25,000 g/mol to about 400,000 g/mol, such as a molecular weight in the range of from 25,000 g/mol to about 350,000 g/mol, for example a molecular weight in the range of from 25,000 g/mol to about 300,000 g/mol, such as a molecular weight in the range of from 25,000 g/mol to about 250,000 g/mol, for example a molecular weight in the range of from 25,000 g/mol to about 200,000 g/mol, such as a molecular weight in the range of from 25,000 g/mol to about 100,000 g/mol, for example a molecular weight in the range of from 25,000 g/mol to about 80,000 g/mol, such as a molecular weight in the range of from 25,000 g/mol to about 60,000 g/mol, for example a molecular weight in the range of from 25,000 g/mol to about 50,000 g/mol, such as a molecular weight in the range of from 25,000 g/mol to about 40,000 g/mol, for example a molecular weight in the range of from 25,000 g/mol to about 35.000 g/mol, such as a molecular weight in the range of from 25,000 g/mol to about 30,000 g/mol, for example a molecular weight in the range of from 30,000 g/mol to about 1,000,000 g/mol, such as a molecular weight in the range of from 30,000 g/mol to about 900,000 g/mol, for example a molecular weight in the range of from 30,000 g/mol to about 800,000 g/mol, such as a molecular weight in the range of from 30,000 g/mol to about 900,000 g/mol, for example a molecular weight in the range of from 30,000 g/mol to about 800,000 g/mol, such as a molecular weight in the range of from 30,000 g/mol to about 750,000 g/mol, for example a molecular weight in the range of from 30,000 g/mol to about 700,000 g/mol, such as a molecular weight in the range of from 30,000 g/mol to about 1270,000 g/mol, for example a molecular weight in the range of from 30,000 g/mol to about 600,000 g/mol, such as a molecular weight in the range of from 30,000 g/mol to about 550,000 g/mol, for example a molecular weight in the range of from 30,000 g/mol to about 500,000 g/mol, such as a molecular weight in the range of from 30,000 g/mol to about 450,000 g/mol, for example a molecular weight in the range of from 30,000 g/mol to about 400,000 g/mol, such as a molecular weight in the range of from 30,000 g/mol to about 350,000 g/mol, for example a molecular weight in the range of from 30,000 g/mol to about 300,000 g/mol, such as a molecular weight in the range of from 30,000 g/mol to about 250,000 g/mol, for example a molecular weight in the range of from 30,000 g/mol to about 200,000 g/mol, such as a molecular weight in the range of from 30,000 g/mol to about 100,000 g/mol, for example a molecular weight in the range of from 30,000 g/mol to about 80,000 g/mol, such as a molecular weight in the range of from 30,000 g/mol to about 60,000 g/mol, for example a molecular weight in the range of from 30,000 g/mol to about 50,000 g/mol, such as a molecular weight in the range of from 30,000 g/mol to about 40,000 g/mol, for example a molecular weight in the range of from 30,000 g/mol to about 35.000 g/mol, such as a molecular weight in the range of from 40,000 g/mol to about 1,000,000 g/mol, such as a molecular weight in the range of from 40,000 g/mol to about 900,000 g/mol, for example a molecular weight in the range of from 40,000 g/mol to about 800,000 g/mol, such as a molecular weight in the range of from 40,000 g/mol to about 900,000 g/mol, for example a molecular weight in the range of from 40,000 g/mol to about 800,000 g/mol, such as a molecular weight in the range of from 40,000 g/mol to about 750,000 g/mol, for example a molecular weight in the range of from 40,000 g/mol to about 700,000 g/mol, such as a molecular weight in the range of from 40,000 g/mol to about 1270,000 g/mol, for example a molecular weight in the range of from 40,000 g/mol to about 600,000 g/mol, such as a molecular weight in the range of from 40,000 g/mol to about 550,000 g/mol, for example a molecular weight in the range of from 40,000 g/mol to about 500,000 g/mol, such as a molecular weight in the range of from 40,000 g/mol to about 450,000 g/mol, for example a molecular weight in the range of from 40,000 g/mol to about 400,000 g/mol, such as a molecular weight in the range of from 40,000 g/mol to about 350,000 g/mol, for example a molecular weight in the range of from 40,000 g/mol to about 300,000 g/mol, such as a molecular weight in the range of from 40,000 g/mol to about 250,000 g/mol, for example a molecular weight in the range of from 40,000 g/mol to about 200,000 g/mol, such as a molecular weight in the range of from 40,000 g/mol to about 100,000 g/mol, for example a molecular weight in the range of from 40,000 g/mol to about 80,000 g/mol, such as a molecular weight in the range of from 40,000 g/mol to about 60,000 g/mol, for example a molecular weight in the range of from 40,000 g/mol to about 50,000 g/mol, such as a molecular weight in the range of from 50,000 g/mol to about 1,000,000 g/mol, such as a molecular weight in the range of from 50,000 g/mol to about 900,000 g/mol, for example a molecular weight in the range of from 50,000 g/mol to about 800,000 g/mol, such as a molecular weight in the range of from 50,000 g/mol to about 900,000 g/mol, for example a molecular weight in the range of from 50,000 g/mol to about 800,000 g/mol, such as a molecular weight in the range of from 50,000 g/mol to about 750,000 g/mol, for example a molecular weight in the range of from 50,000 g/mol to about 700,000 g/mol, such as a molecular weight in the range of from 50,000 g/mol to about 1270,000 g/mol, for example a molecular weight in the range of from 50,000 g/mol to about 600,000 g/mol, such as a molecular weight in the range of from 50,000 g/mol to about 550,000 g/mol, for example a molecular weight in the range of from 50,000 g/mol to about 500,000 g/mol, such as a molecular weight in the range of from 50,000 g/mol to about 450,000 g/mol, for example a molecular weight in the range of from 50,000 g/mol to about 400,000 g/mol, such as a molecular weight in the range of from 50,000 g/mol to about 350,000 g/mol, for example a molecular weight in the range of from 50,000 g/mol to about 300,000 g/mol, such as a molecular weight in the range of from 50,000 g/mol to about 250,000 g/mol, for example a molecular weight in the range of from 50,000 g/mol to about 200,000 g/mol, such as a molecular weight in the range of from 50,000 g/mol to about 100,000 g/mol, for example a molecular weight in the range of from 50,000 g/mol to about 80,000 g/mol, such as a molecular weight in the range of from 50,000 g/mol to about 60,000 g/mol, for example a molecular weight in the range of from 75,000 g/mol to about 1,000,000 g/mol, such as a molecular weight in the range of from 75,000 g/mol to about 900,000 g/mol, for example a molecular weight in the range of from 75,000 g/mol to about 800,000 g/mol, such as a molecular weight in the range of from 75,000 g/mol to about 900,000 g/mol, for example a molecular weight in the range of from 75,000 g/mol to about 800,000 g/mol, such as a molecular weight in the range of from 75,000 g/mol to about 750,000 g/mol, for example a molecular weight in the range of from 75,000 g/mol to about 700,000 g/mol, such as a molecular weight in the range of from 75,000 g/mol to about 1270,000 g/mol, for example a molecular weight in the range of from 75,000 g/mol to about 600,000 g/mol, such as a molecular weight in the range of from 75,000 g/mol to about 550,000 g/mol, for example a molecular weight in the range of from 75,000 g/mol to about 500,000 g/mol, such as a molecular weight in the range of from 75,000 g/mol to about 450,000 g/mol, for example a molecular weight in the range of from 75,000 g/mol to about 400,000 g/mol, such as a molecular weight in the range of from 75,000 g/mol to about 350,000 g/mol, for example a molecular weight in the range of from 75,000 g/mol to about 300,000 g/mol, such as a molecular weight in the range of from 75,000 g/mol to about 250,000 g/mol, for example a molecular weight in the range of from 75,000 g/mol to about 200,000 g/mol, such as a molecular weight in the range of from 75,000 g/mol to about 100,000 g/mol, for example a molecular weight in the range of from 75,000 g/mol to about 80,000 g/mol, a molecular weight in the range of from 100,000 g/mol to about 1,000,000 g/mol, such as a molecular weight in the range of from 100,000 g/mol to about 900,000 g/mol, for example a molecular weight in the range of from 100,000 g/mol to about 800,000 g/mol, such as a molecular weight in the range of from 100,000 g/mol to about 900,000 g/mol, for example a molecular weight in the range of from 100,000 g/mol to about 800,000 g/mol, such as a molecular weight in the range of from 100,000 g/mol to about 750,000 g/mol, for example a molecular weight in the range of from 100,000 g/mol to about 700,000 g/mol, such as a molecular weight in the range of from 100,000 g/mol to about 1270,000 g/mol, for example a molecular weight in the range of from 100,000 g/mol to about 600,000 g/mol, such as a molecular weight in the range of from 100,000 g/mol to about 550,000 g/mol, for example a molecular weight in the range of from 100,000 g/mol to about 500,000 g/mol, such as a molecular weight in the range of from 100,000 g/mol to about 450,000 g/mol, for example a molecular weight in the range of from 100,000 g/mol to about 400,000 g/mol, such as a molecular weight in the range of from 100,000 g/mol to about 350,000 g/mol, for example a molecular weight in the range of from 100,000 g/mol to about 300,000 g/mol, such as a molecular weight in the range of from 100,000 g/mol to about 250,000 g/mol, for example a molecular weight in the range of from 100,000 g/mol to about 200,000 g/mol, such as a molecular weight in the range of from 200,000 g/mol to about 300,000 g/mol, for example a molecular weight in the range of from 300,000 g/mol to about 400,000 g/mol, such as a molecular weight in the range of from 400,000 g/mol to about 500,000 g/mol, for example a molecular weight in the range of from 500,000 g/mol to about 600,000 g/mol, such as a molecular weight in the range of from 700,000 g/mol to about 800,000 g/mol, for example a molecular weight in the range of from 800,000 g/mol to about 900,000 g/mol, such as a molecular weight in the range of from 900,000 g/mol to about 1,000,000 g/mol.
135. The bioactive agent according to item 2, wherein the polysaccharide comprises a structural component selected from the group of components consisting of
   (1-3)-alpha-D-glucan;
   (1-3)-alpha-D-glucan with (1-6)-beta branching;
   (1-3)-alpha-D-glucan with (1-6)-alpha branching;
   (1-3)-alpha-D-glucan with (1-4)-beta branching;
   (1-3)-alpha-D-glucan with (1-4)-alpha branching;
   (1-3)-beta-D-glucan;
   (1-3)-beta-D-glucan with (1-6)-beta branching;
   (1-3)-beta-D-glucan with (1-6)-alpha branching;
   (1-3)-beta-D-glucan with (1-4)-beta branching;
   (1-3)-beta-D-glucan with (1-4)-alpha branching;
   (1-4)-alpha-D-glucan;
   (1-4)-alpha-D-glucan with (1-6)-beta branching;
   (1-4)-alpha-D-glucan with (1-6)-alpha branching;
   (1-4)-alpha-D-glucan with (1-4)-beta branching;
   (1-4)-alpha-D-glucan with (1-4)-alpha branching;
   (1-4)-beta-D-glucan;
   (1-4)-beta-D-glucan with (1-6)-beta branching;
   (1-4)-beta-D-glucan with (1-6)-alpha branching;
   (1-4)-beta-D-glucan with (1-4)-beta branching;
   (1-4)-beta-D-glucan with (1-4)-alpha branching;
   (1-6)-beta-D-glucan;
   (1-6)-beta-D-glucan with (1-6)-beta branching;
   (1-6)-beta-D-glucan with (1-6)-alpha branching;
   (1-6)-beta-D-glucan with (1-4)-beta branching;
   (1-6)-beta-D-glucan with (1-4)-alpha branching;
   (1-6)-alpha-D-glucan;
   (1-6)-alpha-D-glucan with (1-6)-beta branching;
   (1-6)-alpha-D-glucan with (1-6)-alpha branching;
   (1-6)-alpha-D-glucan with (1-4)-beta branching;
   (1-6)-alpha-D-glucan with (1-4)-alpha branching;
136. The bioactive agent according to item 2, wherein the polysaccharide comprises a structural component comprising (1-3)-alpha-D-glucan.
137. The bioactive agent according to item 2, wherein the polysaccharide comprises a structural component comprising (1-3)-alpha-D-glucan with (1-6)-beta branching.
138. The bioactive agent according to item 2, wherein the polysaccharide comprises a structural component comprising (1-3)-alpha-D-glucan with (1-6)-alpha branching.
139. The bioactive agent according to item 2, wherein the polysaccharide comprises a structural component comprising (1-3)-alpha-D-glucan with (1-4)-beta branching.
140. The bioactive agent according to item 2, wherein the polysaccharide comprises a structural component comprising (1-3)-alpha-D-glucan with (1-4)-alpha branching.
141. The bioactive agent according to item 2, wherein the polysaccharide comprises a structural component comprising (1-3)-beta-D-glucan.
142. The bioactive agent according to item 2, wherein the polysaccharide comprises a structural component comprising (1-3)-beta-D-glucan with (1-6)-beta branching.
143. The bioactive agent according to item 2, wherein the polysaccharide comprises a structural component comprising (1-3)-beta-D-glucan with (1-6)-alpha branching.
144. The bioactive agent according to item 2, wherein the polysaccharide comprises a structural component comprising (1-3)-beta-D-glucan with (1-4)-beta branching.
145. The bioactive agent according to item 2, wherein the polysaccharide comprises a structural component comprising (1-3)-beta-D-glucan with (1-4)-alpha branching.
146. The bioactive agent according to item 2, wherein the polysaccharide comprises a structural component comprising (1-4)-alpha-D-glucan.
147. The bioactive agent according to item 2, wherein the polysaccharide comprises a structural component comprising (1-4)-alpha-D-glucan with (1-6)-beta branching.
148. The bioactive agent according to item 2, wherein the polysaccharide comprises a structural component comprising (1-4)-alpha-D-glucan with (1-6)-alpha branching.
149. The bioactive agent according to item 2, wherein the polysaccharide comprises a structural component comprising (1-4)-alpha-D-glucan with (1-4)-beta branching.
150. The bioactive agent according to item 2, wherein the polysaccharide comprises a structural component comprising (1-4)-alpha-D-glucan with (1-4)-alpha branching.
151. The bioactive agent according to item 2, wherein the polysaccharide comprises a structural component comprising (1-4)-beta-D-glucan.
152. The bioactive agent according to item 2, wherein the polysaccharide comprises a structural component comprising (1-4)-beta-D-glucan with (1-6)-beta branching.
153. The bioactive agent according to item 2, wherein the polysaccharide comprises a structural component comprising (1-4)-beta-D-glucan with (1-6)-alpha branching.
154. The bioactive agent according to item 2, wherein the polysaccharide comprises a structural component comprising (1-4)-beta-D-glucan with (1-4)-beta branching.
155. The bioactive agent according to item 2, wherein the polysaccharide comprises a structural component comprising (1-4)-beta-D-glucan with (1-4)-alpha branching.
156. The bioactive agent according to item 2, wherein the polysaccharide comprises a structural component comprising (1-6)-beta-D-glucan.
157. The bioactive agent according to item 2, wherein the polysaccharide comprises a structural component comprising (1-6)-beta-D-glucan with (1-6)-beta branching.
158. The bioactive agent according to item 2, wherein the polysaccharide comprises a structural component comprising (1-6)-beta-D-glucan with (1-6)-alpha branching.
159. The bioactive agent according to item 2, wherein the polysaccharide comprises a structural component comprising (1-6)-beta-D-glucan with (1-4)-beta branching.
160. The bioactive agent according to item 2, wherein the polysaccharide comprises a structural component comprising (1-6)-beta-D-glucan with (1-4)-alpha branching.
161. The bioactive agent according to item 2, wherein the polysaccharide comprises a structural component comprising (1-6)-alpha-D-glucan.
162. The bioactive agent according to item 2, wherein the polysaccharide comprises a structural component comprising (1-6)-alpha-D-glucan with (1-6)-beta branching.
163. The bioactive agent according to item 2, wherein the polysaccharide comprises a structural component comprising (1-6)-alpha-D-glucan with (1-6)-alpha branching.
164. The bioactive agent according to item 2, wherein the polysaccharide comprises a structural component comprising (1-6)-alpha-D-glucan with (1-4)-beta branching.
165. The bioactive agent according to item 2, wherein the polysaccharide comprises a structural component comprising (1-6)-alpha-D-glucan with (1-4)-alpha branching.
166. The bioactive agent according to item 2, wherein the polysaccharide backbone comprises a plurality of monosaccharide units linked by a chemical bond selected from the group consisting of (1-6)-beta bonds, (1-4)-beta bonds, (1-3)-beta bonds, (1-2)-beta bonds, (1-1)-beta bonds, 1-beta-1-alpha bonds, 1-alpha-1-alpha bonds, 1-alpha-1-beta bonds, (1-2)-alpha bonds, (1-3)-alpha bonds, (1-4)-alpha bonds and (1-6)-alpha bonds.
167. The bioactive agent according to item 2, wherein the polysaccharide backbone comprises a plurality of monosaccharide units linked by (1-6)-beta bonds.
168. The bioactive agent according to item 2, wherein the polysaccharide backbone comprises a plurality of monosaccharide units linked by (1-4)-beta bonds.
169. The bioactive agent according to item 2, wherein the polysaccharide backbone comprises a plurality of monosaccharide units linked by (1-3)-beta bonds.
170. The bioactive agent according to item 2, wherein the polysaccharide backbone comprises a plurality of monosaccharide units linked by (1-2)-beta bonds.
171. The bioactive agent according to item 2, wherein the polysaccharide backbone comprises a plurality of monosaccharide units linked by (1-1)-beta bonds.
172. The bioactive agent according to item 2, wherein the polysaccharide backbone comprises a plurality of monosaccharide units linked by 1-beta-1-alpha bonds.
173. The bioactive agent according to item 2, wherein the polysaccharide backbone comprises a plurality of monosaccharide units linked by 1-alpha-1-alpha bonds.
174. The bioactive agent according to item 2, wherein the polysaccharide backbone comprises a plurality of monosaccharide units linked by 1-alpha-1-beta bonds.
175. The bioactive agent according to item 2, wherein the polysaccharide backbone comprises a plurality of monosaccharide units linked by (1-2)-alpha bonds.
176. The bioactive agent according to item 2, wherein the polysaccharide backbone comprises a plurality of monosaccharide units linked by (1-3)-alpha bonds.
177. The bioactive agent according to item 2, wherein the polysaccharide backbone comprises a plurality of monosaccharide units linked by (1-4)-alpha bonds.
178. The bioactive agent according to item 2, wherein the polysaccharide backbone comprises a plurality of monosaccharide units linked by (1-6)-alpha bonds.
179. The bioactive agent according to any of items 166 to 178, wherein the polysaccharide further comprises side chains comprising a plurality of monosaccharides selected from the group consisting of (1-6)-beta bonds, (1-4)-beta bonds, (1-3)-beta bonds, (1-2)-beta bonds, (1-1)-beta bonds, 1-beta-1-alpha bonds, 1-alpha-1-alpha bonds, 1-alpha-1-beta bonds, (1-2)-alpha bonds, (1-3)-alpha bonds, (1-4)-alpha bonds and (1-6)-alpha bonds.
180. The bioactive agent according to any of items 166 to 178, wherein the polysaccharide side chains comprise a plurality of monosaccharide units linked by (1-6)-beta bonds.
181. The bioactive agent according to any of items 166 to 178, wherein the polysaccharide side chains comprise a plurality of monosaccharide units linked by (1-4)-beta bonds.
182. The bioactive agent according to any of items 166 to 178, wherein the polysaccharide side chains comprise a plurality of monosaccharide units linked by (1-3)-beta bonds.
183. The bioactive agent according to any of items 166 to 178, wherein the polysaccharide side chains comprise a plurality of monosaccharide units linked by (1-2)-beta bonds.
184. The bioactive agent according to any of items 166 to 178, wherein the polysaccharide side chains comprise a plurality of monosaccharide units linked by (1-1)-beta bonds.
185. The bioactive agent according to any of items 166 to 178, wherein the polysaccharide side chains comprise a plurality of monosaccharide units linked by 1-beta-1-alpha bonds.
186. The bioactive agent according to any of items 166 to 178, wherein the polysaccharide side chains comprise a plurality of monosaccharide units linked by 1-alpha-1-alpha bonds.
187. The bioactive agent according to any of items 166 to 178, wherein the polysaccharide side chains comprise a plurality of monosaccharide units linked by 1-alpha-1-beta bonds.
188. The bioactive agent according to any of items 166 to 178, wherein the polysaccharide side chains comprise a plurality of monosaccharide units linked by (1-2)-alpha bonds.
189. The bioactive agent according to any of items 166 to 178, wherein the polysaccharide side chains comprise a plurality of monosaccharide units linked by (1-3)-alpha bonds.
190. The bioactive agent according to any of items 166 to 178, wherein the polysaccharide side chains comprise a plurality of monosaccharide units linked by (1-4)-alpha bonds.
191. The bioactive agent according to any of items 166 to 178, wherein the polysaccharide side chains comprise a plurality of monosaccharide units linked by (1-6)-alpha bonds.
192. The bioactive agent according to any of items 2 to 191, wherein the polysaccharide is a heteropolymer comprising two or more different monosaccharides in the main chain, such as 3 different monosaccharides in the main chain, for example 4 different monosaccharides in the main chain, such as different monosaccharides in the main chain, for example 6 different monosaccharides in the main chain.
193. The bioactive agent according to item 192, wherein the polysaccharide further comprises two or more different monosaccharides in the side chains, such as 3 different monosaccharides in the side chains, for example 4 different monosaccharides in the side chains, such as 5 different monosaccharides in the side chains, for example 6 different monosaccharides in the side chains.
194. The bioactive agent according to any of items 7, 38 and 69, wherein the ratio R=a/b between a) the number of glucose monosaccharides and b) the number of further monosaccharides is about 0.0001, for example about 0.0005, such as about 0.001, for example about 0.005, such as about 0.01, for example about 0.05, such as about 0.1, for example about 0.2, such as about 0.3, for example about 0.4, such as about 0.5, for example about 0.6, such as about 0.7, for example about 0.8, such as about 0.9, for example about 1; such as from 1:10000 to 1, such as from 2:10000 to 1; for example from 4:10000 to 1; such as from 10:10000 to 1; for example from 20:10000 to 1; such as from 40:10000 to 1; for example from 80:10000 to 1; such as from 100:10000 to 1; for example from 100:10000 to 1; such as from 200:10000 to 1; for example from 250:10000 to 1; such as from 400:10000 to 1; for example from 500:10000 to 1; such as from 1000:10000 to 1; for example from 2000:10000 to 1; such as from 2500:10000 to 1; for example from 3000:10000 to 1; such as from 4000:10000 to 1; for example from 5000:10000 to 1; such as from 6000:10000 to 1; for example from 7000:10000 to 1; such as from 7500:10000 to 1; for example from 8000:10000 to 1; such as from 9000:10000 to 1; for example from 9500:10000 to 1, such as from 1:10000 to 5:10000; for example from 5:10000 to 20:10000, such as from 20:10000 to 100:10000; for example from 100:10000 to 500:10000; such as from 500:10000 to 1000:10000; for example from 1000:10000 to 2000:10000; such as from 2000:10000 to 3000:10000; for example from 3000:10000 to 4000:10000; such as from 4000:10000 to 5000:10000; for example from 5000:10000 to 6000:10000; such as from 6000:10000 to 7000:10000; for example from 7000:10000 to 8000:10000; such as from 8000:10000 to 9000:10000.
195. The bioactive agent according to any of items 7, 38 and 69, wherein the ratio R=b/a between a) the number of glucose monosaccharides and b) the number of further monosaccharides is about 0.0001, for example about 0.0005, such as about 0.001, for example about 0.005, such as about 0.01, for example about 0.05, such as about 0.1, for example about 0.2, such as about 0.3, for example about 0.4, such as about 0.5, for example about 0.6, such as about 0.7, for example about 0.8, such as about 0.9, for example about 1; for example from 1:10000 to 1, such as from 2:10000 to 1; for example from 4:10000 to 1; such as from 10:10000 to 1; for example from 20:10000 to 1; such as from 40:10000 to 1; for example from 80:10000 to 1; such as from 100:10000 to 1; for example from 100:10000 to 1; such as from 200:10000 to 1; for example from 250:10000 to 1; such as from 400:10000 to 1; for example from 500:10000 to 1; such as from 1000:10000 to 1; for example from 2000:10000 to 1; such as from 2500:10000 to 1; for example from 3000:10000 to 1; such as from 4000:10000 to 1; for example from 5000:10000 to 1; such as from 6000:10000 to 1; for example from 7000:10000 to 1; such as from 7500:10000 to 1; for example from 8000:10000 to 1; such as from 9000:10000 to 1; for example from 9500:10000 to 1; such as from 1:10000 to 5:10000; for example from 5:10000 to 20:10000, such as from 20:10000 to 100:10000; for example from 100:10000 to 500:10000; such as from 500:10000 to 1000:10000; for example from 1000:10000 to 2000:10000; such as from 2000:10000 to 3000:10000; for example from 3000:10000 to 4000:10000; such as from 4000:10000 to 5000:10000; for example from 5000:10000 to 6000:10000; such as from 6000:10000 to 7000:10000; for example from 7000:10000 to 8000:10000; such as from 8000:10000 to 9000:10000.
196. The bioactive agent according to any of items 7, 38 and 69, wherein the ratio R=a/b between a) the number of glucose monosaccharides and b) the number of glucuronic acid monosaccharides is about 0.0001, for example about 0.0005, such as about 0.001, for example about 0.005, such as about 0.01, for example about 0.05, such as about 0.1, for example about 0.2, such as about 0.3, for example about 0.4, such as about 0.5, for example about 0.6, such as about 0.7, for example about 0.8, such as about 0.9, for example about 1; for example from 1:10000 to 1, such as from 2:10000 to 1; for example from 4:10000 to 1; such as from 10:10000 to 1; for example from 20:10000 to 1; such as from 40:10000 to 1; for example from 80:10000 to 1; such as from 100:10000 to 1; for example from 100:10000 to 1; such as from 200:10000 to 1; for example from 250:10000 to 1; such as from 400:10000 to 1; for example from 500:10000 to 1; such as from 1000:10000 to 1; for example from 2000:10000 to 1; such as from 2500:10000 to 1; for example from 3000:10000 to 1; such as from 4000:10000 to 1; for example from 5000:10000 to 1; such as from 6000:10000 to 1; for example from 7000:10000 to 1; such as from 7500:10000 to 1; for example from 8000:10000 to 1; such as from 9000:10000 to 1; for example from 9500:10000 to 1; such as from 1:10000 to 5:10000; for example from 5:10000 to 20:10000, such as from 20:10000 to 100:10000; for example from 100:10000 to 500:10000; such as from 500:10000 to 1000:10000; for example from 1000:10000 to 2000:10000; such as from 2000:10000 to 3000:10000; for example from 3000:10000 to 4000:10000; such as from 4000:10000 to 5000:10000; for example from 5000:10000 to 6000:10000; such as from 6000:10000 to 7000:10000; for example from 7000:10000 to 8000:10000; such as from 8000:10000 to 9000:10000.
197. The bioactive agent according to any of items 7, 38 and 69, wherein the ratio R=b/a between a) the number of glucose monosaccharides and b) the number of glucuronic acid monosaccharides is about 0.0001, for example about 0.0005, such as about 0.001, for example about 0.005, such as about 0.01, for example about 0.05, such as about 0.1, for example about 0.2, such as about 0.3, for example about 0.4, such as about 0.5, for example about 0.6, such as about 0.7, for example about 0.8, such as about 0.9, for example about 1; for example from 1:10000 to 1, such as from 2:10000 to 1; for example from 4:10000 to 1; such as from 10:10000 to 1; for example from 20:10000 to 1; such as from 40:10000 to 1; for example from 80:10000 to 1; such as from 100:10000 to 1; for example from 100:10000 to 1; such as from 200:10000 to 1; for example from 250:10000 to 1; such as from 400:10000 to 1; for example from 500:10000 to 1; such as from 1000:10000 to 1; for example from 2000:10000 to 1; such as from 2500:10000 to 1; for example from 3000:10000 to 1; such as from 4000:10000 to 1; for example from 5000:10000 to 1; such as from 6000:10000 to 1; for example from 7000:10000 to 1; such as from 7500:10000 to 1; for example from 8000:10000 to 1; such as from 9000:10000 to 1; for example from 9500:10000 to 1; such as from 1:10000 to 5:10000; for example from 5:10000 to 20:10000, such as from 20:10000 to 100:10000; for example from 100:10000 to 500:10000; such as from 500:10000 to 1000:10000; for example from 1000:10000 to 2000:10000; such as from 2000:10000 to 3000:10000; for example from 3000:10000 to 4000:10000; such as from 4000:10000 to 5000:10000; for example from 5000:10000 to 6000:10000; such as from 6000:10000 to 7000:10000; for example from 7000:10000 to 8000:10000; such as from 8000:10000 to 9000:10000.
198. The bioactive agent according to any of items 7, 38 and 69, wherein the ratio R=a/b between a) the number of glucose monosaccharides and b) the number of galactose monosaccharides is about 0.0001, for example about 0.0005, such as about 0.001, for example about 0.005, such as about 0.01, for example about 0.05, such as about 0.1, for example about 0.2, such as about 0.3, for example about 0.4, such as about 0.5, for example about 0.6, such as about 0.7, for example about 0.8, such as about 0.9, for example about 1; for example from 1:10000 to 1, such as from 2:10000 to 1; for example from 4:10000 to 1; such as from 10:10000 to 1; for example from 20:10000 to 1; such as from 40:10000 to 1; for example from 80:10000 to 1; such as from 100:10000 to 1; for example from 100:10000 to 1; such as from 200:10000 to 1; for example from 250:10000 to 1; such as from 400:10000 to 1; for example from 500:10000 to 1; such as from 1000:10000 to 1; for example from 2000:10000 to 1; such as from 2500:10000 to 1; for example from 3000:10000 to 1; such as from 4000:10000 to 1; for example from 5000:10000 to 1; such as from 6000:10000 to 1; for example from 7000:10000 to 1; such as from 7500:10000 to 1; for example from 8000:10000 to 1; such as from 9000:10000 to 1; for example from 9500:10000 to 1; such as from 1:10000 to 5:10000; for example from 5:10000 to 20:10000, such as from 20:10000 to 100:10000; for example from 100:10000 to 500:10000; such as from 500:10000 to 1000:10000; for example from 1000:10000 to 2000:10000; such as from 2000:10000 to 3000:10000; for example from 3000:10000 to 4000:10000; such as from 4000:10000 to 5000:10000; for example from 5000:10000 to 6000:10000; such as from 6000:10000 to 7000:10000; for example from 7000:10000 to 8000:10000; such as from 8000:10000 to 9000:10000.
199. The bioactive agent according to any of items 7, 38 and 69, wherein the ratio R=b/a between a) the number of glucose monosaccharides and b) the number of galactose monosaccharides is about 0.0001, for example about 0.0005, such as about 0.001, for example about 0.005, such as about 0.01, for example about 0.05, such as about 0.1, for example about 0.2, such as about 0.3, for example about 0.4, such as about 0.5, for example about 0.6, such as about 0.7, for example about 0.8, such as about 0.9, for example about 1; for example from 1:10000 to 1, such as from 2:10000 to 1; for example from 4:10000 to 1; such as from 10:10000 to 1; for example from 20:10000 to 1; such as from 40:10000 to 1; for example from 80:10000 to 1; such as from 100:10000 to 1; for example from 100:10000 to 1; such as from 200:10000 to 1; for example from 250:10000 to 1; such as from 400:10000 to 1; for example from 500:10000 to 1; such as from 1000:10000 to 1; for example from 2000:10000 to 1; such as from 2500:10000 to 1; for example from 3000:10000 to 1; such as from 4000:10000 to 1; for example from 5000:10000 to 1; such as from 6000:10000 to 1; for example from 7000:10000 to 1; such as from 7500:10000 to 1; for example from 8000:10000 to 1; such as from 9000:10000 to 1; for example from 9500:10000 to 1; such as from 1:10000 to 5:10000; for example from 5:10000 to 20:10000, such as from 20:10000 to 100:10000; for example from 100:10000 to 500:10000; such as from 500:10000 to 1000:10000; for example from 1000:10000 to 2000:10000; such as from 2000:10000 to 3000:10000; for example from 3000:10000 to 4000:10000; such as from 4000:10000 to 5000:10000; for example from 5000:10000 to 6000:10000; such as from 6000:10000 to 7000:10000; for example from 7000:10000 to 8000:10000; such as from 8000:10000 to 9000:10000.
200. The bioactive agent according to any of items 7, 38 and 69, wherein the ratio R=a/b between a) the number of glucose monosaccharides and b) the number of mannose monosaccharides is about 0.0001, for example about 0.0005, such as about 0.001, for example about 0.005, such as about 0.01, for example about 0.05, such as about 0.1, for example about 0.2, such as about 0.3, for example about 0.4, such as about 0.5, for example about 0.6, such as about 0.7, for example about 0.8, such as about 0.9, for example about 1; for example from 1:10000 to 1, such as from 2:10000 to 1; for example from 4:10000 to 1; such as from 10:10000 to 1; for example from 20:10000 to 1; such as from 40:10000 to 1; for example from 80:10000 to 1; such as from 100:10000 to 1; for example from 100:10000 to 1; such as from 200:10000 to 1; for example from 250:10000 to 1; such as from 400:10000 to 1; for example from 500:10000 to 1; such as from 1000:10000 to 1; for example from 2000:10000 to 1; such as from 2500:10000 to 1; for example from 3000:10000 to 1; such as from 4000:10000 to 1; for example from 5000:10000 to 1; such as from 6000:10000 to 1; for example from 7000:10000 to 1; such as from 7500:10000 to 1; for example from 8000:10000 to 1; such as from 9000:10000 to 1; for example from 9500:10000 to 1; such as from 1:10000 to 5:10000; for example from 5:10000 to 20:10000, such as from 20:10000 to 100:10000; for example from 100:10000 to 500:10000; such as from 500:10000 to 1000:10000; for example from 1000:10000 to 2000:10000; such as from 2000:10000 to 3000:10000; for example from 3000:10000 to 4000:10000; such as from 4000:10000 to 5000:10000; for example from 5000:10000 to 6000:10000; such as from 6000:10000 to 7000:10000; for example from 7000:10000 to 8000:10000; such as from 8000:10000 to 9000:10000.
201. The bioactive agent according to any of items 7, 38 and 69, wherein the ratio R=b/a between a) the number of glucose monosaccharides and b) the number of mannose monosaccharides is about 0.0001, for example about 0.0005, such as about 0.001, for example about 0.005, such as about 0.01, for example about 0.05, such as about 0.1, for example about 0.2, such as about 0.3, for example about 0.4, such as about 0.5, for example about 0.6, such as about 0.7, for example about 0.8, such as about 0.9, for example about 1; for example from 1:10000 to 1, such as from 2:10000 to 1; for example from 4:10000 to 1; such as from 10:10000 to 1; for example from 20:10000 to 1; such as from 40:10000 to 1; for example from 80:10000 to 1; such as from 100:10000 to 1; for example from 100:10000 to 1; such as from 200:10000 to 1; for example from 250:10000 to 1; such as from 400:10000 to 1; for example from 500:10000 to 1; such as from 1000:10000 to 1; for example from 2000:10000 to 1; such as from 2500:10000 to 1; for example from 3000:10000 to 1; such as from 4000:10000 to 1; for example from 5000:10000 to 1; such as from 6000:10000 to 1; for example from 7000:10000 to 1; such as from 7500:10000 to 1; for example from 8000:10000 to 1; such as from 9000:10000 to 1; for example from 9500:10000 to 1; such as from 1:10000 to 5:10000; for example from 5:10000 to 20:10000, such as from 20:10000 to 100:10000; for example from 100:10000 to 500:10000; such as from 500:10000 to 1000:10000; for example from 1000:10000 to 2000:10000; such as from 2000:10000 to 3000:10000; for example from 3000:10000 to 4000:10000; such as from 4000:10000 to 5000:10000; for example from 5000:10000 to 6000:10000; such as from 6000:10000 to 7000:10000; for example from 7000:10000 to 8000:10000; such as from 8000:10000 to 9000:10000.
202. The bioactive agent according to any of items 7, 38 and 69, wherein the ratio R=a/b between a) the number of glucose monosaccharides and b) the number of arabinose monosaccharides is about 0.0001, for example about 0.0005, such as about 0.001, for example about 0.005, such as about 0.01, for example about 0.05, such as about 0.1, for example about 0.2, such as about 0.3, for example about 0.4, such as about 0.5, for example about 0.6, such as about 0.7, for example about 0.8, such as about 0.9, for example about 1; for example from 1:10000 to 1, such as from 2:10000 to 1; for example from 4:10000 to 1; such as from 10:10000 to 1; for example from 20:10000 to 1; such as from 40:10000 to 1; for example from 80:10000 to 1; such as from 100:10000 to 1; for example from 100:10000 to 1; such as from 200:10000 to 1; for example from 250:10000 to 1; such as from 400:10000 to 1; for example from 500:10000 to 1; such as from 1000:10000 to 1; for example from 2000:10000 to 1; such as from 2500:10000 to 1; for example from 3000:10000 to 1; such as from 4000:10000 to 1; for example from 5000:10000 to 1; such as from 6000:10000 to 1; for example from 7000:10000 to 1; such as from 7500:10000 to 1; for example from 8000:10000 to 1; such as from 9000:10000 to 1; for example from 9500:10000 to 1; such as from 1:10000 to 5:10000; for example from 5:10000 to 20:10000, such as from 20:10000 to 100:10000; for example from 100:10000 to 500:10000; such as from 500:10000 to 1000:10000; for example from 1000:10000 to 2000:10000; such as from 2000:10000 to 3000:10000; for example from 3000:10000 to 4000:10000; such as from 4000:10000 to 5000:10000; for example from 5000:10000 to 6000:10000; such as from 6000:10000 to 7000:10000; for example from 7000:10000 to 8000:10000; such as from 8000:10000 to 9000:10000.
203. The bioactive agent according to any of items 7, 38 and 69, wherein the ratio R=b/a between a) the number of glucose monosaccharides and b) the number of arabinose monosaccharides is about 0.0001, for example about 0.0005, such as about 0.001, for example about 0.005, such as about 0.01, for example about 0.05, such as about 0.1, for example about 0.2, such as about 0.3, for example about 0.4, such as about 0.5, for example about 0.6, such as about 0.7, for example about 0.8, such as about 0.9, for example about 1; for example from 1:10000 to 1, such as from 2:10000 to 1; for example from 4:10000 to 1; such as from 10:10000 to 1; for example from 20:10000 to 1; such as from 40:10000 to 1; for example from 80:10000 to 1; such as from 100:10000 to 1; for example from 100:10000 to 1; such as from 200:10000 to 1; for example from 250:10000 to 1; such as from 400:10000 to 1; for example from 500:10000 to 1; such as from 1000:10000 to 1; for example from 2000:10000 to 1; such as from 2500:10000 to 1; for example from 3000:10000 to 1; such as from 4000:10000 to 1; for example from 5000:10000 to 1; such as from 6000:10000 to 1; for example from 7000:10000 to 1; such as from 7500:10000 to 1; for example from 8000:10000 to 1; such as from 9000:10000 to 1; for example from 9500:10000 to 1; such as from 1:10000 to 5:10000; for example from 5:10000 to 20:10000, such as from 20:10000 to 100:10000; for example from 100:10000 to 500:10000; such as from 500:10000 to 1000:10000; for example from 1000:10000 to 2000:10000; such as from 2000:10000 to 3000:10000; for example from 3000:10000 to 4000:10000; such as from 4000:10000 to 5000:10000; for example from 5000:10000 to 6000:10000; such as from 6000:10000 to 7000:10000; for example from 7000:10000 to 8000:10000; such as from 8000:10000 to 9000:10000.
204. The bioactive agent according to any of items 7, 38 and 69, wherein the ratio R=a/b between a) the number of glucose monosaccharides and b) the number of xylose monosaccharides is about 0.0001, for example about 0.0005, such as about 0.001, for example about 0.005, such as about 0.01, for example about 0.05, such as about 0.1, for example about 0.2, such as about 0.3, for example about 0.4, such as about 0.5, for example about 0.6, such as about 0.7, for example about 0.8, such as about 0.9, for example about 1; for example from 1:10000 to 1, such as from 2:10000 to 1; for example from 4:10000 to 1; such as from 10:10000 to 1; for example from 20:10000 to 1; such as from 40:10000 to 1; for example from 80:10000 to 1; such as from 100:10000 to 1; for example from 100:10000 to 1; such as from 200:10000 to 1; for example from 250:10000 to 1; such as from 400:10000 to 1; for example from 500:10000 to 1; such as from 1000:10000 to 1; for example from 2000:10000 to 1; such as from 2500:10000 to 1; for example from 3000:10000 to 1; such as from 4000:10000 to 1; for example from 5000:10000 to 1; such as from 6000:10000 to 1; for example from 7000:10000 to 1; such as from 7500:10000 to 1; for example from 8000:10000 to 1; such as from 9000:10000 to 1; for example from 9500:10000 to 1; such as from 1:10000 to 5:10000; for example from 5:10000 to 20:10000, such as from 20:10000 to 100:10000; for example from 100:10000 to 500:10000; such as from 500:10000 to 1000:10000; for example from 1000:10000 to 2000:10000; such as from 2000:10000 to 3000:10000; for example from 3000:10000 to 4000:10000; such as from 4000:10000 to 5000:10000; for example from 5000:10000 to 6000:10000; such as from 6000:10000 to 7000:10000; for example from 7000:10000 to 8000:10000; such as from 8000:10000 to 9000:10000.
205. The bioactive agent according to any of items 7, 38 and 69, wherein the ratio R=b/a between a) the number of glucose monosaccharides and b) the number of xylose monosaccharides is about 0.0001, for example about 0.0005, such as about 0.001, for example about 0.005, such as about 0.01, for example about 0.05, such as about 0.1, for example about 0.2, such as about 0.3, for example about 0.4, such as about 0.5, for example about 0.6, such as about 0.7, for example about 0.8, such as about 0.9, for example about 1; for example from 1:10000 to 1, such as from 2:10000 to 1; for example from 4:10000 to 1; such as from 10:10000 to 1; for example from 20:10000 to 1; such as from 40:10000 to 1; for example from 80:10000 to 1; such as from 100:10000 to 1; for example from 100:10000 to 1; such as from 200:10000 to 1; for example from 250:10000 to 1; such as from 400:10000 to 1; for example from 500:10000 to 1; such as from 1000:10000 to 1; for example from 2000:10000 to 1; such as from 2500:10000 to 1; for example from 3000:10000 to 1; such as from 4000:10000 to 1; for example from 5000:10000 to 1; such as from 6000:10000 to 1; for example from 7000:10000 to 1; such as from 7500:10000 to 1; for example from 8000:10000 to 1; such as from 9000:10000 to 1; for example from 9500:10000 to 1; such as from 1:10000 to 5:10000; for example from 5:10000 to 20:10000, such as from 20:10000 to 100:10000; for example from 100:10000 to 500:10000; such as from 500:10000 to 1000:10000; for example from 1000:10000 to 2000:10000; such as from 2000:10000 to 3000:10000; for example from 3000:10000 to 4000:10000; such as from 4000:10000 to 5000:10000; for example from 5000:10000 to 6000:10000; such as from 6000:10000 to 7000:10000; for example from 7000:10000 to 8000:10000; such as from 8000:10000 to 9000:10000.
206. The bioactive agent according to item 2, wherein the polysaccharide comprises a structural component in the back bone comprising beta-1,2-linked D-mannopyranosyl residues and a structural component in the side chains comprising beta-D-glucopyranosyl-3-O-beta-D-glucopyranosyl residues.
207. The bioactive agent according to item 2, wherein the polysaccharide is a complex comprising a (1,4)-alpha-D-glucan and a (1,6)-beta glucan.
208. The bioactive agent according to item 2, wherein the polysaccharide is a complex comprising a (1,4)-alpha-D-glucan and a (1,6)-alpha glucan.
209. The bioactive agent according to any of the above items 1 to 208, wherein said bioactive agent is produced by liquid cultivation of a Basidiomycete cell selected from the group consisting of cells belonging to the subclasses of Agaricomycetidae, Exobasidiomycetidae, Tremellomycetidae and Ustilaginomycetidae.
210. The bioactive agent according to item 209, wherein the Basidiomycete cell is selected form the subclass of Agaricomycetidae.
211. The bioactive agent according to item 209, wherein the Basidiomycete cell is selected form the subclass of Exobasidiomycetidae.
212. The bioactive agent according to item 209, wherein the Basidiomycete cell is selected form the subclass of Tremellomyceditae.
213. The bioactive agent according to item 209, wherein the Basidiomycete cell is selected form the subclass of Ustilaginomycetidae.
214. The bioactive agent according to item 1 to 208, wherein said bioactive agent is produced by liquid cultivation of a Basidiomycete cell selected from the group consisting of cells belonging to the orders of Agaricales, Boletales, Cantheralles, Ceratobasidiales, Dacrymycetales, Hymenochaetales, Phallales, Polyporales, Poriales, Russulales, Thelphorales, Auriculariales, Christianseniales, Cystofilobasidiales, Filobasidiales, Tremellaleles, Tulasenellales and Urocystales.
215. The bioactive agent according to item 214, wherein the Basidiomycete cell is selected from the order of Agaricales.
216. The bioactive agent according to item 215, wherein said Basidiomycete cell belongs to a family selected from the group consisting of Agaricaceae, Bolbitiaceae, Broomeiaceae, Clavariaceae, Coprinaceae, Cortinariaceae, Entolomataceae, Fistulinaceae, Gigaspermaceae, Hemigasteraceae, Hydnangiaceae, Lycoperdaceae, Marasmiaceae, Mesophelliaceae, Mycenastraceae, Niaceae, Nidulariaceae, Phelloriniaceae, Pleurotaceae, Pluteaceae, Pterulaceae, Schizophyllaceae, Stromatocyphaceae, Strophariaceae, Tricholomataceae, Tulostomataceae, Typhulaceae and Xerulaceae.
217. The bioactive agent according to item 216, wherein Basidiomycete cell is selected from the family of Agaricaceae.
218. The bioactive agent according to item 216, wherein Basidiomycete cell is selected from the family of Bolbitiaceae.
219. The bioactive agent according to item 216, wherein Basidiomycete cell is selected from the family of Broomeiaceae.
220. The bioactive agent according to item 216, wherein Basidiomycete cell is selected from the family of Clavariaceae.
221. The bioactive agent according to item 216, wherein Basidiomycete cell is selected from the family of Coprinaceae.
222. The bioactive agent according to item 216, wherein Basidiomycete cell is selected from the family of Cortinariaceae.
223. The bioactive agent according to item 216, wherein Basidiomycete cell is selected from the family of Entolomataceae.
224. The bioactive agent according to item 216, wherein Basidiomycete cell is selected from the family of Fistulinaceae.
225. The bioactive agent according to item 216, wherein Basidiomycete cell is selected from the family of Gigaspermaceae.
226. The bioactive agent according to item 216, wherein Basidiomycete cell is selected from the family of Hemigasteraceae.
227. The bioactive agent according to item 216, wherein Basidiomycete cell is selected from the family of Hydnangiaceae.
228. The bioactive agent according to item 216, wherein Basidiomycete cell is selected from the family of Lycoperdaceae.
229. The bioactive agent according to item 216, wherein Basidiomycete cell is selected from the family of Marasmiaceae.
230. The bioactive agent according to item 216, wherein Basidiomycete cell is selected from the family of Mesophelliaceae.
231. The bioactive agent according to item 216, wherein Basidiomycete cell is selected from the family of Mycenastraceae.
232. The bioactive agent according to item 216, wherein Basidiomycete cell is selected from the family of Niaceae.
233. The bioactive agent according to item 216, wherein Basidiomycete cell is selected from the family of Nidulariaceae.
234. The bioactive agent according to item 216, wherein Basidiomycete cell is selected from the family of Phelloriniaceae.
235. The bioactive agent according to item 216, wherein Basidiomycete cell is selected from the family of Pleurotaceae.
236. The bioactive agent according to item 216, wherein Basidiomycete cell is selected from the family of Pluteaceae.
237. The bioactive agent according to item 216, wherein Basidiomycete cell is selected from the family of Pterulaceae.
238. The bioactive agent according to item 216, wherein Basidiomycete cell is selected from the family of Schizophyllaceae.
239. The bioactive agent according to item 216, wherein Basidiomycete cell is selected from the family of Stromatocyphaceae.
240. The bioactive agent according to item 216, wherein Basidiomycete cell is selected from the family of Strophariaceae.
241. The bioactive agent according to item 216, wherein Basidiomycete cell is selected from the family of Tricholomataceae.
242. The bioactive agent according to item 216, wherein Basidiomycete cell is selected from the family of Tulostomataceae.
243. The bioactive agent according to item 216, wherein Basidiomycete cell is selected from the family of Typhulaceae.
244. The bioactive agent according to item 216, wherein Basidiomycete cell is selected from the family of Xerulaceae.
245. The bioactive agent according to item 214, wherein the Basidiomycete cell is selected from the order of Polyporales.
246. The bioactive agent according to item 245, wherein said Basidiomycete cell belongs to a family selected from the group consisting of Albatrellaceae, Atheliaceae, Boreostereaceae, Corticiaceae, Cyphellaceae, Cystostereaceae, Epitheliaceae, Fomitopsidaceae, Ganodermataceae, Gloeophyllaceae, Grammotheleaceae, Hapalopilaceae, Hyphodermataceae, Meripilaceae, Meruliaceae, Phanerochaetaceae, Podoscyphaceae, Polyporaceae, Sistotremataceae, Sparassidaceae, Steccherinaceae, Tubulicrinaceae and Xenasmataceae.
247. The bioactive agent according to item 246, wherein Basidiomycete cell is selected from the family of Albatrellaceae.
248. The bioactive agent according to item 246, wherein Basidiomycete cell is selected from the family of Atheliaceae.
249. The bioactive agent according to item 246, wherein Basidiomycete cell is selected from the family of Boreostereaceae.
250. The bioactive agent according to item 246, wherein Basidiomycete cell is selected from the family of Corticiaceae.
251. The bioactive agent according to item 246, wherein Basidiomycete cell is selected from the family of Cyphellaceae.
252. The bioactive agent according to item 246, wherein Basidiomycete cell is selected from the family of Cystostereaceae.
253. The bioactive agent according to item 246, wherein Basidiomycete cell is selected from the family of Epitheliaceae.
254. The bioactive agent according to item 246, wherein Basidiomycete cell is selected from the family of Fomitopsidaceae.
255. The bioactive agent according to item 246, wherein Basidiomycete cell is selected from the family of Ganodermataceae.
256. The bioactive agent according to item 246, wherein Basidiomycete cell is selected from the family of Gloeophyllaceae.
257. The bioactive agent according to item 246, wherein Basidiomycete cell is selected from the family of Grammotheleaceae.
258. The bioactive agent according to item 246, wherein Basidiomycete cell is selected from the family of Hapalopilaceae.
259. The bioactive agent according to item 246, wherein Basidiomycete cell is selected from the family of Hyphodermataceae.
260. The bioactive agent according to item 246, wherein Basidiomycete cell is selected from the family of Meripilaceae.
261. The bioactive agent according to item 246, wherein Basidiomycete cell is selected from the family of Meruliaceae.
262. The bioactive agent according to item 246, wherein Basidiomycete cell is selected from the family of Phanerochaetaceae.
263. The bioactive agent according to item 246, wherein Basidiomycete cell is selected from the family of Podoscyphaceae.
264. The bioactive agent according to item 246, wherein Basidiomycete cell is selected from the family of Polyporaceae.
265. The bioactive agent according to item 246, wherein Basidiomycete cell is selected from the family of Sistotremataceae.
266. The bioactive agent according to item 246, wherein Basidiomycete cell is selected from the family of Sparassidaceae.
267. The bioactive agent according to item 246, wherein Basidiomycete cell is selected from the family of Steccherinaceae.
268. The bioactive agent according to item 246, wherein Basidiomycete cell is selected from the family of Tubulicrinaceae.
269. The bioactive agent according to item 246, wherein Basidiomycete cell is selected from the family of Xenasmataceae.
270. The bioactive agent according to item 214, wherein the Basidiomycete cell is selected from the order of Boletales.
271. The bioactive agent according to item 270, wherein said Basidiomycete cell belongs to a family selected from the group consisting of Boletaceae, Boletinellaceae, Coniophoraceae, Diplocystaceae, Gasterellaceae, Gastrosporiaceae, Gomphidiaceae, Gyroporaceae, Hygrophoropsidaceae, Hymenogasteraceae, Leucogastraceae, Melanogastraceae, Octavianiaceae, Octavianinaceae, Paxillaceae, Protogastraceae, Rhizopogonaceae, Sclerodermataceae and Suillaceae.
272. The bioactive agent according to item 271, wherein Basidiomycete cell is selected from the family of Boletaceae.
273. The bioactive agent according to item 271, wherein Basidiomycete cell is selected from the family of Boletinellaceae.
274. The bioactive agent according to item 271, wherein Basidiomycete cell is selected from the family of Coniophoraceae.
275. The bioactive agent according to item 271, wherein Basidiomycete cell is selected from the family of Diplocystaceae.
276. The bioactive agent according to item 271, wherein Basidiomycete cell is selected from the family of Gasterellaceae.
277. The bioactive agent according to item 271, wherein Basidiomycete cell is selected from the family of Gastrosporiaceae.
278. The bioactive agent according to item 271, wherein Basidiomycete cell is selected from the family of Gomphidiaceae.
279. The bioactive agent according to item 271, wherein Basidiomycete cell is selected from the family of Gyroporaceae.
280. The bioactive agent according to item 271, wherein Basidiomycete cell is selected from the family of Hygrophoropsidaceae.
281. The bioactive agent according to item 271, wherein Basidiomycete cell is selected from the family of Hymenogasteraceae.
282. The bioactive agent according to item 271, wherein Basidiomycete cell is selected from the family of Leucogastraceae.
283. The bioactive agent according to item 271, wherein Basidiomycete cell is selected from the family of Melanogastraceae.
284. The bioactive agent according to item 271, wherein Basidiomycete cell is selected from the family of Octavianiaceae.
285. The bioactive agent according to item 271, wherein Basidiomycete cell is selected from the family of Octavianinaceae.
286. The bioactive agent according to item 271, wherein Basidiomycete cell is selected from the family of Paxillaceae.
287. The bioactive agent according to item 271, wherein Basidiomycete cell is selected from the family of Protogastraceae.
288. The bioactive agent according to item 271, wherein Basidiomycete cell is selected from the family of Rhizopogonaceae.
289. The bioactive agent according to item 271, wherein Basidiomycete cell is selected from the family of Sclerodermataceae.
290. The bioactive agent according to item 271, wherein Basidiomycete cell is selected from the family of Suillaceae.
291. The bioactive agent according to item 214, wherein the Basidiomycete cell is selected from the order of Cantheralles.
292. The bioactive agent according to item 291, wherein said Basidiomycete cell belongs to a family selected from the group consisting of Aphelariaceae, Botryobasidiaceae, Cantharellaceae, Clavulinaceae, and Hydnaceae.
293. The bioactive agent according to item 271, wherein Basidiomycete cell is selected from the family of Aphelariaceae.
294. The bioactive agent according to item 271, wherein Basidiomycete cell is selected from the family of Botryobasidiaceae.
295. The bioactive agent according to item 271, wherein Basidiomycete cell is selected from the family of Cantharellaceae.
296. The bioactive agent according to item 271, wherein Basidiomycete cell is selected from the family of Clavulinaceae.
297. The bioactive agent according to item 271, wherein Basidiomycete cell is selected from the family of Hydnaceae.
298. The bioactive agent according to item 214, wherein the Basidiomycete cell is selected from the order of Ceratobasidiales.
299. The bioactive agent according to item 298, wherein said Basidiomycete cell belongs to a family selected from the group consisting of Ceratobasidiaceae and Oliveoniaceae.
300. The bioactive agent according to item 299, wherein Basidiomycete cell is selected from the family of Ceratobasidiaceae.
301. The bioactive agent according to item 299, wherein Basidiomycete cell is selected from the family of Oliveoniaceae.
302. The bioactive agent according to item 214, wherein the Basidiomycete cell is selected from the order of Dacrymycetales.
303. The bioactive agent according to item 302, wherein said Basidiomycete cell belongs to a family selected from the group consisting of Cerinomycetaceae and Dacrymycetaceae.
304. The bioactive agent according to item 303, wherein Basidiomycete cell is selected from the family of Cerinomycetaceae.
305. The bioactive agent according to item 303, wherein Basidiomycete cell is selected from the family of Dacrymycetaceae.
306. The bioactive agent according to item 214, wherein the Basidiomycete cell is selected from the order of Hymenochaetales.
307. The bioactive agent according to item 306, wherein said Basidiomycete cell belongs to a family selected from the group consisting of Asterostromataceae, Hymenochaetaceae and Schizoporaceae.
308. The bioactive agent according to item 307, wherein Basidiomycete cell is selected from the family of Asterostromataceae.
309. The bioactive agent according to item 307, wherein Basidiomycete cell is selected from the family of Hymenochaetaceae.
310. The bioactive agent according to item 307, wherein Basidiomycete cell is selected from the family of Schizoporaceae.
311. The bioactive agent according to item 214, wherein the Basidiomycete cell is selected from the order of Phallales.
312. The bioactive agent according to item 311, wherein said Basidiomycete cell belongs to a family selected from the group consisting of Geastraceae, Gomphaceae, Hysterangiaceae, Phallaceae and Ramariaceae.
313. The bioactive agent according to item 312, wherein Basidiomycete cell is selected from the family of Geastraceae.
314. The bioactive agent according to item 312, wherein Basidiomycete cell is selected from the family of Gomphaceae.
315. The bioactive agent according to item 312, wherein Basidiomycete cell is selected from the family of Hysterangiaceae.
316. The bioactive agent according to item 312, wherein Basidiomycete cell is selected from the family of Phallaceae.
317. The bioactive agent according to item 312, wherein Basidiomycete cell is selected from the family of Ramariaceae.
318. The bioactive agent according to item 214, wherein the Basidiomycete cell is selected from the order of Poriales.
319. The bioactive agent according to item 318, wherein said Basidiomycete cell belongs to a family of Polyporaceae.
320. The bioactive agent according to item 214, wherein the Basidiomycete cell is selected from the order of Russulales.
321. The bioactive agent according to item 320, wherein said Basidiomycete cell belongs to a family selected from the group consisting of Auriscalpiaceae, Bondarzewiaceae, Echinodontiaceae, Hericiaceae, Hybogasteraceae, Lachnocladiaceae, Peniophoraceae, Phanerochaetaceae, Russulaceae, Stephanosporaceae and Stereaceae.
322. The bioactive agent according to item 321, wherein Basidiomycete cell is selected from the family of Auriscalpiaceae.
323. The bioactive agent according to item 321, wherein Basidiomycete cell is selected from the family of Bondarzewiaceae.
324. The bioactive agent according to item 321, wherein Basidiomycete cell is selected from the family of Echinodontiaceae.
325. The bioactive agent according to item 321, wherein Basidiomycete cell is selected from the family of Hericiaceae.
326. The bioactive agent according to item 321, wherein Basidiomycete cell is selected from the family of Hybogasteraceae.
327. The bioactive agent according to item 321, wherein Basidiomycete cell is selected from the family of Lachnocladiaceae.
328. The bioactive agent according to item 321, wherein Basidiomycete cell is selected from the family of Peniophoraceae.
329. The bioactive agent according to item 321, wherein Basidiomycete cell is selected from the family of Phanerochaetaceae.
330. The bioactive agent according to item 321, wherein Basidiomycete cell is selected from the family of Russulaceae.
331. The bioactive agent according to item 321, wherein Basidiomycete cell is selected from the family of Stephanosporaceae.
332. The bioactive agent according to item 321, wherein Basidiomycete cell is selected from the family of Stereaceae.
333. The bioactive agent according to item 214, wherein the Basidiomycete cell is selected from the order of Thelophorales.
334. The bioactive agent according to item 333, wherein said Basidiomycete cell belongs to a family selected from the group consisting of Bankeraceae and Thelephoraceae.
335. The bioactive agent according to item 334, wherein Basidiomycete cell is selected from the family of Bankeraceae.
336. The bioactive agent according to item 334, wherein Basidiomycete cell is selected from the family of Thelephoraceae.
337. The bioactive agent according to item 214, wherein the Basidiomycete cell is selected from the order of Auriculariales.
338. The bioactive agent according to item 337, wherein Basidiomycete cell is selected from the family of Auriculariaceae.
339. The bioactive agent according to item 214, wherein the Basidiomycete cell is selected from the order of Christianseniales.
340. The bioactive agent according to item 339, wherein Basidiomycete cell is selected from the family of Christianseniaceae.
341. The bioactive agent according to item 214, wherein the Basidiomycete cell is selected from the order of Cystofilobasidiales.
342. The bioactive agent according to item 341, wherein Basidiomycete cell is selected from the family of Cystofilobasidiaceae.
343. The bioactive agent according to item 214, wherein the Basidiomycete cell is selected from the order of Filobasidiales.
344. The bioactive agent according to item 343, wherein Basidiomycete cell is selected from the family of Filobasidiaceae.
345. The bioactive agent according to item 214, wherein the Basidiomycete cell is selected from the order of Tremellales.
346. The bioactive agent according to item 345, wherein said Basidiomycete cell belongs to a family selected from the group consisting of Aporpiaceae, Cuniculitremaceae, Exidiaceae, Hyaloriaceae, Phragmoxenidiaceae, Rhynchogastremataceae, Sirobasidiaceae, Syzygosporaceae, Tetragoniomycetaceae, Tremellaceae and Tremellodendropsidaceae.
347. The bioactive agent according to item 346, wherein Basidiomycete cell is selected from the family of Aporpiaceae.
348. The bioactive agent according to item 346, wherein Basidiomycete cell is selected from the family of Cuniculitremaceae.
349. The bioactive agent according to item 346, wherein Basidiomycete cell is selected from the family of Exidiaceae.
350. The bioactive agent according to item 346, wherein Basidiomycete cell is selected from the family of Hyaloriaceae.
351. The bioactive agent according to item 346, wherein Basidiomycete cell is selected from the family of Phragmoxenidiaceae.
352. The bioactive agent according to item 346, wherein Basidiomycete cell is selected from the family of Rhynchogastremataceae.
353. The bioactive agent according to item 346, wherein Basidiomycete cell is selected from the family of Sirobasidiaceae.
354. The bioactive agent according to item 346, wherein Basidiomycete cell is selected from the family of Syzygosporaceae.
355. The bioactive agent according to item 346, wherein Basidiomycete cell is selected from the family of Tetragoniomycetaceae.
356. The bioactive agent according to item 346, wherein Basidiomycete cell is selected from the family of Tremellaceae.
357. The bioactive agent according to item 346, wherein Basidiomycete cell is selected from the family of Tremellodendropsidaceae.
358. The bioactive agent according to item 214, wherein the Basidiomycete cell is selected from the order of Tulasenellales.
359. The bioactive agent according to item 358, wherein Basidiomycete cell is selected from the family of Tulasnellaceae.
360. The bioactive agent according to item 214, wherein the Basidiomycete cell is selected from the order of Urocystales.
361. The bioactive agent according to item 360, wherein Basidiomycete cell is selected from the family of Urocystaceae.
362. The bioactive agent according to item 217, wherein said Basidiomycete cell belongs to a genus selected from the group consisting of Agaricus, Amanita, Amylolepiota, Araneosa, Artymenium, Attamyces, Barcheria, Cauloglossum, Chainoderma, Chamaemyces, Chitonia, Chitoniella, Chitonis, Chlorolepiota, Chlorophyllum, Chlorosperma, Chlorospora, Clarkeinda, Clavogaster, Coccobotrys, Crucispora, Cystoagaricus, Cystolepiota, Drosella, Endolepiotula, Fungus, Fusispora, Gasterellopsis, Glaucospora, Gymnogaster, Gyrophragmium, Heinemannomyces, Herculea, Hiatulopsis, Holocotylon, Horakia, Hymenagaricus, Hypogaea, Hypophyllum, Lepidotus, Lepiotella, Lepiotula, Leucoagaricus, Leucobolbitius, Leucocoprinus, Longia, Longula, Macrolepiota, Mastocephalus, Melanophyllum, Metraria, Metrodia, Micropsalliota, Montagnea, Montagnites, Morobia, Myces, Neosecotium, Notholepiota, Panaeolopsis, Phaeopholiota, Phlebonema, Phyllogaster, Podaxis, Polyplocium, Pseudoauricularia, Pulverolepiota, Rickella, Rugosospora, Schinzinia, Schulzeria, Schweinitzia, Secotium, Sericeomyces, Singerina, Smithiogaster, Smithiomyces, Stellifera, Termiticola, Verrucospora, Volvigerum, Volvolepiota and Xanthagaricus.
363. The bioactive agent according to item 362, wherein Basidiomycete cell is selected from the genus of Agaricus.
364. The bioactive agent according to item 362, wherein Basidiomycete cell is selected from the genus of Amanita.
365. The bioactive agent according to item 362, wherein Basidiomycete cell is selected from the genus of Amylolepiota.
366. The bioactive agent according to item 362, wherein Basidiomycete cell is selected from the genus of Araneosa.
367. The bioactive agent according to item 362, wherein Basidiomycete cell is selected from the genus of Artymenium.
368. The bioactive agent according to item 362, wherein Basidiomycete cell is selected from the genus of Attamyces.
369. The bioactive agent according to item 362, wherein Basidiomycete cell is selected from the genus of Barcheria.
370. The bioactive agent according to item 362, wherein Basidiomycete cell is selected from the genus of Cauloglossum.
371. The bioactive agent according to item 362, wherein Basidiomycete cell is selected from the genus of Chainoderma.
372. The bioactive agent according to item 362, wherein Basidiomycete cell is selected from the genus of Chamaemyces.
373. The bioactive agent according to item 362, wherein Basidiomycete cell is selected from the genus of Chitonia.
374. The bioactive agent according to item 362, wherein Basidiomycete cell is selected from the genus of Chitoniella.
375. The bioactive agent according to item 362, wherein Basidiomycete cell is selected from the genus of Chitonis.
376. The bioactive agent according to item 362, wherein Basidiomycete cell is selected from the genus of Chlorolepiota.
377. The bioactive agent according to item 362, wherein Basidiomycete cell is selected from the genus of Chlorophyllum.
378. The bioactive agent according to item 362, wherein Basidiomycete cell is selected from the genus of Chlorosperma.
379. The bioactive agent according to item 362, wherein Basidiomycete cell is selected from the genus of Chlorospora.
380. The bioactive agent according to item 362, wherein Basidiomycete cell is selected from the genus of Clarkeinda.
381. The bioactive agent according to item 362, wherein Basidiomycete cell is selected from the genus of Clavogaster.
382. The bioactive agent according to item 362, wherein Basidiomycete cell is selected from the genus of Coccobotrys.
383. The bioactive agent according to item 362, wherein Basidiomycete cell is selected from the genus of Crucispora.
384. The bioactive agent according to item 362, wherein Basidiomycete cell is selected from the genus of Cystoagaricus.
385. The bioactive agent according to item 362, wherein Basidiomycete cell is selected from the genus of Cystolepiota.
386. The bioactive agent according to item 362, wherein Basidiomycete cell is selected from the genus of Drosella.
387. The bioactive agent according to item 362, wherein Basidiomycete cell is selected from the genus of Endolepiotula.
388. The bioactive agent according to item 362, wherein Basidiomycete cell is selected from the genus of Fungus.
389. The bioactive agent according to item 362, wherein Basidiomycete cell is selected from the genus of Fusispora.
390. The bioactive agent according to item 362, wherein Basidiomycete cell is selected from the genus of Gasterellopsis.
391. The bioactive agent according to item 362, wherein Basidiomycete cell is selected from the genus of Glaucospora.
392. The bioactive agent according to item 362, wherein Basidiomycete cell is selected from the genus of Gymnogaster.
393. The bioactive agent according to item 362, wherein Basidiomycete cell is selected from the genus of Gyrophragmium.
394. The bioactive agent according to item 362, wherein Basidiomycete cell is selected from the genus of Heinemannomyces.
395. The bioactive agent according to item 362, wherein Basidiomycete cell is selected from the genus of Herculea.
396. The bioactive agent according to item 362, wherein Basidiomycete cell is selected from the genus of Hiatulopsis.
397. The bioactive agent according to item 362, wherein Basidiomycete cell is selected from the genus of Holocotylon.
398. The bioactive agent according to item 362, wherein Basidiomycete cell is selected from the genus of Horakia.
399. The bioactive agent according to item 362, wherein Basidiomycete cell is selected from the genus of Hymenagaricus.
400. The bioactive agent according to item 362, wherein Basidiomycete cell is selected from the genus of Hypogaea.
401. The bioactive agent according to item 362, wherein Basidiomycete cell is selected from the genus of Hypophyllum.
402. The bioactive agent according to item 362, wherein Basidiomycete cell is selected from the genus of Lepidotus.
403. The bioactive agent according to item 362, wherein Basidiomycete cell is selected from the genus of Lepiotella.
404. The bioactive agent according to item 362, wherein Basidiomycete cell is selected from the genus of Lepiotula.
405. The bioactive agent according to item 362, wherein Basidiomycete cell is selected from the genus of Leucoagaricus.
406. The bioactive agent according to item 362, wherein Basidiomycete cell is selected from the genus of Leucobolbitius.
407. The bioactive agent according to item 362, wherein Basidiomycete cell is selected from the genus of Leucocoprinus.
408. The bioactive agent according to item 362, wherein Basidiomycete cell is selected from the genus of Longia.
409. The bioactive agent according to item 362, wherein Basidiomycete cell is selected from the genus of Longula.
410. The bioactive agent according to item 362, wherein Basidiomycete cell is selected from the genus of Macrolepiota.
411. The bioactive agent according to item 362, wherein Basidiomycete cell is selected from the genus of Mastocephalus.
412. The bioactive agent according to item 362, wherein Basidiomycete cell is selected from the genus of Melanophyllum.
413. The bioactive agent according to item 362, wherein Basidiomycete cell is selected from the genus of Metraria.
414. The bioactive agent according to item 362, wherein Basidiomycete cell is selected from the genus of Metrodia.
415. The bioactive agent according to item 362, wherein Basidiomycete cell is selected from the genus of Micropsalliota.
416. The bioactive agent according to item 362, wherein Basidiomycete cell is selected from the genus of Montagnea.
417. The bioactive agent according to item 362, wherein Basidiomycete cell is selected from the genus of Montagnites.
418. The bioactive agent according to item 362, wherein Basidiomycete cell is selected from the genus of Morobia.
419. The bioactive agent according to item 362, wherein Basidiomycete cell is selected from the genus of Myces.
420. The bioactive agent according to item 362, wherein Basidiomycete cell is selected from the genus of Neosecotium.
421. The bioactive agent according to item 362, wherein Basidiomycete cell is selected from the genus of Notholepiota.
422. The bioactive agent according to item 362, wherein Basidiomycete cell is selected from the genus of Panaeolopsis.
423. The bioactive agent according to item 362, wherein Basidiomycete cell is selected from the genus of Phaeopholiota.
424. The bioactive agent according to item 362, wherein Basidiomycete cell is selected from the genus of Phlebonema.
425. The bioactive agent according to item 362, wherein Basidiomycete cell is selected from the genus of Phyllogaster.
426. The bioactive agent according to item 362, wherein Basidiomycete cell is selected from the genus of Podaxis.
427. The bioactive agent according to item 362, wherein Basidiomycete cell is selected from the genus of Polyplocium.
428. The bioactive agent according to item 362, wherein Basidiomycete cell is selected from the genus of Pseudoauricularia.
429. The bioactive agent according to item 362, wherein Basidiomycete cell is selected from the genus of Pulverolepiota.
430. The bioactive agent according to item 362, wherein Basidiomycete cell is selected from the genus of Rickella.
431. The bioactive agent according to item 362, wherein Basidiomycete cell is selected from the genus of Rugosospora.
432. The bioactive agent according to item 362, wherein Basidiomycete cell is selected from the genus of Schinzinia.
433. The bioactive agent according to item 362, wherein Basidiomycete cell is selected from the genus of Schulzeria.
434. The bioactive agent according to item 362, wherein Basidiomycete cell is selected from the genus of Schweinitzia.
435. The bioactive agent according to item 362, wherein Basidiomycete cell is selected from the genus of Secotium.
436. The bioactive agent according to item 362, wherein Basidiomycete cell is selected from the genus of Sericeomyces.
437. The bioactive agent according to item 362, wherein Basidiomycete cell is selected from the genus of Singerina.
438. The bioactive agent according to item 362, wherein Basidiomycete cell is selected from the genus of Smithiogaster.
439. The bioactive agent according to item 362, wherein Basidiomycete cell is selected from the genus of Smithiomyces.
440. The bioactive agent according to item 362, wherein Basidiomycete cell is selected from the genus of Stellifera.
441. The bioactive agent according to item 362, wherein Basidiomycete cell is selected from the genus of Termiticola.
442. The bioactive agent according to item 362, wherein Basidiomycete cell is selected from the genus of Verrucospora.
443. The bioactive agent according to item 362, wherein Basidiomycete cell is selected from the genus of Volvigerum.
444. The bioactive agent according to item 362, wherein Basidiomycete cell is selected from the genus of Volvolepiota.
445. The bioactive agent according to item 362, wherein Basidiomycete cell is selected from the genus of Xanthagaricus.
446. The bioactive agent according to item 218, wherein said Basidiomycete cell belongs to a genus selected from the group consisting of Acetabularia, Agrocybe, Agrogaster, Alnicola, Anellaria, Bolbitius, Bulla, Campanularius, Chalymmota, Conocybe, Copelandia, Coprinarius, Cyclocybe, Cyclopus, Cyphellopus, Cyttarophyllopsis, Cyttarophyllum, Galerella, Galeropsis, Gastrocybe, Gymnoglossum, Hebeloma, Hebelomatis, Hylophila, Myxocybe, Naucoria, Panaeolina, Panaeolus, Pholiotella, Pholiotina, Picromyces, Pluteolus, Psammomyces, Pseudoconocybe, Pseudodeconica, Ptychella, Raddetes, Roumeguerites, Sarcoloma, Setchelliogaster, Togaria, Tubariella, Tubariopsis, Tympanella and Wielandomyces.
447. The bioactive agent according to item 446, wherein Basidiomycete cell is selected from the genus of Acetabularia.
448. The bioactive agent according to item 446, wherein Basidiomycete cell is selected from the genus of Agrocybe.
449. The bioactive agent according to item 446, wherein Basidiomycete cell is selected from the genus of Agrogaster.
450. The bioactive agent according to item 446, wherein Basidiomycete cell is selected from the genus of Alnicola.
451. The bioactive agent according to item 446, wherein Basidiomycete cell is selected from the genus of Anellaria.
452. The bioactive agent according to item 446, wherein Basidiomycete cell is selected from the genus of Bolbitius.
453. The bioactive agent according to item 446, wherein Basidiomycete cell is selected from the genus of Bulla.
454. The bioactive agent according to item 446, wherein Basidiomycete cell is selected from the genus of Campanularius.
455. The bioactive agent according to item 446, wherein Basidiomycete cell is selected from the genus of Chalymmota.
456. The bioactive agent according to item 446, wherein Basidiomycete cell is selected from the genus of Conocybe.
457. The bioactive agent according to item 446, wherein Basidiomycete cell is selected from the genus of Copelandia.
458. The bioactive agent according to item 446, wherein Basidiomycete cell is selected from the genus of Coprinarius.
459. The bioactive agent according to item 446, wherein Basidiomycete cell is selected from the genus of Cyclocybe.
460. The bioactive agent according to item 446, wherein Basidiomycete cell is selected from the genus of Cyclopus.
461. The bioactive agent according to item 446, wherein Basidiomycete cell is selected from the genus of Cyphellopus.
462. The bioactive agent according to item 446, wherein Basidiomycete cell is selected from the genus of Cyttarophyllopsis.
463. The bioactive agent according to item 446, wherein Basidiomycete cell is selected from the genus of Cyttarophyllum.
464. The bioactive agent according to item 446, wherein Basidiomycete cell is selected from the genus of Galerella.
465. The bioactive agent according to item 446, wherein Basidiomycete cell is selected from the genus of Galeropsis.
466. The bioactive agent according to item 446, wherein Basidiomycete cell is selected from the genus of Gastrocybe.
467. The bioactive agent according to item 446, wherein Basidiomycete cell is selected from the genus of Gymnoglossum.
468. The bioactive agent according to item 446, wherein Basidiomycete cell is selected from the genus of Hebeloma.
469. The bioactive agent according to item 446, wherein Basidiomycete cell is selected from the genus of Hebelomatis.
470. The bioactive agent according to item 446, wherein Basidiomycete cell is selected from the genus of Hylophila.
471. The bioactive agent according to item 446, wherein Basidiomycete cell is selected from the genus of Myxocybe.
472. The bioactive agent according to item 446, wherein Basidiomycete cell is selected from the genus of Naucoria.
473. The bioactive agent according to item 446, wherein Basidiomycete cell is selected from the genus of Panaeolina.
474. The bioactive agent according to item 446, wherein Basidiomycete cell is selected from the genus of Panaeolus.
475. The bioactive agent according to item 446, wherein Basidiomycete cell is selected from the genus of Pholiotella.
476. The bioactive agent according to item 446, wherein Basidiomycete cell is selected from the genus of Pholiotina.
477. The bioactive agent according to item 446, wherein Basidiomycete cell is selected from the genus of Picromyces.
478. The bioactive agent according to item 446, wherein Basidiomycete cell is selected from the genus of Pluteolus.
479. The bioactive agent according to item 446, wherein Basidiomycete cell is selected from the genus of Psammomyces.
480. The bioactive agent according to item 446, wherein Basidiomycete cell is selected from the genus of Pseudoconocybe.
481. The bioactive agent according to item 446, wherein Basidiomycete cell is selected from the genus of Pseudodeconica.
482. The bioactive agent according to item 446, wherein Basidiomycete cell is selected from the genus of Ptychella.
483. The bioactive agent according to item 446, wherein Basidiomycete cell is selected from the genus of Raddetes.
484. The bioactive agent according to item 446, wherein Basidiomycete cell is selected from the genus of Roumeguerites.
485. The bioactive agent according to item 446, wherein Basidiomycete cell is selected from the genus of Sarcoloma.
486. The bioactive agent according to item 446, wherein Basidiomycete cell is selected from the genus of Setchelliogaster.
487. The bioactive agent according to item 446, wherein Basidiomycete cell is selected from the genus of Togaria.
488. The bioactive agent according to item 446, wherein Basidiomycete cell is selected from the genus of Tubariella.
489. The bioactive agent according to item 446, wherein Basidiomycete cell is selected from the genus of Tubariopsis.
490. The bioactive agent according to item 446, wherein Basidiomycete cell is selected from the genus of Tympanella.
491. The bioactive agent according to item 446, wherein Basidiomycete cell is selected from the genus of Wielandomyces.
492. The bioactive agent according to item 219, wherein Basidiomycete cell is selected from the genus of Broomeia.
493. The bioactive agent according to item 220, wherein said Basidiomycete cell belongs to a genus selected from the group consisting of Capitoclavaria, Clavaria, Clavulinopsis, Cornicularia, Donkella, Holocoryne, Macrotyphula, Manina, Multiclavula, Podostrombium, Ramaria, Ramariopsis, Scytinopogon, Setigeroclavula and Stichoclavaria.
494. The bioactive agent according to item 493, wherein Basidiomycete cell is selected from the genus of Capitoclavaria.
495. The bioactive agent according to item 493, wherein Basidiomycete cell is selected from the genus of Clavaria.
496. The bioactive agent according to item 493, wherein Basidiomycete cell is selected from the genus of Clavulinopsis.
497. The bioactive agent according to item 493, wherein Basidiomycete cell is selected from the genus of Cornicularia.
498. The bioactive agent according to item 493, wherein Basidiomycete cell is selected from the genus of Donkella.
499. The bioactive agent according to item 493, wherein Basidiomycete cell is selected from the genus of Holocoryne.
500. The bioactive agent according to item 493, wherein Basidiomycete cell is selected from the genus of Macrotyphula.
501. The bioactive agent according to item 493, wherein Basidiomycete cell is selected from the genus of Manina.
502. The bioactive agent according to item 493, wherein Basidiomycete cell is selected from the genus of Multiclavula.
503. The bioactive agent according to item 493, wherein Basidiomycete cell is selected from the genus of Podostrombium.
504. The bioactive agent according to item 493, wherein Basidiomycete cell is selected from the genus of Ramaria.
505. The bioactive agent according to item 493, wherein Basidiomycete cell is selected from the genus of Ramariopsis.
506. The bioactive agent according to item 493, wherein Basidiomycete cell is selected from the genus of Scytinopogon.
507. The bioactive agent according to item 493, wherein Basidiomycete cell is selected from the genus of Setigeroclavula.
508. The bioactive agent according to item 493, wherein Basidiomycete cell is selected from the genus of Stichoclavaria.
509. The bioactive agent according to item 221, wherein said Basidiomycete cell belongs to a genus selected from the group consisting of Annularius, Astylospora, Coprinellus, Coprinopsis, Coprinus, Coprinusella, Cortiniopsis, Drosophila, Ephemerocybe, Gasteroagaricoides, Glyptospora, Gymnochilus, Homophron, Hypholomopsis, Lacrymaria, Lentispora, Macrometrula, Onchopus, Palaeocybe, Pannucia, Parasola, Pluteopsis, Psalliotina, Psammocoparius, Psathyra, Psathyrella, Pselliophora, Pseudocoprinus, Psilocybe, Rhacophyllus, Xerocoprinus and Zerovaemyces.
510. The bioactive agent according to item 509, wherein Basidiomycete cell is selected from the genus of Annularius.
511. The bioactive agent according to item 509, wherein Basidiomycete cell is selected from the genus of Astylospora.
512. The bioactive agent according to item 509, wherein Basidiomycete cell is selected from the genus of Coprinellus.
513. The bioactive agent according to item 509, wherein Basidiomycete cell is selected from the genus of Coprinopsis.
514. The bioactive agent according to item 509, wherein Basidiomycete cell is selected from the genus of Coprinus.
515. The bioactive agent according to item 509, wherein Basidiomycete cell is selected from the genus of Coprinusella.
516. The bioactive agent according to item 509, wherein Basidiomycete cell is selected from the genus of Cortiniopsis.
517. The bioactive agent according to item 509, wherein Basidiomycete cell is selected from the genus of Drosophila.
518. The bioactive agent according to item 509, wherein Basidiomycete cell is selected from the genus of Ephemerocybe.
519. The bioactive agent according to item 509, wherein Basidiomycete cell is selected from the genus of Gasteroagaricoides.
520. The bioactive agent according to item 509, wherein Basidiomycete cell is selected from the genus of Glyptospora.
521. The bioactive agent according to item 509, wherein Basidiomycete cell is selected from the genus of Gymnochilus.
522. The bioactive agent according to item 509, wherein Basidiomycete cell is selected from the genus of Homophron.
523. The bioactive agent according to item 509, wherein Basidiomycete cell is selected from the genus of Hypholomopsis.
524. The bioactive agent according to item 509, wherein Basidiomycete cell is selected from the genus of Lacrymaria.
525. The bioactive agent according to item 509, wherein Basidiomycete cell is selected from the genus of Lentispora.
526. The bioactive agent according to item 509, wherein Basidiomycete cell is selected from the genus of Macrometrula.
527. The bioactive agent according to item 509, wherein Basidiomycete cell is selected from the genus of Onchopus.
528. The bioactive agent according to item 509, wherein Basidiomycete cell is selected from the genus of Palaeocybe.
529. The bioactive agent according to item 509, wherein Basidiomycete cell is selected from the genus of Pannucia.
530. The bioactive agent according to item 509, wherein Basidiomycete cell is selected from the genus of Parasola.
531. The bioactive agent according to item 509, wherein Basidiomycete cell is selected from the genus of Pluteopsis.
532. The bioactive agent according to item 509, wherein Basidiomycete cell is selected from the genus of Psalliotina.
533. The bioactive agent according to item 509, wherein Basidiomycete cell is selected from the genus of Psammocoparius.
534. The bioactive agent according to item 509, wherein Basidiomycete cell is selected from the genus of Psathyra.
535. The bioactive agent according to item 509, wherein Basidiomycete cell is selected from the genus of Psathyrella.
536. The bioactive agent according to item 509, wherein Basidiomycete cell is selected from the genus of Pselliophora.
537. The bioactive agent according to item 509, wherein Basidiomycete cell is selected from the genus of Pseudocoprinus.
538. The bioactive agent according to item 509, wherein Basidiomycete cell is selected from the genus of Psilocybe.
539. The bioactive agent according to item 509, wherein Basidiomycete cell is selected from the genus of Rhacophyllus.
540. The bioactive agent according to item 509, wherein Basidiomycete cell is selected from the genus of Xerocoprinus.
541. The bioactive agent according to item 509, wherein Basidiomycete cell is selected from the genus of Zerovaemyces.
542. The bioactive agent according to item 222, wherein said Basidiomycete cell belongs to a genus selected from the group consisting of Agmocybe, Anamika, Aroramyces, Astrosporina, Bulbopodium, Calathinus, Cereicium, Chromocyphella, Clypeus, Cortinarius, Crepidotus, Cribbea, Cuphocybe, Cyanicium, Cymbella, Cyphellathelia, Cystocybe, Dermocybe, Descolea, Dochmiopus, Epicorticium, Episphaeria, Flammulaster, Flocculina, Fulvidula, Galera, Galerina, Galerula, Gomphos, Gymnopilus, Hebelomina, Horakomyces, Hydrocybe, Hydrocybium, Hydrotelamonia, Hygramaricium, Hygromyxacium, Inocibium, Inocybe, Inocybella, Inoloma, Kjeldsenia, Leucocortinarius, Leucopus, Locellina, Mackintoshia, Marasmiopsis, Melanomphalia, Meliderma, Mycolevis, Myxacium, Myxopholis, Nanstelocephala, Octojuga, Pellidiscus, Phaeocarpus, Phaeocollybia, Phaeocyphella, Phaeogalera, Phaeoglabrotricha, Phaeomarasmius, Phaeosolenia, Phialocybe, Phlegmacium, Pholidotopsis, Pleurotellus, Pseudodescolea, Pseudogymnopilus, Pyrrhoglossum, Quercella, Ramicola, Rapacea, Raphanozon, Rozites, Sericeocybe, Simocybe, Sphaerotrachys, Squamaphlegma, Stagnicola, Stephanopus, Telamonia, Thaxterogaster, Tremellastrum, Tremellopsis, Tubaria, Velomycena and Weinzettlia.
543. The bioactive agent according to item 542, wherein Basidiomycete cell is selected from the genus of Agmocybe.
544. The bioactive agent according to item 542, wherein Basidiomycete cell is selected from the genus of Anamika.
545. The bioactive agent according to item 542, wherein Basidiomycete cell is selected from the genus of Aroramyces.
546. The bioactive agent according to item 542, wherein Basidiomycete cell is selected from the genus of Astrosporina.
547. The bioactive agent according to item 542, wherein Basidiomycete cell is selected from the genus of Bulbopodium.
548. The bioactive agent according to item 542, wherein Basidiomycete cell is selected from the genus of Calathinus.
549. The bioactive agent according to item 542, wherein Basidiomycete cell is selected from the genus of Cereicium.
550. The bioactive agent according to item 542, wherein Basidiomycete cell is selected from the genus of Chromocyphella.
551. The bioactive agent according to item 542, wherein Basidiomycete cell is selected from the genus of Clypeus.
552. The bioactive agent according to item 542, wherein Basidiomycete cell is selected from the genus of Cortinarius.
553. The bioactive agent according to item 542, wherein Basidiomycete cell is selected from the genus of Crepidotus.
554. The bioactive agent according to item 542, wherein Basidiomycete cell is selected from the genus of Cribbea.
555. The bioactive agent according to item 542, wherein Basidiomycete cell is selected from the genus of Cuphocybe.
556. The bioactive agent according to item 542, wherein Basidiomycete cell is selected from the genus of Cyanicium.
557. The bioactive agent according to item 542, wherein Basidiomycete cell is selected from the genus of Cymbella.
558. The bioactive agent according to item 542, wherein Basidiomycete cell is selected from the genus of Cyphellathelia.
559. The bioactive agent according to item 542, wherein Basidiomycete cell is selected from the genus of Cystocybe.
560. The bioactive agent according to item 542, wherein Basidiomycete cell is selected from the genus of Dermocybe.
561. The bioactive agent according to item 542, wherein Basidiomycete cell is selected from the genus of Descolea.
562. The bioactive agent according to item 542, wherein Basidiomycete cell is selected from the genus of Dochmiopus.
563. The bioactive agent according to item 542, wherein Basidiomycete cell is selected from the genus of Epicorticium.
564. The bioactive agent according to item 542, wherein Basidiomycete cell is selected from the genus of Episphaeria.
565. The bioactive agent according to item 542, wherein Basidiomycete cell is selected from the genus of Flammulaster.
566. The bioactive agent according to item 542, wherein Basidiomycete cell is selected from the genus of Flocculina.
567. The bioactive agent according to item 542, wherein Basidiomycete cell is selected from the genus of Fulvidula.
568. The bioactive agent according to item 542, wherein Basidiomycete cell is selected from the genus of Galera.
569. The bioactive agent according to item 542, wherein Basidiomycete cell is selected from the genus of Galerina.
570. The bioactive agent according to item 542, wherein Basidiomycete cell is selected from the genus of Galerula.
571. The bioactive agent according to item 542, wherein Basidiomycete cell is selected from the genus of Gomphos.
572. The bioactive agent according to item 542, wherein Basidiomycete cell is selected from the genus of Gymnopilus.
573. The bioactive agent according to item 542, wherein Basidiomycete cell is selected from the genus of Hebelomina.
574. The bioactive agent according to item 542, wherein Basidiomycete cell is selected from the genus of Horakomyces.
575. The bioactive agent according to item 542, wherein Basidiomycete cell is selected from the genus of Hydrocybe.
576. The bioactive agent according to item 542, wherein Basidiomycete cell is selected from the genus of Hydrocybium.
577. The bioactive agent according to item 542, wherein Basidiomycete cell is selected from the genus of Hydrotelamonia.
578. The bioactive agent according to item 542, wherein Basidiomycete cell is selected from the genus of Hygramaricium.
579. The bioactive agent according to item 542, wherein Basidiomycete cell is selected from the genus of Hygromyxacium.
580. The bioactive agent according to item 542, wherein Basidiomycete cell is selected from the genus of Inocibium.
581. The bioactive agent according to item 542, wherein Basidiomycete cell is selected from the genus of Inocybe.
582. The bioactive agent according to item 542, wherein Basidiomycete cell is selected from the genus of Inocybella.
583. The bioactive agent according to item 542, wherein Basidiomycete cell is selected from the genus of Inoloma.
584. The bioactive agent according to item 542, wherein Basidiomycete cell is selected from the genus of Kjeldsenia.
585. The bioactive agent according to item 542, wherein Basidiomycete cell is selected from the genus of Leucocortinarius.
586. The bioactive agent according to item 542, wherein Basidiomycete cell is selected from the genus of Leucopus.
587. The bioactive agent according to item 542, wherein Basidiomycete cell is selected from the genus of Locellina.
588. The bioactive agent according to item 542, wherein Basidiomycete cell is selected from the genus of Mackintoshia.
589. The bioactive agent according to item 542, wherein Basidiomycete cell is selected from the genus of Marasmiopsis.
590. The bioactive agent according to item 542, wherein Basidiomycete cell is selected from the genus of Melanomphalia.
591. The bioactive agent according to item 542, wherein Basidiomycete cell is selected from the genus of Meliderma.
592. The bioactive agent according to item 542, wherein Basidiomycete cell is selected from the genus of Mycolevis.
593. The bioactive agent according to item 542, wherein Basidiomycete cell is selected from the genus of Myxacium.
594. The bioactive agent according to item 542, wherein Basidiomycete cell is selected from the genus of Myxopholis.
595. The bioactive agent according to item 542, wherein Basidiomycete cell is selected from the genus of Nanstelocephala.
596. The bioactive agent according to item 542, wherein Basidiomycete cell is selected from the genus of Octojuga.
597. The bioactive agent according to item 542, wherein Basidiomycete cell is selected from the genus of Pellidiscus.
598. The bioactive agent according to item 542, wherein Basidiomycete cell is selected from the genus of Phaeocarpus.
599. The bioactive agent according to item 542, wherein Basidiomycete cell is selected from the genus of Phaeocollybia.
600. The bioactive agent according to item 542, wherein Basidiomycete cell is selected from the genus of Phaeocyphella.
601. The bioactive agent according to item 542, wherein Basidiomycete cell is selected from the genus of Phaeogalera.
602. The bioactive agent according to item 542, wherein Basidiomycete cell is selected from the genus of Phaeoglabrotricha.
603. The bioactive agent according to item 542, wherein Basidiomycete cell is selected from the genus of Phaeomarasmius.
604. The bioactive agent according to item 542, wherein Basidiomycete cell is selected from the genus of Phaeosolenia.
605. The bioactive agent according to item 542, wherein Basidiomycete cell is selected from the genus of Phialocybe.
606. The bioactive agent according to item 542, wherein Basidiomycete cell is selected from the genus of Phlegmacium.
607. The bioactive agent according to item 542, wherein Basidiomycete cell is selected from the genus of Pholidotopsis.
608. The bioactive agent according to item 542, wherein Basidiomycete cell is selected from the genus of Pleurotellus.
609. The bioactive agent according to item 542, wherein Basidiomycete cell is selected from the genus of Pseudodescolea.
610. The bioactive agent according to item 542, wherein Basidiomycete cell is selected from the genus of Pseudogymnopilus.
611. The bioactive agent according to item 542, wherein Basidiomycete cell is selected from the genus of Pyrrhoglossum.
612. The bioactive agent according to item 542, wherein Basidiomycete cell is selected from the genus of Quercella.
613. The bioactive agent according to item 542, wherein Basidiomycete cell is selected from the genus of Ramicola.
614. The bioactive agent according to item 542, wherein Basidiomycete cell is selected from the genus of Rapacea.
615. The bioactive agent according to item 542, wherein Basidiomycete cell is selected from the genus of Raphanozon.
616. The bioactive agent according to item 542, wherein Basidiomycete cell is selected from the genus of Rozites.
617. The bioactive agent according to item 542, wherein Basidiomycete cell is selected from the genus of Sericeocybe.
618. The bioactive agent according to item 542, wherein Basidiomycete cell is selected from the genus of Simocybe.
619. The bioactive agent according to item 542, wherein Basidiomycete cell is selected from the genus of Sphaerotrachys.
620. The bioactive agent according to item 542, wherein Basidiomycete cell is selected from the genus of Squamaphlegma.
621. The bioactive agent according to item 542, wherein Basidiomycete cell is selected from the genus of Stagnicola.
622. The bioactive agent according to item 542, wherein Basidiomycete cell is selected from the genus of Stephanopus.
623. The bioactive agent according to item 542, wherein Basidiomycete cell is selected from the genus of Telamonia.
624. The bioactive agent according to item 542, wherein Basidiomycete cell is selected from the genus of Thaxterogaster.
625. The bioactive agent according to item 542, wherein Basidiomycete cell is selected from the genus of Tremellastrum.
626. The bioactive agent according to item 542, wherein Basidiomycete cell is selected from the genus of Tremellopsis.
627. The bioactive agent according to item 542, wherein Basidiomycete cell is selected from the genus of Tubaria.
628. The bioactive agent according to item 542, wherein Basidiomycete cell is selected from the genus of Velomycena.
629. The bioactive agent according to item 542, wherein Basidiomycete cell is selected from the genus of Weinzettlia.
630. The bioactive agent according to item 223, wherein said Basidiomycete cell belongs to a genus selected from the group consisting of Alboleptonia, Arenicola, Calliderma, Claudopus, Clitopiloidea, Clitopilopsis, Clitopilus, Eccilia, Entoloma, Fibropilus, Hexajuga, Hirneola, Inocephalus, Inopilus, Lanolea, Latzinaea, Leptonia, Leptoniella, Nigropogon, Nolanea, Omphaliopsis, Orcella, Paraeccilia, Paraleptonia, Paxillopsis, Pouzarella, Pouzaromyces, Rhodocybe, Rhodocybella, Rhodogaster, Rhodophana, Rhodophyllus, Richoniella and Trichopilus.
631. The bioactive agent according to item 630, wherein Basidiomycete cell is selected from the genus of Alboleptonia.
632. The bioactive agent according to item 630, wherein Basidiomycete cell is selected from the genus of Arenicola.
633. The bioactive agent according to item 630, wherein Basidiomycete cell is selected from the genus of Calliderma.
634. The bioactive agent according to item 630, wherein Basidiomycete cell is selected from the genus of Claudopus.
635. The bioactive agent according to item 630, wherein Basidiomycete cell is selected from the genus of Clitopiloidea.
636. The bioactive agent according to item 630, wherein Basidiomycete cell is selected from the genus of Clitopilopsis.
637. The bioactive agent according to item 630, wherein Basidiomycete cell is selected from the genus of Clitopilus.
638. The bioactive agent according to item 630, wherein Basidiomycete cell is selected from the genus of Eccilia.
639. The bioactive agent according to item 630, wherein Basidiomycete cell is selected from the genus of Entoloma.
640. The bioactive agent according to item 630, wherein Basidiomycete cell is selected from the genus of Fibropilus.
641. The bioactive agent according to item 630, wherein Basidiomycete cell is selected from the genus of Hexajuga.
642. The bioactive agent according to item 630, wherein Basidiomycete cell is selected from the genus of Hirneola.
643. The bioactive agent according to item 630, wherein Basidiomycete cell is selected from the genus of Inocephalus.
644. The bioactive agent according to item 630, wherein Basidiomycete cell is selected from the genus of Inopilus.
645. The bioactive agent according to item 630, wherein Basidiomycete cell is selected from the genus of Lanolea.
646. The bioactive agent according to item 630, wherein Basidiomycete cell is selected from the genus of Latzinaea.
647. The bioactive agent according to item 630, wherein Basidiomycete cell is selected from the genus of Leptonia.
648. The bioactive agent according to item 630, wherein Basidiomycete cell is selected from the genus of Leptoniella.
649. The bioactive agent according to item 630, wherein Basidiomycete cell is selected from the genus of Nigropogon.
650. The bioactive agent according to item 630, wherein Basidiomycete cell is selected from the genus of Nolanea.
651. The bioactive agent according to item 630, wherein Basidiomycete cell is selected from the genus of Omphaliopsis.
652. The bioactive agent according to item 630, wherein Basidiomycete cell is selected from the genus of Orcella.
653. The bioactive agent according to item 630, wherein Basidiomycete cell is selected from the genus of Paraeccilia.
654. The bioactive agent according to item 630, wherein Basidiomycete cell is selected from the genus of Paraleptonia.
655. The bioactive agent according to item 630, wherein Basidiomycete cell is selected from the genus of Paxillopsis.
656. The bioactive agent according to item 630, wherein Basidiomycete cell is selected from the genus of Pouzarella.
657. The bioactive agent according to item 630, wherein Basidiomycete cell is selected from the genus of Pouzaromyces.
658. The bioactive agent according to item 630, wherein Basidiomycete cell is selected from the genus of Rhodocybe.
659. The bioactive agent according to item 630, wherein Basidiomycete cell is selected from the genus of Rhodocybella.
660. The bioactive agent according to item 630, wherein Basidiomycete cell is selected from the genus of Rhodogaster.
661. The bioactive agent according to item 630, wherein Basidiomycete cell is selected from the genus of Rhodophana.
662. The bioactive agent according to item 630, wherein Basidiomycete cell is selected from the genus of Rhodophyllus.
663. The bioactive agent according to item 630, wherein Basidiomycete cell is selected from the genus of Richoniella.
664. The bioactive agent according to item 630, wherein Basidiomycete cell is selected from the genus of Trichopilus.
665. The bioactive agent according to item 224, wherein said Basidiomycete cell belongs to a genus selected from the group consisting of Agarico-carnis, Buglossus, Confistulina, Fistulina, Hypodrys and Pseudofistulina.
666. The bioactive agent according to item 665, wherein Basidiomycete cell is selected from the genus of Agarico-carnis.
667. The bioactive agent according to item 665, wherein Basidiomycete cell is selected from the genus of Buglossus.
668. The bioactive agent according to item 665, wherein Basidiomycete cell is selected from the genus of Confistulina.
669. The bioactive agent according to item 665, wherein Basidiomycete cell is selected from the genus of Fistulina.
670. The bioactive agent according to item 665, wherein Basidiomycete cell is selected from the genus of Hypodrys.
671. The bioactive agent according to item 665, wherein Basidiomycete cell is selected from the genus of Pseudofistulina.
672. The bioactive agent according to item 225, wherein Basidiomycete cell is selected from the genus of Gigasperma.
673. The bioactive agent according to item 226, wherein Basidiomycete cell is selected from the genus of Hemigaster.
674. The bioactive agent according to item 227, wherein said Basidiomycete cell belongs to a genus selected from the group consisting of Hydnangium, Laccaria, Maccagnia, Podohydnangium and Russuliopsis.
675. The bioactive agent according to item 674, wherein Basidiomycete cell is selected from the genus of Hydnangium.
676. The bioactive agent according to item 674, wherein Basidiomycete cell is selected from the genus of Laccaria.
677, The bioactive agent according to item 674, wherein Basidiomycete cell is selected from the genus of Maccagnia.
678. The bioactive agent according to item 674, wherein Basidiomycete cell is selected from the genus of Podohydnangium.
679. The bioactive agent according to item 674, wherein Basidiomycete cell is selected from the genus of Russuliopsis.
680. The bioactive agent according to item 228, wherein said Basidiomycete cell belongs to a genus selected from the group consisting of Abstoma, Acutocapillitium, Arachnion, Arachniopsis, Bovista, Bovistaria, Bovistella, Bovistina, Calbovista, Calvatia, Calvatiella, Calvatiopsis, Capillaria, Catastoma, Cerophora, Disciseda, Enteromyxa, Eriosphaera, Gastropila, Globaria, Glyptoderma, Handkea, Hippoperdon, Hypoblema, Japonogaster, Langermannia, Lanopila, Lasiosphaera, Lycogalopsis, Lycoperdon, Lycoperdopsis, Morganella, Omalycus, Piemycus, Piesmycus, Pila, Priapus, Pseudolycoperdon, Sackea, Scoleciocarpus, Sufa, Utraria and Vascellum.
681. The bioactive agent according to item 680, wherein Basidiomycete cell is selected from the genus of Abstoma.
682. The bioactive agent according to item 680, wherein Basidiomycete cell is selected from the genus of Acutocapillitium.
683. The bioactive agent according to item 680, wherein Basidiomycete cell is selected from the genus of Arachnion.
684. The bioactive agent according to item 680, wherein Basidiomycete cell is selected from the genus of Arachniopsis.
685. The bioactive agent according to item 680, wherein Basidiomycete cell is selected from the genus of Bovista.
686. The bioactive agent according to item 680, wherein Basidiomycete cell is selected from the genus of Bovistaria.
687. The bioactive agent according to item 680, wherein Basidiomycete cell is selected from the genus of Bovistella.
688. The bioactive agent according to item 680, wherein Basidiomycete cell is selected from the genus of Bovistina.
689. The bioactive agent according to item 680, wherein Basidiomycete cell is selected from the genus of Calbovista.
690. The bioactive agent according to item 680, wherein Basidiomycete cell is selected from the genus of Calvatia.
691. The bioactive agent according to item 680, wherein Basidiomycete cell is selected from the genus of Calvatiella.
692. The bioactive agent according to item 680, wherein Basidiomycete cell is selected from the genus of Calvatiopsis.
693. The bioactive agent according to item 680, wherein Basidiomycete cell is selected from the genus of Capillaria.
694. The bioactive agent according to item 680, wherein Basidiomycete cell is selected from the genus of Catastoma.
695. The bioactive agent according to item 680, wherein Basidiomycete cell is selected from the genus of Cerophora.
696. The bioactive agent according to item 680, wherein Basidiomycete cell is selected from the genus of Disciseda.
697. The bioactive agent according to item 680, wherein Basidiomycete cell is selected from the genus of Enteromyxa.
698. The bioactive agent according to item 680, wherein Basidiomycete cell is selected from the genus of Eriosphaera.
699. The bioactive agent according to item 680, wherein Basidiomycete cell is selected from the genus of Gastropila.
700. The bioactive agent according to item 680, wherein Basidiomycete cell is selected from the genus of Globaria.
701. The bioactive agent according to item 680, wherein Basidiomycete cell is selected from the genus of Glyptoderma.
702. The bioactive agent according to item 680, wherein Basidiomycete cell is selected from the genus of Handkea.
703. The bioactive agent according to item 680, wherein Basidiomycete cell is selected from the genus of Hippoperdon.
704. The bioactive agent according to item 680, wherein Basidiomycete cell is selected from the genus of Hypoblema.
705. The bioactive agent according to item 680, wherein Basidiomycete cell is selected from the genus of Japonogaster.
706. The bioactive agent according to item 680, wherein Basidiomycete cell is selected from the genus of Langermannia.
707. The bioactive agent according to item 680, wherein Basidiomycete cell is selected from the genus of Lanopila.
708. The bioactive agent according to item 680, wherein Basidiomycete cell is selected from the genus of Lasiosphaera.
709. The bioactive agent according to item 680, wherein Basidiomycete cell is selected from the genus of Lycogalopsis.
710. The bioactive agent according to item 680, wherein Basidiomycete cell is selected from the genus of Lycoperdon.
711. The bioactive agent according to item 680, wherein Basidiomycete cell is selected from the genus of Lycoperdopsis.
712. The bioactive agent according to item 680, wherein Basidiomycete cell is selected from the genus of Morganella.
713. The bioactive agent according to item 680, wherein Basidiomycete cell is selected from the genus of Omalycus.
714. The bioactive agent according to item 680, wherein Basidiomycete cell is selected from the genus of Piemycus.
715. The bioactive agent according to item 680, wherein Basidiomycete cell is selected from the genus of Piesmycus.
716. The bioactive agent according to item 680, wherein Basidiomycete cell is selected from the genus of Pila.
717. The bioactive agent according to item 680, wherein Basidiomycete cell is selected from the genus of Priapus.
718. The bioactive agent according to item 680, wherein Basidiomycete cell is selected from the genus of Pseudolycoperdon.
719. The bioactive agent according to item 680, wherein Basidiomycete cell is selected from the genus of Sackea.
720. The bioactive agent according to item 680, wherein Basidiomycete cell is selected from the genus of Scoleciocarpus.
721. The bioactive agent according to item 680, wherein Basidiomycete cell is selected from the genus of Sufa.
722. The bioactive agent according to item 680, wherein Basidiomycete cell is selected from the genus of Utraria.
723. The bioactive agent according to item 680, wherein Basidiomycete cell is selected from the genus of Vascellum.
724. The bioactive agent according to item 229, wherein said Basidiomycete cell belongs to a genus selected from the group consisting of Amyloflagellula, Anastrophella, Androsaceus, Anthracophyllum, Aphotistus, Aphyllotus, Armillaria, Armillariella, Baeospora, Baumanniella, Calathella, Campanella, Cephaloscypha, Chaetocalathus, Chamaeceras, Collybidium, Collybiopsis, Coprinopsis, Cymatella, Cymatellopsis, Cyphellopsis, Cyptotrama, Dactylosporina, Deigloria, Discocyphella, Eoagaricus, Epicnaphus, Favolaschia, Fissolimbus, Flagelloscypha, Flammulina, Galeromycena, Gerronema, Glabrocyphella, Gloiocephala, Heliomyces, Hispidocalyptella, Hologloea, Hormomitaria, Hymenoconidium, Hymenogloea, Hymenomarasmius, Lachnella, Laschia, Lecanocybe, Lentinula, Libellus, Macrocystidia, Macrocystis, Manuripia, Marasmiellus, Marasmius, Merismodes, Micromphale, Monodelphus, Mucidula, Mycetinis, Mycomedusa, Myxocollybia, Nochascypha, Omphalotus, Oudemansia, Oudemansiella, Phaeocyphellopsis, Phaeodepas, Phaeolimacium, Physalacria, Plagiotus, Polymarasmius, Polymyces, Poroauricula, Porolaschia, Protomarasmius, Pseudodasyscypha, Pseudotyphula, Pterospora, Rhizomorpha, Rhodocollybia, Scorteus, Setulipes, Shitaker, Skepperiella, Stipitocyphella, Strobilurus, Stromatocyphella, Sympodia, Tephrophana, Tetrapyrgos, Vanromburghia, Xerula and Xerulina.
725. The bioactive agent according to item 724, wherein Basidiomycete cell is selected from the genus of Amyloflagellula.
726. The bioactive agent according to item 724, wherein Basidiomycete cell is selected from the genus of Anastrophella.
727. The bioactive agent according to item 724, wherein Basidiomycete cell is selected from the genus of Androsaceus.
728. The bioactive agent according to item 724, wherein Basidiomycete cell is selected from the genus of Anthracophyllum.
729. The bioactive agent according to item 724, wherein Basidiomycete cell is selected from the genus of Aphotistus.
730. The bioactive agent according to item 724, wherein Basidiomycete cell is selected from the genus of Aphyllotus.
731. The bioactive agent according to item 724, wherein Basidiomycete cell is selected from the genus of Armillaria.
732. The bioactive agent according to item 724, wherein Basidiomycete cell is selected from the genus of Armillariella.
733. The bioactive agent according to item 724, wherein Basidiomycete cell is selected from the genus of Baeospora.
734. The bioactive agent according to item 724, wherein Basidiomycete cell is selected from the genus of Baumanniella.
735. The bioactive agent according to item 724, wherein Basidiomycete cell is selected from the genus of Calathella.
736. The bioactive agent according to item 724, wherein Basidiomycete cell is selected from the genus of Campanella.
737. The bioactive agent according to item 724, wherein Basidiomycete cell is selected from the genus of Cephaloscypha.
738. The bioactive agent according to item 724, wherein Basidiomycete cell is selected from the genus of Chaetocalathus.
739. The bioactive agent according to item 724, wherein Basidiomycete cell is selected from the genus of Chamaeceras.
740. The bioactive agent according to item 724, wherein Basidiomycete cell is selected from the genus of Collybidium.
741. The bioactive agent according to item 724, wherein Basidiomycete cell is selected from the genus of Collybiopsis.
742. The bioactive agent according to item 724, wherein Basidiomycete cell is selected from the genus of Coprinopsis.
743. The bioactive agent according to item 724, wherein Basidiomycete cell is selected from the genus of Cymatella.
744. The bioactive agent according to item 724, wherein Basidiomycete cell is selected from the genus of Cymatellopsis.
745. The bioactive agent according to item 724, wherein Basidiomycete cell is selected from the genus of Cyphellopsis.
746. The bioactive agent according to item 724, wherein Basidiomycete cell is selected from the genus of Cyptotrama.
747. The bioactive agent according to item 724, wherein Basidiomycete cell is selected from the genus of Dactylosporina.
748. The bioactive agent according to item 724, wherein Basidiomycete cell is selected from the genus of Deigloria.
749. The bioactive agent according to item 724, wherein Basidiomycete cell is selected from the genus of Discocyphella.
750. The bioactive agent according to item 724, wherein Basidiomycete cell is selected from the genus of Eoagaricus.
751. The bioactive agent according to item 724, wherein Basidiomycete cell is selected from the genus of Epicnaphus.
752. The bioactive agent according to item 724, wherein Basidiomycete cell is selected from the genus of Favolaschia.
753. The bioactive agent according to item 724, wherein Basidiomycete cell is selected from the genus of Fissolimbus.
754. The bioactive agent according to item 724, wherein Basidiomycete cell is selected from the genus of Flagelloscypha.
755. The bioactive agent according to item 724, wherein Basidiomycete cell is selected from the genus of Flammulina.
756. The bioactive agent according to item 724, wherein Basidiomycete cell is selected from the genus of Galeromycena.
757. The bioactive agent according to item 724, wherein Basidiomycete cell is selected from the genus of Gerronema.
758. The bioactive agent according to item 724, wherein Basidiomycete cell is selected from the genus of Glabrocyphella.
759. The bioactive agent according to item 724, wherein Basidiomycete cell is selected from the genus of Gloiocephala.
760. The bioactive agent according to item 724, wherein Basidiomycete cell is selected from the genus of Heliomyces.
761. The bioactive agent according to item 724, wherein Basidiomycete cell is selected from the genus of Hispidocalyptella.
762. The bioactive agent according to item 724, wherein Basidiomycete cell is selected from the genus of Hologloea.
763. The bioactive agent according to item 724, wherein Basidiomycete cell is selected from the genus of Hormomitaria.
764. The bioactive agent according to item 724, wherein Basidiomycete cell is selected from the genus of Hymenoconidium.
765. The bioactive agent according to item 724, wherein Basidiomycete cell is selected from the genus of Hymenogloea.
766. The bioactive agent according to item 724, wherein Basidiomycete cell is selected from the genus of Hymenomarasmius.
767. The bioactive agent according to item 724, wherein Basidiomycete cell is selected from the genus of Lachnella.
768. The bioactive agent according to item 724, wherein Basidiomycete cell is selected from the genus of Laschia.
769. The bioactive agent according to item 724, wherein Basidiomycete cell is selected from the genus of Lecanocybe.
770. The bioactive agent according to item 724, wherein Basidiomycete cell is selected from the genus of Lentinula.
771. The bioactive agent according to item 724, wherein Basidiomycete cell is selected from the genus of Libellus.
772. The bioactive agent according to item 724, wherein Basidiomycete cell is selected from the genus of Macrocystidia.
773. The bioactive agent according to item 724, wherein Basidiomycete cell is selected from the genus of Macrocystis.
774. The bioactive agent according to item 724, wherein Basidiomycete cell is selected from the genus of Manuripia.
775. The bioactive agent according to item 724, wherein Basidiomycete cell is selected from the genus of Marasmiellus.
776. The bioactive agent according to item 724, wherein Basidiomycete cell is selected from the genus of Marasmius.
777. The bioactive agent according to item 724, wherein Basidiomycete cell is selected from the genus of Merismodes.
778. The bioactive agent according to item 724, wherein Basidiomycete cell is selected from the genus of Micromphale.
779. The bioactive agent according to item 724, wherein Basidiomycete cell is selected from the genus of Monodelphus.
780. The bioactive agent according to item 724, wherein Basidiomycete cell is selected from the genus of Mucidula.
781. The bioactive agent according to item 724, wherein Basidiomycete cell is selected from the genus of Mycetinis.
782. The bioactive agent according to item 724, wherein Basidiomycete cell is selected from the genus of Mycomedusa.
783. The bioactive agent according to item 724, wherein Basidiomycete cell is selected from the genus of Myxocollybia.
784. The bioactive agent according to item 724, wherein Basidiomycete cell is selected from the genus of Nochascypha.
785. The bioactive agent according to item 724, wherein Basidiomycete cell is selected from the genus of Omphalotus.
786. The bioactive agent according to item 724, wherein Basidiomycete cell is selected from the genus of Oudemansia.
787. The bioactive agent according to item 724, wherein Basidiomycete cell is selected from the genus of Oudemansiella.
788. The bioactive agent according to item 724, wherein Basidiomycete cell is selected from the genus of Phaeocyphellopsis.
789. The bioactive agent according to item 724, wherein Basidiomycete cell is selected from the genus of Phaeodepas.
790. The bioactive agent according to item 724, wherein Basidiomycete cell is selected from the genus of Phaeolimacium.
791. The bioactive agent according to item 724, wherein Basidiomycete cell is selected from the genus of Physalacria.
792. The bioactive agent according to item 724, wherein Basidiomycete cell is selected from the genus of Plagiotus.
793. The bioactive agent according to item 724, wherein Basidiomycete cell is selected from the genus of Polymarasmius.
794. The bioactive agent according to item 724, wherein Basidiomycete cell is selected from the genus of Polymyces.
795. The bioactive agent according to item 724, wherein Basidiomycete cell is selected from the genus of Poroauricula.
796. The bioactive agent according to item 724, wherein Basidiomycete cell is selected from the genus of Porolaschia.
797. The bioactive agent according to item 724, wherein Basidiomycete cell is selected from the genus of Protomarasmius.
798. The bioactive agent according to item 724, wherein Basidiomycete cell is selected from the genus of Pseudodasyscypha.
799. The bioactive agent according to item 724, wherein Basidiomycete cell is selected from the genus of Pseudotyphula.
800. The bioactive agent according to item 724, wherein Basidiomycete cell is selected from the genus of Pterospora.
801. The bioactive agent according to item 724, wherein Basidiomycete cell is selected from the genus of Rhizomorpha.
802. The bioactive agent according to item 724, wherein Basidiomycete cell is selected from the genus of Rhodocollybia.
803. The bioactive agent according to item 724, wherein Basidiomycete cell is selected from the genus of Scorteus.
804. The bioactive agent according to item 724, wherein Basidiomycete cell is selected from the genus of Setulipes.
805. The bioactive agent according to item 724, wherein Basidiomycete cell is selected from the genus of Shitaker.
806. The bioactive agent according to item 724, wherein Basidiomycete cell is selected from the genus of Skepperiella.
807. The bioactive agent according to item 724, wherein Basidiomycete cell is selected from the genus of Stipitocyphella.
808. The bioactive agent according to item 724, wherein Basidiomycete cell is selected from the genus of Strobilurus.
809. The bioactive agent according to item 724, wherein Basidiomycete cell is selected from the genus of Stromatocyphella.
810. The bioactive agent according to item 724, wherein Basidiomycete cell is selected from the genus of Sympodia.
811. The bioactive agent according to item 724, wherein Basidiomycete cell is selected from the genus of Tephrophana.
812. The bioactive agent according to item 724, wherein Basidiomycete cell is selected from the genus of Tetrapyrgos.
813. The bioactive agent according to item 724, wherein Basidiomycete cell is selected from the genus of Vanromburghia.
814. The bioactive agent according to item 724, wherein Basidiomycete cell is selected from the genus of Xerula.
815. The bioactive agent according to item 724, wherein Basidiomycete cell is selected from the genus of Xerulina.
816. The bioactive agent according to item 230, wherein said Basidiomycete cell belongs to a genus selected from the group consisting of Andebbia, Castoreum, Gummiglobus, Gummivena, Inoderma, Malajczukia, Mesophellia, Nothocastoreum and Potoromyces.
817. The bioactive agent according to item 816, wherein Basidiomycete cell is selected from the genus of Andebbia.
818. The bioactive agent according to item 816, wherein Basidiomycete cell is selected from the genus of Castoreum.
819. The bioactive agent according to item 816, wherein Basidiomycete cell is selected from the genus of Gummiglobus.
820. The bioactive agent according to item 816, wherein Basidiomycete cell is selected from the genus of Gummivena.
821. The bioactive agent according to item 816, wherein Basidiomycete cell is selected from the genus of Inoderma.
822. The bioactive agent according to item 816, wherein Basidiomycete cell is selected from the genus of Malajczukia.
823. The bioactive agent according to item 816, wherein Basidiomycete cell is selected from the genus of Mesophellia.
824. The bioactive agent according to item 816, wherein Basidiomycete cell is selected from the genus of Nothocastoreum.
825. The bioactive agent according to item 816, wherein Basidiomycete cell is selected from the genus of Potoromyces.
826. The bioactive agent according to item 231, wherein said Basidiomycete cell belongs to a genus selected from the group consisting of Endonevrum, Mycenastrum and Pachyderma.
827. The bioactive agent according to item 826, wherein Basidiomycete cell is selected from the genus of Endonevrum.
828. The bioactive agent according to item 826, wherein Basidiomycete cell is selected from the genus of Mycenastrum.
829. The bioactive agent according to item 826, wherein Basidiomycete cell is selected from the genus of Pachyderma.
830. The bioactive agent according to item 232, wherein Basidiomycete cell is selected from the genus of Nia.
831. The bioactive agent according to item 233, wherein said Basidiomycete cell belongs to a genus selected from the group consisting of Crucibulum, Cyathia, Cyathodes, Cyathus, Granularia, Mycocalia, Nidula, Nidularia and Peziza.
832. The bioactive agent according to item 831, wherein Basidiomycete cell is selected from the genus of Crucibulum.
833. The bioactive agent according to item 831, wherein Basidiomycete cell is selected from the genus of Cyathia.
834. The bioactive agent according to item 831, wherein Basidiomycete cell is selected from the genus of Cyathodes.
835. The bioactive agent according to item 831, wherein Basidiomycete cell is selected from the genus of Cyathus.
836. The bioactive agent according to item 831, wherein Basidiomycete cell is selected from the genus of Granularia.
837. The bioactive agent according to item 831, wherein Basidiomycete cell is selected from the genus of Mycocalia.
838. The bioactive agent according to item 831, wherein Basidiomycete cell is selected from the genus of Nidula.
839. The bioactive agent according to item 831, wherein Basidiomycete cell is selected from the genus of Nidularia.
840. The bioactive agent according to item 831, wherein Basidiomycete cell is selected from the genus of Peziza.
841. The bioactive agent according to item 234, wherein said Basidiomycete cell belongs to a genus selected from the group consisting of Areolaria, Battarreopsis, Cyphellomyces, Dictyocephalos, Phellorinia, Whetstonia and Xylopodium.
842. The bioactive agent according to item 841, wherein Basidiomycete cell is selected from the genus of Areolaria.
843. The bioactive agent according to item 841, wherein Basidiomycete cell is selected from the genus of Battarreopsis.
844. The bioactive agent according to item 841, wherein Basidiomycete cell is selected from the genus of Cyphellomyces.
845. The bioactive agent according to item 841, wherein Basidiomycete cell is selected from the genus of Dictyocephalos.
846. The bioactive agent according to item 841, wherein Basidiomycete cell is selected from the genus of Phellorinia.
847. The bioactive agent according to item 841, wherein Basidiomycete cell is selected from the genus of Whetstonia.
848. The bioactive agent according to item 841, wherein Basidiomycete cell is selected from the genus of Xylopodium.
849. The bioactive agent according to item 235, wherein said Basidiomycete cell belongs to a genus selected from the group consisting of Acanthocystis, Agaricochaete, Crepidopus, Cyclopleurotus, Gelona, Geopetalum, Hohenbuehelia, Lentodiopsis, Pleurotus, Pterophyllus and Scleroma.
850. The bioactive agent according to item 849, wherein Basidiomycete cell is selected from the genus of Acanthocystis.
851. The bioactive agent according to item 849, wherein Basidiomycete cell is selected from the genus of Agaricochaete.
852. The bioactive agent according to item 849, wherein Basidiomycete cell is selected from the genus of Crepidopus.
853. The bioactive agent according to item 849, wherein Basidiomycete cell is selected from the genus of Cyclopleurotus.
854. The bioactive agent according to item 849, wherein Basidiomycete cell is selected from the genus of Gelona.
855. The bioactive agent according to item 849, wherein Basidiomycete cell is selected from the genus of Geopetalum.
856. The bioactive agent according to item 849, wherein Basidiomycete cell is selected from the genus of Hohenbuehelia.
857. The bioactive agent according to item 849, wherein Basidiomycete cell is selected from the genus of Lentodiopsis.
858. The bioactive agent according to item 849, wherein Basidiomycete cell is selected from the genus of Pleurotus.
859. The bioactive agent according to item 849, wherein Basidiomycete cell is selected from the genus of Pterophyllus.
860. The bioactive agent according to item 849, wherein Basidiomycete cell is selected from the genus of Scleroma.
861. The bioactive agent according to item 236, wherein said Basidiomycete cell belongs to a genus selected from the group consisting of Agaricus, Amanita, Amanitaria, Amanitella, Amanitina, Amanitopsis, Amarrendia, Amidella, Amplariella, Annularia, Ariella, Aspidella, Boletium, Chamaeota, Gilbertia, Hyporrhodius, Lepidella, Leucomyces, Limacella, Myxoderma, Pluteus, Pseudofarinaceus, Rhodosporus, Termitosphaera, Torrendia, Vaginaria, Vaginarius, Vaginata, Venenarius, Volva, Volvaria, Volvariella, Volvariopsis, Volvarius, Volvella, Volvoamanita and Volvoboletus.
862. The bioactive agent according to item 861, wherein Basidiomycete cell is selected from the genus of Agaricus.
863. The bioactive agent according to item 861, wherein Basidiomycete cell is selected from the genus of Amanita.
864. The bioactive agent according to item 861, wherein Basidiomycete cell is selected from the genus of Amanitaria.
865. The bioactive agent according to item 861, wherein Basidiomycete cell is selected from the genus of Amanitella.
866. The bioactive agent according to item 861, wherein Basidiomycete cell is selected from the genus of Amanitina.
867. The bioactive agent according to item 861, wherein Basidiomycete cell is selected from the genus of Amanitopsis.
868. The bioactive agent according to item 861, wherein Basidiomycete cell is selected from the genus of Amarrendia.
869. The bioactive agent according to item 861, wherein Basidiomycete cell is selected from the genus of Amidella.
870. The bioactive agent according to item 861, wherein Basidiomycete cell is selected from the genus of Amplariella.
871. The bioactive agent according to item 861, wherein Basidiomycete cell is selected from the genus of Annularia.
872. The bioactive agent according to item 861, wherein Basidiomycete cell is selected from the genus of Ariella.
873. The bioactive agent according to item 861, wherein Basidiomycete cell is selected from the genus of Aspidella.
874. The bioactive agent according to item 861, wherein Basidiomycete cell is selected from the genus of Boletiuni.
875. The bioactive agent according to item 861, wherein Basidiomycete cell is selected from the genus of Chamaeota.
876. The bioactive agent according to item 861, wherein Basidiomycete cell is selected from the genus of Gilbertia.
877. The bioactive agent according to item 861, wherein Basidiomycete cell is selected from the genus of Hyporrhodius.
878. The bioactive agent according to item 861, wherein Basidiomycete cell is selected from the genus of Lepidella.
879. The bioactive agent according to item 861, wherein Basidiomycete cell is selected from the genus of Leucomyces.
880. The bioactive agent according to item 861, wherein Basidiomycete cell is selected from the genus of Limacella.
881. The bioactive agent according to item 861, wherein Basidiomycete cell is selected from the genus of Myxoderma.
882. The bioactive agent according to item 861, wherein Basidiomycete cell is selected from the genus of Pluteus.
883. The bioactive agent according to item 861, wherein Basidiomycete cell is selected from the genus of Pseudofarinaceus.
884. The bioactive agent according to item 861, wherein Basidiomycete cell is selected from the genus of Rhodosporus.
885. The bioactive agent according to item 861, wherein Basidiomycete cell is selected from the genus of Termitosphaera.
886. The bioactive agent according to item 861, wherein Basidiomycete cell is selected from the genus of Torrendia.
887. The bioactive agent according to item 861, wherein Basidiomycete cell is selected from the genus of Vaginaria.
888. The bioactive agent according to item 861, wherein Basidiomycete cell is selected from the genus of Vaginarius.
889. The bioactive agent according to item 861, wherein Basidiomycete cell is selected from the genus of Vaginata.
890. The bioactive agent according to item 861, wherein Basidiomycete cell is selected from the genus of Venenarius.
891. The bioactive agent according to item 861, wherein Basidiomycete cell is selected from the genus of Volva.
892. The bioactive agent according to item 861, wherein Basidiomycete cell is selected from the genus of Volvaria.
893. The bioactive agent according to item 861, wherein Basidiomycete cell is selected from the genus of Volvariella.
894. The bioactive agent according to item 861, wherein Basidiomycete cell is selected from the genus of Volvariopsis.
895. The bioactive agent according to item 861, wherein Basidiomycete cell is selected from the genus of Volvarius.
896. The bioactive agent according to item 861, wherein Basidiomycete cell is selected from the genus of Volvella.
897. The bioactive agent according to item 861, wherein Basidiomycete cell is selected from the genus of Volvoamanita.
898. The bioactive agent according to item 861, wherein Basidiomycete cell is selected from the genus of Volvoboletus.
899. The bioactive agent according to item 237, wherein said Basidiomycete cell belongs to a genus selected from the group consisting of Actiniceps, Allantula, Ceratella, Deflexula, Dimorphocystis, Parapterulicium, Penicillaria, Phaeopterula, Pterula and Pterulicium.
900. The bioactive agent according to item 899, wherein Basidiomycete cell is selected from the genus of Actiniceps.
901. The bioactive agent according to item 899, wherein Basidiomycete cell is selected from the genus of Allantula.
902. The bioactive agent according to item 899, wherein Basidiomycete cell is selected from the genus of Ceratella.
903. The bioactive agent according to item 899, wherein Basidiomycete cell is selected from the genus of Deflexula.
904. The bioactive agent according to item 899, wherein Basidiomycete cell is selected from the genus of Dimorphocystis.
905. The bioactive agent according to item 899, wherein Basidiomycete cell is selected from the genus of Parapterulicium.
906. The bioactive agent according to item 899, wherein Basidiomycete cell is selected from the genus of Penicillaria.
907. The bioactive agent according to item 899, wherein Basidiomycete cell is selected from the genus of Phaeopterula.
908. The bioactive agent according to item 899, wherein Basidiomycete cell is selected from the genus of Pterula.
909. The bioactive agent according to item 899, wherein Basidiomycete cell is selected from the genus of Pterulicium.
910. The bioactive agent according to item 238, wherein said Basidiomycete cell belongs to a genus selected from the group consisting of Apus, Auriculariopsis, Cytidiella, Ditiola, Flabellaria, Henningsomyces, Hyponevris, Petrona, Phaeoschizophyllum, Porotheleum, Rectipilus, Rhipidium, Scaphophoeum, Schizonia, Schizophyllum and Solenia.
911. The bioactive agent according to item 910, wherein Basidiomycete cell is selected from the genus of Apus.
912. The bioactive agent according to item 910, wherein Basidiomycete cell is selected from the genus of Auriculariopsis.
913. The bioactive agent according to item 910, wherein Basidiomycete cell is selected from the genus of Cytidiella.
914. The bioactive agent according to item 910, wherein Basidiomycete cell is selected from the genus of Ditiola.
915. The bioactive agent according to item 910, wherein Basidiomycete cell is selected from the genus of Flabellaria.
916. The bioactive agent according to item 910, wherein Basidiomycete cell is selected from the genus of Henningsomyces.
917. The bioactive agent according to item 910, wherein Basidiomycete cell is selected from the genus of Hyponevris.
918. The bioactive agent according to item 910, wherein Basidiomycete cell is selected from the genus of Petrona.
919. The bioactive agent according to item 910, wherein Basidiomycete cell is selected from the genus of Phaeoschizophyllum.
920. The bioactive agent according to item 910, wherein Basidiomycete cell is selected from the genus of Porotheleum.
921. The bioactive agent according to item 910, wherein Basidiomycete cell is selected from the genus of Rectipilus.
922. The bioactive agent according to item 910, wherein Basidiomycete cell is selected from the genus of Rhipidium.
923. The bioactive agent according to item 910, wherein Basidiomycete cell is selected from the genus of Scaphophoeum.
924. The bioactive agent according to item 910, wherein Basidiomycete cell is selected from the genus of Schizonia.
925. The bioactive agent according to item 910, wherein Basidiomycete cell is selected from the genus of Schizophyllum.
926. The bioactive agent according to item 910, wherein Basidiomycete cell is selected from the genus of Solenia.
927. The bioactive agent according to item 239, wherein Basidiomycete cell is selected from the genus of Stromatoscypha.
928. The bioactive agent according to item 240, wherein said Basidiomycete cell belongs to a genus selected from the group consisting of Cytophyllopsis, Deconica, Delitescor, Derminus, Dryophila, Flammopsis, Flammula, Galeropsina, Geophila, Gymnocybe, Hemipholiota, Hypholoma, Hypodendrum, Kuehneromyces, Le-Ratia, Leratiomyces, Melanotus, Mythicomyces, Nematoloma, Nemecomyces, Nivatogastrium, Pachylepyrium, Phaeonematoloma, Pholiota, Pleuroflammula, Psilocybe, Ryssospora, Stropharia, Stropholoma, Visculus and Weraroa.
929. The bioactive agent according to item 928, wherein Basidiomycete cell is selected from the genus of Cytophyllopsis.
930. The bioactive agent according to item 928, wherein Basidiomycete cell is selected from the genus of Deconica.
931. The bioactive agent according to item 928, wherein Basidiomycete cell is selected from the genus of Delitescor.
932. The bioactive agent according to item 928, wherein Basidiomycete cell is selected from the genus of Derminus.
933. The bioactive agent according to item 928, wherein Basidiomycete cell is selected from the genus of Dryophila.
934. The bioactive agent according to item 928, wherein Basidiomycete cell is selected from the genus of Flammopsis.
935. The bioactive agent according to item 928, wherein Basidiomycete cell is selected from the genus of Flammula.
936. The bioactive agent according to item 928, wherein Basidiomycete cell is selected from the genus of Galeropsina.
937. The bioactive agent according to item 928, wherein Basidiomycete cell is selected from the genus of Geophila.
938. The bioactive agent according to item 928, wherein Basidiomycete cell is selected from the genus of Gymnocybe.
939. The bioactive agent according to item 928, wherein Basidiomycete cell is selected from the genus of Hemipholiota.
940. The bioactive agent according to item 928, wherein Basidiomycete cell is selected from the genus of Hypholoma.
941. The bioactive agent according to item 928, wherein Basidiomycete cell is selected from the genus of Hypodendrum.
942. The bioactive agent according to item 928, wherein Basidiomycete cell is selected from the genus of Kuehneromyces.
943. The bioactive agent according to item 928, wherein Basidiomycete cell is selected from the genus of Le-Ratia.
944. The bioactive agent according to item 928, wherein Basidiomycete cell is selected from the genus of Leratiomyces.
945. The bioactive agent according to item 928, wherein Basidiomycete cell is selected from the genus of Melanotus.
946. The bioactive agent according to item 928, wherein Basidiomycete cell is selected from the genus of Mythicomyces.
947. The bioactive agent according to item 928, wherein Basidiomycete cell is selected from the genus of Nematoloma.
948. The bioactive agent according to item 928, wherein Basidiomycete cell is selected from the genus of Nemecomyces.
949. The bioactive agent according to item 928, wherein Basidiomycete cell is selected from the genus of Nivatogastrium.
950. The bioactive agent according to item 928, wherein Basidiomycete cell is selected from the genus of Pachylepyrium.
951. The bioactive agent according to item 928, wherein Basidiomycete cell is selected from the genus of Phaeonematoloma.
952. The bioactive agent according to item 928, wherein Basidiomycete cell is selected from the genus of Pholiota.
953. The bioactive agent according to item 928, wherein Basidiomycete cell is selected from the genus of Pleuroflammula.
954. The bioactive agent according to item 928, wherein Basidiomycete cell is selected from the genus of Psilocybe.
955. The bioactive agent according to item 928, wherein Basidiomycete cell is selected from the genus of Ryssospora.
956. The bioactive agent according to item 928, wherein Basidiomycete cell is selected from the genus of Stropharia.
957. The bioactive agent according to item 928, wherein Basidiomycete cell is selected from the genus of Stropholoma.
958. The bioactive agent according to item 928, wherein Basidiomycete cell is selected from the genus of Visculus.
959. The bioactive agent according to item 928, wherein Basidiomycete cell is selected from the genus of Weraroa.
960. The bioactive agent according to item 241, wherein said Basidiomycete cell belongs to a genus selected from the group consisting of Aeruginospora, Amparoina, Ampulloclitocybe, Arrhenia, Arthrosporella, Asproinocybe, Aspropaxillus, Asterophora, Asterotrichum, Asterotus, Austroclitocybe, Austroomphaliaster, Bactroboletus, Basidopus, Bertrandia, Bertrandiella, Biannularia, Boehmia, Botrydina, Caesposus, Callistodermatium, Callistosporium, Calocybe, Calyptella, Camarophyllopsis, Camarophyllus, Campanophyllum, Cantharellopsis, Cantharellula, Cantharocybe, Catathelasma, Catatrama, Caulorhiza, Cellypha, Chemonophyllum, Chromosera, Chrysobostrychodes, Chrysomphalina, Clavicybe, Clavomphalia, Clitocybe, Clitocybula, Collopus, Collybia, Conchomyces, Coolia, Coriscium, Corniola, Corrugaria, Cortinellus, Crinipellis, Cuphophyllus, Cynema, Cyphellocalathus, Cystoderma, Cystodermella, Decapitatus, Delicatula, Dendrocollybia, Dennisiomyces, Dermoloma, Dictyolus, Dictyopanus, Dictyoploca, Dissoderma, Echinosporella, Eomycenella, Fayodia, Filoboletus, Flabellimycena, Floccularia, Galactopus, Gamundia, Geotus, Gerhardtia, Gliophorus, Glutinaster, Godfrinia, Gymnopus, Gyroflexus, Gyrophila, Haasiella, Heimiomyces, Helotium, Hemimycena, Heterosporula, Hiatula, Hodophilus, Humidicutis, Hydrophorus, Hydropus, Hygroaster, Hygrocybe, Hygrophorus, Hygrotrama, Hypsizygus, Infundibulicybe, Insiticia, Jacobia, Lactocollybia, Lampteromyces, Leiopoda, Lepista, Leptoglossum, Leptomyces, Leptotus, Leucoinocybe, Leucopaxillus, Leucopholiota, Lichenomphalia, Limacinus, Limacium, Linopodium, Lulesia, Lyophyllopsis, Lyophyllum, Macrocybe, Maireina, Mastoleucomyces, Megacollybia, Megatricholoma, Melaleuca, Melanoleuca, Metulocyphella, Microcollybia, Microcollybia, Mniopetalum, Moniliophthora, Monomyces, Mycena, Mycenella, Mycenoporella, Mycenopsis, Mycenula, Mycoalvimia, Myxomphalia, Nematoctonus, Neoclitocybe, Neohygrocybe, Neohygrophorus, Neonothopanus, Nothoclavulina, Nothopanus, Nyctalis, Omphalia, Omphalia, Omphaliaster, Omphalina, Omphalius, Omphalopsis, Ossicaulis, Palaeocephala, Panellus, Paralepista, Peglerochaete, Pegleromyces, Perona, Phaeolepiota, Phaeomycena, Phaeotellus, Phalomia, Phlebomarasmius, Phlebomycena, Phlebophora, Phyllotopsis, Phyllotremella, Phyllotus, Physocystidium, Phytoconis, Pleurella, Pleurocollybia, Pleurocybella, Pleuromycenula, Pleurotopsis, Podabrella, Poromycena, Porpoloma, Prunulus, Psammospora, Pseudoarmillariella, Pseudobaeospora, Pseudoclitocybe, Pseudohiatula, Pseudohygrocybe, Pseudohygrophorus, Pseudolyophyllum, Pseudomycena, Pseudoomphalina, Rajapa, Resinomycena, Resupinatus, Retocybe, Rhodocyphella, Rhodopaxillus, Rhodotus, Rickenella, Rimbachia, Ripartitella, Ripartites, Roridomyces, Rubeolarius, Rugosomyces, Sarcomyxa, Sclerostilbum, Scytinotopsis, Scytinotus, Semiomphalina, Singerella, Singerocybe, Sinotermitomyces, Sphaerocephalus, Squamanita, Stachyomphalina, Stanglomyces, Stereopodium, Stigmatolemma, Tectella, Tephrocybe, Termitomyces, Tilachlidiopsis, Tilotus, Tomentifolium, Tricholoma, Tricholomella, Tricholomopsis, Tricholosporum, Trigonipes, Trogia, Ugola, Urceolus, Urospora, Urosporellina, Valentinia, Xeromphalina and Zephirea.
961. The bioactive agent according to item 960, wherein Basidiomycete cell is selected from the genus of Aeruginospora.
962. The bioactive agent according to item 960, wherein Basidiomycete cell is selected from the genus of Amparoina.
963. The bioactive agent according to item 960, wherein Basidiomycete cell is selected from the genus of Ampulloclitocybe.
964. The bioactive agent according to item 960, wherein Basidiomycete cell is selected from the genus of Ampulloclitocybe.
965. The bioactive agent according to item 960, wherein Basidiomycete cell is selected from the genus of Arrhenia.
966. The bioactive agent according to item 960, wherein Basidiomycete cell is selected from the genus of Arthrosporella.
967. The bioactive agent according to item 960, wherein Basidiomycete cell is selected from the genus of Asproinocybe.
968. The bioactive agent according to item 960, wherein Basidiomycete cell is selected from the genus of Aspropaxillus.
969. The bioactive agent according to item 960, wherein Basidiomycete cell is selected from the genus of Asterophora.
970. The bioactive agent according to item 960, wherein Basidiomycete cell is selected from the genus of Asterotrichum.
971. The bioactive agent according to item 960, wherein Basidiomycete cell is selected from the genus of Asterotus.
972. The bioactive agent according to item 960, wherein Basidiomycete cell is selected from the genus of Austroclitocybe.
973. The bioactive agent according to item 960, wherein Basidiomycete cell is selected from the genus of Austroomphaliaster.
974. The bioactive agent according to item 960, wherein Basidiomycete cell is selected from the genus of Bactroboletus.
975. The bioactive agent according to item 960, wherein Basidiomycete cell is selected from the genus of Basidopus.
976. The bioactive agent according to item 960, wherein Basidiomycete cell is selected from the genus of Bertrandia.
977. The bioactive agent according to item 960, wherein Basidiomycete cell is selected from the genus of Bertrandiella.
978. The bioactive agent according to item 960, wherein Basidiomycete cell is selected from the genus Biannularia.
979. The bioactive agent according to item 960, wherein Basidiomycete cell is selected from the genus of Boehmia.
980. The bioactive agent according to item 960, wherein Basidiomycete cell is selected from the genus of Botrydina.
981. The bioactive agent according to item 960, wherein Basidiomycete cell is selected from the genus of Caesposus.
982. The bioactive agent according to item 960, wherein Basidiomycete cell is selected from the genus of Callistodermatium.
983. The bioactive agent according to item 960, wherein Basidiomycete cell is selected from the genus of Callistosporium.
984. The bioactive agent according to item 960, wherein Basidiomycete cell is selected from the genus of Calocybe.
985. The bioactive agent according to item 960, wherein Basidiomycete cell is selected from the genus of Calyptella.
986. The bioactive agent according to item 960, wherein Basidiomycete cell is selected from the genus of Camarophyllopsis.
987. The bioactive agent according to item 960, wherein Basidiomycete cell is selected from the genus of Camarophyllus.
988. The bioactive agent according to item 960, wherein Basidiomycete cell is selected from the genus of Campanophyllum.
989. The bioactive agent according to item 960, wherein Basidiomycete cell is selected from the genus of Cantharellopsis.
990. The bioactive agent according to item 960, wherein Basidiomycete cell is selected from the genus of Cantharellula.
991. The bioactive agent according to item 960, wherein Basidiomycete cell is selected from the genus of Cantharocybe.
992. The bioactive agent according to item 960, wherein Basidiomycete cell is selected from the genus of Catathelasma.
993. The bioactive agent according to item 960, wherein Basidiomycete cell is selected from the genus of Catatrama.
994. The bioactive agent according to item 960, wherein Basidiomycete cell is selected from the genus of Caulorhiza.
995. The bioactive agent according to item 960, wherein Basidiomycete cell is selected from the genus of Cellypha.
996. The bioactive agent according to item 960, wherein Basidiomycete cell is selected from the genus of Chemonophyllum.
997. The bioactive agent according to item 960, wherein Basidiomycete cell is selected from the genus of Chromosera.
998. The bioactive agent according to item 960, wherein Basidiomycete cell is selected from the genus of Chrysobostrychodes.
999. The bioactive agent according to item 960, wherein Basidiomycete cell is selected from the genus of Chrysomphalina.
1000. The bioactive agent according to item 960, wherein Basidiomycete cell is selected from the genus of Clavicybe.
1001. The bioactive agent according to item 960, wherein Basidiomycete cell is selected from the genus of Clavomphalia.
1002. The bioactive agent according to item 960, wherein Basidiomycete cell is selected from the genus of Clitocybe.
1003. The bioactive agent according to item 960, wherein Basidiomycete cell is selected from the genus of Clitocybula.
1004. The bioactive agent according to item 960, wherein Basidiomycete cell is selected from the genus of Collopus.
1005. The bioactive agent according to item 960, wherein Basidiomycete cell is selected from the genus of Collybia.
1006. The bioactive agent according to item 960, wherein Basidiomycete cell is selected from the genus of Conchomyces.
1007. The bioactive agent according to item 960, wherein Basidiomycete cell is selected from the genus of Coolia.
1008. The bioactive agent according to item 960, wherein Basidiomycete cell is selected from the genus of Coriscium.
1009. The bioactive agent according to item 960, wherein Basidiomycete cell is selected from the genus of Corniola.
1010. The bioactive agent according to item 960, wherein Basidiomycete cell is selected from the genus of Corrugaria.
1011. The bioactive agent according to item 960, wherein Basidiomycete cell is selected from the genus of Cortinellus.
1012. The bioactive agent according to item 960, wherein Basidiomycete cell is selected from the genus of Crinipellis.
1013. The bioactive agent according to item 960, wherein Basidiomycete cell is selected from the genus of Cuphophyllus.
1014. The bioactive agent according to item 960, wherein Basidiomycete cell is selected from the genus of Cynema.
1015. The bioactive agent according to item 960, wherein Basidiomycete cell is selected from the genus of Cyphellocalathus.
1016. The bioactive agent according to item 960, wherein Basidiomycete cell is selected from the genus of Cystoderma.
1017. The bioactive agent according to item 960, wherein Basidiomycete cell is selected from the genus of Cystodermella.
1018. The bioactive agent according to item 960, wherein Basidiomycete cell is selected from the genus of Decapitatus.
1019. The bioactive agent according to item 960, wherein Basidiomycete cell is selected from the genus of Delicatula.
1020. The bioactive agent according to item 960, wherein Basidiomycete cell is selected from the genus of Dendrocollybia.
1021. The bioactive agent according to item 960, wherein Basidiomycete cell is selected from the genus of Dennisiomyces.
1022. The bioactive agent according to item 960, wherein Basidiomycete cell is selected from the genus of Dermoloma.
1023. The bioactive agent according to item 960, wherein Basidiomycete cell is selected from the genus of Dictyolus.
1024. The bioactive agent according to item 960, wherein Basidiomycete cell is selected from the genus of Dictyopanus.
1025. The bioactive agent according to item 960, wherein Basidiomycete cell is selected from the genus of Dictyoploca.
1026. The bioactive agent according to item 960, wherein Basidiomycete cell is selected from the genus of Dissoderma.
1027. The bioactive agent according to item 960, wherein Basidiomycete cell is selected from the genus of Echinosporella.
1028. The bioactive agent according to item 960, wherein Basidiomycete cell is selected from the genus of Eomycenella.
1029. The bioactive agent according to item 960, wherein Basidiomycete cell is selected from the genus of Fayodia.
1030. The bioactive agent according to item 960, wherein Basidiomycete cell is selected from the genus of Filoboletus.
1031. The bioactive agent according to item 960, wherein Basidiomycete cell is selected from the genus of Flabellimycena.
1032. The bioactive agent according to item 960, wherein Basidiomycete cell is selected from the genus of Floccularia.
1033. The bioactive agent according to item 960, wherein Basidiomycete cell is selected from the genus of Galactopus.
1034. The bioactive agent according to item 960, wherein Basidiomycete cell is selected from the genus of Gamundia.
1035. The bioactive agent according to item 960, wherein Basidiomycete cell is selected from the genus of Geotus.
1036. The bioactive agent according to item 960, wherein Basidiomycete cell is selected from the genus of Gerhardtia.
1037. The bioactive agent according to item 960, wherein Basidiomycete cell is selected from the genus of Gliophorus.
1038. The bioactive agent according to item 960, wherein Basidiomycete cell is selected from the genus of Glutinaster.
1039. The bioactive agent according to item 960, wherein Basidiomycete cell is selected from the genus of Godfrinia.
1040. The bioactive agent according to item 960, wherein Basidiomycete cell is selected from the genus of Gymnopus.
1041. The bioactive agent according to item 960, wherein Basidiomycete cell is selected from the genus of Gyroflexus.
1042. The bioactive agent according to item 960, wherein Basidiomycete cell is selected from the genus of Gyrophila.
1043. The bioactive agent according to item 960, wherein Basidiomycete cell is selected from the genus of Haasiella.
1044. The bioactive agent according to item 960, wherein Basidiomycete cell is selected from the genus of Heimiomyces.
1045. The bioactive agent according to item 960, wherein Basidiomycete cell is selected from the genus of Helotium.
1046. The bioactive agent according to item 960, wherein Basidiomycete cell is selected from the genus of Hemimycena.
1047. The bioactive agent according to item 960, wherein Basidiomycete cell is selected from the genus of Heterosporula.
1048. The bioactive agent according to item 960, wherein Basidiomycete cell is selected from the genus of Hiatula.
1049. The bioactive agent according to item 960, wherein Basidiomycete cell is selected from the genus of Hodophilus.
1050. The bioactive agent according to item 960, wherein Basidiomycete cell is selected from the genus of Humidicutis.
1051. The bioactive agent according to item 960, wherein Basidiomycete cell is selected from the genus of Hydrophorus.
1052. The bioactive agent according to item 960, wherein Basidiomycete cell is selected from the genus of Hydropus.
1053. The bioactive agent according to item 960, wherein Basidiomycete cell is selected from the genus of Hygroaster.
1054. The bioactive agent according to item 960, wherein Basidiomycete cell is selected from the genus of Hygrocybe.
1055. The bioactive agent according to item 960, wherein Basidiomycete cell is selected from the genus of Hygrophorus.
1056. The bioactive agent according to item 960, wherein Basidiomycete cell is selected from the genus of Hygrotrama.
1057. The bioactive agent according to item 960, wherein Basidiomycete cell is selected from the genus of Hypsizygus.
1058. The bioactive agent according to item 960, wherein Basidiomycete cell is selected from the genus of Infundibulicybe.
1059. The bioactive agent according to item 960, wherein Basidiomycete cell is selected from the genus of Insiticia.
1060. The bioactive agent according to item 960, wherein Basidiomycete cell is selected from the genus of Jacobia.
1061. The bioactive agent according to item 960, wherein Basidiomycete cell is selected from the genus of Lactocollybia.
1062. The bioactive agent according to item 960, wherein Basidiomycete cell is selected from the genus of Lampteromyces.
1063. The bioactive agent according to item 960, wherein Basidiomycete cell is selected from the genus of Leiopoda.
1064. The bioactive agent according to item 960, wherein Basidiomycete cell is selected from the genus of Lepista.
1065. The bioactive agent according to item 960, wherein Basidiomycete cell is selected from the genus of Leptoglossum.
1066. The bioactive agent according to item 960, wherein Basidiomycete cell is selected from the genus of Leptotus.
1067. The bioactive agent according to item 960, wherein Basidiomycete cell is selected from the genus of Leucoinocybe.
1068. The bioactive agent according to item 960, wherein Basidiomycete cell is selected from the genus of Leucopaxillus.
1069. The bioactive agent according to item 960, wherein Basidiomycete cell is selected from the genus of Leucopholiota.
1070. The bioactive agent according to item 960, wherein Basidiomycete cell is selected from the genus of Lichenomphalia.
1071. The bioactive agent according to item 960, wherein Basidiomycete cell is selected from the genus of Limacinus.
1072. The bioactive agent according to item 960, wherein Basidiomycete cell is selected from the genus of Limacium.
1073. The bioactive agent according to item 960, wherein Basidiomycete cell is selected from the genus of Linopodium.
1074. The bioactive agent according to item 960, wherein Basidiomycete cell is selected from the genus of Lulesia.
1075. The bioactive agent according to item 960, wherein Basidiomycete cell is selected from the genus of Lyophyllopsis.
1076. The bioactive agent according to item 960, wherein Basidiomycete cell is selected from the genus of Lyophyllum.
1077. The bioactive agent according to item 960, wherein Basidiomycete cell is selected from the genus of Macrocybe.
1078. The bioactive agent according to item 960, wherein Basidiomycete cell is selected from the genus of Maireina.
1079. The bioactive agent according to item 960, wherein Basidiomycete cell is selected from the genus of Mastoleucomyces.
1080. The bioactive agent according to item 960, wherein Basidiomycete cell is selected from the genus of Megacollybia.
1081. The bioactive agent according to item 960, wherein Basidiomycete cell is selected from the genus of Megatricholoma.
1082. The bioactive agent according to item 960, wherein Basidiomycete cell is selected from the genus of Melaleuca.
1083. The bioactive agent according to item 960, wherein Basidiomycete cell is selected from the genus of Melanoleuca.
1084. The bioactive agent according to item 960, wherein Basidiomycete cell is selected from the genus of Metulocyphella.
1085. The bioactive agent according to item 960, wherein Basidiomycete cell is selected from the genus of Microcollybia.
1086. The bioactive agent according to item 960, wherein Basidiomycete cell is selected from the genus of Mniopetalum.
1087. The bioactive agent according to item 960, wherein Basidiomycete cell is selected from the genus of Moniliophthora.
1088. The bioactive agent according to item 960, wherein Basidiomycete cell is selected from the genus of Monomyces.
1089. The bioactive agent according to item 960, wherein Basidiomycete cell is selected from the genus of Mycena.
1090. The bioactive agent according to item 960, wherein Basidiomycete cell is selected from the genus of Mycenella.
1091. The bioactive agent according to item 960, wherein Basidiomycete cell is selected from the genus of Mycenoporella.
1092. The bioactive agent according to item 960, wherein Basidiomycete cell is selected from the genus of Mycenopsis.
1093. The bioactive agent according to item 960, wherein Basidiomycete cell is selected from the genus of Mycenula.
1094. The bioactive agent according to item 960, wherein Basidiomycete cell is selected from the genus of Mycoalvimia.
1095. The bioactive agent according to item 960, wherein Basidiomycete cell is selected from the genus of Myxomphalia.
1096. The bioactive agent according to item 960, wherein Basidiomycete cell is selected from the genus of Nematoctonus.
1097. The bioactive agent according to item 960, wherein Basidiomycete cell is selected from the genus of Neoclitocybe.
1098. The bioactive agent according to item 960, wherein Basidiomycete cell is selected from the genus of Neohygrocybe.
1099. The bioactive agent according to item 960, wherein Basidiomycete cell is selected from the genus of Neohygrophorus.
1100. The bioactive agent according to item 960, wherein Basidiomycete cell is selected from the genus of Neonothopanus.
1101. The bioactive agent according to item 960, wherein Basidiomycete cell is selected from the genus of Nothoclavulina.
1102. The bioactive agent according to item 960, wherein Basidiomycete cell is selected from the genus of Nothopanus.
1103. The bioactive agent according to item 960, wherein Basidiomycete cell is selected from the genus of Nyctalis.
1104. The bioactive agent according to item 960, wherein Basidiomycete cell is selected from the genus of Omphalia.
1105. The bioactive agent according to item 960, wherein Basidiomycete cell is selected from the genus of Omphaliaster.
1106. The bioactive agent according to item 960, wherein Basidiomycete cell is selected from the genus of Omphalina.
1107. The bioactive agent according to item 960, wherein Basidiomycete cell is selected from the genus of Omphalius.
1108. The bioactive agent according to item 960, wherein Basidiomycete cell is selected from the genus of Omphalopsis.
1109. The bioactive agent according to item 960, wherein Basidiomycete cell is selected from the genus of Ossicaulis.
1110. The bioactive agent according to item 960, wherein Basidiomycete cell is selected from the genus of Palaeocephala.
1111. The bioactive agent according to item 960, wherein Basidiomycete cell is selected from the genus of Panellus.
1112. The bioactive agent according to item 960, wherein Basidiomycete cell is selected from the genus of Paralepista.
1113. The bioactive agent according to item 960, wherein Basidiomycete cell is selected from the genus of Peglerochaete.
1114. The bioactive agent according to item 960, wherein Basidiomycete cell is selected from the genus of Pegleromyces.
1115. The bioactive agent according to item 960, wherein Basidiomycete cell is selected from the genus of Perona.
1116. The bioactive agent according to item 960, wherein Basidiomycete cell is selected from the genus of Phaeolepiota.
1117. The bioactive agent according to item 960, wherein Basidiomycete cell is selected from the genus of Phaeomycena.
1118. The bioactive agent according to item 960, wherein Basidiomycete cell is selected from the genus of Phaeotellus.
1119. The bioactive agent according to item 960, wherein Basidiomycete cell is selected from the genus of Phalomia.
1120. The bioactive agent according to item 960, wherein Basidiomycete cell is selected from the genus of Phlebomarasmius.
1121. The bioactive agent according to item 960, wherein Basidiomycete cell is selected from the genus of Phlebomycena.
1122. The bioactive agent according to item 960, wherein Basidiomycete cell is selected from the genus of Phlebophora.
1123. The bioactive agent according to item 960, wherein Basidiomycete cell is selected from the genus of Phyllotopsis.
1124. The bioactive agent according to item 960, wherein Basidiomycete cell is selected from the genus of Phyllotremella.
1125. The bioactive agent according to item 960, wherein Basidiomycete cell is selected from the genus of Phyllotus.
1126. The bioactive agent according to item 960, wherein Basidiomycete cell is selected from the genus of Physocystidium.
1127. The bioactive agent according to item 960, wherein Basidiomycete cell is selected from the genus of Phytoconis.
1128. The bioactive agent according to item 960, wherein Basidiomycete cell is selected from the genus of Pleurella.
1129. The bioactive agent according to item 960, wherein Basidiomycete cell is selected from the genus of Pleurocollybia.
1130. The bioactive agent according to item 960, wherein Basidiomycete cell is selected from the genus of Pleurocybella.
1131. The bioactive agent according to item 960, wherein Basidiomycete cell is selected from the genus of Pleuromycenula.
1132. The bioactive agent according to item 960, wherein Basidiomycete cell is selected from the genus of Pleurotopsis.
1133. The bioactive agent according to item 960, wherein Basidiomycete cell is selected from the genus of Podabrella.
1134. The bioactive agent according to item 960, wherein Basidiomycete cell is selected from the genus of Poromycena.
1135. The bioactive agent according to item 960, wherein Basidiomycete cell is selected from the genus of Porpoloma.
1136. The bioactive agent according to item 960, wherein Basidiomycete cell is selected from the genus of Prunulus.
1137. The bioactive agent according to item 960, wherein Basidiomycete cell is selected from the genus of Psammospora.
1138. The bioactive agent according to item 960, wherein Basidiomycete cell is selected from the genus of Pseudoarmillariella.
1139. The bioactive agent according to item 960, wherein Basidiomycete cell is selected from the genus of Pseudobaeospora.
1140. The bioactive agent according to item 960, wherein Basidiomycete cell is selected from the genus of Pseudoclitocybe.
1141. The bioactive agent according to item 960, wherein Basidiomycete cell is selected from the genus of Pseudohiatula.
1142. The bioactive agent according to item 960, wherein Basidiomycete cell is selected from the genus of Pseudohygrocybe.
1143. The bioactive agent according to item 960, wherein Basidiomycete cell is selected from the genus of Pseudohygrophorus.
1144. The bioactive agent according to item 960, wherein Basidiomycete cell is selected from the genus of Pseudolyophyllum.
1145. The bioactive agent according to item 960, wherein Basidiomycete cell is selected from the genus of Pseudomycena.
1146. The bioactive agent according to item 960, wherein Basidiomycete cell is selected from the genus of Pseudoomphalina.
1147. The bioactive agent according to item 960, wherein Basidiomycete cell is selected from the genus of Rajapa.
1148. The bioactive agent according to item 960, wherein Basidiomycete cell is selected from the genus of Resinomycena.
1149. The bioactive agent according to item 960, wherein Basidiomycete cell is selected from the genus of Resupinatus.
1150. The bioactive agent according to item 960, wherein Basidiomycete cell is selected from the genus of Retocybe.
1151. The bioactive agent according to item 960, wherein Basidiomycete cell is selected from the genus of Rhodocyphella.
1152. The bioactive agent according to item 960, wherein Basidiomycete cell is selected from the genus of Rhodopaxillus.
1153. The bioactive agent according to item 960, wherein Basidiomycete cell is selected from the genus of Rhodotus.
1154. The bioactive agent according to item 960, wherein Basidiomycete cell is selected from the genus of Rickenella.
1155. The bioactive agent according to item 960, wherein Basidiomycete cell is selected from the genus of Rimbachia.
1156. The bioactive agent according to item 960, wherein Basidiomycete cell is selected from the genus of Ripartitella.
1157. The bioactive agent according to item 960, wherein Basidiomycete cell is selected from the genus of Ripartites.
1158. The bioactive agent according to item 960, wherein Basidiomycete cell is selected from the genus of Roridomyces.
1159. The bioactive agent according to item 960, wherein Basidiomycete cell is selected from the genus of Rubeolarius.
1160. The bioactive agent according to item 960, wherein Basidiomycete cell is selected from the genus of Rugosomyces.
1161. The bioactive agent according to item 960, wherein Basidiomycete cell is selected from the genus of Sarcomyxa.
1162. The bioactive agent according to item 960, wherein Basidiomycete cell is selected from the genus of Sclerostilbum.
1163. The bioactive agent according to item 960, wherein Basidiomycete cell is selected from the genus of Scytinotopsis.
1164. The bioactive agent according to item 960, wherein Basidiomycete cell is selected from the genus of Scytinotus.
1165. The bioactive agent according to item 960, wherein Basidiomycete cell is selected from the genus of Semiomphalina.
1166. The bioactive agent according to item 960, wherein Basidiomycete cell is selected from the genus of Singerella.
1167. The bioactive agent according to item 960, wherein Basidiomycete cell is selected from the genus of Singerocybe.
1168. The bioactive agent according to item 960, wherein Basidiomycete cell is selected from the genus of Sinotermitomyces.
1169. The bioactive agent according to item 960, wherein Basidiomycete cell is selected from the genus of Sphaerocephalus.
1170. The bioactive agent according to item 960, wherein Basidiomycete cell is selected from the genus of Squamanita.
1171. The bioactive agent according to item 960, wherein Basidiomycete cell is selected from the genus of Stachyomphalina.
1172. The bioactive agent according to item 960, wherein Basidiomycete cell is selected from the genus of Stanglomyces.
1173. The bioactive agent according to item 960, wherein Basidiomycete cell is selected from the genus of Stereopodium.
1174. The bioactive agent according to item 960, wherein Basidiomycete cell is selected from the genus of Stigmatolemma.
1175. The bioactive agent according to item 960, wherein Basidiomycete cell is selected from the genus of Tectella.
1176. The bioactive agent according to item 960, wherein Basidiomycete cell is selected from the genus of Tephrocybe.
1177. The bioactive agent according to item 960, wherein Basidiomycete cell is selected from the genus of Termitomyces.
1178. The bioactive agent according to item 960, wherein Basidiomycete cell is selected from the genus of Tilachlidiopsis.
1179. The bioactive agent according to item 960, wherein Basidiomycete cell is selected from the genus of Tilotus.
1180. The bioactive agent according to item 960, wherein Basidiomycete cell is selected from the genus of Tomentifolium.
1181. The bioactive agent according to item 960, wherein Basidiomycete cell is selected from the genus of Tricholoma.
1182. The bioactive agent according to item 960, wherein Basidiomycete cell is selected from the genus of Tricholomella.
1183. The bioactive agent according to item 960, wherein Basidiomycete cell is selected from the genus of Tricholomopsis.
1184. The bioactive agent according to item 960, wherein Basidiomycete cell is selected from the genus of Tricholosporum.
1185. The bioactive agent according to item 960, wherein Basidiomycete cell is selected from the genus of Trigonipes.
1186. The bioactive agent according to item 960, wherein Basidiomycete cell is selected from the genus of Trogia.
1187. The bioactive agent according to item 960, wherein Basidiomycete cell is selected from the genus of Ugola.
1188. The bioactive agent according to item 960, wherein Basidiomycete cell is selected from the genus of Urceolus.
1189. The bioactive agent according to item 960, wherein Basidiomycete cell is selected from the genus of Urospora.
1190. The bioactive agent according to item 960, wherein Basidiomycete cell is selected from the genus of Urosporellina.
1191. The bioactive agent according to item 960, wherein Basidiomycete cell is selected from the genus of Valentinia.
1192. The bioactive agent according to item 960, wherein Basidiomycete cell is selected from the genus of Xeromphalina.
1193. The bioactive agent according to item 960, wherein Basidiomycete cell is selected from the genus of Zephirea.
1194. The bioactive agent according to item 242, wherein said Basidiomycete cell belongs to a genus selected from the group consisting of Battarraeastrum, Battarrea, Battarreoides, Chlamydopus, Dendromyces, Queletia, Schizostoma, Sphaericeps, Tulasnodea and Tulostoma.
1195. The bioactive agent according to item 1195, wherein Basidiomycete cell is selected from the genus of Battarraeastrum.
1196. The bioactive agent according to item 1195, wherein Basidiomycete cell is selected from the genus of Battarrea.
1197. The bioactive agent according to item 1195, wherein Basidiomycete cell is selected from the genus of Battarreoides.
1198. The bioactive agent according to item 1195, wherein Basidiomycete cell is selected from the genus of Chlamydopus.
1199. The bioactive agent according to item 1195, wherein Basidiomycete cell is selected from the genus of Dendromyces.
1200. The bioactive agent according to item 1195, wherein Basidiomycete cell is selected from the genus of Queletia.
1201. The bioactive agent according to item 1195, wherein Basidiomycete cell is selected from the genus of Schizostoma.
1202. The bioactive agent according to item 1195, wherein Basidiomycete cell is selected from the genus of Sphaericeps.
1203. The bioactive agent according to item 1195, wherein Basidiomycete cell is selected from the genus of Tulasnodea.
1204. The bioactive agent according to item 1195, wherein Basidiomycete cell is selected from the genus of Tulostoma.
1205. The bioactive agent according to item 243, wherein said Basidiomycete cell belongs to a genus selected from the group consisting of Apiosporium, Astoma, Bromicolla, Cnazonaria, Coccopleum, Dacryopsella, Gliocoryne, Lutypha, Phacorhiza, Pistillaria, Pistillina, Scleromitra, Sclerotiomyces, Sclerotium, Sphaerula, Typhula and Xylochoeras.
1206. The bioactive agent according to item 1205, wherein Basidiomycete cell is selected from the genus of Apiosporium.
1207. The bioactive agent according to item 1205, wherein Basidiomycete cell is selected from the genus of Astoma.
1208. The bioactive agent according to item 1205, wherein Basidiomycete cell is selected from the genus of Bromicolla.
1209. The bioactive agent according to item 1205, wherein Basidiomycete cell is selected from the genus of Cnazonaria.
1210. The bioactive agent according to item 1205, wherein Basidiomycete cell is selected from the genus of Coccopleum.
1211. The bioactive agent according to item 1205, wherein Basidiomycete cell is selected from the genus of Dacryopsella.
1212. The bioactive agent according to item 1205, wherein Basidiomycete cell is selected from the genus of Gliocoryne.
1213. The bioactive agent according to item 1205, wherein Basidiomycete cell is selected from the genus of Lutypha.
1214. The bioactive agent according to item 1205, wherein Basidiomycete cell is selected from the genus of Phacorhiza.
1215. The bioactive agent according to item 1205, wherein Basidiomycete cell is selected from the genus of Pistillaria.
1216. The bioactive agent according to item 1205, wherein Basidiomycete cell is selected from the genus of Pistillina.
1217. The bioactive agent according to item 1205, wherein Basidiomycete cell is selected from the genus of Scleromitra.
1218. The bioactive agent according to item 1205, wherein Basidiomycete cell is selected from the genus of Sclerotiomyces.
1219. The bioactive agent according to item 1205, wherein Basidiomycete cell is selected from the genus of Sclerotium.
1220. The bioactive agent according to item 1205, wherein Basidiomycete cell is selected from the genus of Sphaerula.
1221. The bioactive agent according to item 1205, wherein Basidiomycete cell is selected from the genus of Typhula.
1222. The bioactive agent according to item 1205, wherein Basidiomycete cell is selected from the genus of Xylochoeras.
1223. The bioactive agent according to item 244, wherein Basidiomycete cell is selected from the genus of Rhizomarasmius.
1224. The bioactive agent according to item 247, wherein said Basidiomycete cell belongs to a genus selected from the group consisting of Albatrellopsis, Albatrellus, Jahnoporus, Ovinus, Polyporoletus and Scutiger.
1225. The bioactive agent according to item 1225, wherein Basidiomycete cell is selected from the genus of Albatrellopsis.
1226. The bioactive agent according to item 1225, wherein Basidiomycete cell is selected from the genus of Albatrellus.
1227. The bioactive agent according to item 1225, wherein Basidiomycete cell is selected from the genus of Jahnoporus.
1228. The bioactive agent according to item 1225, wherein Basidiomycete cell is selected from the genus of Ovinus.
1229. The bioactive agent according to item 1225, wherein Basidiomycete cell is selected from the genus of Polyporoletus.
1230. The bioactive agent according to item 1225, wherein Basidiomycete cell is selected from the genus of Scutiger.
1231. The bioactive agent according to item 248, wherein said Basidiomycete cell belongs to a genus selected from the group consisting of Amphinema, Amyloathelia, Amylocorticium, Athelia, Athelicium, Athelidium, Athelopsis, Butlerelfia, Byssocorticium, Byssocristella, Byssoporia, Caerulicium, Cora, Coraemyces, Corella, Cristinia, Dacryobasidium, Dichonema, Dictyonema, Dictyonematomyces, Digitatispora, Diplonema, Fibulomyces, Fibulorhizoctonia, Gyrolophium, Hypochnella, Hypochniciellum, Irpicodon, Laudatea, Leptosporomyces, Lobulicium, Luellia, Melzericium, Mycostigma, Piloderma, Plicatura, Plicaturopsis, Rhipidonema, Rhipidonematomyces, Rhizonema, Taeniospora, Tomentellopsis, Tylosperma, Tylospora and Wainiocora.
1232. The bioactive agent according to item 1231, wherein Basidiomycete cell is selected from the genus of Amphinema.
1233. The bioactive agent according to item 1231, wherein Basidiomycete cell is selected from the genus of Amyloathelia.
1234. The bioactive agent according to item 1231, wherein Basidiomycete cell is selected from the genus of Amylocorticium.
1235. The bioactive agent according to item 1231, wherein Basidiomycete cell is selected from the genus of Athelia.
1236. The bioactive agent according to item 1231, wherein Basidiomycete cell is selected from the genus of Athelicium.
1237. The bioactive agent according to item 1231, wherein Basidiomycete cell is selected from the genus of Athelidium.
1238. The bioactive agent according to item 1231, wherein Basidiomycete cell is selected from the genus of Athelopsis.
1239. The bioactive agent according to item 1231, wherein Basidiomycete cell is selected from the genus of Butlerelfia.
1240. The bioactive agent according to item 1231, wherein Basidiomycete cell is selected from the genus of Byssocorticium.
1241. The bioactive agent according to item 1231, wherein Basidiomycete cell is selected from the genus of Byssocristella.
1242. The bioactive agent according to item 1231, wherein Basidiomycete cell is selected from the genus of Byssoporia.
1243. The bioactive agent according to item 1231, wherein Basidiomycete cell is selected from the genus of Caerulicium.
1244. The bioactive agent according to item 1231, wherein Basidiomycete cell is selected from the genus of Cora.
1245. The bioactive agent according to item 1231, wherein Basidiomycete cell is selected from the genus of Coraemyces.
1246. The bioactive agent according to item 1231, wherein Basidiomycete cell is selected from the genus of Corella.
1247. The bioactive agent according to item 1231, wherein Basidiomycete cell is selected from the genus of Cristinia.
1248. The bioactive agent according to item 1231, wherein Basidiomycete cell is selected from the genus of Dacryobasidium.
1249. The bioactive agent according to item 1231, wherein Basidiomycete cell is selected from the genus of Dichonema.
1250. The bioactive agent according to item 1231, wherein Basidiomycete cell is selected from the genus of Dictyonema.
1251. The bioactive agent according to item 1231, wherein Basidiomycete cell is selected from the genus of Dictyonematomyces.
1252. The bioactive agent according to item 1231, wherein Basidiomycete cell is selected from the genus of Digitatispora.
1253. The bioactive agent according to item 1231, wherein Basidiomycete cell is selected from the genus of Diplonema.
1254. The bioactive agent according to item 1231, wherein Basidiomycete cell is selected from the genus of Fibulomyces.
1255. The bioactive agent according to item 1231, wherein Basidiomycete cell is selected from the genus of Fibulorhizoctonia.
1256. The bioactive agent according to item 1231, wherein Basidiomycete cell is selected from the genus of Gyrolophium.
1257. The bioactive agent according to item 1231, wherein Basidiomycete cell is selected from the genus of Hypochnella.
1258. The bioactive agent according to item 1231, wherein Basidiomycete cell is selected from the genus of Hypochniciellum.
1259. The bioactive agent according to item 1231, wherein Basidiomycete cell is selected from the genus of Irpicodon.
1260. The bioactive agent according to item 1231, wherein Basidiomycete cell is selected from the genus of Laudatea.
1261. The bioactive agent according to item 1231, wherein Basidiomycete cell is selected from the genus of Amphinema, Amyloathelia, Amylocorticium, Athelia, Leptosporomyces.
1262. The bioactive agent according to item 1231, wherein Basidiomycete cell is selected from the genus of Lobulicium.
1263. The bioactive agent according to item 1231, wherein Basidiomycete cell is selected from the genus of Luellia.
1264. The bioactive agent according to item 1231, wherein Basidiomycete cell is selected from the genus of Melzericium.
1265. The bioactive agent according to item 1231, wherein Basidiomycete cell is selected from the genus of Mycostigma.
1266. The bioactive agent according to item 1231, wherein Basidiomycete cell is selected from the genus of Piloderma.
1267. The bioactive agent according to item 1231, wherein Basidiomycete cell is selected from the genus of Plicatura.
1268. The bioactive agent according to item 1231, wherein Basidiomycete cell is selected from the genus of Plicaturopsis.
1269. The bioactive agent according to item 1231, wherein Basidiomycete cell is selected from the genus of Rhipidonema.
1270. The bioactive agent according to item 1231, wherein Basidiomycete cell is selected from the genus of Rhipidonematomyces.
1271. The bioactive agent according to item 1231, wherein Basidiomycete cell is selected from the genus of Rhizonema.
1272. The bioactive agent according to item 1231, wherein Basidiomycete cell is selected from the genus of Taeniospora.
1273. The bioactive agent according to item 1231, wherein Basidiomycete cell is selected from the genus of Tomentellopsis.
1274. The bioactive agent according to item 1231, wherein Basidiomycete cell is selected from the genus of Tylosperma.
1275. The bioactive agent according to item 1231, wherein Basidiomycete cell is selected from the genus of Tylospora.
1276. The bioactive agent according to item 1231, wherein Basidiomycete cell is selected from the genus of Wainiocora.
1277. The bioactive agent according to item 249, wherein said Basidiomycete cell belongs to a genus selected from the group consisting Boreostereum, Chaetocarpus, Chaetodermella, Columnocystis, Grandinioides, Hirneola, Mycobonia, Mycothele and Veluticeps.
1278. The bioactive agent according to item 1277, wherein Basidiomycete cell is selected from the genus of Boreostereum.
1279. The bioactive agent according to item 1277, wherein Basidiomycete cell is selected from the genus of Chaetocarpus.
1280. The bioactive agent according to item 1277, wherein Basidiomycete cell is selected from the genus of Chaetodermella.
1281. The bioactive agent according to item 1277, wherein Basidiomycete cell is selected from the genus of Columnocystis.
1282. The bioactive agent according to item 1277, wherein Basidiomycete cell is selected from the genus of Grandinioides.
1283. The bioactive agent according to item 1277, wherein Basidiomycete cell is selected from the genus of Hirneola.
1284. The bioactive agent according to item 1277, wherein Basidiomycete cell is selected from the genus of Mycobonia.
1285. The bioactive agent according to item 1277, wherein Basidiomycete cell is selected from the genus Mycothele.
1286. The bioactive agent according to item 1277, wherein Basidiomycete cell is selected from the genus of Veluticeps.
1287. The bioactive agent according to item 250, wherein said Basidiomycete cell belongs to a genus selected from the group consisting Acantholichen, Aleurocorticium, Allosphaerium, Ambivina, Amylobasidium, Auricula, Bryochysium, Corticirama, Corticium, Cyanobasidium, Cytidia, Dendrocorticium, Dendrodontia, Dendrophysellum, Dendrothele, Dextrinodontia, Hemmesomyces, Laeticorticium, Laetisaria, Leptocorticium, Licrostroma, Limonomyces, Lindtneria, Lomatia, Lomatina, Lyomyces, Matula, Melzerodontia, Merulicium, Moniliopsis, Mutatoderma, Mycinema, Mycolindtneria, Necator, Nothocorticium, Papyrodiscus, Phaeophlebia, Pulcherricium, Punctularia, Rhizoctonia, Ripexicium, Thanatophytum and Vuilleminia.
1288. The bioactive agent according to item 1287, wherein Basidiomycete cell is selected from the genus of Acantholichen.
1289. The bioactive agent according to item 1287, wherein Basidiomycete cell is selected from the genus of Aleurocorticium.
1290. The bioactive agent according to item 1287, wherein Basidiomycete cell is selected from the genus of Allosphaerium.
1291. The bioactive agent according to item 1287, wherein Basidiomycete cell is selected from the genus of Ambivina.
1292. The bioactive agent according to item 1287, wherein Basidiomycete cell is selected from the genus of Amylobasidium.
1293. The bioactive agent according to item 1287, wherein Basidiomycete cell is selected from the genus of Auricula.
1294. The bioactive agent according to item 1287, wherein Basidiomycete cell is selected from the genus of Bryochysium.
1295. The bioactive agent according to item 1287, wherein Basidiomycete cell is selected from the genus of Corticirama.
1296. The bioactive agent according to item 1287, wherein Basidiomycete cell is selected from the genus of Corticium.
1297. The bioactive agent according to item 1287, wherein Basidiomycete cell is selected from the genus of Cyanobasidium.
1298. The bioactive agent according to item 1287, wherein Basidiomycete cell is selected from the genus of Cytidia.
1299. The bioactive agent according to item 1287, wherein Basidiomycete cell is selected from the genus of Dendrocorticium.
1300. The bioactive agent according to item 1287, wherein Basidiomycete cell is selected from the genus of Dendrodontia.
1301. The bioactive agent according to item 1287, wherein Basidiomycete cell is selected from the genus of Dendrophysellum.
1302. The bioactive agent according to item 1287, wherein Basidiomycete cell is selected from the genus of Dendrothele.
1303. The bioactive agent according to item 1287, wherein Basidiomycete cell is selected from the genus of Dextrinodontia.
1304. The bioactive agent according to item 1287, wherein Basidiomycete cell is selected from the genus of Hemmesomyces.
1305. The bioactive agent according to item 1287, wherein Basidiomycete cell is selected from the genus of Laeticorticium.
1306. The bioactive agent according to item 1287, wherein Basidiomycete cell is selected from the genus of Laetisaria.
1307. The bioactive agent according to item 1287, wherein Basidiomycete cell is selected from the genus of Leptocorticium.
1308. The bioactive agent according to item 1287, wherein Basidiomycete cell is selected from the genus of Licrostroma.
1309. The bioactive agent according to item 1287, wherein Basidiomycete cell is selected from the genus of Limonomyces.
1310. The bioactive agent according to item 1287, wherein Basidiomycete cell is selected from the genus of Lindtneria.
1311. The bioactive agent according to item 1287, wherein Basidiomycete cell is selected from the genus of Lomatia.
1312. The bioactive agent according to item 1287, wherein Basidiomycete cell is selected from the genus of Lomatina.
1313. The bioactive agent according to item 1287, wherein Basidiomycete cell is selected from the genus of Lyomyces.
1314. The bioactive agent according to item 1287, wherein Basidiomycete cell is selected from the genus of Matula.
1315. The bioactive agent according to item 1287, wherein Basidiomycete cell is selected from the genus of Melzerodontia.
1316. The bioactive agent according to item 1287, wherein Basidiomycete cell is selected from the genus of Merulicium.
1317. The bioactive agent according to item 1287, wherein Basidiomycete cell is selected from the genus of Moniliopsis.
1318. The bioactive agent according to item 1287, wherein Basidiomycete cell is selected from the genus of Mutatoderma.
1319. The bioactive agent according to item 1287, wherein Basidiomycete cell is selected from the genus of Mycinema.
1320. The bioactive agent according to item 1287, wherein Basidiomycete cell is selected from the genus of Mycolindtneria.
1321. The bioactive agent according to item 1287, wherein Basidiomycete cell is selected from the genus of Necator.
1322. The bioactive agent according to item 1287, wherein Basidiomycete cell is selected from the genus of Nothocorticium.
1323. The bioactive agent according to item 1287, wherein Basidiomycete cell is selected from the genus of Papyrodiscus.
1324. The bioactive agent according to item 1287, wherein Basidiomycete cell is selected from the genus of Phaeophlebia.
1325. The bioactive agent according to item 1287, wherein Basidiomycete cell is selected from the genus of Pulcherricium.
1326. The bioactive agent according to item 1287, wherein Basidiomycete cell is selected from the genus of Punctularia.
1327. The bioactive agent according to item 1287, wherein Basidiomycete cell is selected from the genus of Rhizoctonia.
1328. The bioactive agent according to item 1287, wherein Basidiomycete cell is selected from the genus of Ripexicium.
1329. The bioactive agent according to item 1287, wherein Basidiomycete cell is selected from the genus of Thanatophytum.
1330. The bioactive agent according to item 1287, wherein Basidiomycete cell is selected from the genus of Vuilleminia.
1331. The bioactive agent according to item 251, wherein said Basidiomycete cell belongs to a genus selected from the group consisting of Adustomyces, Asterocyphella, Catilla, Cyphella, Dendrocyphella, Flavophlebia, Globulicium, Gloeocorticium, Halocyphina, Hyphoradulum, Incrustocalyptella, Limnoperdon, Oxydontia, Phaeoporotheleum, Pseudolagarobasidium, Radulodon, Radulomyces, Rhodoarrhenia, Sarcodontia, Seticyphella, Sphaerobasidioscypha, Thujacorticium, Wiesnerina, and Woldmaria.
1332. The bioactive agent according to item 1331, wherein Basidiomycete cell is selected from the genus of Adustomyces.
1333. The bioactive agent according to item 1331, wherein Basidiomycete cell is selected from the genus of Asterocyphella.
1334. The bioactive agent according to item 1331, wherein Basidiomycete cell is selected from the genus of Catilla.
1335. The bioactive agent according to item 1331, wherein Basidiomycete cell is selected from the genus of Cyphella.
1336. The bioactive agent according to item 1331, wherein Basidiomycete cell is selected from the genus of Dendrocyphella.
1337. The bioactive agent according to item 1331, wherein Basidiomycete cell is selected from the genus of Flavophlebia.
1338. The bioactive agent according to item 1331, wherein Basidiomycete cell is selected from the genus of Globulicium.
1339. The bioactive agent according to item 1331, wherein Basidiomycete cell is selected from the genus of Gloeocorticium.
1340. The bioactive agent according to item 1331, wherein Basidiomycete cell is selected from the genus of Halocyphina.
1341. The bioactive agent according to item 1331, wherein Basidiomycete cell is selected from the genus of Hyphoradulum.
1342. The bioactive agent according to item 1331, wherein Basidiomycete cell is selected from the genus of Incrustocalyptella.
1343. The bioactive agent according to item 1331, wherein Basidiomycete cell is selected from the genus of Limnoperdon.
1344. The bioactive agent according to item 1331, wherein Basidiomycete cell is selected from the genus of Oxydontia.
1345. The bioactive agent according to item 1331, wherein Basidiomycete cell is selected from the genus of Phaeoporotheleum.
1346. The bioactive agent according to item 1331, wherein Basidiomycete cell is selected from the genus of Pseudolagarobasidium.
1347. The bioactive agent according to item 1331, wherein Basidiomycete cell is selected from the genus of Radulodon.
1348. The bioactive agent according to item 1331, wherein Basidiomycete cell is selected from the genus of Radulomyces.
1349. The bioactive agent according to item 1331, wherein Basidiomycete cell is selected from the genus of Rhodoarrhenia.
1350. The bioactive agent according to item 1331, wherein Basidiomycete cell is selected from the genus of Sarcodontia.
1351. The bioactive agent according to item 1331, wherein Basidiomycete cell is selected from the genus of Seticyphella.
1352. The bioactive agent according to item 1331, wherein Basidiomycete cell is selected from the genus of Sphaerobasidioscypha.
1353. The bioactive agent according to item 1331, wherein Basidiomycete cell is selected from the genus of Thujacorticium.
1354. The bioactive agent according to item 1331, wherein Basidiomycete cell is selected from the genus of Wiesnerina.
1355. The bioactive agent according to item 1331, wherein Basidiomycete cell is selected from the genus of Woldmaria.
1356. The bioactive agent according to item 252, wherein said Basidiomycete cell belongs to a genus selected from the group consisting of Cericium, Crustomyces, Cystidiodontia, Cystostereum, Dentocorticium, Parvobasidium, Physodontia and Pteridomyces.
1357. The bioactive agent according to item 1356, wherein Basidiomycete cell is selected from the genus of Cericium.
1358. The bioactive agent according to item 1356, wherein Basidiomycete cell is selected from the genus of Crustomyces.
1359. The bioactive agent according to item 1356, wherein Basidiomycete cell is selected from the genus of Cystidiodontia.
1360. The bioactive agent according to item 1356, wherein Basidiomycete cell is selected from the genus of Cystostereum.
1361. The bioactive agent according to item 1356, wherein Basidiomycete cell is selected from the genus of Dentocorticium.
1362. The bioactive agent according to item 1356, wherein Basidiomycete cell is selected from the genus of Parvobasidium.
1363. The bioactive agent according to item 1356, wherein Basidiomycete cell is selected from the genus of Physodontia.
1364. The bioactive agent according to item 1356, wherein Basidiomycete cell is selected from the genus of Pteridomyces.
1365. The bioactive agent according to item 253, wherein said Basidiomycete cell belongs to a genus selected from the group consisting of Epithele, Epithelopsis and Skeletohydnum.
1366. The bioactive agent according to item 1365, wherein Basidiomycete cell is selected from the genus of Epithele.
1367. The bioactive agent according to item 1365, wherein Basidiomycete cell is selected from the genus of Epithelopsis.
1368. The bioactive agent according to item 1365, wherein Basidiomycete cell is selected from the genus of Skeletohydnum.
1369. The bioactive agent according to item 254, wherein said Basidiomycete cell belongs to a genus selected from the group consisting of Agaricon, Agarico-pulpa, Agarico-suber, Agaricum, Agaricus, Amylocystis, Anomoporia, Auriporia, Buglossoporus, Daedalea, Donkioporia, Fomitopsis, Gilbertsonia, Hemidiscia, Laricifomes, Osteina, Parmastomyces, Phaeodaedalea, Pilatoporus, Piptoporus, Placoderma, Podoporia, Postia, Rhodofomes, Spelaeomyces, Spongiporus, Strangulidium, Striglia, Ungularia, Wolfiporia and Xylostroma.
1370. The bioactive agent according to item 1369, wherein Basidiomycete cell is selected from the genus of Agaricon.
1371. The bioactive agent according to item 1369, wherein Basidiomycete cell is selected from the genus of Agarico-pulpa.
1372. The bioactive agent according to item 1369, wherein Basidiomycete cell is selected from the genus of Agarico-suber.
1373. The bioactive agent according to item 1369, wherein Basidiomycete cell is selected from the genus of Agaricum.
1374. The bioactive agent according to item 1369, wherein Basidiomycete cell is selected from the genus of Agaricus.
1375. The bioactive agent according to item 1369, wherein Basidiomycete cell is selected from the genus of Amylocystis.
1376. The bioactive agent according to item 1369, wherein Basidiomycete cell is selected from the genus of Anomoporia.
1377. The bioactive agent according to item 1369, wherein Basidiomycete cell is selected from the genus of Auriporia.
1378. The bioactive agent according to item 1369, wherein Basidiomycete cell is selected from the genus of Buglossoporus.
1379. The bioactive agent according to item 1369, wherein Basidiomycete cell is selected from the genus of Daedalea.
1380. The bioactive agent according to item 1369, wherein Basidiomycete cell is selected from the genus of Donkioporia.
1381. The bioactive agent according to item 1369, wherein Basidiomycete cell is selected from the genus of Fomitopsis.
1382. The bioactive agent according to item 1369, wherein Basidiomycete cell is selected from the genus of Gilbertsonia.
1383. The bioactive agent according to item 1369, wherein Basidiomycete cell is selected from the genus of Hemidiscia.
1384. The bioactive agent according to item 1369, wherein Basidiomycete cell is selected from the genus of Laricifomes.
1385. The bioactive agent according to item 1369, wherein Basidiomycete cell is selected from the genus of Osteina.
1386. The bioactive agent according to item 1369, wherein Basidiomycete cell is selected from the genus of Parmastomyces.
1387. The bioactive agent according to item 1369, wherein Basidiomycete cell is selected from the genus of Phaeodaedalea.
1388. The bioactive agent according to item 1369, wherein Basidiomycete cell is selected from the genus of Pilatoporus.
1389. The bioactive agent according to item 1369, wherein Basidiomycete cell is selected from the genus of Piptoporus.
1390. The bioactive agent according to item 1369, wherein Basidiomycete cell is selected from the genus of Placoderma.
1391. The bioactive agent according to item 1369, wherein Basidiomycete cell is selected from the genus of Podoporia.
1392. The bioactive agent according to item 1369, wherein Basidiomycete cell is selected from the genus of Postia.
1393. The bioactive agent according to item 1369, wherein Basidiomycete cell is selected from the genus of Rhodofomes.
1394. The bioactive agent according to item 1369, wherein Basidiomycete cell is selected from the genus of Spelaeomyces.
1395. The bioactive agent according to item 1369, wherein Basidiomycete cell is selected from the genus of Spongiporus.
1396. The bioactive agent according to item 1369, wherein Basidiomycete cell is selected from the genus of Strangulidium.
1397. The bioactive agent according to item 1369, wherein Basidiomycete cell is selected from the genus of Striglia.
1398. The bioactive agent according to item 1369, wherein Basidiomycete cell is selected from the genus of Ungularia.
1399. The bioactive agent according to item 1369, wherein Basidiomycete cell is selected from the genus of Wolfiporia.
1400. The bioactive agent according to item 1369, wherein Basidiomycete cell is selected from the genus of Xylostroma.
1401. The bioactive agent according to item 255, wherein said Basidiomycete cell belongs to a genus selected from the group consisting of Amauroderma, Dendrophagus, Elfvingia, Friesia, Ganoderma, Haddowia, Humphreya, Lazulinospora, Magoderna, Thermophymatospora, Tomophagus, Trachyderma and Whitfordia.
1402. The bioactive agent according to item 1401, wherein Basidiomycete cell is selected from the genus of Amauroderma.
1403. The bioactive agent according to item 1401, wherein Basidiomycete cell is selected from the genus of Dendrophagus.
1404. The bioactive agent according to item 1401, wherein Basidiomycete cell is selected from the genus of Elfvingia.
1405. The bioactive agent according to item 1401, wherein Basidiomycete cell is selected from the genus of Friesia.
1406. The bioactive agent according to item 1401, wherein Basidiomycete cell is selected from the genus of Ganoderma.
1407. The bioactive agent according to item 1401, wherein Basidiomycete cell is selected from the genus of Haddowia.
1408. The bioactive agent according to item 1401, wherein Basidiomycete cell is selected from the genus of Humphreya.
1409. The bioactive agent according to item 1401, wherein Basidiomycete cell is selected from the genus of Lazulinospora.
1410. The bioactive agent according to item 1401, wherein Basidiomycete cell is selected from the genus of Magoderna.
1411. The bioactive agent according to item 1401, wherein Basidiomycete cell is selected from the genus of Thermophymatospora.
1412. The bioactive agent according to item 1401, wherein Basidiomycete cell is selected from the genus of Tomophagus.
1413. The bioactive agent according to item 1401, wherein Basidiomycete cell is selected from the genus of Trachyderma.
1414. The bioactive agent according to item 1401, wherein Basidiomycete cell is selected from the genus of Whitfordia.
1415. The bioactive agent according to item 256, wherein said Basidiomycete cell belongs to a genus selected from the group consisting of Anisomyces, Ceratophora, Gloeophyllum, Griseoporia, Lenzitina, Phaeocoriolellus, Reisneria, Serda and Sesia.
1416. The bioactive agent according to item 1415, wherein Basidiomycete cell is selected from the genus of Anisomyces.
1417. The bioactive agent according to item 1415, wherein Basidiomycete cell is selected from the genus of Ceratophora.
1418. The bioactive agent according to item 1415, wherein Basidiomycete cell is selected from the genus of Gloeophyllum.
1419. The bioactive agent according to item 1415, wherein Basidiomycete cell is selected from the genus of Griseoporia.
1420. The bioactive agent according to item 1415, wherein Basidiomycete cell is selected from the genus of Lenzitina.
1421. The bioactive agent according to item 1415, wherein Basidiomycete cell is selected from the genus of Phaeocoriolellus.
1422. The bioactive agent according to item 1415, wherein Basidiomycete cell is selected from the genus of Reisneria.
1423. The bioactive agent according to item 1415, wherein Basidiomycete cell is selected from the genus of Serda.
1424. The bioactive agent according to item 1415, wherein Basidiomycete cell is selected from the genus of Sesia.
1425. The bioactive agent according to item 257, wherein said Basidiomycete cell belongs to a genus selected from the group consisting of Grammothele, Hymenogramme, Porogramme, Theleporus and Tinctoporia.
1426. The bioactive agent according to item 1425, wherein Basidiomycete cell is selected from the genus of Grammothele.
1427. The bioactive agent according to item 1425, wherein Basidiomycete cell is selected from the genus of Hymenogramme.
1428. The bioactive agent according to item 1425, wherein Basidiomycete cell is selected from the genus of Porogramme.
1429. The bioactive agent according to item 1425, wherein Basidiomycete cell is selected from the genus of Theleporus.
1430. The bioactive agent according to item 1425, wherein Basidiomycete cell is selected from the genus of Tinctoporia.
1431. The bioactive agent according to item 258, wherein said Basidiomycete cell belongs to a genus selected from the group consisting of Aurantiporus, Bjerkandera, Ceraporus, Ceriporia, Ceriporiopsis, Climacocystis, Gelatoporia, Hapalopilus, Irpiciporus, Ischnoderma, Leptoporus, Myriadoporus, Porpomyces, Pouzaroporia, Sarcoporia, Somion and Spongipellis.
1432. The bioactive agent according to item 1431, wherein Basidiomycete cell is selected from the genus of Aurantiporus.
1433. The bioactive agent according to item 1431, wherein Basidiomycete cell is selected from the genus of Bjerkandera.
1434. The bioactive agent according to item 1431, wherein Basidiomycete cell is selected from the genus of Ceraporus.
1435. The bioactive agent according to item 1431, wherein Basidiomycete cell is selected from the genus of Ceriporia.
1436. The bioactive agent according to item 1431, wherein Basidiomycete cell is selected from the genus Ceriporiopsis.
1437. The bioactive agent according to item 1431, wherein Basidiomycete cell is selected from the genus of Climacocystis.
1438. The bioactive agent according to item 1431, wherein Basidiomycete cell is selected from the genus of Gelatoporia.
1439. The bioactive agent according to item 1431, wherein Basidiomycete cell is selected from the genus of Hapalopilus.
1440. The bioactive agent according to item 1431, wherein Basidiomycete cell is selected from the genus of Irpiciporus.
1441. The bioactive agent according to item 1431, wherein Basidiomycete cell is selected from the genus of Ischnoderma.
1442. The bioactive agent according to item 1431, wherein Basidiomycete cell is selected from the genus of Leptoporus.
1443. The bioactive agent according to item 1431, wherein Basidiomycete cell is selected from the genus of Myriadoporus.
1444. The bioactive agent according to item 1431, wherein Basidiomycete cell is selected from the genus of Porpomyces.
1445. The bioactive agent according to item 1431, wherein Basidiomycete cell is selected from the genus of Pouzaroporia.
1446. The bioactive agent according to item 1431, wherein Basidiomycete cell is selected from the genus of Sarcoporia.
1447. The bioactive agent according to item 1431, wherein Basidiomycete cell is selected from the genus of Somion.
1448. The bioactive agent according to item 1431, wherein Basidiomycete cell is selected from the genus of Spongipellis.
1449. The bioactive agent according to item 259, wherein said Basidiomycete cell belongs to a genus selected from the group consisting of Aegerita, Aegeritina, Aegeritopsis, Amaurohydnum, Amauromyces, Atheloderma, Brevicellicium, Bulbillomyces, Cerocorticium, Chrysoderma, Conohypha, Coronicium, Crocysporium, Cyanodontia, Dermosporium, Elaphocephala, Galzinia, Gloeohypochnicium, Hydnellum, Hyphoderma, Hyphodontiastra, Hyphodontiella, Hypochnicium, Intextomyces, Kneiffia, Kneiffiella, Lyomyces, Metulodontia, Neokneiffia, Nodotia, Odontiopsis, Pirex, Pycnodon, Subulicium, Subulicystidium, Uncobasidium and Xylodon.
1450. The bioactive agent according to item 1449, wherein Basidiomycete cell is selected from the genus of Aegerita.
1451. The bioactive agent according to item 1449, wherein Basidiomycete cell is selected from the genus of Aegeritina.
1452. The bioactive agent according to item 1449, wherein Basidiomycete cell is selected from the genus of Aegeritopsis.
1453. The bioactive agent according to item 1449, wherein Basidiomycete cell is selected from the genus of Amaurohydnum.
1454. The bioactive agent according to item 1449, wherein Basidiomycete cell is selected from the genus of Amauromyces.
1455. The bioactive agent according to item 1449, wherein Basidiomycete cell is selected from the genus of Atheloderma.
1456. The bioactive agent according to item 1449, wherein Basidiomycete cell is selected from the genus of Brevicellicium.
1457. The bioactive agent according to item 1449, wherein Basidiomycete cell is selected from the genus of Bulbillomyces.
1458. The bioactive agent according to item 1449, wherein Basidiomycete cell is selected from the genus of Cerocorticium.
1459. The bioactive agent according to item 1449, wherein Basidiomycete cell is selected from the genus of Chrysoderma.
1460. The bioactive agent according to item 1449, wherein Basidiomycete cell is selected from the genus of Conohypha.
1461. The bioactive agent according to item 1449, wherein Basidiomycete cell is selected from the genus of Coronicium.
1462. The bioactive agent according to item 1449, wherein Basidiomycete cell is selected from the genus of Crocysporium.
1463. The bioactive agent according to item 1449, wherein Basidiomycete cell is selected from the genus of Cyanodontia.
1464. The bioactive agent according to item 1449, wherein Basidiomycete cell is selected from the genus of Dermosporium.
1465. The bioactive agent according to item 1449, wherein Basidiomycete cell is selected from the genus of Elaphocephala.
1466. The bioactive agent according to item 1449, wherein Basidiomycete cell is selected from the genus of Galzinia.
1467. The bioactive agent according to item 1449, wherein Basidiomycete cell is selected from the genus of Gloeohypochnicium.
1468. The bioactive agent according to item 1449, wherein Basidiomycete cell is selected from the genus of Hydnellum.
1469. The bioactive agent according to item 1449, wherein Basidiomycete cell is selected from the genus of Hyphoderma.
1470. The bioactive agent according to item 1449, wherein Basidiomycete cell is selected from the genus of Hyphodontiastra.
1471. The bioactive agent according to item 1449, wherein Basidiomycete cell is selected from the genus of Hyphodontiella.
1472. The bioactive agent according to item 1449, wherein Basidiomycete cell is selected from the genus of Hypochnicium.
1473. The bioactive agent according to item 1449, wherein Basidiomycete cell is selected from the genus of Intextomyces.
1474. The bioactive agent according to item 1449, wherein Basidiomycete cell is selected from the genus of Kneiffia.
1475. The bioactive agent according to item 1449, wherein Basidiomycete cell is selected from the genus of Kneiffiella.
1476. The bioactive agent according to item 1449, wherein Basidiomycete cell is selected from the genus of Lyomyces.
1477. The bioactive agent according to item 1449, wherein Basidiomycete cell is selected from the genus of Metulodontia.
1478. The bioactive agent according to item 1449, wherein Basidiomycete cell is selected from the genus of Neokneiffia.
1479. The bioactive agent according to item 1449, wherein Basidiomycete cell is selected from the genus of Nodotia.
1480. The bioactive agent according to item 1449, wherein Basidiomycete cell is selected from the genus of Odontiopsis.
1481. The bioactive agent according to item 1449, wherein Basidiomycete cell is selected from the genus of Pirex.
1482. The bioactive agent according to item 1449, wherein Basidiomycete cell is selected from the genus of Pycnodon.
1483. The bioactive agent according to item 1449, wherein Basidiomycete cell is selected from the genus of Subulicium.
1484. The bioactive agent according to item 1449, wherein Basidiomycete cell is selected from the genus of Subulicystidium.
1485. The bioactive agent according to item 1449, wherein Basidiomycete cell is selected from the genus of Uncobasidium.
1486. The bioactive agent according to item 1449, wherein Basidiomycete cell is selected from the genus of Xylodon.
1487. The bioactive agent according to item 260, wherein said Basidiomycete cell belongs to a genus selected from the group consisting of Abortiporus, Antrodia, Bornetina, Cartilosoma, Cautinia, Cladodendron, Cladomeris, Coriolellus, Diacanthodes, Flabellopilus, Grifola, Henningsia, Heteroporus, Hydnopolyporus, Irpicium, Leucofomes, Loweomyces, Meripilus, Merisma, Physisporinus, Polypilus and Rigidoporus.
1488. The bioactive agent according to item 1487, wherein Basidiomycete cell is selected from the genus of Abortiporus.
1489. The bioactive agent according to item 1487, wherein Basidiomycete cell is selected from the genus of Antrodia.
1490. The bioactive agent according to item 1487, wherein Basidiomycete cell is selected from the genus of Bornetina.
1491. The bioactive agent according to item 1487, wherein Basidiomycete cell is selected from the genus of Cartilosoma.
1492. The bioactive agent according to item 1487, wherein Basidiomycete cell is selected from the genus of Cautinia.
1493. The bioactive agent according to item 1487, wherein Basidiomycete cell is selected from the genus of Cladodendron.
1494. The bioactive agent according to item 1487, wherein Basidiomycete cell is selected from the genus of Cladomeris.
1495. The bioactive agent according to item 1487, wherein Basidiomycete cell is selected from the genus of Coriolellus.
1496. The bioactive agent according to item 1487, wherein Basidiomycete cell is selected from the genus of Diacanthodes.
1497. The bioactive agent according to item 1487, wherein Basidiomycete cell is selected from the genus of Flabellopilus.
1498. The bioactive agent according to item 1487, wherein Basidiomycete cell is selected from the genus of Grifola.
1499. The bioactive agent according to item 1487, wherein Basidiomycete cell is selected from the genus of Henningsia.
1500. The bioactive agent according to item 1487, wherein Basidiomycete cell is selected from the genus of Heteroporus.
1501. The bioactive agent according to item 1487, wherein Basidiomycete cell is selected from the genus of Hydnopolyporus.
1502. The bioactive agent according to item 1487, wherein Basidiomycete cell is selected from the genus of Irpicium.
1503. The bioactive agent according to item 1487, wherein Basidiomycete cell is selected from the genus of Leucofomes.
1504. The bioactive agent according to item 1487, wherein Basidiomycete cell is selected from the genus of Loweomyces.
1505. The bioactive agent according to item 1487, wherein Basidiomycete cell is selected from the genus of Meripilus.
1506. The bioactive agent according to item 1487, wherein Basidiomycete cell is selected from the genus of Merisma.
1507. The bioactive agent according to item 1487, wherein Basidiomycete cell is selected from the genus of Physisporinus.
1508. The bioactive agent according to item 1487, wherein Basidiomycete cell is selected from the genus of Polypilus.
1509. The bioactive agent according to item 1487, wherein Basidiomycete cell is selected from the genus of Rigidoporus.
1510. The bioactive agent according to item 261, wherein said Basidiomycete cell belongs to a genus selected from the group consisting of Acia, Byssomerulius, Caloporia, Caloporus, Castanoporus, Ceraceohydnum, Ceraceomerulius, Chondrostereum, Climacodon, Columnodontia, Crustoderma, Cylindrobasidium, Dacryobolus, Donkia, Gloeocystidium, Gloeoporus, Gloeostereum, Himantia, Jacksonomyces, Meruliopsis, Merulius, Mycoacia, Mycoaciella, Phlebia, Resinicium, Ricnophora, Scopuloides, Skvortzovia and Trabecularia.
1511. The bioactive agent according to item 1510, wherein Basidiomycete cell is selected from the genus of Acia.
1512. The bioactive agent according to item 1510, wherein Basidiomycete cell is selected from the genus of Byssomerulius.
1513. The bioactive agent according to item 1510, wherein Basidiomycete cell is selected from the genus of Caloporia.
1514. The bioactive agent according to item 1510, wherein Basidiomycete cell is selected from the genus of Caloporus.
1515. The bioactive agent according to item 1510, wherein Basidiomycete cell is selected from the genus of Castanoporus.
1516. The bioactive agent according to item 1510, wherein Basidiomycete cell is selected from the genus of Ceraceohydnum.
1517. The bioactive agent according to item 1510, wherein Basidiomycete cell is selected from the genus of Ceraceomerulius.
1518. The bioactive agent according to item 1510, wherein Basidiomycete cell is selected from the genus of Chondrostereum.
1519. The bioactive agent according to item 1510, wherein Basidiomycete cell is selected from the genus of Climacodon.
1520. The bioactive agent according to item 1510, wherein Basidiomycete cell is selected from the genus of Columnodontia.
1521. The bioactive agent according to item 1510, wherein Basidiomycete cell is selected from the genus of Crustoderma.
1522. The bioactive agent according to item 1510, wherein Basidiomycete cell is selected from the genus of Cylindrobasidium.
1523. The bioactive agent according to item 1510, wherein Basidiomycete cell is selected from the genus of Dacryobolus.
1524. The bioactive agent according to item 1510, wherein Basidiomycete cell is selected from the genus of Donkia.
1525. The bioactive agent according to item 1510, wherein Basidiomycete cell is selected from the genus of Gloeocystidium.
1526. The bioactive agent according to item 1510, wherein Basidiomycete cell is selected from the genus of Gloeoporus.
1527. The bioactive agent according to item 1510, wherein Basidiomycete cell is selected from the genus of Gloeostereum.
1528. The bioactive agent according to item 1510, wherein Basidiomycete cell is selected from the genus of Himantia.
1529. The bioactive agent according to item 1510, wherein Basidiomycete cell is selected from the genus of Jacksonomyces.
1530. The bioactive agent according to item 1510, wherein Basidiomycete cell is selected from the genus of Meruliopsis.
1531. The bioactive agent according to item 1510, wherein Basidiomycete cell is selected from the genus of Merulius.
1532. The bioactive agent according to item 1510, wherein Basidiomycete cell is selected from the genus of Mycoacia.
1533. The bioactive agent according to item 1510, wherein Basidiomycete cell is selected from the genus of Mycoaciella.
1534. The bioactive agent according to item 1510, wherein Basidiomycete cell is selected from the genus of Phlebia.
1535. The bioactive agent according to item 1510, wherein Basidiomycete cell is selected from the genus of Resinicium.
1536. The bioactive agent according to item 1510, wherein Basidiomycete cell is selected from the genus of Ricnophora.
1537. The bioactive agent according to item 1510, wherein Basidiomycete cell is selected from the genus of Scopuloides.
1538. The bioactive agent according to item 1510, wherein Basidiomycete cell is selected from the genus of Skvortzovia.
1539. The bioactive agent according to item 1510, wherein Basidiomycete cell is selected from the genus of Trabecularia.
1540. The bioactive agent according to item 262, wherein said Basidiomycete cell belongs to a genus selected from the group consisting of Australicium, Botryodontia, Candelabrochaete, Ceraceomyces, Corticium, Efibula, Erythricium, Grandiniella, Gyrophanopsis, Hjortstamia, Hydnophlebia, Hyphodermella, Hyphodermopsis, Licentia, Lloydella, Lopharia, Membranicium, Odonticium, Phanerochaete, Phlebiopsis, Porostereum, Terana, Thwaitesiella and Xerocarpus.
1541. The bioactive agent according to item 1540, wherein Basidiomycete cell is selected from the genus of Australicium.
1542. The bioactive agent according to item 1540, wherein Basidiomycete cell is selected from the genus of Botryodontia.
1543. The bioactive agent according to item 1540, wherein Basidiomycete cell is selected from the genus of Candelabrochaete.
1544. The bioactive agent according to item 1540, wherein Basidiomycete cell is selected from the genus of Ceraceomyces.
1545. The bioactive agent according to item 1540, wherein Basidiomycete cell is selected from the genus of Corticium.
1546. The bioactive agent according to item 1540, wherein Basidiomycete cell is selected from the genus of Efibula.
1547. The bioactive agent according to item 1540, wherein Basidiomycete cell is selected from the genus of Erythricium.
1548. The bioactive agent according to item 1540, wherein Basidiomycete cell is selected from the genus of Grandiniella.
1549. The bioactive agent according to item 1540, wherein Basidiomycete cell is selected from the genus of Gyrophanopsis.
1550. The bioactive agent according to item 1540, wherein Basidiomycete cell is selected from the genus of Hjortstamia.
1551. The bioactive agent according to item 1540, wherein Basidiomycete cell is selected from the genus of Hydnophlebia.
1552. The bioactive agent according to item 1540, wherein Basidiomycete cell is selected from the genus of Hyphodermella.
1553. The bioactive agent according to item 1540, wherein Basidiomycete cell is selected from the genus of Hyphodermopsis.
1554. The bioactive agent according to item 1540, wherein Basidiomycete cell is selected from the genus of Licentia.
1555. The bioactive agent according to item 1540, wherein Basidiomycete cell is selected from the genus of Lloydella.
1556. The bioactive agent according to item 1540, wherein Basidiomycete cell is selected from the genus of Lopharia.
1557. The bioactive agent according to item 1540, wherein Basidiomycete cell is selected from the genus of Membranicium.
1558. The bioactive agent according to item 1540, wherein Basidiomycete cell is selected from the genus of Odonticium.
1559. The bioactive agent according to item 1540, wherein Basidiomycete cell is selected from the genus of Phanerochaete.
1560. The bioactive agent according to item 1540, wherein Basidiomycete cell is selected from the genus of Phlebiopsis.
1561. The bioactive agent according to item 1540, wherein Basidiomycete cell is selected from the genus of Porostereum.
1562. The bioactive agent according to item 1540, wherein Basidiomycete cell is selected from the genus of Terana.
1563. The bioactive agent according to item 1540, wherein Basidiomycete cell is selected from the genus of Thwaitesiella.
1564. The bioactive agent according to item 1540, wherein Basidiomycete cell is selected from the genus of Xerocarpus.
1565. The bioactive agent according to item 263, wherein said Basidiomycete cell belongs to a genus selected from the group consisting of Actinostroma, Aquascypha, Beccaria, Beccariella, Bresadolina, Caripia, Cladoderris, Coralloderma, Cotylidia, Craterella, Cymatoderma, Cyphellostereum, Granulobasidium, Inflatostereum, Podoscypha, Pseudolasiobolus, Stereogloeocystidium, Stereophyllum and Stereopsis.
1566. The bioactive agent according to item 1565, wherein Basidiomycete cell is selected from the genus of Actinostroma.
1567. The bioactive agent according to item 1565, wherein Basidiomycete cell is selected from the genus of Aquascypha.
1568. The bioactive agent according to item 1565, wherein Basidiomycete cell is selected from the genus of Beccaria.
1569. The bioactive agent according to item 1565, wherein Basidiomycete cell is selected from the genus of Beccariella.
1570. The bioactive agent according to item 1565, wherein Basidiomycete cell is selected from the genus of Bresadolina.
1571. The bioactive agent according to item 1565, wherein Basidiomycete cell is selected from the genus of Caripia.
1572. The bioactive agent according to item 1565, wherein Basidiomycete cell is selected from the genus of Cladoderris.
1573. The bioactive agent according to item 1565, wherein Basidiomycete cell is selected from the genus of Coralloderma.
1574. The bioactive agent according to item 1565, wherein Basidiomycete cell is selected from the genus of Cotylidia.
1575. The bioactive agent according to item 1565, wherein Basidiomycete cell is selected from the genus of Craterella.
1576. The bioactive agent according to item 1565, wherein Basidiomycete cell is selected from the genus of Cymatoderma.
1577. The bioactive agent according to item 1565, wherein Basidiomycete cell is selected from the genus of Cyphellostereum.
1578. The bioactive agent according to item 1565, wherein Basidiomycete cell is selected from the genus of Granulobasidium.
1579. The bioactive agent according to item 1565, wherein Basidiomycete cell is selected from the genus of Inflatostereum.
1580. The bioactive agent according to item 1565, wherein Basidiomycete cell is selected from the genus Podoscypha.
1581. The bioactive agent according to item 1565, wherein Basidiomycete cell is selected from the genus of Pseudolasiobolus.
1582. The bioactive agent according to item 1565, wherein Basidiomycete cell is selected from the genus of Stereogloeocystidium.
1583. The bioactive agent according to item 1565, wherein Basidiomycete cell is selected from the genus of Stereophyllum.
1584. The bioactive agent according to item 1565, wherein Basidiomycete cell is selected from the genus of Stereopsis.
1585. The bioactive agent according to item 264, wherein said Basidiomycete cell belongs to a genus selected from the group consisting of Abundisporus, Agarico-igniarium, Agaricum, Amyloporia, Amyloporiella, Antromycopsis, Apoxona, Artolenzites, Asterochaete, Atroporus, Aurantiporellus, Australoporus, Austrolentinus, Bresadolia, Bridgeoporus, Bulliardia, Burgoa, Caloporus, Cellularia, Ceriomyces, Cerioporus, Cerrena, Choriphyllum, Cladoporus, Coriolopsis, Coriolus, Cryptomphalina, Cryptoporus, Cubamyces, Cyanosporus, Cystidiophorus, Cystostiptoporus, Daedaleopsis, Datronia, Dendrochaete, Dendropolyporus, Dextrinosporium, Dichomitus, Digitellus, Earliella, Echinochaete, Elfvingiella, Enslinia, Fabisporus, Faerberia, Favolus, Fibroporia, Flabellophora, Fomes, Fomitella, Funalia, Fuscocerrena, Gemmularia, Geopetalum, Globifomes, Grammothelopsis, Hansenia, Haploporus, Heliocybe, Hexagonia, Hirschioporus, Hornodermoporus, Incrustoporia, Laccocephalum, Laetifomes, Laetiporus, Lasiochlaena, Lentinopanus, Lentinus, Lentodiellum, Lentodium, Lentus, Lenzites, Leptopora, Leptoporellus, Leptotrimitus, Leucolenzites, Leucoporus, Lignosus, Lithopolyporales, Loweporus, Macrohyporia, Macroporia, Megasporoporia, Melanoporella, Melanoporia, Melanopus, Merulioporia, Microporellus, Microporus, Mollicarpus, Mycelithe, Navisporus, Neolentinus, Neolentiporus, Nigrofomes, Nigroporus, Oligoporus, Osmoporus, Pachykytospora, Pachyma, Panus, Paramyces, Perenniporia, Perenniporiella, Persooniana, Petaloides, Phaeolus, Phaeotrametes, Pherima, Phorima, Phyllodontia, Physisporus, Piloporia, Placodes, Pleuropus, Pocillaria, Podofomes, Pogonomyces, Polyporellus, Polyporus, Polyporus, Polyporus, Poria, Porodisculus, Porodiscus, Poronidulus, Poroptyche, Pseudofavolus, Pseudophaeolus, Pseudopiptoporus, Pseudotrametes, Ptychogaster, Pycnoporellus, Pycnoporus, Pyrofomes, Riopa, Romellia, Royoporus, Rubroporus, Ryvardenia, Scenidium, Sclerodepsis, Sistotrema, Skeletocutis, Sparsitubus, Spongiosus, Stiptophyllum, Tinctoporellus, Tomentoporus, Trametella, Trametes, Trichaptum, Truncospora, Tuberaster, Tyromyces, Ungulina, Vanderbylia, Velolentinus, Xerotinus, Xerotus, Xylometron and Xylopilus.
1586. The bioactive agent according to item 1585, wherein Basidiomycete cell is selected from the genus of Abundisporus.
1587. The bioactive agent according to item 1585, wherein Basidiomycete cell is selected from the genus of Agarico-igniarium.
1588. The bioactive agent according to item 1585, wherein Basidiomycete cell is selected from the genus of Agaricum.
1589. The bioactive agent according to item 1585, wherein Basidiomycete cell is selected from the genus of Amyloporia.
1590. The bioactive agent according to item 1585, wherein Basidiomycete cell is selected from the genus of Amyloporiella.
1591. The bioactive agent according to item 1585, wherein Basidiomycete cell is selected from the genus of Antromycopsis.
1592. The bioactive agent according to item 1585, wherein Basidiomycete cell is selected from the genus of Apoxona.
1593. The bioactive agent according to item 1585, wherein Basidiomycete cell is selected from the genus of Artolenzites.
1594. The bioactive agent according to item 1585, wherein Basidiomycete cell is selected from the genus of Asterochaete.
1595. The bioactive agent according to item 1585, wherein Basidiomycete cell is selected from the genus of Atroporus.
1596. The bioactive agent according to item 1585, wherein Basidiomycete cell is selected from the genus of Aurantiporellus.
1597. The bioactive agent according to item 1585, wherein Basidiomycete cell is selected from the genus of Australoporus.
1598. The bioactive agent according to item 1585, wherein Basidiomycete cell is selected from the genus of Austrolentinus.
1599. The bioactive agent according to item 1585, wherein Basidiomycete cell is selected from the genus of Bresadolia.
1600. The bioactive agent according to item 1585, wherein Basidiomycete cell is selected from the genus of Bridgeoporus.
1601. The bioactive agent according to item 1585, wherein Basidiomycete cell is selected from the genus of Bulliardia.
1602. The bioactive agent according to item 1585, wherein Basidiomycete cell is selected from the genus of Burgoa.
1603. The bioactive agent according to item 1585, wherein Basidiomycete cell is selected from the genus of Caloporus.
1604. The bioactive agent according to item 1585, wherein Basidiomycete cell is selected from the genus of Cellularia.
1605. The bioactive agent according to item 1585, wherein Basidiomycete cell is selected from the genus of Ceriomyces.
1606. The bioactive agent according to item 1585, wherein Basidiomycete cell is selected from the genus of Cerioporus.
1607. The bioactive agent according to item 1585, wherein Basidiomycete cell is selected from the genus of Cerrena.
1608. The bioactive agent according to item 1585, wherein Basidiomycete cell is selected from the genus of Choriphyllum.
1609. The bioactive agent according to item 1585, wherein Basidiomycete cell is selected from the genus of Cladoporus.
1610. The bioactive agent according to item 1585, wherein Basidiomycete cell is selected from the genus of Coriolopsis.
1611. The bioactive agent according to item 1585, wherein Basidiomycete cell is selected from the genus of Coriolus.
1612. The bioactive agent according to item 1585, wherein Basidiomycete cell is selected from the genus of Cryptomphalina.
1613. The bioactive agent according to item 1585, wherein Basidiomycete cell is selected from the genus of Cryptoporus.
1614. The bioactive agent according to item 1585, wherein Basidiomycete cell is selected from the genus of Cubamyces.
1615. The bioactive agent according to item 1585, wherein Basidiomycete cell is selected from the genus of Cyanosporus.
1616. The bioactive agent according to item 1585, wherein Basidiomycete cell is selected from the genus of Cystidiophorus.
1617. The bioactive agent according to item 1585, wherein Basidiomycete cell is selected from the genus of Cystostiptoporus.
1618. The bioactive agent according to item 1585, wherein Basidiomycete cell is selected from the genus of Daedaleopsis.
1619. The bioactive agent according to item 1585, wherein Basidiomycete cell is selected from the genus of Datronia.
1620. The bioactive agent according to item 1585, wherein Basidiomycete cell is selected from the genus of Dendrochaete.
1621. The bioactive agent according to item 1585, wherein Basidiomycete cell is selected from the genus of Dendropolyporus.
1622. The bioactive agent according to item 1585, wherein Basidiomycete cell is selected from the genus of Dextrinosporium.
1623. The bioactive agent according to item 1585, wherein Basidiomycete cell is selected from the genus of Dichomitus.
1624. The bioactive agent according to item 1585, wherein Basidiomycete cell is selected from the genus of Digitellus.
1625. The bioactive agent according to item 1585, wherein Basidiomycete cell is selected from the genus of Earliella.
1626. The bioactive agent according to item 1585, wherein Basidiomycete cell is selected from the genus Echinochaete.
1627. The bioactive agent according to item 1585, wherein Basidiomycete cell is selected from the genus Elfvingiella.
1628. The bioactive agent according to item 1585, wherein Basidiomycete cell is selected from the genus of Enslinia.
1629. The bioactive agent according to item 1585, wherein Basidiomycete cell is selected from the genus of Fabisporus.
1630. The bioactive agent according to item 1585, wherein Basidiomycete cell is selected from the genus of Faerberia.
1631. The bioactive agent according to item 1585, wherein Basidiomycete cell is selected from the genus of Favolus.
1632. The bioactive agent according to item 1585, wherein Basidiomycete cell is selected from the genus of Fibroporia.
1633. The bioactive agent according to item 1585, wherein Basidiomycete cell is selected from the genus Flabellophora.
1634. The bioactive agent according to item 1585, wherein Basidiomycete cell is selected from the genus of Fomes.
1635. The bioactive agent according to item 1585, wherein Basidiomycete cell is selected from the genus of Fomitella.
1636. The bioactive agent according to item 1585, wherein Basidiomycete cell is selected from the genus of Funalia.
1637. The bioactive agent according to item 1585, wherein Basidiomycete cell is selected from the genus of Fuscocerrena.
1638. The bioactive agent according to item 1585, wherein Basidiomycete cell is selected from the genus of Gemmularia.
1639. The bioactive agent according to item 1585, wherein Basidiomycete cell is selected from the genus of Geopetalum.
1640. The bioactive agent according to item 1585, wherein Basidiomycete cell is selected from the genus Globifomes.
1641. The bioactive agent according to item 1585, wherein Basidiomycete cell is selected from the genus of Grammothelopsis.
1642. The bioactive agent according to item 1585, wherein Basidiomycete cell is selected from the genus of Hansenia.
1643. The bioactive agent according to item 1585, wherein Basidiomycete cell is selected from the genus of Haploporus.
1644. The bioactive agent according to item 1585, wherein Basidiomycete cell is selected from the genus of Heliocybe.
1645. The bioactive agent according to item 1585, wherein Basidiomycete cell is selected from the genus Hexagonia.
1646. The bioactive agent according to item 1585, wherein Basidiomycete cell is selected from the genus of Hirschioporus.
1647. The bioactive agent according to item 1585, wherein Basidiomycete cell is selected from the genus of Hornodermoporus.
1648. The bioactive agent according to item 1585, wherein Basidiomycete cell is selected from the genus of Incrustoporia.
1649. The bioactive agent according to item 1585, wherein Basidiomycete cell is selected from the genus of Laccocephalum.
1650. The bioactive agent according to item 1585, wherein Basidiomycete cell is selected from the genus of Laetifomes.
1651. The bioactive agent according to item 1585, wherein Basidiomycete cell is selected from the genus of Laetiporus.
1652. The bioactive agent according to item 1585, wherein Basidiomycete cell is selected from the genus of Lasiochlaena.
1653. The bioactive agent according to item 1585, wherein Basidiomycete cell is selected from the genus Lentinopanus.
1654. The bioactive agent according to item 1585, wherein Basidiomycete cell is selected from the genus Lentinus.
1655. The bioactive agent according to item 1585, wherein Basidiomycete cell is selected from the genus of Lentodiellum.
1656. The bioactive agent according to item 1585, wherein Basidiomycete cell is selected from the genus of Lentodium.
1657. The bioactive agent according to item 1585, wherein Basidiomycete cell is selected from the genus of Lentus.
1658. The bioactive agent according to item 1585, wherein Basidiomycete cell is selected from the genus of Lenzites.
1659. The bioactive agent according to item 1585, wherein Basidiomycete cell is selected from the genus of Leptopora.
1660. The bioactive agent according to item 1585, wherein Basidiomycete cell is selected from the genus of Leptoporellus.
1661. The bioactive agent according to item 1585, wherein Basidiomycete cell is selected from the genus of Leptotrimitus.
1662. The bioactive agent according to item 1585, wherein Basidiomycete cell is selected from the genus of Leucolenzites.
1663. The bioactive agent according to item 1585, wherein Basidiomycete cell is selected from the genus of Leucoporus.
1664. The bioactive agent according to item 1585, wherein Basidiomycete cell is selected from the genus Lignosus.
1665. The bioactive agent according to item 1585, wherein Basidiomycete cell is selected from the genus of Lithopolyporales.
1666. The bioactive agent according to item 1585, wherein Basidiomycete cell is selected from the genus of Loweporus.
1667. The bioactive agent according to item 1585, wherein Basidiomycete cell is selected from the genus of Macrohyporia.
1668. The bioactive agent according to item 1585, wherein Basidiomycete cell is selected from the genus of Macroporia.
1669. The bioactive agent according to item 1585, wherein Basidiomycete cell is selected from the genus of Megasporoporia.
1670. The bioactive agent according to item 1585, wherein Basidiomycete cell is selected from the genus of Melanoporella.
1671. The bioactive agent according to item 1585, wherein Basidiomycete cell is selected from the genus of Melanoporia.
1672. The bioactive agent according to item 1585, wherein Basidiomycete cell is selected from the genus of Melanopus.
1673. The bioactive agent according to item 1585, wherein Basidiomycete cell is selected from the genus of Merulioporia.
1674. The bioactive agent according to item 1585, wherein Basidiomycete cell is selected from the genus of Meruliporia.
1675. The bioactive agent according to item 1585, wherein Basidiomycete cell is selected from the genus of Microporellus.
1676. The bioactive agent according to item 1585, wherein Basidiomycete cell is selected from the genus of Microporus.
1677. The bioactive agent according to item 1585, wherein Basidiomycete cell is selected from the genus of Mollicarpus.
1678. The bioactive agent according to item 1585, wherein Basidiomycete cell is selected from the genus of Mycelithe.
1679. The bioactive agent according to item 1585, wherein Basidiomycete cell is selected from the genus of Navisporus.
1680. The bioactive agent according to item 1585, wherein Basidiomycete cell is selected from the genus of Neolentinus.
1681. The bioactive agent according to item 1585, wherein Basidiomycete cell is selected from the genus of Neolentiporus.
1682. The bioactive agent according to item 1585, wherein Basidiomycete cell is selected from the genus of Nigrofomes.
1683. The bioactive agent according to item 1585, wherein Basidiomycete cell is selected from the genus of Nigroporus.
1684. The bioactive agent according to item 1585, wherein Basidiomycete cell is selected from the genus Oligoporus.
1685. The bioactive agent according to item 1585, wherein Basidiomycete cell is selected from the genus of Osmoporus.
1686. The bioactive agent according to item 1585, wherein Basidiomycete cell is selected from the genus of Pachykytospora.
1687. The bioactive agent according to item 1585, wherein Basidiomycete cell is selected from the genus of Pachyma.
1688. The bioactive agent according to item 1585, wherein Basidiomycete cell is selected from the genus of Panus.
1689. The bioactive agent according to item 1585, wherein Basidiomycete cell is selected from the genus of Paramyces.
1690. The bioactive agent according to item 1585, wherein Basidiomycete cell is selected from the genus of Perenniporia.
1691. The bioactive agent according to item 1585, wherein Basidiomycete cell is selected from the genus of Perenniporiella.
1692. The bioactive agent according to item 1585, wherein Basidiomycete cell is selected from the genus of Persooniana.
1693. The bioactive agent according to item 1585, wherein Basidiomycete cell is selected from the genus of Petaloides.
1694. The bioactive agent according to item 1585, wherein Basidiomycete cell is selected from the genus of Phaeolus.
1695. The bioactive agent according to item 1585, wherein Basidiomycete cell is selected from the genus of Phaeotrametes.
1696. The bioactive agent according to item 1585, wherein Basidiomycete cell is selected from the genus of Pherima.
1697. The bioactive agent according to item 1585, wherein Basidiomycete cell is selected from the genus of Phorima.
1698. The bioactive agent according to item 1585, wherein Basidiomycete cell is selected from the genus of Phyllodontia.
1699. The bioactive agent according to item 1585, wherein Basidiomycete cell is selected from the genus of Physisporus.
1700. The bioactive agent according to item 1585, wherein Basidiomycete cell is selected from the genus of Piloporia.
1701. The bioactive agent according to item 1585, wherein Basidiomycete cell is selected from the genus of Piloporia.
1702. The bioactive agent according to item 1585, wherein Basidiomycete cell is selected from the genus of Placodes.
1703. The bioactive agent according to item 1585, wherein Basidiomycete cell is selected from the genus of Pleuropus.
1704. The bioactive agent according to item 1585, wherein Basidiomycete cell is selected from the genus of Pocillaria.
1705. The bioactive agent according to item 1585, wherein Basidiomycete cell is selected from the genus of Podofomes.
1706. The bioactive agent according to item 1585, wherein Basidiomycete cell is selected from the genus Pogonomyces.
1707. The bioactive agent according to item 1585, wherein Basidiomycete cell is selected from the genus of Polyporellus.
1708. The bioactive agent according to item 1585, wherein Basidiomycete cell is selected from the genus of Polyporus.
1709. The bioactive agent according to item 1585, wherein Basidiomycete cell is selected from the genus of Poria.
1710. The bioactive agent according to item 1585, wherein Basidiomycete cell is selected from the genus Porodisculus.
1711. The bioactive agent according to item 1585, wherein Basidiomycete cell is selected from the genus of Porodiscus.
1712. The bioactive agent according to item 1585, wherein Basidiomycete cell is selected from the genus of Poronidulus.
1713. The bioactive agent according to item 1585, wherein Basidiomycete cell is selected from the genus of Poroptyche.
1714. The bioactive agent according to item 1585, wherein Basidiomycete cell is selected from the genus of Pseudofavolus.
1715. The bioactive agent according to item 1585, wherein Basidiomycete cell is selected from the genus of Pseudophaeolus.
1716. The bioactive agent according to item 1585, wherein Basidiomycete cell is selected from the genus of Pseudopiptoporus.
1717. The bioactive agent according to item 1585, wherein Basidiomycete cell is selected from the genus Pseudotrametes.
1718. The bioactive agent according to item 1585, wherein Basidiomycete cell is selected from the genus of Ptychogaster.
1719. The bioactive agent according to item 1585, wherein Basidiomycete cell is selected from the genus of Pycnoporellus.
1720. The bioactive agent according to item 1585, wherein Basidiomycete cell is selected from the genus of Pycnoporus.
1721. The bioactive agent according to item 1585, wherein Basidiomycete cell is selected from the genus of Pyrofomes.
1722. The bioactive agent according to item 1585, wherein Basidiomycete cell is selected from the genus Riopa.
1723. The bioactive agent according to item 1585, wherein Basidiomycete cell is selected from the genus of Romellia.
1724. The bioactive agent according to item 1585, wherein Basidiomycete cell is selected from the genus of Royoporus.
1725. The bioactive agent according to item 1585, wherein Basidiomycete cell is selected from the genus of Rubroporus.
1726. The bioactive agent according to item 1585, wherein Basidiomycete cell is selected from the genus of Ryvardenia.
1727. The bioactive agent according to item 1585, wherein Basidiomycete cell is selected from the genus of Scenidium.
1728. The bioactive agent according to item 1585, wherein Basidiomycete cell is selected from the genus of Sclerodepsis.
1729. The bioactive agent according to item 1585, wherein Basidiomycete cell is selected from the genus of Sistotrema.
1730. The bioactive agent according to item 1585, wherein Basidiomycete cell is selected from the genus of Skeletocutis.
1731. The bioactive agent according to item 1585, wherein Basidiomycete cell is selected from the genus of Sparsitubus.
1732. The bioactive agent according to item 1585, wherein Basidiomycete cell is selected from the genus of Spongiosus.
1733. The bioactive agent according to item 1585, wherein Basidiomycete cell is selected from the genus of Stiptophyllum.
1734. The bioactive agent according to item 1585, wherein Basidiomycete cell is selected from the genus of Tinctoporellus.
1735. The bioactive agent according to item 1585, wherein Basidiomycete cell is selected from the genus Tomentoporus.
1736. The bioactive agent according to item 1585, wherein Basidiomycete cell is selected from the genus of Trametella.
1737. The bioactive agent according to item 1585, wherein Basidiomycete cell is selected from the genus of Trametes.
1738. The bioactive agent according to item 1585, wherein Basidiomycete cell is selected from the genus of Trichaptum.
1739. The bioactive agent according to item 1585, wherein Basidiomycete cell is selected from the genus of Truncospora.
1740. The bioactive agent according to item 1585, wherein Basidiomycete cell is selected from the genus of Tuberaster.
1741. The bioactive agent according to item 1585, wherein Basidiomycete cell is selected from the genus of Tyromyces.
1742. The bioactive agent according to item 1585, wherein Basidiomycete cell is selected from the genus of Ungulina.
1743. The bioactive agent according to item 1585, wherein Basidiomycete cell is selected from the genus of Vanderbylia.
1744. The bioactive agent according to item 1585, wherein Basidiomycete cell is selected from the genus of Velolentinus.
1745. The bioactive agent according to item 1585, wherein Basidiomycete cell is selected from the genus of Xerotinus.
1746. The bioactive agent according to item 1585, wherein Basidiomycete cell is selected from the genus of Xerotus.
1747. The bioactive agent according to item 1585, wherein Basidiomycete cell is selected from the genus of Xylometron.
1748. The bioactive agent according to item 1585, wherein Basidiomycete cell is selected from the genus of Xylopilus.
1749. The bioactive agent according to item 265, wherein said Basidiomycete cell belongs to a genus selected from the group consisting of Cristelloporia, Echinotrema, Fibriciellum, Fibuloporia, Galziniella, Heptasporium, Hydnotrema, Ingoldiella, Minimedusa, Osteomorpha, Paullicorticium, Repetobasidiellurn, Repetobasidium, Sistotrema, Sistotremastrum, Sistotremella, Sphaerobasidium, Tomentella, Trechispora and Urnobasidium.
1750. The bioactive agent according to item 1749, wherein Basidiomycete cell is selected from the genus of Cristelloporia.
1751. The bioactive agent according to item 1749, wherein Basidiomycete cell is selected from the genus of Echinotrema.
1752. The bioactive agent according to item 1749, wherein Basidiomycete cell is selected from the genus of Fibriciellum.
1753. The bioactive agent according to item 1749, wherein Basidiomycete cell is selected from the genus of Fibuloporia.
1754. The bioactive agent according to item 1749, wherein Basidiomycete cell is selected from the genus of Gaiziniella.
1755. The bioactive agent according to item 1749, wherein Basidiomycete cell is selected from the genus of Heptasporium.
1756. The bioactive agent according to item 1749, wherein Basidiomycete cell is selected from the genus of Hydnotrema.
1757. The bioactive agent according to item 1749, wherein Basidiomycete cell is selected from the genus Ingoldiella.
1758. The bioactive agent according to item 1749, wherein Basidiomycete cell is selected from the genus of Minimedusa.
1759. The bioactive agent according to item 1749, wherein Basidiomycete cell is selected from the genus of Osteomorpha.
1760. The bioactive agent according to item 1749, wherein Basidiomycete cell is selected from the genus of Paullicorticium.
1761. The bioactive agent according to item 1749, wherein Basidiomycete cell is selected from the genus of Repetobasidiellum.
1762. The bioactive agent according to item 1749, wherein Basidiomycete cell is selected from the genus of Repetobasidium.
1763. The bioactive agent according to item 1749, wherein Basidiomycete cell is selected from the genus of Sistotrema.
1764. The bioactive agent according to item 1749, wherein Basidiomycete cell is selected from the genus of Sistotremastrum.
1765. The bioactive agent according to item 1749, wherein Basidiomycete cell is selected from the genus of Sistotremella.
1766. The bioactive agent according to item 1749, wherein Basidiomycete cell is selected from the genus of Sphaerobasidium.
1767. The bioactive agent according to item 1749, wherein Basidiomycete cell is selected from the genus of Tomentella.
1768. The bioactive agent according to item 1749, wherein Basidiomycete cell is selected from the genus of Trechispora.
1769. The bioactive agent according to item 1749, wherein Basidiomycete cell is selected from the genus of Urnobasidium.
1770. The bioactive agent according to item 266, wherein said Basidiomycete cell belongs to a genus selected from the group consisting of Bondarcevomyces, Masseeola, Sparassiella and Sparassis.
1771. The bioactive agent according to item 1770, wherein Basidiomycete cell is selected from the genus of Bondarcevomyces.
1772. The bioactive agent according to item 1770, wherein Basidiomycete cell is selected from the genus of Masseeola.
1773. The bioactive agent according to item 1770, wherein Basidiomycete cell is selected from the genus of Sparassiella.
1774. The bioactive agent according to item 1770, wherein Basidiomycete cell is selected from the genus of Sparassis.
1775. The bioactive agent according to item 267, wherein said Basidiomycete cell belongs to a genus selected from the group consisting of Amethicium, Antrodiella, Aschersonia, Australohydnum, Baeostratoporus, Chaetoporus, Cinereomyces, Diplomitoporus, Etheirodon, Fibricium, Flaviporus, Flavodon, Irpex, Junghuhnia, Lamelloporus, Laschia, Leptodon, Metuloidea, Mycoleptodon, Mycoleptodonoides, Mycorrhaphium, Odontia, Odontina, Spathulina, Steccherinum and Stegiacantha.
1776. The bioactive agent according to item 1775, wherein Basidiomycete cell is selected from the genus of Amethicium.
1777. The bioactive agent according to item 1775, wherein Basidiomycete cell is selected from the genus of Antrodiella.
1778. The bioactive agent according to item 1775, wherein Basidiomycete cell is selected from the genus of Aschersonia.
1779. The bioactive agent according to item 1775, wherein Basidiomycete cell is selected from the genus of Australohydnum.
1780. The bioactive agent according to item 1775, wherein Basidiomycete cell is selected from the genus of Baeostratoporus.
1781. The bioactive agent according to item 1775, wherein Basidiomycete cell is selected from the genus of Chaetoporus.
1782. The bioactive agent according to item 1775, wherein Basidiomycete cell is selected from the genus of Cinereomyces.
1783. The bioactive agent according to item 1775, wherein Basidiomycete cell is selected from the genus of Diplomitoporus.
1784. The bioactive agent according to item 1775, wherein Basidiomycete cell is selected from the genus of Etheirodon.
1785. The bioactive agent according to item 1775, wherein Basidiomycete cell is selected from the genus of Fibricium.
1786. The bioactive agent according to item 1775, wherein Basidiomycete cell is selected from the genus of Flaviporus.
1787. The bioactive agent according to item 1775, wherein Basidiomycete cell is selected from the genus of Flavodon.
1788. The bioactive agent according to item 1775, wherein Basidiomycete cell is selected from the genus of Irpex.
1789. The bioactive agent according to item 1775, wherein Basidiomycete cell is selected from the genus of Junghuhnia.
1790. The bioactive agent according to item 1775, wherein Basidiomycete cell is selected from the genus of Lamelloporus.
1791. The bioactive agent according to item 1775, wherein Basidiomycete cell is selected from the genus of Laschia.
1792. The bioactive agent according to item 1775, wherein Basidiomycete cell is selected from the genus of Leptodon.
1793. The bioactive agent according to item 1775, wherein Basidiomycete cell is selected from the genus of Metuloidea.
1794. The bioactive agent according to item 1775, wherein Basidiomycete cell is selected from the genus of Mycoleptodon.
1795. The bioactive agent according to item 1775, wherein Basidiomycete cell is selected from the genus of Mycoleptodonoides.
1796. The bioactive agent according to item 1775, wherein Basidiomycete cell is selected from the genus of Mycorrhaphium.
1797. The bioactive agent according to item 1775, wherein Basidiomycete cell is selected from the genus of Odontia.
1798. The bioactive agent according to item 1775, wherein Basidiomycete cell is selected from the genus of Odontina.
1799. The bioactive agent according to item 1775, wherein Basidiomycete cell is selected from the genus of Spathulina.
1800. The bioactive agent according to item 1775, wherein Basidiomycete cell is selected from the genus Steccherinum.
1801. The bioactive agent according to item 1775, wherein Basidiomycete cell is selected from the genus of Stegiacantha.
1802. The bioactive agent according to item 268, wherein said Basidiomycete cell belongs to a genus selected from the group consisting of Granulocystis, Leifia, Litschauerella, Tubulicium, Tubulicrinis and Tubulixenasma.
1803. The bioactive agent according to item 1802, wherein Basidiomycete cell is selected from the genus of Granulocystis.
1804. The bioactive agent according to item 1802, wherein Basidiomycete cell is selected from the genus of Leifia.
1805. The bioactive agent according to item 1802, wherein Basidiomycete cell is selected from the genus of Litschauerella.
1806. The bioactive agent according to item 1802, wherein Basidiomycete cell is selected from the genus of Tubulicium.
1807. The bioactive agent according to item 1802, wherein Basidiomycete cell is selected from the genus of Tubulicrinis.
1808. The bioactive agent according to item 1802, wherein Basidiomycete cell is selected from the genus of Tubulixenasma.
1809. The bioactive agent according to item 268, wherein said Basidiomycete cell belongs to a genus selected from the group consisting of Aphanobasidium, Clitopilina, Cunninghammyces, Lepidomyces, Phlebiella, Xenasma, Xenasmatella and Xenosperma.
1810. The bioactive agent according to item 1809, wherein Basidiomycete cell is selected from the genus of Aphanobasidium.
1811. The bioactive agent according to item 1809, wherein Basidiomycete cell is selected from the genus of Clitopilina.
1812. The bioactive agent according to item 1809, wherein Basidiomycete cell is selected from the genus of Cunninghammyces.
1813. The bioactive agent according to item 1809, wherein Basidiomycete cell is selected from the genus of Lepidomyces.
1814. The bioactive agent according to item 1809, wherein Basidiomycete cell is selected from the genus of Phlebiella.
1815. The bioactive agent according to item 1809, wherein Basidiomycete cell is selected from the genus of Xenasma.
1816. The bioactive agent according to item 1809, wherein Basidiomycete cell is selected from the genus of Xenasmatella.
1817. The bioactive agent according to item 1809, wherein Basidiomycete cell is selected from the genus of Xenosperma.
1818. The bioactive agent according to item 363, wherein said Basidiomycete cell belongs to a species selected from the group consisting of Agaricus arorae, Agaricus arvensis, Agaricus augustus, Agaricus benesi, Agaricus bernardii, Agaricus bitorquis, Agaricus californicus, Agaricus campestris, Agaricus comptulus, Agaricus cupreo-brunneus, Agaricus diminutivus, Agaricus fusco-fibrillosus, Agaricus fuscovelatus, Agaricus hondensis, Agaricus lilaceps, Agaricus micromegathus, Agaricus praeclaresquamosus, Agaricus pattersonae, Agaricus perobscurus, Agaricus semotus, Agaricus silvicola, Agaricus subrutilescens and Agaricus xanthodermus.
1819. The bioactive agent according to item 1819, wherein Basidiomycete cell is Agaricus arorae.
1820. The bioactive agent according to item 1819, wherein Basidiomycete cell is Agaricus arvensis.
1821. The bioactive agent according to item 1819, wherein Basidiomycete cell is Agaricus augustus.
1822. The bioactive agent according to item 1819, wherein Basidiomycete cell is Agaricus benesi.
1823. The bioactive agent according to item 1819, wherein Basidiomycete cell is Agaricus bernardii.
1824. The bioactive agent according to item 1819, wherein Basidiomycete cell is Agaricus bitorquis.
1825. The bioactive agent according to item 1819, wherein Basidiomycete cell is Agaricus californicus.
1826. The bioactive agent according to item 1819, wherein Basidiomycete cell is Agaricus campestris.
1827. The bioactive agent according to item 1819, wherein Basidiomycete cell is Agaricus comptulus.
1828. The bioactive agent according to item 1819, wherein Basidiomycete cell is Agaricus cupreo-brunneus.
1829. The bioactive agent according to item 1819, wherein Basidiomycete cell is Agaricus diminutivus.
1830. The bioactive agent according to item 1819, wherein Basidiomycete cell is Agaricus fusco-fibrillosus.
1831. The bioactive agent according to item 1819, wherein Basidiomycete cell is Agaricus fuscovelatus.
1832. The bioactive agent according to item 1819, wherein Basidiomycete cell is Agaricus hondensis.
1833. The bioactive agent according to item 1819, wherein Basidiomycete cell is Agaricus lilaceps.
1834. The bioactive agent according to item 1819, wherein Basidiomycete cell is Agaricus micromegathus.
1835. The bioactive agent according to item 1819, wherein Basidiomycete cell is Agaricus praeclaresquamosus.
1836. The bioactive agent according to item 1819, wherein Basidiomycete cell is Agaricus pattersonae.
1837. The bioactive agent according to item 1819, wherein Basidiomycete cell is Agaricus perobscurus.
1838. The bioactive agent according to item 1819, wherein Basidiomycete cell is Agaricus semotus.
1839. The bioactive agent according to item 1819, wherein Basidiomycete cell is Agaricus silvicola.
1840. The bioactive agent according to item 1819, wherein Basidiomycete cell is Agaricus subrutilescens.
1841. The bioactive agent according to item 1819, wherein Basidiomycete cell is Agaricus xanthodermus.
1842. The bioactive agent according to item 925, wherein said Basidiomycete cell belongs to a species selected from the group consisting of Schizophyllum album, Schizophyllum alneum, Schizophyllum alneum, Schizophyllum amplum, Schizophyllum brasiliense, Schizophyllum brevilamellatum, Schizophyllum commune, Schizophyllum egelingianum, Schizophyllum exiguum, Schizophyllum fasciatum, Schizophyllum flabellare, Schizophyllum leprieurii, Schizophyllum lobatum, Schizophyllum mexicanum, Schizophyllum multifidum, Schizophyllum murrayi, Schizophyllum mya, Schizophyllum palmatum, Schizophyllum radiatum, Schizophyllum umbrinum and Schizophyllum variabile.
1843. The bioactive agent according to item 1842, wherein Basidiomycete cell is Schizophyllum album.
1844. The bioactive agent according to item 1842, wherein Basidiomycete cell is Schizophyllum alneum.
1845. The bioactive agent according to item 1842, wherein Basidiomycete cell is Schizophyllum alneum.
1846. The bioactive agent according to item 1842, wherein Basidiomycete cell is Schizophyllum amplum.
1847. The bioactive agent according to item 1842, wherein Basidiomycete cell is Schizophyllum brasiliense.
1848. The bioactive agent according to item 1842, wherein Basidiomycete cell is Schizophyllum brevilamellatum.
1849. The bioactive agent according to item 1842, wherein Basidiomycete cell is Schizophyllum commune.
1850. The bioactive agent according to item 1842, wherein Basidiomycete cell is Schizophyllum egelingianum.
1851. The bioactive agent according to item 1842, wherein Basidiomycete cell is Schizophyllum exiguum.
1852. The bioactive agent according to item 1842, wherein Basidiomycete cell is Schizophyllum fasciatum.
1853. The bioactive agent according to item 1842, wherein Basidiomycete cell is Schizophyllum flabellare.
1854. The bioactive agent according to item 1842, wherein Basidiomycete cell is Schizophyllum leprieurii.
1855. The bioactive agent according to item 1842, wherein Basidiomycete cell is Schizophyllum lobatum.
1856. The bioactive agent according to item 1842, wherein Basidiomycete cell is Schizophyllum mexicanum.
1857. The bioactive agent according to item 1842, wherein Basidiomycete cell is Schizophyllum multifidum.
1858. The bioactive agent according to item 1842, wherein Basidiomycete cell is Schizophyllum murrayi.
1859. The bioactive agent according to item 1842, wherein Basidiomycete cell is Schizophyllum mya.
1860. The bioactive agent according to item 1842, wherein Basidiomycete cell is Schizophyllum palmatum.
1861. The bioactive agent according to item 1842, wherein Basidiomycete cell is Schizophyllum radiatum.
1862. The bioactive agent according to item 1842, wherein Basidiomycete cell is Schizophyllum umbrinum.
1863. The bioactive agent according to item 1842, wherein Basidiomycete cell is Schizophyllum variabile.
1864. The bioactive agent according to item 1406, wherein said Basidiomycete cell belongs to a species selected from the group consisting of Ganoderma adspersum, Ganoderma africanum, Ganoderma applanatum, Ganoderma arcuatum, Ganoderma areolatum, Ganoderma bakeri, Ganoderma balabacense, Ganoderma cacainum, Ganoderma calcigenum, Ganoderma calidophilum, Ganoderma camphoratum, Ganoderma cantharelloideum, Ganoderma capense, Ganoderma carnosum, Ganoderma cehengense, Ganoderma cervinum, Ganoderma chaffangeonii, Ganoderma chalceum, Ganoderma chaperi, Ganoderma chenhaiense, Ganoderma chilense, Ganoderma chiungchungense, Ganoderma chonoides, Ganoderma cochlear, Ganoderma coffeatum, Ganoderma colossus, Ganoderma comorense, Ganoderma comphoratum, Ganoderma concinnum, Ganoderma conicus, Ganoderma corrugatum, Ganoderma costatus, Ganoderma crebrostriatum, Ganoderma cupreolaccatum, Ganoderma cupreum, Ganoderma cupulatiprocerum, Ganoderma curranii, Ganoderma curtisii, Ganoderma dahlii, Ganoderma daiqingshanense, Ganoderma dejongii, Ganoderma densizonatum, Ganoderma diaoluoshanense, Ganoderma donkii, Ganoderma dorsale, Ganoderma dubio-cochlear, Ganoderma dussii, Ganoderma elmeri, Ganoderma elmerianum, Ganoderma eminii, Ganoderma endochrum, Ganoderma europaeum, Ganoderma exile, Ganoderma expallens, Ganoderma fasciatum, Ganoderma fasciculatum, Ganoderma fassii, Ganoderma fassioides, Ganoderma fici, Ganoderma flabelliforme, Ganoderma flaviporum, Ganoderma flexipes, Ganoderma formosanum, Ganoderma formosissimum, Ganoderma formicatum, Ganoderma frondosum, Ganoderma fulvellum, Ganoderma fuscum, Ganoderma galegense, Ganoderma gelsicola, Ganoderma ghesquierei, Ganoderma gibbosum, Ganoderma gilletii, Ganoderma guadelupense, Ganoderma guinanense, Ganoderma guizhouense, Ganoderma hainanense, Ganoderma henningsii, Ganoderma hildebrandii, Ganoderma hinnuleum, Ganoderma hoehnelianum, Ganoderma hollidayi, Ganoderma hoploides, Ganoderma hypoxanthum, Ganoderma impolitum, Ganoderma incrassatum, Ganoderma incrustatum, Ganoderma infulgens, Ganoderma infundibuliforme, Ganoderma insulare, Ganoderma intermedium, Ganoderma japonicum, Ganoderma jianfenglingense, Ganoderma koningsbergii, Ganoderma kosteri, Ganoderma kunmingense, Ganoderma laccatum, Ganoderma lamaoense, Ganoderma leptopum, Ganoderma leucocreas, Ganoderma leucophaeum, Ganoderma leytense, Ganoderma lignosum, Ganoderma limushanense, Ganoderma lingua, Ganoderma linhartii, Ganoderma lionnetii, Ganoderma lipsiense, Ganoderma lloydii, Ganoderma lobatoideum, Ganoderma lobatum, Ganoderma longipes, Ganoderma longistipatum, Ganoderma longistipitatum, Ganoderma lorenzianum, Ganoderma lucidum, Ganoderma lusambilaense, Ganoderma luteicinctum, Ganoderma luteomarginatum, Ganoderma luteum, Ganoderma macer, Ganoderma magniporum, Ganoderma maitlandil, Ganoderma malayanum, Ganoderma malosporum, Ganoderma mangiferae, Ganoderma manoutchehrii, Ganoderma mastoporum, Ganoderma mediosinense, Ganoderma megaloma, Ganoderma megalosporum, Ganoderma meijangense, Ganoderma melanophaeum, Ganoderma meredithiae, Ganoderma microsporum, Ganoderma miniatocinctum, Ganoderma mirabile, Ganoderma mirivelutinum, Ganoderma mongolicum, Ganoderma multicornum, Ganoderma multipileum, Ganoderma multiplicatum, Ganoderma namutambalaense, Ganoderma neglectus, Ganoderma neojaponicum, Ganoderma neurosporum, Ganoderma nevadense, Ganoderma nigrolucidum, Ganoderma nitens, Ganoderma nitidum, Ganoderma noukahivense, Ganoderma nutans, Ganoderma obockense, Ganoderma obokensis, Ganoderma ochrolaccatum, Ganoderma oerstedii, Ganoderma omphalodes, Ganoderma opacum, Ganoderma orbiforme, Ganoderma oregonense, Ganoderma oroflavum, Ganoderma oroleucum, Ganoderma ostracodes, Ganoderma ostreatum, Ganoderma papillatum, Ganoderma parviungulatum, Ganoderma parvulum, Ganoderma pernanum, Ganoderma personatum, Ganoderma perturbatum, Ganoderma petchii, Ganoderma pfeifferi, Ganoderma philippii, Ganoderma piatense, Ganoderma plicatum, Ganoderma polychromum, Ganoderma polymorphum, Ganoderma praelongum Murrill, Ganoderma praetervisum, Ganoderma preussii, Ganoderma pseudoboletus, Ganoderma pseudoferreum, Ganoderma puberulum, Ganoderma puglisii, Ganoderma pulchella, Ganoderma pullatum, Ganoderma pulverulentum, Ganoderma pygmoideum, Ganoderma ramosissimum, Ganoderma ravenelii, Ganoderma renidens, Ganoderma renii, Ganoderma resinaceum, Ganoderma reticulatosporum, Ganoderma rhacodes, Ganoderma rivulosum, Ganoderma rothwellii, Ganoderma rotundatum, Ganoderma rubeolum, Ganoderma rude, Ganoderma rufoalbum, Ganoderma rufobadium, Ganoderma rugosissimus, Ganoderma rugosum, Ganoderma sanmingense, Ganoderma sarasinii, Ganoderma schomburgkii, Ganoderma scuipturatum, Ganoderma septatum, Ganoderma sequoiae, Ganoderma sessile, Ganoderma sessiliforme, Ganoderma shandongense, Ganoderma shangsiens, Ganoderma sichuanense, Ganoderma sikorae, Ganoderma silveirae, Ganoderma simaoense, Ganoderma simulans, Ganoderma sinense, Ganoderma soniense, Ganoderma soyeri, Ganoderma sprucei, Ganoderma staneri, Ganoderma steyaertanum, Ganoderma stipitatum, Ganoderma stratoideum, Ganoderma subamboinense, Ganoderma subformicatum, Ganoderma subfulvum, Ganoderma subincrustatum, Ganoderma sublucidum, Ganoderma subperforatum, Ganoderma subrenatum, Ganoderma subresinosum, Ganoderma subrugosus, Ganoderma substipitata, Ganoderma subtornatum, Ganoderma subtuberculosum, Ganoderma subumbraculum, Ganoderma sulcatum, Ganoderma tenue, Ganoderma testaceum, Ganoderma theaecolumn, Ganoderma tibetanum, Ganoderma tornatum, Ganoderma torosum, Ganoderma torrendii, Ganoderma trengganuense, Ganoderma triangulum, Ganoderma triviale, Ganoderma tropicum, Ganoderma trulla, Ganoderma trulliforme, Ganoderma tsugae, Ganoderma tsunodae, Ganoderma tuberculosum, Ganoderma tumidum, Ganoderma umbraculum, Ganoderma umbrinum, Ganoderma ungulatum, Ganoderma valesiacum, Ganoderma vanheurnii, Ganoderma vanmeelii, Ganoderma variabile, Ganoderma weberianum, Ganoderma williamsianum, Ganoderma wuhuense, Ganoderma wynaadense, Ganoderma xanthocreas, Ganoderma xingyiense, Ganoderma xylodes, Ganoderma xylonoides, Ganoderma zhenningense and Ganoderma zonatum.
1865. The bioactive agent according to item 1864, wherein Basidiomycete cell is Ganoderma adspersum.
1866. The bioactive agent according to item 1864, wherein Basidiomycete cell is Ganoderma africanum.
1867. The bioactive agent according to item 1864, wherein Basidiomycete cell is Ganoderma applanatum.
1868. The bioactive agent according to item 1864, wherein Basidiomycete cell is Ganoderma arcuatum.
1869. The bioactive agent according to item 1864, wherein Basidiomycete cell is Ganoderma areolatum.
1870. The bioactive agent according to item 1864, wherein Basidiomycete cell is Ganoderma bakeri.
1871. The bioactive agent according to item 1864, wherein Basidiomycete cell is Ganoderma balabacense.
1872. The bioactive agent according to item 1864, wherein Basidiomycete cell is Ganoderma cacainum.
1873. The bioactive agent according to item 1864, wherein Basidiomycete cell is Ganoderma cacainum.
1874. The bioactive agent according to item 1864, wherein Basidiomycete cell is Ganoderma calcigenum.
1875. The bioactive agent according to item 1864, wherein Basidiomycete cell is Ganoderma calidophilum.
1876. The bioactive agent according to item 1864, wherein Basidiomycete cell is Ganoderma camphoratum.
1877. The bioactive agent according to item 1864, wherein Basidiomycete cell is Ganoderma cantharelloideum.
1878. The bioactive agent according to item 1864, wherein Basidiomycete cell is Ganoderma capense.
1879. The bioactive agent according to item 1864, wherein Basidiomycete cell is Ganoderma carnosum.
1880. The bioactive agent according to item 1864, wherein Basidiomycete cell is Ganoderma cehengense.
1881. The bioactive agent according to item 1864, wherein Basidiomycete cell is Ganoderma cervinum.
1882. The bioactive agent according to item 1864, wherein Basidiomycete cell is Ganoderma chaffangeonii.
1883. The bioactive agent according to item 1864, wherein Basidiomycete cell is Ganoderma chalceum.
1884. The bioactive agent according to item 1864, wherein Basidiomycete cell is Ganoderma chaperi.
1885. The bioactive agent according to item 1864, wherein Basidiomycete cell is Ganoderma chenhaiense.
1886. The bioactive agent according to item 1864, wherein Basidiomycete cell is Ganoderma chilense.
1887. The bioactive agent according to item 1864, wherein Basidiomycete cell is Ganoderma chiungchungense.
1888. The bioactive agent according to item 1864, wherein Basidiomycete cell is Ganoderma chonoides.
1889. The bioactive agent according to item 1864, wherein Basidiomycete cell is Ganoderma cochlear.
1890. The bioactive agent according to item 1864, wherein Basidiomycete cell is Ganoderma coffeatum.
1891. The bioactive agent according to item 1864, wherein Basidiomycete cell is Ganoderma colossus.
1892. The bioactive agent according to item 1864, wherein Basidiomycete cell is Ganoderma comorense.
1893. The bioactive agent according to item 1864, wherein Basidiomycete cell is Ganoderma comphoratum.
1894. The bioactive agent according to item 1864, wherein Basidiomycete cell is Ganoderma concinnum.
1895. The bioactive agent according to item 1864, wherein Basidiomycete cell is Ganoderma conicus.
1896. The bioactive agent according to item 1864, wherein Basidiomycete cell is Ganoderma corrugatum.
1897. The bioactive agent according to item 1864, wherein Basidiomycete cell is Ganoderma costatus.
1898. The bioactive agent according to item 1864, wherein Basidiomycete cell is Ganoderma crebrostriatum.
1899. The bioactive agent according to item 1864, wherein Basidiomycete cell is Ganoderma cupreolaccatum.
1900. The bioactive agent according to item 1864, wherein Basidiomycete cell is Ganoderma curranii.
1901. The bioactive agent according to item 1864, wherein Basidiomycete cell is Ganoderma curtisii.
1902. The bioactive agent according to item 1864, wherein Basidiomycete cell is Ganoderma dahlii.
1903. The bioactive agent according to item 1864, wherein Basidiomycete cell is Ganoderma daiqingshanense.
1904. The bioactive agent according to item 1864, wherein Basidiomycete cell is Ganoderma dejongii.
1905. The bioactive agent according to item 1864, wherein Basidiomycete cell is Ganoderma densizonatum.
1906. The bioactive agent according to item 1864, wherein Basidiomycete cell is Ganoderma diaoluoshanense.
1907. The bioactive agent according to item 1864, wherein Basidiomycete cell is Ganoderma donkii.
1908. The bioactive agent according to item 1864, wherein Basidiomycete cell is Ganoderma dorsale.
1909. The bioactive agent according to item 1864, wherein Basidiomycete cell is Ganoderma dubio-cochlear.
1910. The bioactive agent according to item 1864, wherein Basidiomycete cell is Ganoderma dussii.
1911. The bioactive agent according to item 1864, wherein Basidiomycete cell is Ganoderma elmeri.
1912. The bioactive agent according to item 1864, wherein Basidiomycete cell is Ganoderma elmerianum.
1913. The bioactive agent according to item 1864, wherein Basidiomycete cell is Ganoderma eminii.
1914. The bioactive agent according to item 1864, wherein Basidiomycete cell is Ganoderma endochrum.
1915. The bioactive agent according to item 1864, wherein Basidiomycete cell is Ganoderma europaeum.
1916. The bioactive agent according to item 1864, wherein Basidiomycete cell is Ganoderma exile.
1917. The bioactive agent according to item 1864, wherein Basidiomycete cell is Ganoderma expallens.
1918. The bioactive agent according to item 1864, wherein Basidiomycete cell is Ganoderma fasciatum.
1919. The bioactive agent according to item 1864, wherein Basidiomycete cell is Ganoderma fassii.
1920. The bioactive agent according to item 1864, wherein Basidiomycete cell is Ganoderma fassioides.
1921. The bioactive agent according to item 1864, wherein Basidiomycete cell is Ganoderma fici.
1922. The bioactive agent according to item 1864, wherein Basidiomycete cell is Ganoderma flabelliforme.
1923. The bioactive agent according to item 1864, wherein Basidiomycete cell is Ganoderma flaviporum.
1924. The bioactive agent according to item 1864, wherein Basidiomycete cell is Ganoderma flexipes.
1925. The bioactive agent according to item 1864, wherein Basidiomycete cell is Ganoderma formosanum.
1926. The bioactive agent according to item 1864, wherein Basidiomycete cell is Ganoderma formosissimum.
1927. The bioactive agent according to item 1864, wherein Basidiomycete cell is Ganoderma formicatum.
1928. The bioactive agent according to item 1864, wherein Basidiomycete cell is Ganoderma frondosum.
1929. The bioactive agent according to item 1864, wherein Basidiomycete cell is Ganoderma fulvellum.
1930. The bioactive agent according to item 1864, wherein Basidiomycete cell is Ganoderma fuscum.
1931. The bioactive agent according to item 1864, wherein Basidiomycete cell is Ganoderma galegense.
1932. The bioactive agent according to item 1864, wherein Basidiomycete cell is Ganoderma gelsicola.
1933. The bioactive agent according to item 1864, wherein Basidiomycete cell is Ganoderma ghesquierei.
1934. The bioactive agent according to item 1864, wherein Basidiomycete cell is Ganoderma gibbosum.
1935. The bioactive agent according to item 1864, wherein Basidiomycete cell is Ganoderma gilletii.
1936. The bioactive agent according to item 1864, wherein Basidiomycete cell is Ganoderma guadelupense.
1937. The bioactive agent according to item 1864, wherein Basidiomycete cell is Ganoderma guinanense.
1938. The bioactive agent according to item 1864, wherein Basidiomycete cell is Ganoderma guizhouense.
1939. The bioactive agent according to item 1864, wherein Basidiomycete cell is Ganoderma hainanense.
1940. The bioactive agent according to item 1864, wherein Basidiomycete cell is Ganoderma henningsii.
1941. The bioactive agent according to item 1864, wherein Basidiomycete cell is Ganoderma hildebrandii.
1942. The bioactive agent according to item 1864, wherein Basidiomycete cell is Ganoderma hinnuleum.
1943. The bioactive agent according to item 1864, wherein Basidiomycete cell is Ganoderma hoehnelianum.
1944. The bioactive agent according to item 1864, wherein Basidiomycete cell is Ganoderma hollidayi.
1945. The bioactive agent according to item 1864, wherein Basidiomycete cell is Ganoderma hoploides.
1946. The bioactive agent according to item 1864, wherein Basidiomycete cell is Ganoderma hypoxanthum.
1947. The bioactive agent according to item 1864, wherein Basidiomycete cell is Ganoderma impolitum.
1948. The bioactive agent according to item 1864, wherein Basidiomycete cell is Ganoderma incrassatum.
1949. The bioactive agent according to item 1864, wherein Basidiomycete cell is Ganoderma incrustatum.
1950. The bioactive agent according to item 1864, wherein Basidiomycete cell is Ganoderma infulgens.
1951. The bioactive agent according to item 1864, wherein Basidiomycete cell is Ganoderma infundibuliforme.
1952. The bioactive agent according to item 1864, wherein Basidiomycete cell is Ganoderma insulare.
1953. The bioactive agent according to item 1864, wherein Basidiomycete cell is Ganoderma intermedium.
1954. The bioactive agent according to item 1864, wherein Basidiomycete cell is Ganoderma japonicum.
1955. The bioactive agent according to item 1864, wherein Basidiomycete cell is Ganoderma jianfenglingense.
1956. The bioactive agent according to item 1864, wherein Basidiomycete cell is Ganoderma koningsbergii.
1957. The bioactive agent according to item 1864, wherein Basidiomycete cell is Ganoderma kosteri.
1958. The bioactive agent according to item 1864, wherein Basidiomycete cell is Ganoderma kunmingense.
1959. The bioactive agent according to item 1864, wherein Basidiomycete cell is Ganoderma laccatum.
1960. The bioactive agent according to item 1864, wherein Basidiomycete cell is Ganoderma lamaoense.
1961. The bioactive agent according to item 1864, wherein Basidiomycete cell is Ganoderma leptopum.
1962. The bioactive agent according to item 1864, wherein Basidiomycete cell is Ganoderma leucocreas.
1963. The bioactive agent according to item 1864, wherein Basidiomycete cell is Ganoderma leucophaeum.
1964. The bioactive agent according to item 1864, wherein Basidiomycete cell is Ganoderma leytense.
1965. The bioactive agent according to item 1864, wherein Basidiomycete cell is Ganoderma lignosum.
1966. The bioactive agent according to item 1864, wherein Basidiomycete cell is Ganoderma limushanense.
1967. The bioactive agent according to item 1864, wherein Basidiomycete cell is Ganoderma lingua.
1968. The bioactive agent according to item 1864, wherein Basidiomycete cell is Ganoderma linhartii.
1969. The bioactive agent according to item 1864, wherein Basidiomycete cell is Ganoderma lionnetii.
1970. The bioactive agent according to item 1864, wherein Basidiomycete cell is Ganoderma lipsiense.
1971. The bioactive agent according to item 1864, wherein Basidiomycete cell is Ganoderma lloydii.
1972. The bioactive agent according to item 1864, wherein Basidiomycete cell is Ganoderma lobatoideum.
1973. The bioactive agent according to item 1864, wherein Basidiomycete cell is Ganoderma lobatum.
1974. The bioactive agent according to item 1864, wherein Basidiomycete cell is Ganoderma longipes.
1975. The bioactive agent according to item 1864, wherein Basidiomycete cell is Ganoderma longistipatum.
1976. The bioactive agent according to item 1864, wherein Basidiomycete cell is Ganoderma lorenzianum.
1977. The bioactive agent according to item 1864, wherein Basidiomycete cell is Ganoderma lucidum.
1978. The bioactive agent according to item 1864, wherein Basidiomycete cell is Ganoderma lusambilaense.
1979. The bioactive agent according to item 1864, wherein Basidiomycete cell is Ganoderma luteicinctum.
1980. The bioactive agent according to item 1864, wherein Basidiomycete cell is Ganoderma luteomarginatum.
1981. The bioactive agent according to item 1864, wherein Basidiomycete cell is Ganoderma luteum.
1982. The bioactive agent according to item 1864, wherein Basidiomycete cell is Ganoderma macer.
1983. The bioactive agent according to item 1864, wherein Basidiomycete cell is Ganoderma magniporum.
1984. The bioactive agent according to item 1864, wherein Basidiomycete cell is Ganoderma maitlandii.
1985. The bioactive agent according to item 1864, wherein Basidiomycete cell is Ganoderma malayanum.
1986. The bioactive agent according to item 1864, wherein Basidiomycete cell is Ganoderma malosporum.
1987. The bioactive agent according to item 1864, wherein Basidiomycete cell is Ganoderma mangiferae.
1988. The bioactive agent according to item 1864, wherein Basidiomycete cell is Ganoderma manoutchehrii.
1989. The bioactive agent according to item 1864, wherein Basidiomycete cell is Ganoderma mastoporum.
1990. The bioactive agent according to item 1864, wherein Basidiomycete cell is Ganoderma mediosinense.
1991. The bioactive agent according to item 1864, wherein Basidiomycete cell is Ganoderma megaloma.
1992. The bioactive agent according to item 1864, wherein Basidiomycete cell is Ganoderma megalosporum.
1993. The bioactive agent according to item 1864, wherein Basidiomycete cell is Ganoderma meijangense.
1994. The bioactive agent according to item 1864, wherein Basidiomycete cell is Ganoderma melanophaeum.
1995. The bioactive agent according to item 1864, wherein Basidiomycete cell is Ganoderma meredithiae.
1996. The bioactive agent according to item 1864, wherein Basidiomycete cell is Ganoderma microsporum.
1997. The bioactive agent according to item 1864, wherein Basidiomycete cell is Ganoderma miniatocinctum.
1998. The bioactive agent according to item 1864, wherein Basidiomycete cell is Ganoderma mirabile.
1999. The bioactive agent according to item 1864, wherein Basidiomycete cell is Ganoderma mirivelutinum.
2000. The bioactive agent according to item 1864, wherein Basidiomycete cell is Ganoderma mongolicum.
2001. The bioactive agent according to item 1864, wherein Basidiomycete cell is Ganoderma multicornum.
2002. The bioactive agent according to item 1864, wherein Basidiomycete cell is Ganoderma multipileum.
2003. The bioactive agent according to item 1864, wherein Basidiomycete cell is Ganoderma multiplicatum.
2004. The bioactive agent according to item 1864, wherein Basidiomycete cell is Ganoderma namutambalaense.
2005. The bioactive agent according to item 1864, wherein Basidiomycete cell is Ganoderma neglectus.
2006. The bioactive agent according to item 1864, wherein Basidiomycete cell is Ganoderma neojaponicum.
2007. The bioactive agent according to item 1864, wherein Basidiomycete cell is Ganoderma neurosporum.
2008. The bioactive agent according to item 1864, wherein Basidiomycete cell is Ganoderma nevadense.
2009. The bioactive agent according to item 1864, wherein Basidiomycete cell is Ganoderma nigrolucidum.
2010. The bioactive agent according to item 1864, wherein Basidiomycete cell is Ganoderma nitens.
2011. The bioactive agent according to item 1864, wherein Basidiomycete cell is Ganoderma nitidum.
2012. The bioactive agent according to item 1864, wherein Basidiomycete cell Ganoderma noukahivense.
2013. The bioactive agent according to item 1864, wherein Basidiomycete cell is Ganoderma nutans.
2014. The bioactive agent according to item 1864, wherein Basidiomycete cell is Ganoderma obockense.
2015. The bioactive agent according to item 1864, wherein Basidiomycete cell is Ganoderma obokensis.
2016. The bioactive agent according to item 1864, wherein Basidiomycete cell is Ganoderma ochrolaccatum.
2017. The bioactive agent according to item 1864, wherein Basidiomycete cell is Ganoderma oerstedii.
2018. The bioactive agent according to item 1864, wherein Basidiomycete cell is Ganoderma omphalodes.
2019. The bioactive agent according to item 1864, wherein Basidiomycete cell is Ganoderma opacum.
2020. The bioactive agent according to item 1864, wherein Basidiomycete cell is Ganoderma orbiforme.
2021. The bioactive agent according to item 1864, wherein Basidiomycete cell is Ganoderma oregonense.
2022. The bioactive agent according to item 1864, wherein Basidiomycete cell is Ganoderma oroflavum.
2023. The bioactive agent according to item 1864, wherein Basidiomycete cell is Ganoderma oroleucum.
2024. The bioactive agent according to item 1864, wherein Basidiomycete cell is Ganoderma ostracodes.
2025. The bioactive agent according to item 1864, wherein Basidiomycete cell is ostreatum.
2026. The bioactive agent according to item 1864, wherein Basidiomycete cell is Ganoderma papillatum.
2027. The bioactive agent according to item 1864, wherein Basidiomycete cell is Ganoderma parviungulatum.
2028. The bioactive agent according to item 1864, wherein Basidiomycete cell is Ganoderma parvulum.
2029. The bioactive agent according to item 1864, wherein Basidiomycete cell is Ganoderma pernanum.
2030. The bioactive agent according to item 1864, wherein Basidiomycete cell is Ganoderma personatum.
2031. The bioactive agent according to item 1864, wherein Basidiomycete cell is Ganoderma perturbatum.
2032. The bioactive agent according to item 1864, wherein Basidiomycete cell is Ganoderma petchii.
2033. The bioactive agent according to item 1864, wherein Basidiomycete cell is Ganoderma pfeifferi.
2034. The bioactive agent according to item 1864, wherein Basidiomycete cell is Ganoderma philippii.
2035. The bioactive agent according to item 1864, wherein Basidiomycete cell is Ganoderma platense.
2036. The bioactive agent according to item 1864, wherein Basidiomycete cell is Ganoderma plicatum.
2037. The bioactive agent according to item 1864, wherein Basidiomycete cell is Ganoderma polychromum.
2038. The bioactive agent according to item 1864, wherein Basidiomycete cell is Ganoderma polymorphum.
2039. The bioactive agent according to item 1864, wherein Basidiomycete cell is Ganoderma praelongum.
2040. The bioactive agent according to item 1864, wherein Basidiomycete cell is Ganoderma praetervisum.
2041. The bioactive agent according to item 1864, wherein Basidiomycete cell is Ganoderma preussii.
2042. The bioactive agent according to item 1864, wherein Basidiomycete cell is Ganoderma pseudoboletus.
2043. The bioactive agent according to item 1864, wherein Basidiomycete cell is Ganoderma pseudoferreum.
2044. The bioactive agent according to item 1864, wherein Basidiomycete cell is Ganoderma puberulum.
2045. The bioactive agent according to item 1864, wherein Basidiomycete cell is Ganoderma puglisii.
2046. The bioactive agent according to item 1864, wherein Basidiomycete cell is Ganoderma pulchella.
2047. The bioactive agent according to item 1864, wherein Basidiomycete cell is Ganoderma pullatum.
2048. The bioactive agent according to item 1864, wherein Basidiomycete cell is Ganoderma pulverulentum.
2049. The bioactive agent according to item 1864, wherein Basidiomycete cell is Ganoderma pygmoideum.
2050. The bioactive agent according to item 1864, wherein Basidiomycete cell is Ganoderma ramosissimum.
2051. The bioactive agent according to item 1864, wherein Basidiomycete cell is Ganoderma ravenelii.
2052. The bioactive agent according to item 1864, wherein Basidiomycete cell is Ganoderma renidens.
2053. The bioactive agent according to item 1864, wherein Basidiomycete cell is Ganoderma renii.
2054. The bioactive agent according to item 1864, wherein Basidiomycete cell is Ganoderma resinaceum.
2055. The bioactive agent according to item 1864, wherein Basidiomycete cell is Ganoderma reticulatosporum.
2056. The bioactive agent according to item 1864, wherein Basidiomycete cell is Ganoderma rhacode.
2057. The bioactive agent according to item 1864, wherein Basidiomycete cell is Ganoderma rivulosum.
2058. The bioactive agent according to item 1864, wherein Basidiomycete cell is Ganoderma rothwellii.
2059. The bioactive agent according to item 1864, wherein Basidiomycete cell is Ganoderma rotundatum.
2060. The bioactive agent according to item 1864, wherein Basidiomycete cell is Ganoderma rubeolum.
2061. The bioactive agent according to item 1864, wherein Basidiomycete cell is Ganoderma rude.
2062. The bioactive agent according to item 1864, wherein Basidiomycete cell is Ganoderma rufoalbum.
2063. The bioactive agent according to item 1864, wherein Basidiomycete cell is Ganoderma rufobadium.
2064. The bioactive agent according to item 1864, wherein Basidiomycete cell is Ganoderma rugosissimus.
2065. The bioactive agent according to item 1864, wherein Basidiomycete cell is Ganoderma rugosum.
2066. The bioactive agent according to item 1864, wherein Basidiomycete cell is Ganoderma sanmingense.
2067. The bioactive agent according to item 1864, wherein Basidiomycete cell is Ganoderma sarasinii.
2068. The bioactive agent according to item 1864, wherein Basidiomycete cell is Ganoderma schomburgkii.
2069. The bioactive agent according to item 1864, wherein Basidiomycete cell is Ganoderma sculpturatum.
2070. The bioactive agent according to item 1864, wherein Basidiomycete cell is Ganoderma septatum.
2071. The bioactive agent according to item 1864, wherein Basidiomycete cell is Ganoderma sequoiae.
2072. The bioactive agent according to item 1864, wherein Basidiomycete cell is Ganoderma sessile.
2073. The bioactive agent according to item 1864, wherein Basidiomycete cell is Ganoderma sessiliforme.
2074. The bioactive agent according to item 1864, wherein Basidiomycete cell is Ganoderma shandongense.
2075. The bioactive agent according to item 1864, wherein Basidiomycete cell is Ganoderma shangsiens.
2076. The bioactive agent according to item 1864, wherein Basidiomycete cell is Ganoderma sichuanense.
2077. The bioactive agent according to item 1864, wherein Basidiomycete cell is Ganoderma sikorae.
2078. The bioactive agent according to item 1864, wherein Basidiomycete cell is Ganoderma silveirae.
2079. The bioactive agent according to item 1864, wherein Basidiomycete cell is Ganoderma simaoense.
2080. The bioactive agent according to item 1864, wherein Basidiomycete cell is Ganoderma simulans.
2081. The bioactive agent according to item 1864, wherein Basidiomycete cell is Ganoderma sinense.
2082. The bioactive agent according to item 1864, wherein Basidiomycete cell is Ganoderma soniense.
2083. The bioactive agent according to item 1864, wherein Basidiomycete cell is Ganoderma soyeri.
2084. The bioactive agent according to item 1864, wherein Basidiomycete cell is Ganoderma sprucei.
2085. The bioactive agent according to item 1864, wherein Basidiomycete cell is Ganoderma staneri.
2086. The bioactive agent according to item 1864, wherein Basidiomycete cell is Ganoderma steyaertanum.
2087. The bioactive agent according to item 1864, wherein Basidiomycete cell is Ganoderma stipitatum.
2088. The bioactive agent according to item 1864, wherein Basidiomycete cell is Ganoderma stratoideum.
2089. The bioactive agent according to item 1864, wherein Basidiomycete cell is Ganoderma subamboinense.
2090. The bioactive agent according to item 1864, wherein Basidiomycete cell is Ganoderma subformicatum.
2091. The bioactive agent according to item 1864, wherein Basidiomycete cell is Ganoderma subfulvum.
2092. The bioactive agent according to item 1864, wherein Basidiomycete cell is Ganoderma subincrustatum.
2093. The bioactive agent according to item 1864, wherein Basidiomycete cell is Ganoderma sublucidum.
2094. The bioactive agent according to item 1864, wherein Basidiomycete cell is Ganoderma subperforatum.
2095. The bioactive agent according to item 1864, wherein Basidiomycete cell is Ganoderma subrenatum.
2096. The bioactive agent according to item 1864, wherein Basidiomycete cell is Ganoderma subresinosum.
2097. The bioactive agent according to item 1864, wherein Basidiomycete cell is Ganoderma subrugosus.
2098. The bioactive agent according to item 1864, wherein Basidiomycete cell is Ganoderma substipitata.
2099. The bioactive agent according to item 1864, wherein Basidiomycete cell is Ganoderma subtornatum.
2100. The bioactive agent according to item 1864, wherein Basidiomycete cell is Ganoderma subtuberculosum.
2101. The bioactive agent according to item 1864, wherein Basidiomycete cell is Ganoderma subumbraculum.
2102. The bioactive agent according to item 1864, wherein Basidiomycete cell is Ganoderma sulcatum.
2103. The bioactive agent according to item 1864, wherein Basidiomycete cell is Ganoderma tenue.
2104. The bioactive agent according to item 1864, wherein Basidiomycete cell is Ganoderma testaceum.
2105. The bioactive agent according to item 1864, wherein Basidiomycete cell is Ganoderma theaecolum.
2106. The bioactive agent according to item 1864, wherein Basidiomycete cell is Ganoderma tibetanum.
2107. The bioactive agent according to item 1864, wherein Basidiomycete cell is Ganoderma tornatum.
2108. The bioactive agent according to item 1864, wherein Basidiomycete cell is Ganoderma torosum.
2109. The bioactive agent according to item 1864, wherein Basidiomycete cell is Ganoderma torrendii.
2110. The bioactive agent according to item 1864, wherein Basidiomycete cell is Ganoderma trengganuense.
2111. The bioactive agent according to item 1864, wherein Basidiomycete cell is Ganoderma triangulum.
2112. The bioactive agent according to item 1864, wherein Basidiomycete cell is Ganoderma triviale.
2113. The bioactive agent according to item 1864, wherein Basidiomycete cell is Ganoderma tropicum.
2114. The bioactive agent according to item 1864, wherein Basidiomycete cell is Ganoderma trulla.
2115. The bioactive agent according to item 1864, wherein Basidiomycete cell is Ganoderma trulliforme.
2116. The bioactive agent according to item 1864, wherein Basidiomycete cell is Ganoderma tsugae.
2117. The bioactive agent according to item 1864, wherein Basidiomycete cell is Ganoderma tsunodae.
2118. The bioactive agent according to item 1864, wherein Basidiomycete cell is Ganoderma tuberculosum.
2119. The bioactive agent according to item 1864, wherein Basidiomycete cell is Ganoderma tumidum.
2120. The bioactive agent according to item 1864, wherein Basidiomycete cell is Ganoderma umbraculum.
2121. The bioactive agent according to item 1864, wherein Basidiomycete cell is Ganoderma umbrinum.
2122. The bioactive agent according to item 1864, wherein Basidiomycete cell is Ganoderma ungulatum.
2123. The bioactive agent according to item 1864, wherein Basidiomycete cell is Ganoderma valesiacum.
2124. The bioactive agent according to item 1864, wherein Basidiomycete cell is Ganoderma vanheurnii.
2125. The bioactive agent according to item 1864, wherein Basidiomycete cell is Ganoderma vanmeelii.
2126. The bioactive agent according to item 1864, wherein Basidiomycete cell is Ganoderma variabile.
2127. The bioactive agent according to item 1864, wherein Basidiomycete cell is Ganoderma weberianum.
2128. The bioactive agent according to item 1864, wherein Basidiomycete cell is Ganoderma williamsianum.
2129. The bioactive agent according to item 1864, wherein Basidiomycete cell is Ganoderma wuhuense.
2130. The bioactive agent according to item 1864, wherein Basidiomycete cell is Ganoderma wynaadense.
2131. The bioactive agent according to item 1864, wherein Basidiomycete cell is Ganoderma xanthocreas.
2132. The bioactive agent according to item 1864, wherein Basidiomycete cell is Ganoderma xingyiense.
2133. The bioactive agent according to item 1864, wherein Basidiomycete cell is Ganoderma xylodes.
2134. The bioactive agent according to item 1864, wherein Basidiomycete cell is Ganoderma xylonoides.
2135. The bioactive agent according to item 1864, wherein Basidiomycete cell is Ganoderma zhenningense.
2136. The bioactive agent according to item 1864, wherein Basidiomycete cell is Ganoderma zonatum.
2137. The bioactive agent according to item 1498, wherein said Basidiomycete cell belongs to a species selected from the group consisting of Grifola acanthoides, Grifola albicans, Grifola armeniaca, Grifola badia, Grifola colensoi, Grifola eos, Grifola fractipes, Grifola frondosa, Grifola gargal, Grifola gigantea, Grifola intybacea, Grifola lentifrondosa, Grifola obducta, Grifola platypora, Grifola rosularis, Grifola sordulenta, Grifola suiphurea, Grifola sumstinei and Grifola tuckahoe.
2138. The bioactive agent according to item 2137, wherein Basidiomycete cell is Grifola acanthoides.
2139. The bioactive agent according to item 2137, wherein Basidiomycete cell is Grifola albicans.
2140. The bioactive agent according to item 2137, wherein Basidiomycete cell is Grifola armeniaca.
2141. The bioactive agent according to item 2137, wherein Basidiomycete cell is Grifola badia.
2142. The bioactive agent according to item 2137, wherein Basidiomycete cell is Grifola colensoi.
2143. The bioactive agent according to item 2137, wherein Basidiomycete cell is Grifola eos.
2144. The bioactive agent according to item 2137, wherein Basidiomycete cell is Grifola fractipes.
2145. The bioactive agent according to item 2137, wherein Basidiomycete cell is Grifola frondosa.
2146. The bioactive agent according to item 2137, wherein Basidiomycete cell is Grifola gargal.
2147. The bioactive agent according to item 2137, wherein Basidiomycete cell is Grifola gigantea.
2148. The bioactive agent according to item 2137, wherein Basidiomycete cell is Grifola intybacea.
2149. The bioactive agent according to item 2137, wherein Basidiomycete cell is Grifola lentifrondosa.
2150. The bioactive agent according to item 2137, wherein Basidiomycete cell is Grifola obducta.
2151. The bioactive agent according to item 2137, wherein Basidiomycete cell is Grifola platypora.
2152. The bioactive agent according to item 2137, wherein Basidiomycete cell is Grifola rosularis.
2153. The bioactive agent according to item 2137, wherein Basidiomycete cell is Grifola sordulenta.
2154. The bioactive agent according to item 2137, wherein Basidiomycete cell is Grifola sulphurea.
2155. The bioactive agent according to item 2137, wherein Basidiomycete cell is Grifola sumstinei.
2156. The bioactive agent according to item 2137, wherein Basidiomycete cell is Grifola tuckahoe.
2157. The bioactive agent according to item 1654, wherein said Basidiomycete cell belongs to a species selected from the group consisting of Lentinus albovelutinus, Lentinus anthocephalus, Lentinus badius, Lentinus castoreus, Lentinus chrysopeplus, Lentinus cochleatus, Lentinus concinnus, Lentinus delicatus, Lentinus edodes, Lentinus fasciatus, Lentinus hyracinus, Lentinus lepideus sensu, Lentinus lepideus, Lentinus novaezelandiae, Lentinus pulvinulus, Lentinus punctaticeps, Lentinus punctaticeps, Lentinus pygmaeus, Lentinus sajor-caju, Lentinus squarrulosus, Lentinus strigosus, Lentinus suffrutescens, Lentinus tuber-regium and Lentinus zelandicus.
2158. The bioactive agent according to item 2157, wherein Basidiomycete cell is Lentinus albovelutinus.
2159. The bioactive agent according to item 2157, wherein Basidiomycete cell is Lentinus albovelutinus.
2160. The bioactive agent according to item 2157, wherein Basidiomycete cell is Lentinus anthocephalus.
2161. The bioactive agent according to item 2157, wherein Basidiomycete cell is Lentinus badius.
2162. The bioactive agent according to item 2157, wherein Basidiomycete cell is Lentinus castoreus.
2163. The bioactive agent according to item 2157, wherein Basidiomycete cell is Lentinus chrysopeplus.
2164. The bioactive agent according to item 2157, wherein Basidiomycete cell is Lentinus cochleatus.
2165. The bioactive agent according to item 2157, wherein Basidiomycete cell is Lentinus concinnus.
2166. The bioactive agent according to item 2157, wherein Basidiomycete cell is Lentinus delicatus.
2167. The bioactive agent according to item 2157, wherein Basidiomycete cell is Lentinus edodes.
2168. The bioactive agent according to item 2157, wherein Basidiomycete cell is Lentinus fasciatus.
2169. The bioactive agent according to item 2157, wherein Basidiomycete cell is Lentinus hyracinus.
2170. The bioactive agent according to item 2157, wherein Basidiomycete cell is Lentinus lepideus sensu.
2171. The bioactive agent according to item 2157, wherein Basidiomycete cell is Lentinus lepideus.
2172. The bioactive agent according to item 2157, wherein Basidiomycete cell is Lentinus novaezelandiae.
2173. The bioactive agent according to item 2157, wherein Basidiomycete cell is Lentinus pulvinulus.
2174. The bioactive agent according to item 2157, wherein Basidiomycete cell is Lentinus punctaticeps.
2175. The bioactive agent according to item 2157, wherein Basidiomycete cell is Lentinus pygmaeus.
2176. The bioactive agent according to item 2157, wherein Basidiomycete cell is Lentinus sajor-caju.
2177. The bioactive agent according to item 2157, wherein Basidiomycete cell is Lentinus squarrulosus.
2178. The bioactive agent according to item 2157, wherein Basidiomycete cell is Lentinus strigosus.
2179. The bioactive agent according to item 2157, wherein Basidiomycete cell is Lentinus suffrutescens.
2180. The bioactive agent according to item 2157, wherein Basidiomycete cell is Lentinus tuber-regium.
2181. The bioactive agent according to item 2157, wherein Basidiomycete cell is Lentinus zelandicus.
2182. The bioactive agent according to item 1737, wherein said Basidiomycete cell belongs to a species selected from the group consisting of Trametes cervina, Trametes cingulata, Trametes cotonea, Trametes gibbosa, Trametes hirsuta, Trametes incerta, Trametes lactine, Trametes maxima, Trametes meyenii, Trametes morganii, Trametes ochracea, Trametes pubescens, Trametes robiniophila, Trametes suaveolens, Trametes subsinuosa, Trametes tegularis, Trametes tenuis, Trametes trabea, Trametes umbrina, Trametes unicolor, Trametes versicolor, Trametes villosa and Trametes zonata.
2183. The bioactive agent according to item 2182, wherein Basidiomycete cell is Trametes cervina.
2184. The bioactive agent according to item 2182, wherein Basidiomycete cell is Trametes cingulata.
2185. The bioactive agent according to item 2182, wherein Basidiomycete cell is Trametes cotonea.
2186. The bioactive agent according to item 2182, wherein Basidiomycete cell is Trametes gibbosa.
2187. The bioactive agent according to item 2182, wherein Basidiomycete cell is Trametes hirsuta.
2188. The bioactive agent according to item 2182, wherein Basidiomycete cell is Trametes incerta.
2189. The bioactive agent according to item 2182, wherein Basidiomycete cell is Trametes lactine.
2190. The bioactive agent according to item 2182, wherein Basidiomycete cell is Trametes maxima.
2191. The bioactive agent according to item 2182, wherein Basidiomycete cell is Trametes meyenii.
2192. The bioactive agent according to item 2182, wherein Basidiomycete cell is Trametes morganii.
2193. The bioactive agent according to item 2182, wherein Basidiomycete cell is Trametes ochracea.
2194. The bioactive agent according to item 2182, wherein Basidiomycete cell is Trametes pubescens.
2195. The bioactive agent according to item 2182, wherein Basidiomycete cell is Trametes robiniophila.
2196. The bioactive agent according to item 2182, wherein Basidiomycete cell is Trametes suaveolens.
2197. The bioactive agent according to item 2182, wherein Basidiomycete cell is Trametes subsinuosa.
2198. The bioactive agent according to item 2182, wherein Basidiomycete cell is Trametes tegularis.
2199. The bioactive agent according to item 2182, wherein Basidiomycete cell is Trametes tenuis.
2200. The bioactive agent according to item 2182, wherein Basidiomycete cell is Trametes trabea.
2201. The bioactive agent according to item 2182, wherein Basidiomycete cell is Trametes umbrina.
2202. The bioactive agent according to item 2182, wherein Basidiomycete cell is Trametes unicolor.
2203. The bioactive agent according to item 2182, wherein Basidiomycete cell is Trametes versicolor.
2204. The bioactive agent according to item 2182, wherein Basidiomycete cell is Trametes villosa.
2205. The bioactive agent according to item 2182, wherein Basidiomycete cell is Trametes zonata.
2206. A composition comprising the bioactive agent according to any of the items above and a physiologically acceptable carrier.
2207. A pharmaceutical composition comprising the bioactive agent according to any of the items above and a pharmaceutically acceptable carrier.
2208. Use of the pharmaceutical composition according to item 2207 in the manufacture of a medicament.

### Preparing Feed or Food Product

In general the feed or food product is prepared by admixing a biomass, a composition isolated from biomass, an extracellular liquid or an extracellular composition according to the invention to a conventional feed or food product either during preparation of said food or feed product or after said food or feed product has been prepared. For example the admixing may be performed immediately prior to feeding.

In embodiments of the invention, wherein a dry product is preferable, then dried biomass may be employed. It is however also possible to use the extracellular liquid. The extracellular composition may for example be isolated from the extracellular liquid or from a liquid composition isolated from the extracellular liquid by precipitation, such as by alcohol precipitation and the precipitate can be used after drying. It is also possible to adsorb a liquid onto a solid. Any solid capable of adsorbing large amounts of liquids may be used, provided that the solid is not toxic to the animal or human being to be fed with the product. For example, the solid may comprise cellulose. Thus the solid may for example be microcrystalline cellulose.

Zooplankton feed products, such as artemia, Calanus or rotifer feed products may be prepared by preparing an emulsion of an organic phase in water, wherein the organic phase comprises the survival enhancing agent. The survival enhancing agent may be introduced to the organic phase by different methods. In the event that the survival enhancing agent is comprised in a dry composition, then the dry composition may be grinded to a particle size of in the range of 1 to 500 μm, preferably in the range of 5 to 100 μm, more preferably in the range of 10 to 50 μm. The grinded particles may then be mixed with the organic phase. Methods for preparing dry compositions are described herein above in this section. The organic phase may be any organic solvent, preferably an organic solvent which is not toxic to zooplankton. The organic phase may thus for example be marine oil, such as fish oil or train oil, such as cod liver oil or whale oil or vegetable oil, such as soy oil or calamus oil. The aqueous phase may for example be water, such as sea water or lake water.

One kind of marine fish or shell fish feed product may be prepared by feeding above-mentioned zooplankton feed products to zooplankton, wherein said zooplankton, either living or dead constitutes the marine fish or shell fish product. In one embodiment the feed products do not comprise living animals apart from microorganisms. Zooplankton may be incubated with above-mentioned emulsion. A relatively short time interval, such as 1 hour to 7 days may be sufficient for incubation, however longer time may also be employed. For example, zooplankton may continuously hatch and be removed and fresh emulsion may continuously be added

### Method of Feeding Animals

The invention also relates to methods of feeding animals, wherein the methods involve obtaining a food or feed product as described herein above and feed said product to an animal. The feed or food product should be adjusted to the specific animal and may thus be a conventional feed or food product conventionally fed said animal further comprising the extracellular liquid, the extracellular composition or the biomass according to the invention.

The animals may be fed the feed products of the invention from day 0, such as day 1, for example day 2, such as week 1, for example week 2, such as week 3, for example week 4, such as month 2 after birth or hatching. For mammals, the feed products of the invention are preferably fed from day 0, such as day 1, for example day 2, such as week 1, for example week 2, such as week 3, for example week 4, such as month 2 after weaning.

The products may be fed continuously, or they may be fed only for one or more predetermined time intervals. Said predetermined time interval could be for one day, such as 2 days, for example in the range of 3 to 7 days, such as in the range of 1 to 2 weeks, for example in the range of 2 weeks to 1 month, such as for more than 1 month.

By way of example, chickens may be fed with the feed products from day 7 to day 14 after hatching and with conventional feed for the remaining period.

Depending on the animals, various amounts of the product may be fed. Chickens may be fed from 0.1 to 1000 ml, such as from 0.5 to 100 ml, such as from 0.5 to 10 ml extracellular liquid per day or with an amount of extracellular composition corresponding thereto. Pigs may for example be fed in the range of 0.1 to 1000 mg, such as from 0.5 to 100 mg, for example from 0.5 to 10 mg dried biomass per kg per day.

In one embodiment the method comprises feeding a prey organism the feed product and subsequently feeding said prey organism to an animal. This embodiment is in particular relevant for animals preferring living feed.

Thus if the animal is a marine fish or shell fish then the feed product may be fed to zoo plankton, such as artemia or rotifer and said artemia or rotifer may subsequently be fed said marine fish or shellfish, such as marine fish larvae.

### EXAMPLES

The following examples describe illustrative embodiments of the invention and should not be regarded as limiting for the invention.

### Example 1

Protocol for cultivation of Basidiomycete cells according to the present invention. The protocol is used in the further examples unless otherwise stated.

### Cultivation Conditions:

### Temperature: 25° C.±1

### pH: Medium pH

Water: Tap water
Medium: Glucose 30 g/l;
Mycological peptone 10 g/l;
Yeast extract 6 g/l
Malt extract 6 g/l

### Plate Cultivation of Basidiomycete Cells

15 cm Petri dishes containing about 60 ml of the medium+agar at a concentration corresponding to 15 g/l. Inoculate the plates by scraping off the top layer of mycelium on a Petri dish using a sterile scalpel and spread it onto the new plate. One Petri dish will yield enough mycelium to inoculate three new plates. Cultivate the plates at 25° C. for at least three weeks prior to use. They can be kept at this temperature for a total of 7 more weeks before they should be discarded.

### Shake Flask Cultivation of Basidiomycete Cells

500 ml Ehrlenmeyer flasks containing 200 ml of medium. Scrape off the top layer of mycelium on two plates using a sterile scalpel and place in a 300 ml Ehrlenmeyer flask containing 100 ml of medium. Homogenise the resulting mixture. Inoculate the 500 ml flasks with 50 ml of the homogenised material per flask. Put on orbital shaker at 25° C. and 140 rpm and leave for 7-10 days. If required, longer fermentation periods can also be used, such as e.g. 15-30 days.

### Fermenter (3 Litres) Cultivation of Basidiomycete Cells

Place 1.7 litres of the medium in the fermenter and sterilise at 121° C. for 20 mins. Set the fermentation conditions: 25° C., 200-300 rpm and air at 0.2-0.5 vvm. Decant as much liquid as possible from two shake flasks and inoculate the fermenter with the remaining broth (this will normally amount to 300-500 ml). Add a suitable antifoam agent when required (normally throughout the run). Harvests after 6-8 days. If required, longer fermentation periods can also be used, such as e.g. 15-30 days.

### Harvesting of Basidiomycete Cells

Biomass: Remove the biomass from the broth using a nylon cloth with pore size 45 as a filter medium. Wash the biomass thoroughly with water and dry in a microwave oven set at defrost until dry (normal sample size will require about 15 mins). Store in a desiccator until cool and weigh.

Fermentation liquor: The concentration of bioactive agent in the fermentation liquor is determined by precipitation with abs ethanol. Sterile, distilled water is added if necessary to adjust the concentration to the desired level. The resulting liquid is autoclave and stored.

Medical grade: Pass the biomass-free fermentation liquor through a UF filter having a suitable cut-off value, such as e.g. a cut-off value of 300 kD. When 70-80% of the liquid has been removed add water to the retentate to wash the solution. Repeat until the solution has lost much (at least most of) its colour and appears clean.

### Example 2

### Carbohydrate Composition

Lentinus edodes was cultivated in liquid culture in medium comprising 15 g/l glucose, 3 g/1 malt extract, 3 g/l yeast extract and 5 g/l peptone in shake flasks at 25° C. After cultivation the biomass was separated from the rest of the fermentation broth by filtration. The supernatant was subjected to ultrafiltration using a membrane with a nominal pore size of 50,000 Da. The retentate (herein designated the “MediMush product”) was used for analysis of monosaccharide content.

Samples were hydrolysed to their constituent monosaccharides in 1N HCl at 100° C. The hydrolysates were analysed by HPLC using a Dionex MA-1 column with 600 mM NaOH as the mobile phase; detection was by pulsed amperometry.

The major monosaccharides of the MediMush product was mannose and glucose and some galactose. Table 1 displays the relative monosaccharide content of the MediMush product and as well as of another product, Eureka (Lentinan for injection, Eureka, Australia).

TABLE 2

| SAMPLE | MANNOSE | GLUCOSE | GALACTOSE |
|---|---|---|---|
| MediMush | 13.4 ± 0.4 | 12.6 ± 1.3 | 1 |

### Example 3

### Molecular Weight Fractionation

For molecular weight characterisation of their biological activity, the MediMush sample was centrifuged through Microsep (Pall Life Sciences) centrifugal ultrafilters at 6° C. and 7,500×g. Filters with nominal MWCO values of 50,000-1,000,000 were used. Analytical results (see table 2) showed that material yielding mannose, glucose and galactose on acid hydrolysis passed through all four MWCO membranes used:

TABLE 3

| MWCO | MANNOSE | GLUCOSE | GALACTOSE |
|---|---|---|---|
| 1000000 | 10.3 | 2.9 | 1 |
| 300000 | 9.9 | 3.1 | 1 |
| 100000 | 7.3 | 2.9 | 1 |
| 50000 | 8.9 | 2.9 | 1 |

### Example 4

### Soluble Protein

Soluble protein in the samples was measured by a microscale version of the Coomassie dye-binding assay (Mousdale, D. M, Campbell, M. S., Coggins, J. R.

“Purification and characterisation of bifunctional dehydroquinase-shikimate:NADP dehydrogenase from pea seedlings”. Phytochemistry 367, 217-222 (1987). The amounts of soluble protein in the two products are shown below:

| SAMPLE | SOLUBLE PROTEIN (μg/L) |
|---|---|
| MediMush sample | 31.5 +/− 3.43 |

### Example 5

### Breeding of Piglets

5a.

Tests in piglets are performed in models with diarrhoea provoked with E. coli. Piglets are being fed for 4 weeks (from weaning) with different feed products of the invention, wherein the different feed products vary e.g. 10 fold in their content of extracellular or biomass material from liquid culture (for example starting from 1 mg/kg/day dried biomass). A negative control is included in the experiment wherein the piglets are fed with the same feed without fungal material. Each study group consist of 8 piglets with individual habitation.

Intestinal sampling is performed on 2 piglets of each group on day 7, and again on day 28. Blood samples are taken upon completion of the study.

The following parameters are assessed upon completion of the study:

Growth, Survival, Diarrhoea health status, Clinical health status, Relevant systemic and intestinal immunological parameters: Faecal concentration of IgA, macrophages, attachment of bacteria (E. coli), Blood parameters: T and B lymphocytes. TGF-beta, heptoglobin, cytokines. Faeces: E. coli, intestinal flora composition.
5b.

In this example it is demonstrated that the bioactive agent obtained by the method as described in example 1 (precipitated from Fermentation liquor) has a positive effect on the weight increase and feed factor in pigs.

Method: The effect on the weight increase and feed factor in pigs of the bioactive agent was determined by weekly registering the feed uptake and life weight of each pig. Weaned NOROC piglets were feed with feed containing different amounts of the bioactive agent according to the invention from day 1 after weaning to day 35. Feeds 1-4 (see Table 4) were each given to one group of 32 pigs. Pigs were feed according to appetite via automat.

TABLE 4

| Feed No. | bioactive agent/Feed (mg/kg) |
|---|---|
| 1 | 0.0 (control) |
| 2 | 0.02 |
| 3 | 0.2 |
| 4 | 2.0 |

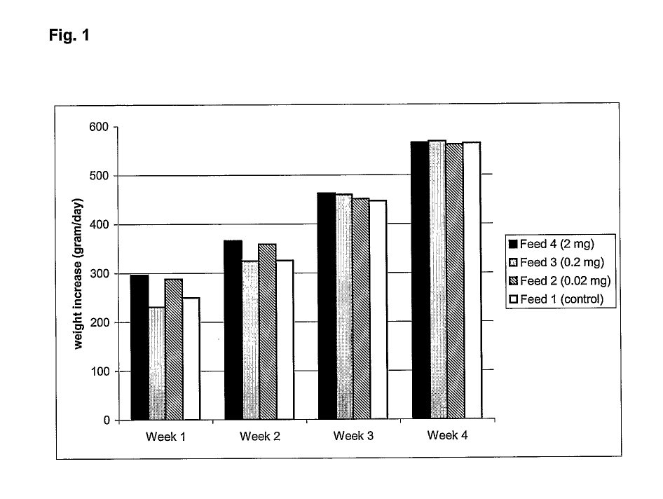

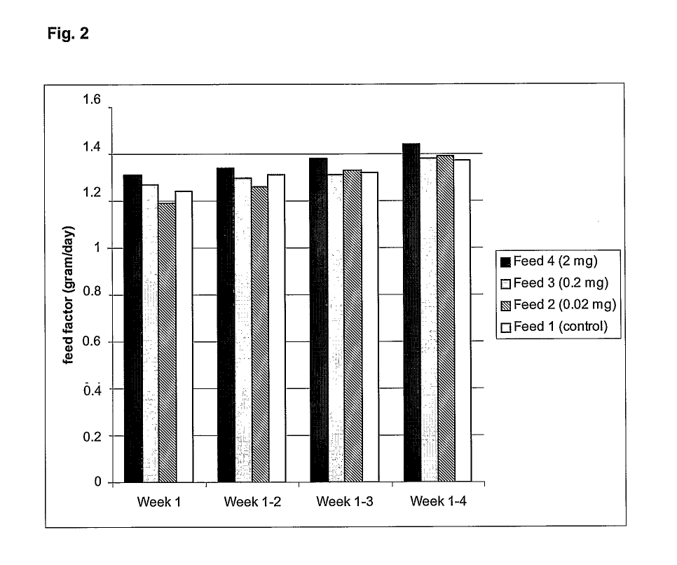

Results: Results are shown in Tables 5 and 6 and in FIGS. 1 and 2.

TABLE 5
Effect of the bioactive agent on average weight increase in
piglets (g/day)

| | Week 1 | Week 2 | Week 3 | Week 4 |
|---|---|---|---|---|
| Feed 4 (2 mg) | 297 | 366 | 463 | 566 |
| Feed 3 (0.2 mg) | 231 | 325 | 460 | 569 |
| Feed 2 (0.02 mg) | 288 | 359 | 451 | 562 |
| Feed 1 (control) | 250 | 326 | 447 | 565 |

TABLE 6
Effect of the bioactive agent on the feed factor in piglets (g/day)

| | Week 1 | Week 1-2 | Week 1-3 | Week 1-4 |
|---|---|---|---|---|
| Feed 4 (2 mg) | 1.31 | 1.34 | 1.38 | 1.44 |
| Feed 3 (0.2 mg) | 1.27 | 1.30 | 1.31 | 1.38 |
| Feed 2 (0.02 mg) | 1.19 | 1.26 | 1.33 | 1.39 |
| Feed 1 (control) | 1.24 | 1.31 | 1.32 | 1.37 |

Conclusion: In weeks 1-3 the weight increase in pigs feed with feeds 2 and 4 is higher than in the control. This is also the case for pigs feed with feed 3 in weeks 3 and 4 (FIG. 1). In addition feed 2 also has a lower feed factor in weeks 1-2, where its effect on the weight increase is most significant (FIG. 2). Thus the bioactive agent is shown to have a positive effect on the weight increase and feed factor in pigs.

### Example 6

### Bacteriostatic Effect

In this example it is demonstrated that the bioactive agent obtained by the method as described in example 1 (precipitated from Fermentation liquor) has a bacteriostatic effect on E. coli K12.

Method: The bacteriostatic effect of the bioactive agent was determined by measuring the cell-density of E. coli K12 cultures grown in Antibiotic assay medium 3 with different dilutions of the bioactive agent. A culture without the bioactive agent in the medium was used as control.

Cells were grown in a 50 ml conical flask at 34° C. for 26 h. The dilutions of the bioactive agent in the growth medium were 1:10, 1:20 and 1:40. The optical density was measured robotically every 2 h at 660 nm.

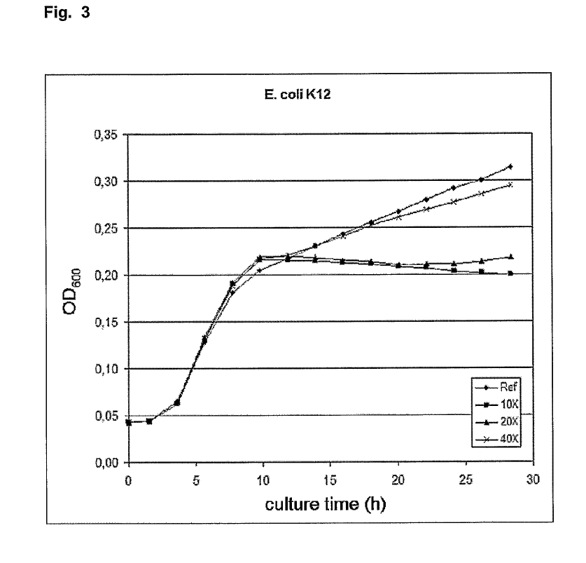

Results: Results are shown in FIG. 3. The optical density significantly decreased in the cultures with a 1:10 and 1:20 dilution of the bioactive agent in the stationary phase (between 15 and 26 h). The incubation with a 1:40 dilution of the bioactive agent does not lead to a significant decrease in optical density in comparison with the control.

Conclusion: The bioactive agent is shown to have a bacteriostatic effect on E. coli K12.

### Example 7

### Anti-Tumor Effect

In this example it is demonstrated that human and mouse cancer cell lines are sensitive to treatment with bioactive agent obtained by the method as described in example 1 (precipitated from Fermentation liquor).

Method: The anti-tumor effect of the bioactive agent was determined by measuring the cell-viability of different human and mouse cell lines after exposure to different concentrations of Lentinex. The MRC-5 cell line from normal human fetal lung fibroblasts was used as control.

Cells were grown in a 96 well dish to a sub confluent cell layer. The medium was removed and the cells washed with PBS. Fresh medium without the bioactive agent (negative control) or containing 0.1; 0.2; 0, 3 or 0.4 mg/ml bioactive agent was added and cells were incubated for 24 h at 37° C.

A MTT-Assay, which measures the activity of the mitochondrial succinate-dehydrogenase, was used to determine the cytotoxic effect of the bioactive agent. In living cells this enzyme converts the yellow water-soluble 3-(4,5-dimethylthiazol-2yl)-2,5-diphenyl-tetrazolium-bromide (MTT) to blue water-insoluble formazan, whereas there is no conversion in dead cells. Thus the amount of formazan directly correlates to the number of living cells.

10 μl MTT solution was added to each well and the plates were incubated for additional 2 h. 70 μl of supernatant were removed from each well and 100 μl acidic isopropanol was added to extract the formazan. After 1 h the absorption was measured at 590 nm.

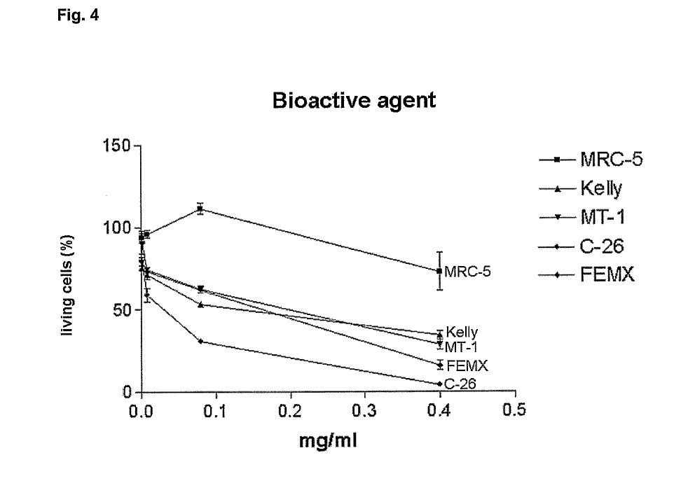

Results: Results are shown in FIG. 4. The number of viable cells was significantly decreased in all cancer cell lines after incubation with the bioactive agent for 24 h. This effect increased with the concentration of the bioactive agent in the medium. In all cancer cell lines, fewer than 50% of the cells were viable after incubation with 0.4 mg/ml bioactive agent for 24 h. The most severe effect of the bioactive agent was observed in the mouse colon cancer cell line C-26, where there were almost no viable cells after the incubation with 0.4 mg/ml bioactive agent for 24 h.

Conclusion: The bioactive agent is shown to have a cytotoxic effect specifically directed against cancer cells, and not normal cells.

### Example 8

Tests in cod larvae is performed through feeding larvae with Artemia or rotifers, which are fed with an oil in water emulsion. The organic phase comprises particles (10-50 μm) and is prepared by one of the following methods.
an extracellular composition is prepared as described in example 1 herein above and precipitated with ethanol. The precipitate is dried and grinded to small particles.
biomass is prepared by fermenting Lentinus edodes as described in example 1. The biomass is air dried and grinded to small particles.

Artemia or rotifer is fed to the larvae continuously throughout the life-feed phase (35-40 days after hatching).

The cod larvae are evaluated according to the following parameters: improved survival, improved growth or reduction in occurrence of deformations.

### Example 9

Tests in cod larvae is performed where cod larvae are fed with feed particles containing proteins, lipids and carbohydrates in addition to particles (80-500 μm) prepared by one of the following methods.
an extracellular composition is prepared as described in example 1 herein above and precipitated with ethanol. The precipitate is dried and grinded to particles.
biomass is prepared by fermenting Lentinus edodes as described in example 1. The biomass is air dried and grinded to particles.

The larvae are continuously fed with above mentioned particles.

The cod larvae are evaluated according to the following parameters: improved survival, improved growth or reduction in occurrence of deformations.

### Example of Functional Food Products

The invention will now be further illustrated by the description of suitable embodiments of the preferred functional food products for use in the invention. It is believed to be well within the ability of the skilled person to use the teaching provided therewith to prepare other products of the invention.

### Example 10

### Bar

75 g of dark chocolate are melted at 70 degrees C. and subsequently mixed with 600 mg of Medimush product. The mixture is poured into a bar shaped mold and cooled overnight.

### Example 11

### Milkshake

100 ml of vanilla flavoured ice-cream are mixed with 100 ml of cooled milk, 10 ml of strawberry syrup and 1 ml extracellular liquid from mushroom cultivation. The mixture is fed through a blender and immediately served.

### Example 12

### Nougat Bar

Ingredient weight (g)

### Water 70

Hyfoama (emulsifier) 3.5

### Gelatin 2.0

### Sugar 515

Glucose syrup 60DE 250
Glucose syrup 35DE 250
Skimmed milk powder 115

### Fat 50

1 g extracellular liquid from mushroom cultivation

Method of preparation: dissolve hyfoama and gelatin in water add 150 g of sugar and beat to foam, heat remaining sugar to 130 degrees C. and add slowly to foam. Add fat, glucose syrup, milkpowder and 1 g extracellular liquid from mushroom cultivation. Allow to cool and divide in bars of 50 g.

### Example 13

### Fruit Drink

11 Ingredients 200 g fruit juice concentrate (banana, pine apple, orange, grape, apricot, lemon, passion fruit, guava, mango) 5 g fructose 1 g inulin 10 g, 0.1 g Medimush product, 0.9 g plant sterol fatty acid ester (=0.54 g sterol equivalents) 5 g lecithin, 1.5 g calcium lactate 775 g water

1. A feed or food product comprising extracellular material derived from a liquid culture of a fungus of the class of Basidiomycetes, wherein said extracellular material comprises a survival enhancing agent and is obtainable by a method comprising the steps of:
i) fermenting a fungus of the class of Basidiomycetes in a liquid culture,
ii) isolating the extracellular material by removal of the biomass, thereby obtaining an extracellular liquid,
iii) optionally isolating an extracellular composition comprising the survival enhancing agent from the extracellular liquid.

2. The feed or food product according to claim 1, wherein said survival enhancing agent comprises polysaccharides.

3. The feed or food product according to claim 1, wherein the survival enhancing agent comprises polypeptides.

4. The feed or food product according to claim 2, wherein at least part of the polysaccharides have a molecular weight of at least 30,000 Da.

5. The feed product according to claim 1, wherein the feed product is an aquatic animal feed product.

6. The feed product according to claim 1, wherein the feed product is a crustacean feed product.

7. The feed product according to claim 1, wherein said extracellular material is comprised within a living microorganism to which the extracellular material has been fed, or administered in another way.

8. The feed product according to claim 1, wherein said extracellular material is comprised within a dead organism to which the extracellular material has been fed or administered in another way.

9. The feed product according to claim 7, wherein said organisms are artemia, rotifer and/or calanus.

10. The feed product according to claim 1, wherein the feed product is a farm animal feed product.

11. The feed product according to claim 1, wherein the feed product is an artemia or rotifer feed product.

12. The feed product of claim 11, wherein the feed product is in the form of an emulsion comprising an organic phase and a water soluble phase.

13. The food or feed product of claim 1, wherein the fungus is a species of Tricholomatales.

14. The food or feed product of claim 1, wherein the fungus is a species of Agaricales.

15. The food or feed product of claim 1, wherein the fungus is a species of Marasmiaceae.

16. The food or feed product of claim 1, wherein the fungus is a species of Lentinus (Lentinula).

17. The food or feed product of claim 1, wherein the fungus is Lentinus (Lentinula) edodes.

18. The food or feed product of claim 1, wherein the culturing in step i) is performed at 25° C., pH 3 to 7 for 50 to 300 hours.

19. The food or feed product of claim 1, wherein the liquid culture comprises malt extract and optionally glucose, yeast extract and peptone.

20. The food or feed product of claim 1, where step ii) comprises centrifugation and/or filtration of the liquid culture.

21. The food or feed product of claim 1, wherein isolation of the extracellular composition comprising the survival enhancing agent, comprises one or more of the following steps:
size fractionation and recovery of molecules above 30,000 kD
ethanol precipitation and recovery of the pellet
drying, such as freeze drying or lyophilisation
spraying and drying
removal of low molecular weight material
removal of oxidants
isolation of polysaccharides
production of an emulsion comprising of an organic phase and a watersoluble phase.

22. The food or feed product according to claim 21, wherein size fractionation is a method involving ultracentrifugation, ultrafiltration, microfiltration or gelfiltration.

23. The food or feed product according to claim 1, wherein only fungal mycelium of said Basidiomycete is fermented.

24. The food or feed product according to claim 1, wherein said Basidiomycete is selected from the group consisting of Agaricus bisporus, Cordiceps sinensis, Flammulina velutipes, Ganoderma lucidum, Grifola frondosa, Lentinus edodes, Pleurotus ostreatus, Schizophyllum commune, Sclerotina sclerotium, Trametes (Coriolus) versicolor, Tremella fuciformis, Agaricus blazei, Agrocybe aegerita, Agrocybe cylindracea, Albatrellus confluens, Armillariella mellea, Auricularia auricula-judae, Auricularia polytricha, Collybia maracula, Cordiceps militari, Dendropolyporus umbellatus, Fomes fomentarius, Fomes pinicola, Ganoderma applanatum, Ganoderma tsugae, Hericium erinaceus, Hypsizygus marmoreus, Inonotus obliquus, Laetiporus sulphurous, Lenzites betulinus, Leucopaxilllus giganteus, Lyophyllum cinerascens, Omphalina epichysium, Oudemansiella mucida, Panellus serotinus, Piptoporus betulinus, Phellinus linteus, Phellinus pini, Pholiota nameko, Pleurotus citrinopileatus, Pleurotus pulmonarius, Sarcedon asparatus, Trametes suavolens, Volvariella volvacea and Wolfiporia cocos.

25. The food or feed product according to claim 24, wherein said Basidiomycete is selected from the group consisting of Agaricus bisporus, Cordiceps sinensis, Flammulina velutipes, Ganoderma lucidum, Grifola frondosa, Lentinus edodes, Pleurotus ostreatus, Schizophyllum commune, Sclerotina sclerotium, Trametes (Coriolus) versicolor and Tremella fuciformis.

26. The food or feed product according to claim 1, wherein the survival enhancing agent comprises a mixture of polysaccharides comprising the monosaccharides galactose, mannose, and glucose in the ratio 1:0 to 25:1 to 50.

27. The food or feed product according to claim 1, wherein the survival enhancing agent comprises at least 10 μg/L soluble polypeptide.

28. The food or feed product according to claim 1, wherein said survival enhancing agent comprises in the range of 10 to 1000 μg/L soluble polypeptide.

29. A method for producing a food or feed product of claim 1 comprising the steps of
i) fermenting a fungus of the class of Basidiomycetes in a liquid culture,
ii) isolating the extracellular material by removal of the biomass,
iii) optionally performing one or more of the steps specified in claim 21 and 22, and
iv) incorporating the product of step ii) or iii) into a food or feed product.

30. A method of feeding an animal comprising feeding said animal a feed product as defined in claim 1.

31. The method of claim 30, wherein the feed product is an aquatic or farm animal feed product.

32. The method according to claim 30, wherein the method comprises feeding a prey organism the feed product and subsequently feeding said prey organism to an animal.

33. The method according to claim 32, wherein the prey organism is zoo plankton and the animal is a marine fish or a shell fish.

34. A farm animal feed product comprising biomass derived from a liquid culture of a fungus of the class of Basidiomycetes, wherein said biomass comprises a survival enhancing agent and is obtainable by a method comprising the steps of:
i) fermenting a fungus of the class of Basidiomycetes in a liquid culture,
ii) isolating the biomass by removal of the extracellular material
iii) optionally isolating a composition comprising the survival enhancing agent.

35. An aquatic animal feed product comprising biomass derived from a liquid culture of a fungus of the class of Basidiomycetes, wherein said biomass comprises a survival enhancing agent and is obtainable by a method comprising the steps of:
i) fermenting a fungus of the class of Basidiomycetes in a liquid culture,
ii) isolating the biomass by removal of the extracellular material
iii) optionally isolating a composition comprising the survival enhancing agent.

36. (canceled)

37. A food or feed product comprising biomass derived from a liquid culture of a fungus of the class of Basidiomycetes, wherein said biomass comprises a survival enhancing agent and is obtainable by a method comprising the steps of:
i) fermenting a fungus of the class of Basidiomycetes in a liquid culture in the presence of malt extract,
ii) isolating the biomass by removal of the extracellular material
iii) optionally isolating a composition comprising the survival enhancing agent.

38. The feed product according to claim 34, wherein the survival enhancing agent comprises polysaccharides.

39. The feed product according to claim 34, wherein the survival enhancing agent comprises polypeptides.

40. The feed product according to claim 34, wherein at least part of the polysaccharides have a molecular weight of at least 30,000 Da.

41. The feed or food product according to claim 1, wherein said product is a functional food suitable for consumption by humans beings.

42. The functional food according to claim 41, wherein said functional food is a dairy product.

43. The functional food according to claim 41, wherein said functional food is a bread.

44. The functional food according to claim 41, wherein said functional food is a fruit juice-based product.
